SEQUENCE LISTING (789 sequences)

SEQ ID NO: 1 - protein, 1263 aa, Unknown
  Description of Unknown: MAD7 sequence
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 2 - protein, 1334 aa, Unknown
  Description of Unknown: MAD2 sequence
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 3 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 4 - protein, 1228 aa, Unknown
  Description of Unknown: Lachnospiraceae bacterium ND2006 sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 6 - protein, 1257 aa, Prevotella bryantii
MQINNLKIIYMKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 7 - protein, 1154 aa, Proteocatella sphenisci
MENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 8 - protein, 1235 aa, Acidaminococcus sp.
MVAFIDEFVGQYPVSKTLRFEARPVPETKKWLESDQCSVLFNDQKRNEYYGVLKELLDDYYRAYIEDALTSFTLDKALLENAYDLYCNRDTNAFSSCCEKLRKDLVKAFGNLKDYLLGSDQLKDLVKLKAKVDAPAGKGKKKIEVDSRLINWLNNNAKYSAEDREKYIKAIESFEGFVTYLTNYKQARENMFSSEDKSTAIAFRVIDQNMVTYFGNIRIYEKIKAKYPELYSALKGFEKFFSPTAYSEILSQSKIDEYNYQCIGRPIDDADFKGVNSLINEYRQKNGIKARELPVMSMLYKQILSDRDNSFMSEVINRNEEAIECAKNGYKVSYALFNELLQLYKKIFTEDNYGNIYVKTQPLTELSQALFGDWSILRNALDNGKYDKDIINLAELEKYFSEYCKVLDADDAAKIQDKFNLKDYFIQKNALDATLPDLDKITQYKPHLDAMLQAIRKYKLFSMYNGRKKMDVPENGIDFSNEFNAIYDKLSEFSILYDRIRNFATKKPYSDEKMKLSFNMPTMLAGWDYNNETANGCFLFIKDGKYFLGVADSKSKNIFDFKKNPHLLDKYSSKDIYYKVKYKQVSGSAKMLPKVVFAGSNEKIFGHLISKRILEIREKKLYTAAAGDRKAVAEWIDFMKSAIAIHPEWNEYFKFKFKNTAEYDNANKFYEDIDKQTYSLEKVEIPTEYIDEMVSQHKLYLFQLYTKDFSDKKKKKGTDNLHTMYWHGVFSDENLKAVTEGTQPIIKLNGEAEMFMRNPSIEFQVTHEHNKPIANKNPLNTKKESVFNYDLIKDKRYTERKFYFHCPITLNFRADKPIKYNEKINRFVENNPDVCIIGIDRGERHLLYYTVINQTGDILEQGSLNKISGSYTNDKGEKVNKETDYHDLLDRKEKGKHVAQQAWETIENIKELKAGYLSQVVYKLTQLMLQYNAVIVLENLNVGFKRGRTKVEKQVYQKFEKAMIDKLNYLVFKDRGYEMNGSYAKGLQLTDKFESFDKIGKQTGCIYYVIPSYTSHIDPKTGFVNLLNAKLRYENITKAQDTIRKFDSISYNAKADYFEFAFDYRSFGVDMARNEWVVCTCGDLRWEYSAKTRETKAYSVTDRLKELFKAHGIDYVGGENLVSHITEVADKHFLSTLLFYLRLVLKMRYTVSGTENENDFILSPVEYAPGKFFDSREATSTEPMNADANGAYHIALKGLMTIRGIEDGKLHNYGKGGENAAWFKFMQNQEYKNNG

SEQ ID NO: 9 - protein, 1264 aa, Moraxella caprae
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 10 - protein, 1230 aa, Unknown
  Description of Unknown: Lachnospiraceae bacterium ND2006 sequence
MHENNGKIADNFIGIYPVSKTLRFELKPVGKTQEYIEKHGILDEDLKRAGDYKSVKKIIDAYHKYFIDEALNGIQLDGLKNYYELYEKKRDNNEEKEFQKIQMSLRKQIVKRFSEHPQYKYLFKKELIKNVLPEFTKDNAEEQTLVKSFQEFTTYFEGFHQNRKNMYSDEEKSTAIAYRVVHQNLPKYIDNMRIFSMILNTDIRSDLTELFNNLKTKMDITIVEEYFAIDGFNKVVNQKGIDVYNTILGAFSTDDNTKIKGLNEYINLYNQKNKAKLPKLKPLFKQILSDRDKISFIPEQFDSDTEVLEAVDMFYNRLLQFVIENEGQITISKLLTNFSAYDLNKIYVKNDTTISAISNDLFDDWSYISKAVRENYDSENVDKNKRAAAYEEKKEKALSKIKMYSIEELNFFVKKYSCNECHIEGYFERRILEILDKMRYAYESCKILHDKGLINNISLCQDRQAISELKDFLDSIKEVQWLLKPLMIGQEQADKEEAFYTELLRIWEELEPITLLYNKVRNYVTKKPYTLEKVKLNFYKSTLLDGWDKNKEKDNLGIILLKDGQYYLGIMNRRNNKIADDAPLAKTDNVYRKMEYKLLTKVSANLPRIFLKDKYNPSEEMLEKYEKGTHLKGENFCIDDCRELIDFFKKGIKQYEDWGQFDFKFSDTESYDDISAFYKEVEHQGYKITFRDIDETYIDSLVNEGKLYLFQIYNKDFSPYSKGTKNLHTLYWEMLFSQQNLQNIVYKLNGNAEIFYRKASINQKDVVVHKADLPIKNKDPQNSKKESMFDYDIIKDKRFTCDKYQFHVPITMNFKALGENHFNRKVNRLIHDAENMHIIGIDRGERNLIYLCMIDMKGNIVKQISLNEIISYDKNKLEHKRNYHQLLKTREDENKSARQSWQTIHTIKELKEGYLSQVIHVITDLMVEYNAIVVLEDLNFGFKQGRQKFERQVYQKFEKMLIDKLNYLVDKSKGMDEDGGLLHAYQLTDEFKSFKQLGKQSGFLYYIPAWNTSKLDPTTGFVNLFYTKYESVEKSKEFINNFTSILYNQEREYFEFLFDYSAFTSKAEGSRLKWTVCSKGERVETYRNPKKNNEWDTQKIDLTFELKKLFNDYSISLLDGDLREQMGKIDKADFYKKFMKLFALIVQMRNSDEREDKLISPVLNKYGAFFETGKNERMPLDADANGAYNIARKGLWIIEKIKNTDVEQLDKVKLTISNKEWLQYAQEHIL

SEQ ID NO: 11 - protein, 1261 aa, Eubacterium coprostanoligenes
MDFFKNDMYFLCINGIIVISKLFAYLFLMYKRGVVMIKDNFVNVYSLSKTIRMALIPWGKTEDNFYKKFLLEEDEERAKNYIKVKGYMDEYHKNFIESALNSVVLNGVDEYCELYFKQNKSDSEVKKIESLEASMRKQISKAMKEYTVDGVKIYPLLSKKEFIRELLPEFLTQDEEIETLEQFNDFSTYFQGFWENRKNIYTDEEKSTGVPYRCINDNLPKFLDNVKSFEKVILALPQKAVDELNANFNGVYNVDVQDVFSVDYFNFVLSQSGIEKYNNIIGGYSNSDASKVQGLNEKINLYNQQIAKSDKSKKLPLLKPLYKQILSDRSSLSFIPEKFKDDNEVLNSINVLYDNIAESLEKANDLMSDIANYNTDNIFISSGVAVTDISKKVFGDWSLIRNNWNDEYESTHKKGKNEEKFYEKEDKEFKKIKSFSVSELQRLANSDLSIVDYLVDESASLYADIKTAYNNAKDLLSNEYSHSKRLSKNDDAIELIKSFLDSIKNYEAFLKPLCGTGKEESKDNAFYGAFLECFEEIRQVDAVYNKVRNHITQKPYSNDKIKLNFQNPQFLAGWDKNKERAYRSVLLRNGEKYYLAIMEKGKSKLFEDFPEDESSPFEKIDYKLLPEPSKMLPKVFFATSNKDLFNPSDEILNIRATGSFKKGDSFNLDDCHKFIDFYKASIENHPDWSKFDFDFSETNDYEDISKFFKEVSDQGYSIGYRKISESYLEEMVDNGSLYMFQLYNKDFSENRKSKGTPNLHTLYFKMLFDERNLEDVVYKLSGGAEMFYRKPSIDKNEMIVHPKNQPIDNKNPNNVKKTSTFEYDIVKDMRYTKPQFQLHLPIVLNFKANSKGYINDDVRNVLKNSEDTYVIGIDRGERNLVYACVVDGNGKLVEQVPLNVIEADNGYKTDYHKLLNDREEKRNEARKSWKTIGNIKELKEGYISQVVHKICQLVVKYDAVIAMEDLNSGFVNSRKKVEKQVYQKFERMLTQKLNYLVDKKLDPNEMGGLLNAYQLTNEATKVRNGRQDGIIFYIPAWLTSKIDPTTGFVNLLKPKYNSVSASKEFFSKFDEIRYNEKENYFEFSFNYDNFPKCNADFKREWTVCTYGDRIRTFRDPENNNKFNSEVVVLNDEFKNLFVEFDIDYTDNLKEQILAMDEKSFYKKLMGLLSLTLQMRNSISKNVDVDYLISPVKNSNGEFYDSRNYDITSSLPCDADSNGAYNIARKGLWAINQIKQADDETKANISIKNSEWLQYAQNCDEV

SEQ ID NO: 12 - protein, 1064 aa, Smithella sp.
MEKYKITKTIRFKLLPDKIQDISRQVAVLQNSTNAEKKNNLLRLVQRGQELPKLLNEYIRYSDNHKLKSNVTVHFRWLRLFTKDLFYNWKKDNTEKKIKISDVVYLSHVFEAFLKEWESTIERVNADCNKPEESKTRDAEIALSIRKLGIKHQLPFIKGFVDNSNDKNSEDTKSKLTALLSEFEAVLKICEQNYLPSQSSGIAIAKASFNYYTINKKQKDFEAEIVALKKQLHARYGNKKYDQLLRELNLIPLKELPLKELPLIEFYSEIKKRKSTKKSEFLEAVSNGLVFDDLKSKFPLFQTESNKYDEYLKLSNKITQKSTAKSLLSKDSPEAQKLQTEITKLKKNRGEYFKKAFGKYVQLCELYKEIAGKRGKLKGQIKGIENERIDSQRLQYWALVLEDNLKHSLILIPKEKTNELYRKVWGAKDDGASSSSSSTLYYFESMTYRALRKLCFGINGNTFLPEIQKELPQYNQKEFGEFCFHKSNDDKEIDEPKLISFYQSVLKTDFVKNTLALPQSVFNEVAIQSFETRQDFQIALEKCCYAKKQIISESLKKEILENYNTQIFKITSLDLQRSEQKNLKGHTRIWNRFWTKQNEEINYNLRLNPEIAIVWRKAKKTRIEKYGERSVLYEPEKRNRYLHEQYTLCTTVTDNALNNEITFAFEDTKKKGTEIVKYNEKINQTLKKEFNKNQLWFYGIDAGEIELATLALMNKDKEPQLFTVYELKKLDFFKHGYIYNKERELVIREKPYKAIQNLSYFLNEELYEKTFRDGKFNETYNELFKEKHVSAIDLTTAKVINGKIILNGDMITFLNLRILHAQRKIYEELIENPHAELKEKDYKLYFEIEGKDKDIYISRLDFEYIKPYQEISNYLFAYFASQQINEAREEEQINQTKRALAGNMIGVIYYLYQKYRGIISIEDLKQTKVESDRNKFEGNIERPLEWALYRKFQQEGYVPPISELIKLRELEKFPLKDVKQPKYENIQQFGIIKFVSPEETSTTCPKCLRRFKDYDKNKQEGFCKCQCGFDTRNDLKGFEGLNDPDKVAAFNIAKRGFEDLQKYK

SEQ ID NO: 13 - protein, 1232 aa, Sulfuricurvum sp.
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 14 - protein, 1057 aa, Unknown
  Description of Unknown: Microgenomates (Roizmanbacteria) bacterium sequence
MEIQELKNLYEVKKTVRFELKPSKKKIFEGGDVIKLQKDFEKVQKFFLDIFVYKNEHTKLEFKKKREIKYTWLRTNTKNEFYNWRGKSDTGKNYALNKIGFLAEEILRWLNEWQELTKSLKDLTQREEHKQERKSDIAFVLRNFLKRQNLPFIKDFFNAVIDIQGKQGKESDDKIRKFREEIKEIEKNLNACSREYLPTQSNGVLLYKASFSYYTLNKTPKEYEDLKKEKESELSSVLLKEIYRRKRFNRTTNQKDTLFECTSDWLVKIKLGKDIYEWTLDEAYQKMKIWKANQKSNFIEAVAGDKLTHQNFRKQFPLFDASDEDFETFYRLTKALDKNPENAKKIAQKRGKFFNAPNETVQTKNYHELCELYKRIAVKRGKIIAEIKGIENEEVQSQLLTHWAVIAEERDKKFIVLIPRKNGGKLENHKNAHAFLQEKDRKEPNDIKVYHFKSLTLRSLEKLCFKEAKNTFAPEIKKETNPKIWFPTYKQEWNSTPERLIKFYKQVLQSNYAQTYLDLVDFGNLNTFLETHFTTLEEFESDLEKTCYTKVPVYFAKKELETFADEFEAEVFEITTRSISTESKRKENAHAEIWRDFWSRENEEENHITRLNPEVSVLYRDEIKEKSNTSRKNRKSNANNRFSDPRFTLATTITLNADKKKSNLAFKTVEDINIHIDNFNKKFSKNFSGEWVYGIDRGLKELATLNVVKFSDVKNVFGVSQPKEFAKIPIYKLRDEKAILKDENGLSLKNAKGEARKVIDNISDVLEEGKEPDSTLFEKREVSSIDLTRAKLIKGHIISNGDQKTYLKLKETSAKRRIFELFSTAKIDKSSQFHVRKTIELSGTKIYWLCEWQRQDSWRTEKVSLRNTLKGYLQNLDLKNRFENIETIEKINHLRDAITANMVGILSHLQNKLEMQGVIALENLDTVREQSNKKMIDEHFEQSNEHVSRRLEWALYCKFANTGEVPPQIKESIFLRDEFKVCQIGILNFIDVKGTSSNCPNCDQESRKTGSHFICNFQNNCIFSSKENRNLLEQNLHNSDDVAAFNIAKRGLEIVKV

SEQ ID NO: 15 - RNA, 19 nt, Artificial Sequence
  Description of Artificial Sequence: Synthetic oligonucleotide
uaauuucuacucuuguaga

SEQ ID NO: 16 - RNA, 15 nt, Artificial Sequence
  Description of Artificial Sequence: Synthetic oligonucleotide
aucuacaacaguaga

SEQ ID NO: 17 - RNA, 15 nt, Artificial Sequence
  Description of Artificial Sequence: Synthetic oligonucleotide
aucuacaaaaguaga

SEQ ID NO: 18 - RNA, 20 nt, Artificial Sequence
  Description of Artificial Sequence: Synthetic oligonucleotide
ggaauuucuacucuuguaga

SEQ ID NO: 19 - RNA, 19 nt, Artificial Sequence
  Description of Artificial Sequence: Synthetic oligonucleotide
uaauucccacucuuguggg

SEQ ID NO: 20 - RNA, 15 nt, Artificial Sequence
  Description of Artificial Sequence: Synthetic oligonucleotide
aucuacaagaguaga

SEQ ID NO: 21 - RNA, 15 nt, Artificial Sequence
  Description of Artificial Sequence: Synthetic oligonucleotide
aucuacacuaguaga

SEQ ID NO: 22 - RNA, 20 nt, Artificial Sequence
  Description of Artificial Sequence: Synthetic oligonucleotide
uaauuucuacuaaguguaga

SEQ ID NO: 23 - RNA, 21 nt, Artificial Sequence
  Description of Artificial Sequence: Synthetic oligonucleotide
uaauuuucuacuuguuguaga

SEQ ID NO: 24 - RNA, 18 nt, Artificial Sequence
  Description of Artificial Sequence: Synthetic oligonucleotide
aauuucuacuguuguaga

SEQ ID NO: 25 - RNA, 19 nt, Artificial Sequence
  Description of Artificial Sequence: Synthetic oligonucleotide
gaauuucuacuguuguaga

SEQ ID NO: 26 - RNA, 10 nt, Artificial Sequence
  Description of Artificial Sequence: Synthetic oligonucleotide
uaauuucuac

SEQ ID NO: 27 - RNA, 6 nt, Artificial Sequence
  Description of Artificial Sequence: Synthetic oligonucleotide
aucuac

SEQ ID NO: 28 - RNA, 11 nt, Artificial Sequence
  Description of Artificial Sequence: Synthetic oligonucleotide
ggaauuucuac

SEQ ID NO: 29 - RNA, 10 nt, Artificial Sequence
  Description of Artificial Sequence: Synthetic oligonucleotide
uaauucccac

SEQ ID NO: 30 - RNA, 12 nt, Artificial Sequence
  Description of Artificial Sequence: Synthetic oligonucleotide
uaauuuucuacu

SEQ ID NO: 31 - RNA, 9 nt, Artificial Sequence
  Description of Artificial Sequence: Synthetic oligonucleotide
aauuucuac

SEQ ID NO: 32 - RNA, 10 nt, Artificial Sequence
  Description of Artificial Sequence: Synthetic oligonucleotide
gaauuucuac

SEQ ID NO: 33 - protein, 17 aa, Artificial Sequence
  Description of Artificial Sequence: Synthetic peptide
RKCLQAGMNLEARKTKK

SEQ ID NO: 34 - protein, 9 aa, Artificial Sequence
  Description of Artificial Sequence: Synthetic peptide
PAAKKKKLD

SEQ ID NO: 35 - protein, 7 aa, Artificial Sequence
  Description of Artificial Sequence: Synthetic peptide
PKKKRKV

SEQ ID NO: 36 - protein, 16 aa, Artificial Sequence
  Description of Artificial Sequence: Synthetic peptide
KRPAATKKAGQAKKKK

SEQ ID NO: 37 - protein, 9 aa, Artificial Sequence
  Description of Artificial Sequence: Synthetic peptide
PAAKRVKLD

SEQ ID NO: 38 - protein, 11 aa, Artificial Sequence
  Description of Artificial Sequence: Synthetic peptide
RQRRNELKRSP

SEQ ID NO: 39 - protein, 38 aa, Artificial Sequence
  Description of Artificial Sequence: Synthetic polypeptide
NQSSNFGPMKGGNFGGRSSGPYGGGGQYFAKPRNQGGY

SEQ ID NO: 40 - protein, 42 aa, Artificial Sequence
  Description of Artificial Sequence: Synthetic polypeptide
RMRIZFKNKGKDTAELRRRRVEVSVELRKAKKDEQILKRRNV

SEQ ID NO: 41 - protein, 8 aa, Artificial Sequence
  Description of Artificial Sequence: Synthetic peptide
VSRKRPRP

SEQ ID NO: 42 - protein, 8 aa, Artificial Sequence
  Description of Artificial Sequence: Synthetic peptide
PPKKARED

SEQ ID NO: 43 - protein, 8 aa, Artificial Sequence
  Description of Artificial Sequence: Synthetic peptide
PQPKKKPL

SEQ ID NO: 44 - protein, 12 aa, Artificial Sequence
  Description of Artificial Sequence: Synthetic peptide
SALIKKKKKMAP

SEQ ID NO: 45 - protein, 5 aa, Artificial Sequence
  Description of Artificial Sequence: Synthetic peptide
DRLRR

SEQ ID NO: 46 - protein, 7 aa, Artificial Sequence
  Description of Artificial Sequence: Synthetic peptide
PKQKKRK

SEQ ID NO: 47 - protein, 10 aa, Artificial Sequence
  Description of Artificial Sequence: Synthetic peptide
RKLKKKIKKL

SEQ ID NO: 48 - protein, 10 aa, Artificial Sequence
  Description of Artificial Sequence: Synthetic peptide
REKKKFLKRR

SEQ ID NO: 49 - protein, 20 aa, Artificial Sequence
  Description of Artificial Sequence: Synthetic peptide
KRKGDEVDGVDEVAKKKSKK

SEQ ID NO: 50 - RNA, 20 nt, Artificial Sequence
  Description of Artificial Sequence: Synthetic oligonucleotide
uaauuucuacucuuguagau

SEQ ID NO: 51 - DNA, 21 bp, Artificial Sequence
  Description of Artificial Sequence: Synthetic oligonucleotide
aggatgtggtccccatgaact

SEQ ID NO: 52 - DNA, 21 bp, Artificial Sequence
  Description of Artificial Sequence: Synthetic oligonucleotide
ctcacgtcatccagcagagaa

SEQ ID NO: 53 - DNA, 21 bp, Artificial Sequence
  Description of Artificial Sequence: Synthetic oligonucleotide
ctcttcctcctactcaccatc

SEQ ID NO: 54 - DNA, 21 bp, Artificial Sequence
  Description of Artificial Sequence: Synthetic oligonucleotide
taggggccccaactccatggt

SEQ ID NO: 55 - DNA, 21 bp, Artificial Sequence
  Description of Artificial Sequence: Synthetic oligonucleotide
agcggcacaaggctcagctga

SEQ ID NO: 56 - DNA, 21 bp, Artificial Sequence
  Description of Artificial Sequence: Synthetic oligonucleotide
cggaggctccttaccgatgtt

SEQ ID NO: 57 - DNA, 21 bp, Artificial Sequence
  Description of Artificial Sequence: Synthetic oligonucleotide
gtgttgctggtgagtgtgcat

SEQ ID NO: 58 - DNA, 21 bp, Artificial Sequence
  Description of Artificial Sequence: Synthetic oligonucleotide
cttgtaagtagtagcagcagc

SEQ ID NO: 59 - DNA, 21 bp, Artificial Sequence
  Description of Artificial Sequence: Synthetic oligonucleotide
gggtgcatacctgtctggctg

SEQ ID NO: 60 - DNA, 21 bp, Artificial Sequence
  Description of Artificial Sequence: Synthetic oligonucleotide
tctgcagggacaataggagcc

SEQ ID NO: 61 - DNA, 21 bp, Artificial Sequence
  Description of Artificial Sequence: Synthetic oligonucleotide
tatgacctgtatggaggggag

SEQ ID NO: 62 - DNA, 21 bp, Artificial Sequence
  Description of Artificial Sequence: Synthetic oligonucleotide
aggccttacctgaggcgaggg

SEQ ID NO: 63 - DNA, 21 bp, Artificial Sequence
  Description of Artificial Sequence: Synthetic oligonucleotide
tgagggtgaaggatagacgct

SEQ ID NO: 64 - DNA, 21 bp, Artificial Sequence
  Description of Artificial Sequence: Synthetic oligonucleotide
actttccattctctgctggat

SEQ ID NO: 65 - DNA, 21 bp, Artificial Sequence
  Description of Artificial Sequence: Synthetic oligonucleotide
tggcctggaggctatccagcg

SEQ ID NO: 66 - DNA, 21 bp, Artificial Sequence
  Description of Artificial Sequence: Synthetic oligonucleotide
tatctcttgtactacactgaa

SEQ ID NO: 67 - DNA, 21 bp, Artificial Sequence
  Description of Artificial Sequence: Synthetic oligonucleotide
cctggagatgcatactcacac

SEQ ID NO: 68 - DNA, 21 bp, Artificial Sequence
  Description of Artificial Sequence: Synthetic oligonucleotide
cagaagacagggatgaagaga

SEQ ID NO: 69 - DNA, 21 bp, Artificial Sequence
  Description of Artificial Sequence: Synthetic oligonucleotide
cactggaggtgcccgtgcaga

SEQ ID NO: 70 - DNA, 21 bp, Artificial Sequence
  Description of Artificial Sequence: Synthetic oligonucleotide
tgtgtgagtatgcatctccag

SEQ ID NO: 71 - DNA, 21 bp, Artificial Sequence
  Description of Artificial Sequence: Synthetic oligonucleotide
tcagccagctctgaggggacc

SEQ ID NO: 72 - DNA, 21 bp, Artificial Sequence
  Description of Artificial Sequence: Synthetic oligonucleotide
ctcacaacagctgcagggaag

SEQ ID NO: 73 - DNA, 21 bp, Artificial Sequence
  Description of Artificial Sequence: Synthetic oligonucleotide
agcttgccattcagagaggca

SEQ ID NO: 74 - DNA, 21 bp, Artificial Sequence
  Description of Artificial Sequence: Synthetic oligonucleotide
tacatacctgtcactatacac

SEQ ID NO: 75 - DNA, 21 bp, Artificial Sequence
  Description of Artificial Sequence: Synthetic oligonucleotide
tttttctagatgtgaacatgg

SEQ ID NO: 76 - DNA, 21 bp, Artificial Sequence
  Description of Artificial Sequence: Synthetic oligonucleotide
atgattccatgttcacatcta

SEQ ID NO: 77 - DNA, 21 bp, Artificial Sequence
  Description of Artificial Sequence: Synthetic oligonucleotide
gtgtaacatacctggaggaca

SEQ ID NO: 78 - DNA, 21 bp, Artificial Sequence
  Description of Artificial Sequence: Synthetic oligonucleotide
ggaggattgctcaacaaccat

SEQ ID NO: 79 - DNA, 21 bp, Artificial Sequence
  Description of Artificial Sequence: Synthetic oligonucleotide
taggaaacagtggcaataaat

SEQ ID NO: 80 - DNA, 21 bp, Artificial Sequence
  Description of Artificial Sequence: Synthetic oligonucleotide
caaggatgcttaccaccaggg

SEQ ID NO: 81 - DNA, 21 bp, Artificial Sequence
  Description of Artificial Sequence: Synthetic oligonucleotide
taagtagtagcagcagcagca

SEQ ID NO: 82 - DNA, 21 bp, Artificial Sequence
  Description of Artificial Sequence: Synthetic oligonucleotide
tatcagggaggctccccagtg

SEQ ID NO: 83 - DNA, 21 bp, Artificial Sequence
  Description of Artificial Sequence: Synthetic oligonucleotide
ccaccaggggacatggcccag

SEQ ID NO: 84 - DNA, 21 bp, Artificial Sequence
  Description of Artificial Sequence: Synthetic oligonucleotide
aatgtggcaacgtggtgctca

SEQ ID NO: 85 - DNA, 21 bp, Artificial Sequence
  Description of Artificial Sequence: Synthetic oligonucleotide
tgacattagccaaggtcaccc

SEQ ID NO: 86 - DNA, 21 bp, Artificial Sequence
  Description of Artificial Sequence: Synthetic oligonucleotide
cgcaaaggagatgtgtccctg

SEQ ID NO: 87 - DNA, 21 bp, Artificial Sequence
  Description of Artificial Sequence: Synthetic oligonucleotide
tcaggaccttggctggaggca

SEQ ID NO: 88 - DNA, 21 bp, Artificial Sequence
  Description of Artificial Sequence: Synthetic oligonucleotide
ggtcacctggatccctgggga

SEQ ID NO: 89 - DNA, 21 bp, Artificial Sequence
  Description of Artificial Sequence: Synthetic oligonucleotide
ccttccgctcacctccgcctg

SEQ ID NO: 90 - DNA, 21 bp, Artificial Sequence
  Description of Artificial Sequence: Synthetic oligonucleotide
gcacgaagctctccgatgtgt

SEQ ID NO: 91 - DNA, 21 bp, Artificial Sequence
  Description of Artificial Sequence: Synthetic oligonucleotide
ctagcggaatgggcacctcat

SEQ ID NO: 92 - DNA, 21 bp, Artificial Sequence
  Description of Artificial Sequence: Synthetic oligonucleotide
cagtggcgagagaagaccccg

SEQ ID NO: 93 - DNA, 21 bp, Artificial Sequence
  Description of Artificial Sequence: Synthetic oligonucleotide
actgccccccacccaggcctg

SEQ ID NO: 94 - DNA, 21 bp, Artificial Sequence
  Description of Artificial Sequence: Synthetic oligonucleotide
cgactctgacagagctggtgg

SEQ ID NO: 95 - DNA, 21 bp, Artificial Sequence
  Description of Artificial Sequence: Synthetic oligonucleotide
cagaagcaggaggtgaagaac

SEQ ID NO: 96 - DNA, 21 bp, Artificial Sequence
  Description of Artificial Sequence: Synthetic oligonucleotide
accgagacctcagtgggctgg

SEQ ID NO: 97 - DNA, 21 bp, Artificial Sequence
  Description of Artificial Sequence: Synthetic oligonucleotide
tgggccctactctgtgaccaa

SEQ ID NO: 98 - DNA, 21 bp, Artificial Sequence
  Description of Artificial Sequence: Synthetic oligonucleotide
tgtgctcagtgaccagcccaa

SEQ ID NO: 99 - DNA, 21 bp, Artificial Sequence
  Description of Artificial Sequence: Synthetic oligonucleotide
cccacccacatctcagagttt

SEQ ID NO: 100 - DNA, 21 bp, Artificial Sequence
  Description of Artificial Sequence: Synthetic oligonucleotide
ttgtgcgtgagagcctcagcc

SEQ ID NO: 101 - DNA, 21 bp, Artificial Sequence
  Description of Artificial Sequence: Synthetic oligonucleotide
aagaagacggggattgaggag

SEQ ID NO: 102 - DNA, 21 bp, Artificial Sequence
  Description of Artificial Sequence: Synthetic oligonucleotide
tcccctccatacaggtcatag

SEQ ID NO: 103 - DNA, 21 bp, Artificial Sequence
  Description of Artificial Sequence: Synthetic oligonucleotide
gctccccccagggtggacgct

SEQ ID NO: 104 - DNA, 21 bp, Artificial Sequence
  Description of Artificial Sequence: Synthetic oligonucleotide
ggctggtcactgagcacagaa

SEQ ID NO: 105 - DNA, 21 bp, Artificial Sequence
  Description of Artificial Sequence: Synthetic oligonucleotide
gtcctccctctagtggctgag

SEQ ID NO: 106 - DNA, 21 bp, Artificial Sequence
  Description of Artificial Sequence: Synthetic oligonucleotide
gcagacagggagaaataagga

SEQ ID NO: 107 - DNA, 21 bp, Artificial Sequence
  Description of Artificial Sequence: Synthetic oligonucleotide
caggtgaaattcctgagatgt

SEQ ID NO: 108 - DNA, 21 bp, Artificial Sequence
  Description of Artificial Sequence: Synthetic oligonucleotide
gacatcattgaccagagctct

SEQ ID NO: 109 - DNA, 21 bp, Artificial Sequence
  Description of Artificial Sequence: Synthetic oligonucleotide
ccagctcactaagtcagtctc

SEQ ID NO: 110 - DNA, 21 bp, Artificial Sequence
  Description of Artificial Sequence: Synthetic oligonucleotide
tatggagaagctctcatttct

SEQ ID NO: 111 - DNA, 21 bp, Artificial Sequence
  Description of Artificial Sequence: Synthetic oligonucleotide
taagatgctatttcccgtata

SEQ ID NO: 112 - DNA, 21 bp, Artificial Sequence
  Description of Artificial Sequence: Synthetic oligonucleotide
ccgtgtcattctctggactgc

SEQ ID NO: 113 - DNA, 21 bp, Artificial Sequence
  Description of Artificial Sequence: Synthetic oligonucleotide
attcctccacttcaacacctg

SEQ ID NO: 114 - DNA, 21 bp, Artificial Sequence
  Description of Artificial Sequence: Synthetic oligonucleotide
tacgggaaatagcatcttaga

SEQ ID NO: 115 - DNA, 21 bp, Artificial Sequence
  Description of Artificial Sequence: Synthetic oligonucleotide
gtggcaatggataaggccgag

SEQ ID NO: 116 - DNA, 21 bp, Artificial Sequence
  Description of Artificial Sequence: Synthetic oligonucleotide
cttgcttcaggaatggccagg

SEQ ID NO: 117 - DNA, 21 bp, Artificial Sequence
  Description of Artificial Sequence: Synthetic oligonucleotide
tagttcaaaacctctatcaat

SEQ ID NO: 118 - DNA, 21 bp, Artificial Sequence
  Description of Artificial Sequence: Synthetic oligonucleotide
tctgtgatatacacatcagaa

SEQ ID NO: 119 - DNA, 21 bp, Artificial Sequence
  Description of Artificial Sequence: Synthetic oligonucleotide
ggcagacagggagaaataagg

SEQ ID NO: 120 - DNA, 21 bp, Artificial Sequence
  Description of Artificial Sequence: Synthetic oligonucleotide
ctcgatataaggccttgagca

SEQ ID NO: 121 - DNA, 21 bp, Artificial Sequence
  Description of Artificial Sequence: Synthetic oligonucleotide
gagtctctcagctggtacacg

SEQ ID NO: 122 - DNA, 21 bp, Artificial Sequence
  Description of Artificial Sequence: Synthetic oligonucleotide
tggcagacagggagaaataag

SEQ ID NO: 123 - DNA, 21 bp, Artificial Sequence
  Description of Artificial Sequence: Synthetic oligonucleotide
ccagctgacagatgggctccc

SEQ ID NO: 124 - DNA, 21 bp, Artificial Sequence
  Description of Artificial Sequence: Synthetic oligonucleotide
ccgtataaagcatgagaccgt

SEQ ID NO: 125 - DNA, 21 bp, Artificial Sequence
  Description of Artificial Sequence: Synthetic oligonucleotide
ccccaacccaggctggagtcc

SEQ ID NO: 126 - DNA, 21 bp, Artificial Sequence
  Description of Artificial Sequence: Synthetic oligonucleotide
ttggcagacagggagaaataa

SEQ ID NO: 127 - DNA, 21 bp, Artificial Sequence
  Description of Artificial Sequence: Synthetic oligonucleotide
tcagaagagcctggctaggaa

SEQ ID NO: 128 - DNA, 21 bp, Artificial Sequence
  Description of Artificial Sequence: Synthetic oligonucleotide
ctctgccagagttatattgct

SEQ ID NO: 129 - DNA, 21 bp, Artificial Sequence
  Description of Artificial Sequence: Synthetic oligonucleotide
ccatgcctgcctttactctgc

SEQ ID NO: 130 - DNA, 21 bp, Artificial Sequence
  Description of Artificial Sequence: Synthetic oligonucleotide
gtctgtgatatacacatcaga

SEQ ID NO: 131 - DNA, 21 bp, Artificial Sequence
  Description of Artificial Sequence: Synthetic oligonucleotide
cggatcttcctggcggcgcga

SEQ ID NO: 132 - DNA, 21 bp, Artificial Sequence
  Description of Artificial Sequence: Synthetic oligonucleotide
aaggcagctggcaccagtgcc

SEQ ID NO: 133 - DNA, 21 bp, Artificial Sequence
  Description of Artificial Sequence: Synthetic oligonucleotide
tggtgtcactggcggcggccg

SEQ ID NO: 134 - DNA, 21 bp, Artificial Sequence
  Description of Artificial Sequence: Synthetic oligonucleotide
ttctgccccgactgcagccac

SEQ ID NO: 135 - DNA, 21 bp, Artificial Sequence
  Description of Artificial Sequence: Synthetic oligonucleotide
gtgaccggcacgagggctaag

SEQ ID NO: 136 - DNA, 21 bp, Artificial Sequence
  Description of Artificial Sequence: Synthetic oligonucleotide
ccatcggcctgactcccatgc

SEQ ID NO: 137 - DNA, 21 bp, Artificial Sequence
  Description of Artificial Sequence: Synthetic oligonucleotide
ccatcaccatcagcaccgggt

SEQ ID NO: 138 - DNA, 21 bp, Artificial Sequence
  Description of Artificial Sequence: Synthetic oligonucleotide
tcacagcccaagatagttaag

SEQ ID NO: 139 - DNA, 21 bp, Artificial Sequence
  Description of Artificial Sequence: Synthetic oligonucleotide
ctgaattgctatgtgtctggg

SEQ ID NO: 140 - DNA, 21 bp, Artificial Sequence
  Description of Artificial Sequence: Synthetic oligonucleotide
ctgaagaatggagagagaatt

SEQ ID NO: 141 - DNA, 21 bp, Artificial Sequence
  Description of Artificial Sequence: Synthetic oligonucleotide
tcagtgggggtgaattcagtg

SEQ ID NO: 142 - DNA, 21 bp, Artificial Sequence
  Description of Artificial Sequence: Synthetic oligonucleotide
cattctctgctggatgacgtg

SEQ ID NO: 143 - DNA, 21 bp, Artificial Sequence
  Description of Artificial Sequence: Synthetic oligonucleotide
atccatccgacattgaagttg

SEQ ID NO: 144 - DNA, 21 bp, Artificial Sequence
  Description of Artificial Sequence: Synthetic oligonucleotide
ccccacttaactatcttgggc

SEQ ID NO: 145 - DNA, 21 bp, Artificial Sequence
  Description of Artificial Sequence: Synthetic oligonucleotide
gctgtgctcgcgctactctct

SEQ ID NO: 146 - DNA, 21 bp, Artificial Sequence
  Description of Artificial Sequence: Synthetic oligonucleotide
gctggtgtcgttttgtcctga

SEQ ID NO: 147 - DNA, 21 bp, Artificial Sequence
  Description of Artificial Sequence: Synthetic oligonucleotide
tgctggcatctccatactctc

SEQ ID NO: 148 - DNA, 21 bp, Artificial Sequence
  Description of Artificial Sequence: Synthetic oligonucleotide
gtctcttgcagtgcctttctc

SEQ ID NO: 149 - DNA, 21 bp, Artificial Sequence
  Description of Artificial Sequence: Synthetic oligonucleotide
ctgttgcagatccagaaccgt

SEQ ID NO: 150 - DNA, 21 bp, Artificial Sequence
  Description of Artificial Sequence: Synthetic oligonucleotide
acagctaaagaaaagaagccc

SEQ ID NO: 151 - DNA, 21 bp, Artificial Sequence
  Description of Artificial Sequence: Synthetic oligonucleotide
tcaattgatgggaataaaata

SEQ ID NO: 152 - DNA, 21 bp, Artificial Sequence
  Description of Artificial Sequence: Synthetic oligonucleotide
ctcctctggatccttgcagca

SEQ ID NO: 153 - DNA, 21 bp, Artificial Sequence
  Description of Artificial Sequence: Synthetic oligonucleotide
cacatagacccctgttgtaag

SEQ ID NO: 154 - DNA, 21 bp, Artificial Sequence
  Description of Artificial Sequence: Synthetic oligonucleotide
ctagatgattccatctgcacg

SEQ ID NO: 155 - DNA, 21 bp, Artificial Sequence
  Description of Artificial Sequence: Synthetic oligonucleotide
ttcttctcttcatccctgtct

SEQ ID NO: 156 - DNA, 21 bp, Artificial Sequence
  Description of Artificial Sequence: Synthetic oligonucleotide
aggatattcacaaatgttgac

SEQ ID NO: 157 - DNA, 21 bp, Artificial Sequence
  Description of Artificial Sequence: Synthetic oligonucleotide
gaaggtaaaagaccatcgttc

SEQ ID NO: 158 - DNA, 21 bp, Artificial Sequence
  Description of Artificial Sequence: Synthetic oligonucleotide
tcatacatcatctgaagaaca

SEQ ID NO: 159 - DNA, 21 bp, Artificial Sequence
  Description of Artificial Sequence: Synthetic oligonucleotide
atctttcctcacaacagctgc

SEQ ID NO: 160 - DNA, 21 bp, Artificial Sequence
  Description of Artificial Sequence: Synthetic oligonucleotide
agtgaagagaaaggaagtaca

SEQ ID NO: 161 - DNA, 21 bp, Artificial Sequence
  Description of Artificial Sequence: Synthetic oligonucleotide
tttgttctttcagtgaagaga

SEQ ID NO: 162 - DNA, 21 bp, Artificial Sequence
  Description of Artificial Sequence: Synthetic oligonucleotide
ctaggcttagaagtggaaata

SEQ ID NO: 163 - DNA, 21 bp, Artificial Sequence
  Description of Artificial Sequence: Synthetic oligonucleotide
gaaggcctgcatcatgatggc

SEQ ID NO: 164 - DNA, 21 bp, Artificial Sequence
  Description of Artificial Sequence: Synthetic oligonucleotide
gtgcaagggtcacagtgttca

SEQ ID NO: 165 - DNA, 21 bp, Artificial Sequence
  Description of Artificial Sequence: Synthetic oligonucleotide
ggacgataatctagcaacaga

SEQ ID NO: 166 - DNA, 21 bp, Artificial Sequence
  Description of Artificial Sequence: Synthetic oligonucleotide
ttccttgggcaggtgaaagga

SEQ ID NO: 167 - DNA, 21 bp, Artificial Sequence
  Description of Artificial Sequence: Synthetic oligonucleotide
gtttacatctgcacttggtat

SEQ ID NO: 168 - DNA, 21 bp, Artificial Sequence
  Description of Artificial Sequence: Synthetic oligonucleotide
cttggtgcaagggtcacagtg

SEQ ID NO: 169 - DNA, 21 bp, Artificial Sequence
  Description of Artificial Sequence: Synthetic oligonucleotide
ctgttctgctgtgtcttggac

SEQ ID NO: 170 - DNA, 21 bp, Artificial Sequence
  Description of Artificial Sequence: Synthetic oligonucleotide
ctctttgcacttggtgttgct

SEQ ID NO: 171 - DNA, 21 bp, Artificial Sequence
  Description of Artificial Sequence: Synthetic oligonucleotide
tgttctgctgtgtcttggaca

SEQ ID NO: 172 - DNA, 21 bp, Artificial Sequence
  Description of Artificial Sequence: Synthetic oligonucleotide
acaggttcttacgtctgttgc

SEQ ID NO: 173 - DNA, 21 bp, Artificial Sequence
  Description of Artificial Sequence: Synthetic oligonucleotide
ggcaggtgaaaggaaagctag

SEQ ID NO: 174 - DNA, 21 bp, Artificial Sequence
  Description of Artificial Sequence: Synthetic oligonucleotide
ctagggtattctcatagcaaa

SEQ ID NO: 175 - DNA, 21 bp, Artificial Sequence
  Description of Artificial Sequence: Synthetic oligonucleotide
ccccagcagacgggcacgagg

SEQ ID NO: 176 - DNA, 21 bp, Artificial Sequence
  Description of Artificial Sequence: Synthetic oligonucleotide
gccaacctccctccctcagga

SEQ ID NO: 177 - DNA, 21 bp, Artificial Sequence
  Description of Artificial Sequence: Synthetic oligonucleotide
tgtttccatagcaaatatcca

SEQ ID NO: 178 - DNA, 21 bp, Artificial Sequence
  Description of Artificial Sequence: Synthetic oligonucleotide
gatccggcagcagtagatccc

SEQ ID NO: 179 - DNA, 21 bp, Artificial Sequence
  Description of Artificial Sequence: Synthetic oligonucleotide
ccaatcctgagggagggaggt

SEQ ID NO: 180 - DNA, 21 bp, Artificial Sequence
  Description of Artificial Sequence: Synthetic oligonucleotide
cgggactctggagcaaccatc

SEQ ID NO: 181 - DNA, 21 bp, Artificial Sequence
  Description of Artificial Sequence: Synthetic oligonucleotide
gccagtatctggatgtccaat

SEQ ID NO: 182 - DNA, 21 bp, Artificial Sequence
  Description of Artificial Sequence: Synthetic oligonucleotide
actgcagcctttccaaggatg

SEQ ID NO: 183 - DNA, 21 bp, Artificial Sequence
  Description of Artificial Sequence: Synthetic oligonucleotide
ccccttactagggtattctca

SEQ ID NO: 184 - DNA, 21 bp, Artificial Sequence
  Description of Artificial Sequence: Synthetic oligonucleotide
acctgaagttggtcatcaaac

SEQ ID NO: 185 - DNA, 21 bp, Artificial Sequence
  Description of Artificial Sequence: Synthetic oligonucleotide
ccaaggatgcttaccaccagg

SEQ ID NO: 186 - DNA, 21 bp, Artificial Sequence
  Description of Artificial Sequence: Synthetic oligonucleotide
gtttcccccttactagggtat

SEQ ID NO: 187 - DNA, 21 bp, Artificial Sequence
  Description of Artificial Sequence: Synthetic oligonucleotide
atcagtcctgagcaccacgtt

SEQ ID NO: 188 - DNA, 21 bp, Artificial Sequence
  Description of Artificial Sequence: Synthetic oligonucleotide
tgaggtgactccagtatctgg

SEQ ID NO: 189 - DNA, 21 bp, Artificial Sequence
  Description of Artificial Sequence: Synthetic oligonucleotide
ccagccttggcaatgccagct

SEQ ID NO: 190 - DNA, 21 bp, Artificial Sequence
  Description of Artificial Sequence: Synthetic oligonucleotide
tgtggagctctctggacaccc

SEQ ID NO: 191 - DNA, 21 bp, Artificial Sequence
  Description of Artificial Sequence: Synthetic oligonucleotide
gggcaggaagaggaagctttc

SEQ ID NO: 192 - DNA, 21 bp, Artificial Sequence
  Description of Artificial Sequence: Synthetic oligonucleotide
tccataggtgcccaacgctct

SEQ ID NO: 193 - DNA, 21 bp, Artificial Sequence
  Description of Artificial Sequence: Synthetic oligonucleotide
ccacctgaggctgacctgtga

SEQ ID NO: 194 - DNA, 21 bp, Artificial Sequence
  Description of Artificial Sequence: Synthetic oligonucleotide
cccagggatccaggtgaccca

SEQ ID NO: 195 - DNA, 21 bp, Artificial Sequence
  Description of Artificial Sequence: Synthetic oligonucleotide
acctggagccacccaaagcgg

SEQ ID NO: 196 - DNA, 21 bp, Artificial Sequence
  Description of Artificial Sequence: Synthetic oligonucleotide
cccttcgactagaggatgtga

SEQ ID NO: 197 - DNA, 21 bp, Artificial Sequence
  Description of Artificial Sequence: Synthetic oligonucleotide
cgctaagtggtgatgggggga

SEQ ID NO: 198 - DNA, 21 bp, Artificial Sequence
  Description of Artificial Sequence: Synthetic oligonucleotide
gcagtgaggaaagaccgggtc

SEQ ID NO: 199 - DNA, 21 bp, Artificial Sequence
  Description of Artificial Sequence: Synthetic oligonucleotide
cagagagaagggcagaagtgc

SEQ ID NO: 200 - DNA, 21 bp, Artificial Sequence
  Description of Artificial Sequence: Synthetic oligonucleotide
gaactggccggctggcctggg

SEQ ID NO: 201 - DNA, 21 bp, Artificial Sequence
  Description of Artificial Sequence: Synthetic oligonucleotide
gtgcccttccagagagaaggg

SEQ ID NO: 202 - DNA, 21 bp, Artificial Sequence
  Description of Artificial Sequence: Synthetic oligonucleotide
gagaccttcgacagcctcacg

SEQ ID NO: 203 - DNA, 21 bp, Artificial Sequence
  Description of Artificial Sequence: Synthetic oligonucleotide
ctggaccagatcaaccagcgg

SEQ ID NO: 204 - DNA, 21 bp, Artificial Sequence
  Description of Artificial Sequence: Synthetic oligonucleotide
cccccctgcacccggctgcag

SEQ ID NO: 205 - DNA, 21 bp, Artificial Sequence
  Description of Artificial Sequence: Synthetic oligonucleotide
caccagcgtctggaagggcag

SEQ ID NO: 206 - DNA, 21 bp, Artificial Sequence
  Description of Artificial Sequence: Synthetic oligonucleotide
ctgccgctggttgatctggtc

SEQ ID NO: 207 - DNA, 21 bp, Artificial Sequence
  Description of Artificial Sequence: Synthetic oligonucleotide
tggcagatggcgtggcaggag

SEQ ID NO: 208 - DNA, 21 bp, Artificial Sequence
  Description of Artificial Sequence: Synthetic oligonucleotide
ctggctcggcccagtcgcaag

SEQ ID NO: 209 - DNA, 21 bp, Artificial Sequence
  Description of Artificial Sequence: Synthetic oligonucleotide
aggtggatgatggtgccgtcg

SEQ ID NO: 210 - DNA, 21 bp, Artificial Sequence
  Description of Artificial Sequence: Synthetic oligonucleotide
gggagacctgattcgggagat

SEQ ID NO: 211 - DNA, 21 bp, Artificial Sequence
  Description of Artificial Sequence: Synthetic oligonucleotide
aatgaactgggcgatggccac

SEQ ID NO: 212 - DNA, 21 bp, Artificial Sequence
  Description of Artificial Sequence: Synthetic oligonucleotide
tctaggtggtaccatggccac

SEQ ID NO: 213 - DNA, 21 bp, Artificial Sequence
  Description of Artificial Sequence: Synthetic oligonucleotide
caggtctccccgctggacaat

SEQ ID NO: 214 - DNA, 21 bp, Artificial Sequence
  Description of Artificial Sequence: Synthetic oligonucleotide
gggtggcacatctccccatcc

SEQ ID NO: 215 - DNA, 21 bp, Artificial Sequence
  Description of Artificial Sequence: Synthetic oligonucleotide
tgcagagaaaggtggctctat

SEQ ID NO: 216 - DNA, 21 bp, Artificial Sequence
  Description of Artificial Sequence: Synthetic oligonucleotide
taatgctgacttggggtggca

SEQ ID NO: 217 - DNA, 21 bp, Artificial Sequence
  Description of Artificial Sequence: Synthetic oligonucleotide
ctcctgaggtcaccttccaca

SEQ ID NO: 218 - DNA, 21 bp, Artificial Sequence
  Description of Artificial Sequence: Synthetic oligonucleotide
ctaagaaacagtgagccttgt

SEQ ID NO: 219 - DNA, 21 bp, Artificial Sequence
  Description of Artificial Sequence: Synthetic oligonucleotide
cctctttgccccaacccaggc

SEQ ID NO: 220 - DNA, 21 bp, Artificial Sequence
  Description of Artificial Sequence: Synthetic oligonucleotide
aggtttccttgagtggcaggc

SEQ ID NO: 221 - DNA, 21 bp, Artificial Sequence
  Description of Artificial Sequence: Synthetic oligonucleotide
agaatcaaaatcggtgaatag

SEQ ID NO: 222 - DNA, 21 bp, Artificial Sequence
  Description of Artificial Sequence: Synthetic oligonucleotide
ccccttactgctcttctaggc

SEQ ID NO: 223 - DNA, 21 bp, Artificial Sequence
  Description of Artificial Sequence: Synthetic oligonucleotide
ggtggcaatggataaggccga

SEQ ID NO: 224 - DNA, 21 bp, Artificial Sequence
  Description of Artificial Sequence: Synthetic oligonucleotide
gaactataaatcagaacacct

SEQ ID NO: 225 - DNA, 21 bp, Artificial Sequence
  Description of Artificial Sequence: Synthetic oligonucleotide
tttctcagaagagcctggcta

SEQ ID NO: 226 - DNA, 21 bp, Artificial Sequence
  Description of Artificial Sequence: Synthetic oligonucleotide
cccgtgtcattctctggactg

SEQ ID NO: 227 - DNA, 21 bp, Artificial Sequence
  Description of Artificial Sequence: Synthetic oligonucleotide
ctgggcctttttcccatgcct

SEQ ID NO: 228 - DNA, 21 bp, Artificial Sequence
  Description of Artificial Sequence: Synthetic oligonucleotide
aactataaatcagaacacctg

SEQ ID NO: 229 - DNA, 21 bp, Artificial Sequence
  Description of Artificial Sequence: Synthetic oligonucleotide
attctcaaacaaatgtgtcac

SEQ ID NO: 230 - DNA, 21 bp, Artificial Sequence
  Description of Artificial Sequence: Synthetic oligonucleotide
cttgagtggcaggccaggcct

SEQ ID NO: 231 - DNA, 21 bp, Artificial Sequence
  Description of Artificial Sequence: Synthetic oligonucleotide
catgtgcaaacgccttcaaca

SEQ ID NO: 232 - DNA, 21 bp, Artificial Sequence
  Description of Artificial Sequence: Synthetic oligonucleotide
tactaagaaacagtgagcctt

SEQ ID NO: 233 - DNA, 21 bp, Artificial Sequence
  Description of Artificial Sequence: Synthetic oligonucleotide
ctcagactgtttgccccttac

SEQ ID NO: 234 - DNA, 21 bp, Artificial Sequence
  Description of Artificial Sequence: Synthetic oligonucleotide
taattcctccacttcaacacc

SEQ ID NO: 235 - DNA, 21 bp, Artificial Sequence
  Description of Artificial Sequence: Synthetic oligonucleotide
ataggatcttcttcaaaaccc

SEQ ID NO: 236 - DNA, 21 bp, Artificial Sequence
  Description of Artificial Sequence: Synthetic oligonucleotide
gaagaagatcctattaaataa

SEQ ID NO: 237 - DNA, 21 bp, Artificial Sequence
  Description of Artificial Sequence: Synthetic oligonucleotide
tgtttttaatgtgactctcat

SEQ ID NO: 238 - DNA, 21 bp, Artificial Sequence
  Description of Artificial Sequence: Synthetic oligonucleotide
gtactttacagtttattaaat

SEQ ID NO: 239 - DNA, 21 bp, Artificial Sequence
  Description of Artificial Sequence: Synthetic oligonucleotide
ataaactgtaaagtaccaaac

SEQ ID NO: 240 - DNA, 21 bp, Artificial Sequence
  Description of Artificial Sequence: Synthetic oligonucleotide
gacttttcccagctgacagat

SEQ ID NO: 241 - DNA, 21 bp, Artificial Sequence
  Description of Artificial Sequence: Synthetic oligonucleotide
cccagctgacagatgggctcc

SEQ ID NO: 242 - DNA, 21 bp, Artificial Sequence
  Description of Artificial Sequence: Synthetic oligonucleotide
gtggtgtcactggcggcggcc

SEQ ID NO: 243 - DNA, 21 bp, Artificial Sequence
  Description of Artificial Sequence: Synthetic oligonucleotide
gccatcaccatcagcaccggg

SEQ ID NO: 244 - DNA, 21 bp, Artificial Sequence
  Description of Artificial Sequence: Synthetic oligonucleotide
gtcctggtcctcacgcagagc

SEQ ID NO: 245 - DNA, 21 bp, Artificial Sequence
  Description of Artificial Sequence: Synthetic oligonucleotide
gccctcgtgccggtcaccaag

SEQ ID NO: 246 - DNA, 21 bp, Artificial Sequence
  Description of Artificial Sequence: Synthetic oligonucleotide
gctgaccgcagttgttccaac

SEQ ID NO: 247 - DNA, 21 bp, Artificial Sequence
  Description of Artificial Sequence: Synthetic oligonucleotide
ggctgaccgcagttgttccaa

SEQ ID NO: 248 - DNA, 21 bp, Artificial Sequence
  Description of Artificial Sequence: Synthetic oligonucleotide
gccctccccgcagccctggga

SEQ ID NO: 249 - DNA, 21 bp, Artificial Sequence
  Description of Artificial Sequence: Synthetic oligonucleotide
aacttctttgcctgtgtgctg

SEQ ID NO: 250 - DNA, 21 bp, Artificial Sequence
  Description of Artificial Sequence: Synthetic oligonucleotide
tttgcctgtgtgctggtgccc

SEQ ID NO: 251 - DNA, 21 bp, Artificial Sequence
  Description of Artificial Sequence: Synthetic oligonucleotide
cctgtgtgctggtgcccctgc

SEQ ID NO: 252 - DNA, 21 bp, Artificial Sequence
  Description of Artificial Sequence: Synthetic oligonucleotide
agctgtcgtcgcgccgccagg

SEQ ID NO: 253 - DNA, 21 bp, Artificial Sequence
  Description of Artificial Sequence: Synthetic oligonucleotide
tgcagtgtggaccgtgcccgc

SEQ ID NO: 254 - DNA, 21 bp, Artificial Sequence
  Description of Artificial Sequence: Synthetic oligonucleotide
gcagcatggacctccttctgc

SEQ ID NO: 255 - DNA, 21 bp, Artificial Sequence
  Description of Artificial Sequence: Synthetic oligonucleotide
ccctctgctggctgcccctac

SEQ ID NO: 256 - DNA, 21 bp, Artificial Sequence
  Description of Artificial Sequence: Synthetic oligonucleotide
actttcttctgccccgactgc

SEQ ID NO: 257 - DNA, 21 bp, Artificial Sequence
  Description of Artificial Sequence: Synthetic oligonucleotide
cttctgccccgactgcagcca

SEQ ID NO: 258 - DNA, 21 bp, Artificial Sequence
  Description of Artificial Sequence: Synthetic oligonucleotide
atctacgcctaccgtatccgc

SEQ ID NO: 259 - DNA, 21 bp, Artificial Sequence
  Description of Artificial Sequence: Synthetic oligonucleotide
cgcaagatcattcgcagccac

SEQ ID NO: 260 - DNA, 21 bp, Artificial Sequence
  Description of Artificial Sequence: Synthetic oligonucleotide
aaaggttcttgctgcctcagg

SEQ ID NO: 261 - DNA, 21 bp, Artificial Sequence
  Description of Artificial Sequence: Synthetic oligonucleotide
caaggcagctggcaccagtgc

SEQ ID NO: 262 - DNA, 21 bp, Artificial Sequence
  Description of Artificial Sequence: Synthetic oligonucleotide
agctcatggctaaggagctcc

SEQ ID NO: 263 - DNA, 21 bp, Artificial Sequence
  Description of Artificial Sequence: Synthetic oligonucleotide
gccatgagctcaagggagtgt

SEQ ID NO: 264 - DNA, 21 bp, Artificial Sequence
  Description of Artificial Sequence: Synthetic oligonucleotide
cccgatattcctcaggtactc

SEQ ID NO: 265 - DNA, 21 bp, Artificial Sequence
  Description of Artificial Sequence: Synthetic oligonucleotide
ccattctctgctggatgacgt

SEQ ID NO: 266 - DNA, 21 bp, Artificial Sequence
  Description of Artificial Sequence: Synthetic oligonucleotide
aatgtcggatggatgaaaccc

SEQ ID NO: 267 - DNA, 21 bp, Artificial Sequence
  Description of Artificial Sequence: Synthetic oligonucleotide
tcaattctctctccattcttc

SEQ ID NO: 268 - DNA, 21 bp, Artificial Sequence
  Description of Artificial Sequence: Synthetic oligonucleotide
ttcaattctctctccattctt

SEQ ID NO: 269 - DNA, 21 bp, Artificial Sequence
  Description of Artificial Sequence: Synthetic oligonucleotide
ctgaaagacaagtctgaatgc

SEQ ID NO: 270 - DNA, 21 bp, Artificial Sequence
  Description of Artificial Sequence: Synthetic oligonucleotide
tctttcagcaaggactggtct

SEQ ID NO: 271 - DNA, 21 bp, Artificial Sequence
  Description of Artificial Sequence: Synthetic oligonucleotide
agcaaggactggtctttctat

SEQ ID NO: 272 - DNA, 21 bp, Artificial Sequence
  Description of Artificial Sequence: Synthetic oligonucleotide
actatcttgggctgtgacaaa

SEQ ID NO: 273 - DNA, 21 bp, Artificial Sequence
  Description of Artificial Sequence: Synthetic oligonucleotide
gtcacagcccaagatagttaa

SEQ ID NO: 274 - DNA, 21 bp, Artificial Sequence
  Description of Artificial Sequence: Synthetic oligonucleotide
tcctcctacagatacaaactg

SEQ ID NO: 275 - DNA, 21 bp, Artificial Sequence
  Description of Artificial Sequence: Synthetic oligonucleotide
gtcctgagagtccagtttgta

SEQ ID NO: 276 - DNA, 21 bp, Artificial Sequence
  Description of Artificial Sequence: Synthetic oligonucleotide
tgttgctggatgctgaggggc

SEQ ID NO: 277 - DNA, 21 bp, Artificial Sequence
  Description of Artificial Sequence: Synthetic oligonucleotide
ccttttcttcgtggccaatgc

SEQ ID NO: 278 - DNA, 21 bp, Artificial Sequence
  Description of Artificial Sequence: Synthetic oligonucleotide
tcttcgtggccaatgccataa

SEQ ID NO: 279 - DNA, 21 bp, Artificial Sequence
  Description of Artificial Sequence: Synthetic oligonucleotide
cttcgtggccaatgccataat

SEQ ID NO: 280 - DNA, 21 bp, Artificial Sequence
  Description of Artificial Sequence: Synthetic oligonucleotide
gggctctgacaggtaggaccc

SEQ ID NO: 281 - DNA, 21 bp, Artificial Sequence
  Description of Artificial Sequence: Synthetic oligonucleotide
taccttggggctctgacaggt

SEQ ID NO: 282 - DNA, 21 bp, Artificial Sequence
  Description of Artificial Sequence: Synthetic oligonucleotide
ttaccttggggctctgacagg

SEQ ID NO: 283 - skipped

SEQ ID NO: 284 - DNA, 21 bp, Artificial Sequence
  Description of Artificial Sequence: Synthetic oligonucleotide
ttaacagcgatgctgaccccc

SEQ ID NO: 285 - DNA, 21 bp, Artificial Sequence
  Description of Artificial Sequence: Synthetic oligonucleotide
tatgaccagatggacctggct

SEQ ID NO: 286 - DNA, 21 bp, Artificial Sequence
  Description of Artificial Sequence: Synthetic oligonucleotide
tcctcccagaacccgacacag

SEQ ID NO: 287 - DNA, 21 bp, Artificial Sequence
  Description of Artificial Sequence: Synthetic oligonucleotide
cctcccagaacccgacacaga

SEQ ID NO: 288 - DNA, 21 bp, Artificial Sequence
  Description of Artificial Sequence: Synthetic oligonucleotide
catgtcacacaacagcctgct

SEQ ID NO: 289 - DNA, 21 bp, Artificial Sequence
  Description of Artificial Sequence: Synthetic oligonucleotide
ctcaccgatattggcataagc

SEQ ID NO: 290 - DNA, 21 bp, Artificial Sequence
  Description of Artificial Sequence: Synthetic oligonucleotide
tccttgtctgggcagcggaac

SEQ ID NO: 291 - DNA, 21 bp, Artificial Sequence
  Description of Artificial Sequence: Synthetic oligonucleotide
ccttgtctgggcagcggaact

SEQ ID NO: 292 - DNA, 21 bp, Artificial Sequence
  Description of Artificial Sequence: Synthetic oligonucleotide
tctgggcagcggaactggacc

SEQ ID NO: 293 - DNA, 21 bp, Artificial Sequence
  Description of Artificial Sequence: Synthetic oligonucleotide
ctcaggccctccagctgggag

SEQ ID NO: 294 - DNA, 21 bp, Artificial Sequence
  Description of Artificial Sequence: Synthetic oligonucleotide
ctgaaaatgtccttgctcagg

SEQ ID NO: 295 - DNA, 21 bp, Artificial Sequence
  Description of Artificial Sequence: Synthetic oligonucleotide
tctcaaagtagagcacatagg

SEQ ID NO: 296 - DNA, 21 bp, Artificial Sequence
  Description of Artificial Sequence: Synthetic oligonucleotide
atctggtcctatgtgctctac

SEQ ID NO: 297 - DNA, 21 bp, Artificial Sequence
  Description of Artificial Sequence: Synthetic oligonucleotide
ctgcccaacttctgctggcat

SEQ ID NO: 298 - DNA, 21 bp, Artificial Sequence
  Description of Artificial Sequence: Synthetic oligonucleotide
tctgcccaacttctgctggca

SEQ ID NO: 299 - DNA, 21 bp, Artificial Sequence
  Description of Artificial Sequence: Synthetic oligonucleotide
ctgacttttctgcccaacttc

SEQ ID NO: 300 - DNA, 21 bp, Artificial Sequence
  Description of Artificial Sequence: Synthetic oligonucleotide
ctctgcagccttcccagagga

SEQ ID NO: 301 - DNA, 21 bp, Artificial Sequence
  Description of Artificial Sequence: Synthetic oligonucleotide
ccagaggagcttccggcagac

SEQ ID NO: 302 - DNA, 21 bp, Artificial Sequence
  Description of Artificial Sequence: Synthetic oligonucleotide
aggtctgccggaagctcctct

SEQ ID NO: 303 - DNA, 21 bp, Artificial Sequence
  Description of Artificial Sequence: Synthetic oligonucleotide
cagtgcttcaggtctgccgga

SEQ ID NO: 304 - DNA, 21 bp, Artificial Sequence
  Description of Artificial Sequence: Synthetic oligonucleotide
cggcagacctgaagcactgga

SEQ ID NO: 305 - DNA, 21 bp, Artificial Sequence
  Description of Artificial Sequence: Synthetic oligonucleotide
ctcacagctgagccccccact

SEQ ID NO: 306 - DNA, 21 bp, Artificial Sequence
  Description of Artificial Sequence: Synthetic oligonucleotide
ctccaggcgcatctggccgga

SEQ ID NO: 307 - DNA, 21 bp, Artificial Sequence
  Description of Artificial Sequence: Synthetic oligonucleotide
tctcttgcagtgcctttctcc

SEQ ID NO: 308 - DNA, 21 bp, Artificial Sequence
  Description of Artificial Sequence: Synthetic oligonucleotide
ctccagttcctcgttgagctg

SEQ ID NO: 309 - DNA, 21 bp, Artificial Sequence
  Description of Artificial Sequence: Synthetic oligonucleotide
tgccgctgaaatccaaggcaa

SEQ ID NO: 310 - DNA, 21 bp, Artificial Sequence
  Description of Artificial Sequence: Synthetic oligonucleotide
ccttggatttcagcggcacaa

SEQ ID NO: 311 - DNA, 21 bp, Artificial Sequence
  Description of Artificial Sequence: Synthetic oligonucleotide
gatttcagcggcacaaggctc

SEQ ID NO: 312 - DNA, 21 bp, Artificial Sequence
  Description of Artificial Sequence: Synthetic oligonucleotide
gcagaagacagggatgaagag

SEQ ID NO: 313 - DNA, 21 bp, Artificial Sequence
  Description of Artificial Sequence: Synthetic oligonucleotide
ggcttttccatgctagcaatg

SEQ ID NO: 314 - DNA, 21 bp, Artificial Sequence
  Description of Artificial Sequence: Synthetic oligonucleotide
gcttttccatgctagcaatgc

SEQ ID NO: 315 - DNA, 21 bp, Artificial Sequence
  Description of Artificial Sequence: Synthetic oligonucleotide
tccatgctagcaatgcacgtg

SEQ ID NO: 316 - DNA, 21 bp, Artificial Sequence
  Description of Artificial Sequence: Synthetic oligonucleotide
ccatgctagcaatgcacgtgg

SEQ ID NO: 317 - DNA, 21 bp, Artificial Sequence
  Description of Artificial Sequence: Synthetic oligonucleotide
gtgtgtgagtatgcatctcca

SEQ ID NO: 318 - DNA, 21 bp, Artificial Sequence
  Description of Artificial Sequence: Synthetic oligonucleotide
gcctggagatgcatactcaca

SEQ ID NO: 319 - DNA, 21 bp, Artificial Sequence
  Description of Artificial Sequence: Synthetic oligonucleotide
ggcaggctgacagccaggtga

SEQ ID NO: 320 - DNA, 21 bp, Artificial Sequence
  Description of Artificial Sequence: Synthetic oligonucleotide
agtcacctggctgtcagcctg

SEQ ID NO: 321 - DNA, 21 bp, Artificial Sequence
  Description of Artificial Sequence: Synthetic oligonucleotide
atttccactggaggtgcccgt

SEQ ID NO: 322 - DNA, 21 bp, Artificial Sequence
  Description of Artificial Sequence: Synthetic oligonucleotide
gatagtgaggttcacttgatt

SEQ ID NO: 323 - DNA, 21 bp, Artificial Sequence
  Description of Artificial Sequence: Synthetic oligonucleotide
cagatgtagagtcccgtgtcc

SEQ ID NO: 324 - DNA, 21 bp, Artificial Sequence
  Description of Artificial Sequence: Synthetic oligonucleotide
ctcaccaattacataaatctg

SEQ ID NO: 325 - DNA, 21 bp, Artificial Sequence
  Description of Artificial Sequence: Synthetic oligonucleotide
gctcaccaattacataaatct

SEQ ID NO: 326 - DNA, 21 bp, Artificial Sequence
  Description of Artificial Sequence: Synthetic oligonucleotide
gttttctgttgcagatccaga

SEQ ID NO: 327 - DNA, 21 bp, Artificial Sequence
  Description of Artificial Sequence: Synthetic oligonucleotide
ttttctgttgcagatccagaa

SEQ ID NO: 328 - DNA, 21 bp, Artificial Sequence
  Description of Artificial Sequence: Synthetic oligonucleotide
cagcagttagttcggggttgt

SEQ ID NO: 329 - DNA, 21 bp, Artificial Sequence
  Description of Artificial Sequence: Synthetic oligonucleotide
tttatagctttctcctcacag

SEQ ID NO: 330 - DNA, 21 bp, Artificial Sequence
  Description of Artificial Sequence: Synthetic oligonucleotide
ctcctcacagctgtttctttg

SEQ ID NO: 331 - DNA, 21 bp, Artificial Sequence
  Description of Artificial Sequence: Synthetic oligonucleotide
tcctcacagctgtttctttga

SEQ ID NO: 332 - DNA, 21 bp, Artificial Sequence
  Description of Artificial Sequence: Synthetic oligonucleotide
gctcaaagaaacagctgtgag

SEQ ID NO: 333 - DNA, 21 bp, Artificial Sequence
  Description of Artificial Sequence: Synthetic oligonucleotide
tttttgtgtttgacagctaaa

SEQ ID NO: 334 - DNA, 21 bp, Artificial Sequence
  Description of Artificial Sequence: Synthetic oligonucleotide
tgtgtttgacagctaaagaaa

SEQ ID NO: 335 - DNA, 21 bp, Artificial Sequence
  Description of Artificial Sequence: Synthetic oligonucleotide
cacattctggctctgttgggg

SEQ ID NO: 336 - DNA, 21 bp, Artificial Sequence
  Description of Artificial Sequence: Synthetic oligonucleotide
tcacattctggctctgttggg

SEQ ID NO: 337 - DNA, 21 bp, Artificial Sequence
  Description of Artificial Sequence: Synthetic oligonucleotide
agccttattttattcccatca

SEQ ID NO: 338 - DNA, 21 bp, Artificial Sequence
  Description of Artificial Sequence: Synthetic oligonucleotide
tcttgggcggggtggccattc

SEQ ID NO: 339 - DNA, 21 bp, Artificial Sequence
  Description of Artificial Sequence: Synthetic oligonucleotide
cttgatgcgggtcccctcaga

SEQ ID NO: 340 - DNA, 21 bp, Artificial Sequence
  Description of Artificial Sequence: Synthetic oligonucleotide
gatggagattttcttgatgcg

SEQ ID NO: 341 - DNA, 21 bp, Artificial Sequence
  Description of Artificial Sequence: Synthetic oligonucleotide
ccgatgttcccttcgatggag

SEQ ID NO: 342 - DNA, 21 bp, Artificial Sequence
  Description of Artificial Sequence: Synthetic oligonucleotide
tgaatatccttaaacaattgt

SEQ ID NO: 343 - DNA, 21 bp, Artificial Sequence
  Description of Artificial Sequence: Synthetic oligonucleotide
ccaatcttcacacaattgttt

SEQ ID NO: 344 - DNA, 21 bp, Artificial Sequence
  Description of Artificial Sequence: Synthetic oligonucleotide
aacaattgtgtgaagattggg

SEQ ID NO: 345 - DNA, 21 bp, Artificial Sequence
  Description of Artificial Sequence: Synthetic oligonucleotide
aacattgcaccatctggcaac

SEQ ID NO: 346 - DNA, 21 bp, Artificial Sequence
  Description of Artificial Sequence: Synthetic oligonucleotide
gaacattgcaccatctggcaa

SEQ ID NO: 347 - DNA, 21 bp, Artificial Sequence
  Description of Artificial Sequence: Synthetic oligonucleotide
catacctcaaattcatcttga

SEQ ID NO: 348 - DNA, 21 bp, Artificial Sequence
  Description of Artificial Sequence: Synthetic oligonucleotide
attttcatacctcaaattcat

SEQ ID NO: 349 - DNA, 21 bp, Artificial Sequence
  Description of Artificial Sequence: Synthetic oligonucleotide
aattttattttcatacctcaa

SEQ ID NO: 350 - DNA, 21 bp, Artificial Sequence
  Description of Artificial Sequence: Synthetic oligonucleotide
tgcacattcaaaataggaact

SEQ ID NO: 351 - DNA, 21 bp, Artificial Sequence
  Description of Artificial Sequence: Synthetic oligonucleotide
tctgagacattgtaagttcct

SEQ ID NO: 352 - DNA, 21 bp, Artificial Sequence
  Description of Artificial Sequence: Synthetic oligonucleotide
caatgtctcagaaaaatggtg

SEQ ID NO: 353 - DNA, 21 bp, Artificial Sequence
  Description of Artificial Sequence: Synthetic oligonucleotide
accttccaaacatatgcctgt

SEQ ID NO: 354 - DNA, 21 bp, Artificial Sequence
  Description of Artificial Sequence: Synthetic oligonucleotide
caaacatatgcctgtctcagt

SEQ ID NO: 355 - DNA, 21 bp, Artificial Sequence
  Description of Artificial Sequence: Synthetic oligonucleotide
ccattcagagaggcaagctga

SEQ ID NO: 356 - DNA, 21 bp, Artificial Sequence
  Description of Artificial Sequence: Synthetic oligonucleotide
cctctctgaatggcaagctca

SEQ ID NO: 357 - DNA, 21 bp, Artificial Sequence
  Description of Artificial Sequence: Synthetic oligonucleotide
tctgcatctttgagcttgcca

SEQ ID NO: 358 - DNA, 21 bp, Artificial Sequence
  Description of Artificial Sequence: Synthetic oligonucleotide
ttgaacgatctgtgtatagtg

SEQ ID NO: 359 - DNA, 21 bp, Artificial Sequence
  Description of Artificial Sequence: Synthetic oligonucleotide
aggtatatttttgcatctaat

SEQ ID NO: 360 - DNA, 21 bp, Artificial Sequence
  Description of Artificial Sequence: Synthetic oligonucleotide
tattttacaggttcttacgtc

SEQ ID NO: 361 - DNA, 21 bp, Artificial Sequence
  Description of Artificial Sequence: Synthetic oligonucleotide
attttacaggttcttacgtct

SEQ ID NO: 362 - DNA, 21 bp, Artificial Sequence
  Description of Artificial Sequence: Synthetic oligonucleotide
tggacgataatctagcaacag

SEQ ID NO: 363 - DNA, 21 bp, Artificial Sequence
  Description of Artificial Sequence: Synthetic oligonucleotide
ggcattaacacttttggacga

SEQ ID NO: 364 - DNA, 21 bp, Artificial Sequence
  Description of Artificial Sequence: Synthetic oligonucleotide
gagttgatgtcagtcacttgg

SEQ ID NO: 365 - DNA, 21 bp, Artificial Sequence
  Description of Artificial Sequence: Synthetic oligonucleotide
caagttctgagtctcaactgt

SEQ ID NO: 366 - DNA, 21 bp, Artificial Sequence
  Description of Artificial Sequence: Synthetic oligonucleotide
tggcagaattggccatcatga

SEQ ID NO: 367 - DNA, 21 bp, Artificial Sequence
  Description of Artificial Sequence: Synthetic oligonucleotide
tttccttgggcaggtgaaagg

SEQ ID NO: 368 - DNA, 21 bp, Artificial Sequence
  Description of Artificial Sequence: Synthetic oligonucleotide
ttggcagggcacgcagtctgg

SEQ ID NO: 369 - DNA, 21 bp, Artificial Sequence
  Description of Artificial Sequence: Synthetic oligonucleotide
ccttcttggcagggcacgcag

SEQ ID NO: 370 - DNA, 21 bp, Artificial Sequence
  Description of Artificial Sequence: Synthetic oligonucleotide
tctgtgtactccttcccttct

SEQ ID NO: 371 - DNA, 21 bp, Artificial Sequence
  Description of Artificial Sequence: Synthetic oligonucleotide
gtctgtgtactccttcccttc

SEQ ID NO: 372 - DNA, 21 bp, Artificial Sequence
  Description of Artificial Sequence: Synthetic oligonucleotide
gaagaaaaatgggctttgtct

SEQ ID NO: 373 - DNA, 21 bp, Artificial Sequence
  Description of Artificial Sequence: Synthetic oligonucleotide
tcttccaaatgcagaagatgt

SEQ ID NO: 374 - DNA, 21 bp, Artificial Sequence
  Description of Artificial Sequence: Synthetic oligonucleotide
atcacacaatctacatcttct

SEQ ID NO: 375 - DNA, 21 bp, Artificial Sequence
  Description of Artificial Sequence: Synthetic oligonucleotide
aagactcttaccatgtccttc

SEQ ID NO: 376 - DNA, 21 bp, Artificial Sequence
  Description of Artificial Sequence: Synthetic oligonucleotide
caaactgattttctaggctta

SEQ ID NO: 377 - DNA, 21 bp, Artificial Sequence
  Description of Artificial Sequence: Synthetic oligonucleotide
gaagtggaaataaactgcacc

SEQ ID NO: 378 - DNA, 21 bp, Artificial Sequence
  Description of Artificial Sequence: Synthetic oligonucleotide
gtattctgggtccgggtgcag

SEQ ID NO: 379 - DNA, 21 bp, Artificial Sequence
  Description of Artificial Sequence: Synthetic oligonucleotide
catctgcacttggtattctgg

SEQ ID NO: 380 - DNA, 21 bp, Artificial Sequence
  Description of Artificial Sequence: Synthetic oligonucleotide
ttttgtaactctactgtatgt

SEQ ID NO: 381 - DNA, 21 bp, Artificial Sequence
  Description of Artificial Sequence: Synthetic oligonucleotide
tttgtaactctactgtatgtg

SEQ ID NO: 382 - DNA, 21 bp, Artificial Sequence
  Description of Artificial Sequence: Synthetic oligonucleotide
cacttggtgttgctggtgagt

SEQ ID NO: 383 - DNA, 21 bp, Artificial Sequence
  Description of Artificial Sequence: Synthetic oligonucleotide
gggtggctttgtcttcttctt

SEQ ID NO: 384 - DNA, 21 bp, Artificial Sequence
  Description of Artificial Sequence: Synthetic oligonucleotide
gtcttcttcttttgccaattc

SEQ ID NO: 385 - DNA, 21 bp, Artificial Sequence
  Description of Artificial Sequence: Synthetic oligonucleotide
tcttcttcttttgccaattcc

SEQ ID NO: 386 - DNA, 21 bp, Artificial Sequence
  Description of Artificial Sequence: Synthetic oligonucleotide
gccaattccactaattgtttg

SEQ ID NO: 387 - DNA, 21 bp, Artificial Sequence
  Description of Artificial Sequence: Synthetic oligonucleotide
ccccaaacaattagtggaatt

SEQ ID NO: 388 - DNA, 21 bp, Artificial Sequence
  Description of Artificial Sequence: Synthetic oligonucleotide
aacaaagcaagaacttacccc

SEQ ID NO: 389 - DNA, 21 bp, Artificial Sequence
  Description of Artificial Sequence: Synthetic oligonucleotide
ttctttcagtgaagagaaagg

SEQ ID NO: 390 - DNA, 21 bp, Artificial Sequence
  Description of Artificial Sequence: Synthetic oligonucleotide
ctgtacttcctttctcttcac

SEQ ID NO: 391 - DNA, 21 bp, Artificial Sequence
  Description of Artificial Sequence: Synthetic oligonucleotide
tgcatgttttctgtacttcct

SEQ ID NO: 392 - DNA, 21 bp, Artificial Sequence
  Description of Artificial Sequence: Synthetic oligonucleotide
ctgcatgttttctgtacttcc

SEQ ID NO: 393 - DNA, 21 bp, Artificial Sequence
  Description of Artificial Sequence: Synthetic oligonucleotide
tgtgctttctgcatgttttct

SEQ ID NO: 394 - DNA, 21 bp, Artificial Sequence
  Description of Artificial Sequence: Synthetic oligonucleotide
ctgtgctttctgcatgttttc

SEQ ID NO: 395 - DNA, 21 bp, Artificial Sequence
  Description of Artificial Sequence: Synthetic oligonucleotide
cctttctgtgctttctgcatg

SEQ ID NO: 396 - DNA, 21 bp, Artificial Sequence
  Description of Artificial Sequence: Synthetic oligonucleotide
gttttcctttctgtgctttct

SEQ ID NO: 397 - DNA, 21 bp, Artificial Sequence
  Description of Artificial Sequence: Synthetic oligonucleotide
aagttggagattcatgagaac

SEQ ID NO: 398 - DNA, 21 bp, Artificial Sequence
  Description of Artificial Sequence: Synthetic oligonucleotide
aatacctacaggatttaaagt

SEQ ID NO: 399 - DNA, 21 bp, Artificial Sequence
  Description of Artificial Sequence: Synthetic oligonucleotide
ttgctttctaggaaacagtgg

SEQ ID NO: 400 - DNA, 21 bp, Artificial Sequence
  Description of Artificial Sequence: Synthetic oligonucleotide
ctaggaaacagtggcaataaa

SEQ ID NO: 401 - DNA, 21 bp, Artificial Sequence
  Description of Artificial Sequence: Synthetic oligonucleotide
ccagataaatttattgccact

SEQ ID NO: 402 - DNA, 21 bp, Artificial Sequence
  Description of Artificial Sequence: Synthetic oligonucleotide
ctatttttcagatgttgactt

SEQ ID NO: 403 - DNA, 21 bp, Artificial Sequence
  Description of Artificial Sequence: Synthetic oligonucleotide
tcagatgttgacttgagtaaa

SEQ ID NO: 404 - DNA, 21 bp, Artificial Sequence
  Description of Artificial Sequence: Synthetic oligonucleotide
agtaaatatatcaccactatt

SEQ ID NO: 405 - DNA, 21 bp, Artificial Sequence
  Description of Artificial Sequence: Synthetic oligonucleotide
aacttgacttagtgtcatgac

SEQ ID NO: 406 - DNA, 21 bp, Artificial Sequence
  Description of Artificial Sequence: Synthetic oligonucleotide
gaacaaagcctttaacttgac

SEQ ID NO: 407 - DNA, 21 bp, Artificial Sequence
  Description of Artificial Sequence: Synthetic oligonucleotide
gttcgaaagaatggtgtcaat

SEQ ID NO: 408 - DNA, 21 bp, Artificial Sequence
  Description of Artificial Sequence: Synthetic oligonucleotide
attgacaccattctttcgaac

SEQ ID NO: 409 - DNA, 21 bp, Artificial Sequence
  Description of Artificial Sequence: Synthetic oligonucleotide
ttcgaaagaatggtgtcaatg

SEQ ID NO: 410 - DNA, 21 bp, Artificial Sequence
  Description of Artificial Sequence: Synthetic oligonucleotide
ggcttcattgacaccattctt

SEQ ID NO: 411 - DNA, 21 bp, Artificial Sequence
  Description of Artificial Sequence: Synthetic oligonucleotide
gtaattggcatcaacttcatg

SEQ ID NO: 412 - DNA, 21 bp, Artificial Sequence
  Description of Artificial Sequence: Synthetic oligonucleotide
catgaagttgatgccaattac

SEQ ID NO: 413 - DNA, 21 bp, Artificial Sequence
  Description of Artificial Sequence: Synthetic oligonucleotide
tttccatgaagttgatgccaa

SEQ ID NO: 414 - DNA, 21 bp, Artificial Sequence
  Description of Artificial Sequence: Synthetic oligonucleotide
tttctttccatgaagttgatg

SEQ ID NO: 415 - DNA, 21 bp, Artificial Sequence
  Description of Artificial Sequence: Synthetic oligonucleotide
atggaaagaaagaagcgtatg

SEQ ID NO: 416 - DNA, 21 bp, Artificial Sequence
  Description of Artificial Sequence: Synthetic oligonucleotide
atcaatgtgtcatacgcttct

SEQ ID NO: 417 - DNA, 21 bp, Artificial Sequence
  Description of Artificial Sequence: Synthetic oligonucleotide
ttgagatctttaatcaatgtg

SEQ ID NO: 418 - DNA, 21 bp, Artificial Sequence
  Description of Artificial Sequence: Synthetic oligonucleotide
tttgagatctttaatcaatgt

SEQ ID NO: 419 - DNA, 21 bp, Artificial Sequence
  Description of Artificial Sequence: Synthetic oligonucleotide
ctctgcaagagtacaaagatt

SEQ ID NO: 420 - DNA, 21 bp, Artificial Sequence
  Description of Artificial Sequence: Synthetic oligonucleotide
tactcttgcagagaaaattca

SEQ ID NO: 421 - DNA, 21 bp, Artificial Sequence
  Description of Artificial Sequence: Synthetic oligonucleotide
aggatgatagtctgaattttc

SEQ ID NO: 422 - DNA, 21 bp, Artificial Sequence
  Description of Artificial Sequence: Synthetic oligonucleotide
ctgagtcactagtaatgtcct

SEQ ID NO: 423 - DNA, 21 bp, Artificial Sequence
  Description of Artificial Sequence: Synthetic oligonucleotide
aattttctgagtcactagtaa

SEQ ID NO: 424 - DNA, 21 bp, Artificial Sequence
  Description of Artificial Sequence: Synthetic oligonucleotide
tgaagtttgaattttctgagt

SEQ ID NO: 425 - DNA, 21 bp, Artificial Sequence
  Description of Artificial Sequence: Synthetic oligonucleotide
atttctgaagtttgaattttc

SEQ ID NO: 426 - DNA, 21 bp, Artificial Sequence
  Description of Artificial Sequence: Synthetic oligonucleotide
gatttcatttctgaagtttga

SEQ ID NO: 427 - DNA, 21 bp, Artificial Sequence
  Description of Artificial Sequence: Synthetic oligonucleotide
ggatttcatttctgaagtttg

SEQ ID NO: 428 - DNA, 21 bp, Artificial Sequence
  Description of Artificial Sequence: Synthetic oligonucleotide
agaaatgaaatccaaagcttg

SEQ ID NO: 429 - DNA, 21 bp, Artificial Sequence
  Description of Artificial Sequence: Synthetic oligonucleotide
tcactctagaccaagctttgg

SEQ ID NO: 430 - DNA, 21 bp, Artificial Sequence
  Description of Artificial Sequence: Synthetic oligonucleotide
ttgtttttcactctagaccaa

SEQ ID NO: 431 - DNA, 21 bp, Artificial Sequence
  Description of Artificial Sequence: Synthetic oligonucleotide
gtctagagtgaaaaacaacaa

SEQ ID NO: 432 - DNA, 21 bp, Artificial Sequence
  Description of Artificial Sequence: Synthetic oligonucleotide
tcttctgcaagctccatgttt

SEQ ID NO: 433 - DNA, 21 bp, Artificial Sequence
  Description of Artificial Sequence: Synthetic oligonucleotide
tcttctgcaagctccatgttt

SEQ ID NO: 434 - DNA, 21 bp, Artificial Sequence
  Description of Artificial Sequence: Synthetic oligonucleotide
cttctgcaagctccatgtttt

SEQ ID NO: 435 - DNA, 21 bp, Artificial Sequence
  Description of Artificial Sequence: Synthetic oligonucleotide
cacatcttccctttgactgtg

SEQ ID NO: 436 - DNA, 21 bp, Artificial Sequence
  Description of Artificial Sequence: Synthetic oligonucleotide
gactgtgtcctgctgctgctg

SEQ ID NO: 437 - DNA, 21 bp, Artificial Sequence
  Description of Artificial Sequence: Synthetic oligonucleotide
tctctctatgcagggtcctca

SEQ ID NO: 438 - DNA, 21 bp, Artificial Sequence
  Description of Artificial Sequence: Synthetic oligonucleotide
tacaccccagccgccccaggg

SEQ ID NO: 439 - DNA, 21 bp, Artificial Sequence
  Description of Artificial Sequence: Synthetic oligonucleotide
gccccagcagacgggcacgag

SEQ ID NO: 440 - DNA, 21 bp, Artificial Sequence
  Description of Artificial Sequence: Synthetic oligonucleotide
catcagtcctgagcaccacgt

SEQ ID NO: 441 - DNA, 21 bp, Artificial Sequence
  Description of Artificial Sequence: Synthetic oligonucleotide
cggaaatccccatttagccag

SEQ ID NO: 442 - DNA, 21 bp, Artificial Sequence
  Description of Artificial Sequence: Synthetic oligonucleotide
gcggaaatccccatttagcca

SEQ ID NO: 443 - DNA, 21 bp, Artificial Sequence
  Description of Artificial Sequence: Synthetic oligonucleotide
tcatcattcattatgcctggg

SEQ ID NO: 444 - DNA, 21 bp, Artificial Sequence
  Description of Artificial Sequence: Synthetic oligonucleotide
aggttaaatttttcatcattc

SEQ ID NO: 445 - DNA, 21 bp, Artificial Sequence
  Description of Artificial Sequence: Synthetic oligonucleotide
atgaccaacttcaggttaaat

SEQ ID NO: 446 - DNA, 21 bp, Artificial Sequence
  Description of Artificial Sequence: Synthetic oligonucleotide
tgttgtttctgacattagcca

SEQ ID NO: 447 - DNA, 21 bp, Artificial Sequence
  Description of Artificial Sequence: Synthetic oligonucleotide
gaaaggctgcagtgaagtctc

SEQ ID NO: 448 - DNA, 21 bp, Artificial Sequence
  Description of Artificial Sequence: Synthetic oligonucleotide
tatagcagagacacagacact

SEQ ID NO: 449 - DNA, 21 bp, Artificial Sequence
  Description of Artificial Sequence: Synthetic oligonucleotide
ctgttagatttatatcaggga

SEQ ID NO: 450 - DNA, 21 bp, Artificial Sequence
  Description of Artificial Sequence: Synthetic oligonucleotide
catagcaaatatccacattgg

SEQ ID NO: 451 - DNA, 21 bp, Artificial Sequence
  Description of Artificial Sequence: Synthetic oligonucleotide
aaaattaaagcgccgaagata

SEQ ID NO: 452 - DNA, 21 bp, Artificial Sequence
  Description of Artificial Sequence: Synthetic oligonucleotide
catttgaaaattaaagcgccg

SEQ ID NO: 453 - DNA, 21 bp, Artificial Sequence
  Description of Artificial Sequence: Synthetic oligonucleotide
tgtttcccccttactagggta

SEQ ID NO: 454 - DNA, 21 bp, Artificial Sequence
  Description of Artificial Sequence: Synthetic oligonucleotide
aattctgtatcttctctttgc

SEQ ID NO: 455 - DNA, 21 bp, Artificial Sequence
  Description of Artificial Sequence: Synthetic oligonucleotide
atttccacagcctcatctctt

SEQ ID NO: 456 - DNA, 21 bp, Artificial Sequence
  Description of Artificial Sequence: Synthetic oligonucleotide
tttccacagcctcatctcttt

SEQ ID NO: 457 - DNA, 21 bp, Artificial Sequence
  Description of Artificial Sequence: Synthetic oligonucleotide
cacagcctcatctctttggcc

SEQ ID NO: 458 - DNA, 21 bp, Artificial Sequence
  Description of Artificial Sequence: Synthetic oligonucleotide
cttctgagcgaattccctctg

SEQ ID NO: 459 - DNA, 21 bp, Artificial Sequence
  Description of Artificial Sequence: Synthetic oligonucleotide
atatacgttctcttcaatggt

SEQ ID NO: 460 - DNA, 21 bp, Artificial Sequence
  Description of Artificial Sequence: Synthetic oligonucleotide
gggttgtcgctttgcaatgcc

SEQ ID NO: 461 - DNA, 21 bp, Artificial Sequence
  Description of Artificial Sequence: Synthetic oligonucleotide
ctgtttctgcagccgctttgg

SEQ ID NO: 462 - DNA, 21 bp, Artificial Sequence
  Description of Artificial Sequence: Synthetic oligonucleotide
tgcagccgctttgggtggctc

SEQ ID NO: 463 - DNA, 21 bp, Artificial Sequence
  Description of Artificial Sequence: Synthetic oligonucleotide
gctcacctagtgaagcctctc

SEQ ID NO: 464 - DNA, 21 bp, Artificial Sequence
  Description of Artificial Sequence: Synthetic oligonucleotide
tgcgaagagcaggggtcactt

SEQ ID NO: 465 - DNA, 21 bp, Artificial Sequence
  Description of Artificial Sequence: Synthetic oligonucleotide
ccgcccagtggcccgcccgct

SEQ ID NO: 466 - DNA, 21 bp, Artificial Sequence
  Description of Artificial Sequence: Synthetic oligonucleotide
tcgctatggctgcgcccagcc

SEQ ID NO: 467 - DNA, 21 bp, Artificial Sequence
  Description of Artificial Sequence: Synthetic oligonucleotide
tccttgcacagtgactgccag

SEQ ID NO: 468 - DNA, 21 bp, Artificial Sequence
  Description of Artificial Sequence: Synthetic oligonucleotide
cacagtgactgccagcccccc

SEQ ID NO: 469 - DNA, 21 bp, Artificial Sequence
  Description of Artificial Sequence: Synthetic oligonucleotide
gaactgctccttcagccgccc

SEQ ID NO: 470 - DNA, 21 bp, Artificial Sequence
  Description of Artificial Sequence: Synthetic oligonucleotide
agccgccctgaccgcccagcc

SEQ ID NO: 471 - DNA, 21 bp, Artificial Sequence
  Description of Artificial Sequence: Synthetic oligonucleotide
ccgctaagtggtgatgggggg

SEQ ID NO: 472 - DNA, 21 bp, Artificial Sequence
  Description of Artificial Sequence: Synthetic oligonucleotide
gcggaaagcttcctcttcctg

SEQ ID NO: 473 - DNA, 21 bp, Artificial Sequence
  Description of Artificial Sequence: Synthetic oligonucleotide
ctcttcctgccccaagtcagc

SEQ ID NO: 474 - DNA, 21 bp, Artificial Sequence
  Description of Artificial Sequence: Synthetic oligonucleotide
aacgtctccatcatgtataac

SEQ ID NO: 475 - DNA, 21 bp, Artificial Sequence
  Description of Artificial Sequence: Synthetic oligonucleotide
cttttctcttcaggtctggag

SEQ ID NO: 476 - DNA, 21 bp, Artificial Sequence
  Description of Artificial Sequence: Synthetic oligonucleotide
ctcttcaggtctggagccccc

SEQ ID NO: 477 - DNA, 21 bp, Artificial Sequence
  Description of Artificial Sequence: Synthetic oligonucleotide
acagtgtacgctggagcaggt

SEQ ID NO: 478 - DNA, 21 bp, Artificial Sequence
  Description of Artificial Sequence: Synthetic oligonucleotide
ctcactgccaagtggactcct

SEQ ID NO: 479 - DNA, 21 bp, Artificial Sequence
  Description of Artificial Sequence: Synthetic oligonucleotide
acccttcgactagaggatgtg

SEQ ID NO: 480 - DNA, 21 bp, Artificial Sequence
  Description of Artificial Sequence: Synthetic oligonucleotide
gactagaggatgtgagccagg

SEQ ID NO: 481 - DNA, 21 bp, Artificial Sequence
  Description of Artificial Sequence: Synthetic oligonucleotide
cccacctgaggctgacctgtg

SEQ ID NO: 482 - DNA, 21 bp, Artificial Sequence
  Description of Artificial Sequence: Synthetic oligonucleotide
tactcttttcagtgactccca

SEQ ID NO: 483 - DNA, 21 bp, Artificial Sequence
  Description of Artificial Sequence: Synthetic oligonucleotide
cagtgactcccaaatcctttg

SEQ ID NO: 484 - DNA, 21 bp, Artificial Sequence
  Description of Artificial Sequence: Synthetic oligonucleotide
gggtcacctggatccctgggg

SEQ ID NO: 485 - DNA, 21 bp, Artificial Sequence
  Description of Artificial Sequence: Synthetic oligonucleotide
gtgaggtgactccagtatctg

SEQ ID NO: 486 - DNA, 21 bp, Artificial Sequence
  Description of Artificial Sequence: Synthetic oligonucleotide
gtgtggagctctctggacacc

SEQ ID NO: 487 - DNA, 21 bp, Artificial Sequence
  Description of Artificial Sequence: Synthetic oligonucleotide
gctggaggcacaggaggccca

SEQ ID NO: 488 - DNA, 21 bp, Artificial Sequence
  Description of Artificial Sequence: Synthetic oligonucleotide
cccagccttggcaatgccagc

SEQ ID NO: 489 - DNA, 21 bp, Artificial Sequence
  Description of Artificial Sequence: Synthetic oligonucleotide
gcaatgccagctgtaccaggg

SEQ ID NO: 490 - DNA, 21 bp, Artificial Sequence
  Description of Artificial Sequence: Synthetic oligonucleotide
ttggagcagcagtgtacttca

SEQ ID NO: 491 - DNA, 21 bp, Artificial Sequence
  Description of Artificial Sequence: Synthetic oligonucleotide
acagagctgtctagcccaggt

SEQ ID NO: 492 - DNA, 21 bp, Artificial Sequence
  Description of Artificial Sequence: Synthetic oligonucleotide
ctccataggtgcccaacgctc

SEQ ID NO: 493 - DNA, 21 bp, Artificial Sequence
  Description of Artificial Sequence: Synthetic oligonucleotide
tcatccttggtgtcctttctc

SEQ ID NO: 494 - DNA, 21 bp, Artificial Sequence
  Description of Artificial Sequence: Synthetic oligonucleotide
gtgtcctttctctgctccttt

SEQ ID NO: 495 - DNA, 21 bp, Artificial Sequence
  Description of Artificial Sequence: Synthetic oligonucleotide
ctctgctccttttggtgactg

SEQ ID NO: 496 - DNA, 21 bp, Artificial Sequence
  Description of Artificial Sequence: Synthetic oligonucleotide
tctgctccttttggtgactgg

SEQ ID NO: 497 - DNA, 21 bp, Artificial Sequence
  Description of Artificial Sequence: Synthetic oligonucleotide
tggtgactggagcctttggct

SEQ ID NO: 498 - DNA, 21 bp, Artificial Sequence
  Description of Artificial Sequence: Synthetic oligonucleotide
ggtgactggagcctttggctt

SEQ ID NO: 499 - DNA, 21 bp, Artificial Sequence
  Description of Artificial Sequence: Synthetic oligonucleotide
ggctttcacctttggagaaga

SEQ ID NO: 500 - DNA, 21 bp, Artificial Sequence
  Description of Artificial Sequence: Synthetic oligonucleotide
gctttcacctttggagaagac

SEQ ID NO: 501 - DNA, 21 bp, Artificial Sequence
  Description of Artificial Sequence: Synthetic oligonucleotide
ctctaaggcagaaaatcgtct

SEQ ID NO: 502 - DNA, 21 bp, Artificial Sequence
  Description of Artificial Sequence: Synthetic oligonucleotide
ctgccttagagcaagggattc

SEQ ID NO: 503 - DNA, 21 bp, Artificial Sequence
  Description of Artificial Sequence: Synthetic oligonucleotide
gagcaagggattcaccctccg

SEQ ID NO: 504 - DNA, 21 bp, Artificial Sequence
  Description of Artificial Sequence: Synthetic oligonucleotide
aacctgacctgggacagtttc

SEQ ID NO: 505 - DNA, 21 bp, Artificial Sequence
  Description of Artificial Sequence: Synthetic oligonucleotide
cgctcacctccgcctgagcag

SEQ ID NO: 506 - DNA, 21 bp, Artificial Sequence
  Description of Artificial Sequence: Synthetic oligonucleotide
tccactgctcaggcggaggtg

SEQ ID NO: 507 - DNA, 21 bp, Artificial Sequence
  Description of Artificial Sequence: Synthetic oligonucleotide
tccccagccctgctcgtggtg

SEQ ID NO: 508 - DNA, 21 bp, Artificial Sequence
  Description of Artificial Sequence: Synthetic oligonucleotide
ggtcaccacgagcagggctgg

SEQ ID NO: 509 - DNA, 21 bp, Artificial Sequence
  Description of Artificial Sequence: Synthetic oligonucleotide
acctgcagcttctccaacaca

SEQ ID NO: 510 - DNA, 21 bp, Artificial Sequence
  Description of Artificial Sequence: Synthetic oligonucleotide
tccaacacatcggagagcttc

SEQ ID NO: 511 - DNA, 21 bp, Artificial Sequence
  Description of Artificial Sequence: Synthetic oligonucleotide
gtgctaaactggtaccgcatg

SEQ ID NO: 512 - DNA, 21 bp, Artificial Sequence
  Description of Artificial Sequence: Synthetic oligonucleotide
tccgtctggttgctggggctc

SEQ ID NO: 513 - DNA, 21 bp, Artificial Sequence
  Description of Artificial Sequence: Synthetic oligonucleotide
cccgaggaccgcagccagccc

SEQ ID NO: 514 - DNA, 21 bp, Artificial Sequence
  Description of Artificial Sequence: Synthetic oligonucleotide
cgtgtcacacaactgcccaac

SEQ ID NO: 515 - DNA, 21 bp, Artificial Sequence
  Description of Artificial Sequence: Synthetic oligonucleotide
cacatgagcgtggtcagggcc

SEQ ID NO: 516 - DNA, 21 bp, Artificial Sequence
  Description of Artificial Sequence: Synthetic oligonucleotide
gatctgcgccttgggggccag

SEQ ID NO: 517 - DNA, 21 bp, Artificial Sequence
  Description of Artificial Sequence: Synthetic oligonucleotide
atctgcgccttgggggccagg

SEQ ID NO: 518 - DNA, 21 bp, Artificial Sequence
  Description of Artificial Sequence: Synthetic oligonucleotide
ggggccagggagatggcccca

SEQ ID NO: 519 - DNA, 21 bp, Artificial Sequence
  Description of Artificial Sequence: Synthetic oligonucleotide
tgcccttccagagagaagggc

SEQ ID NO: 520 - DNA, 21 bp, Artificial Sequence
  Description of Artificial Sequence: Synthetic oligonucleotide
tgcccttctctctggaagggc

SEQ ID NO: 521 - DNA, 21 bp, Artificial Sequence
  Description of Artificial Sequence: Synthetic oligonucleotide
ctcctcaaagaaggaggaccc

SEQ ID NO: 522 - DNA, 21 bp, Artificial Sequence
  Description of Artificial Sequence: Synthetic oligonucleotide
tcctcaaagaaggaggacccc

SEQ ID NO: 523 - DNA, 21 bp, Artificial Sequence
  Description of Artificial Sequence: Synthetic oligonucleotide
tctcgccactggaaatccagc

SEQ ID NO: 524 - DNA, 21 bp, Artificial Sequence
  Description of Artificial Sequence: Synthetic oligonucleotide
cctagcggaatgggcacctca

SEQ ID NO: 525 - DNA, 21 bp, Artificial Sequence
  Description of Artificial Sequence: Synthetic oligonucleotide
gcccctctgaccggcttcctt

SEQ ID NO: 526 - DNA, 21 bp, Artificial Sequence
  Description of Artificial Sequence: Synthetic oligonucleotide
agcagggtctctgcatccagc

SEQ ID NO: 527 - DNA, 21 bp, Artificial Sequence
  Description of Artificial Sequence: Synthetic oligonucleotide
cctgacgctgccttctctagg

SEQ ID NO: 528 - DNA, 21 bp, Artificial Sequence
  Description of Artificial Sequence: Synthetic oligonucleotide
gcctgcagcagcgtctctgcc

SEQ ID NO: 529 - DNA, 21 bp, Artificial Sequence
  Description of Artificial Sequence: Synthetic oligonucleotide
gtgctttctgtgctcagtgac

SEQ ID NO: 530 - DNA, 21 bp, Artificial Sequence
  Description of Artificial Sequence: Synthetic oligonucleotide
ctgtgctcagtgaccagccca

SEQ ID NO: 531 - DNA, 21 bp, Artificial Sequence
  Description of Artificial Sequence: Synthetic oligonucleotide
atgtgggtgaccctgagcggg

SEQ ID NO: 532 - DNA, 21 bp, Artificial Sequence
  Description of Artificial Sequence: Synthetic oligonucleotide
cctcgcacatgaccttgatgt

SEQ ID NO: 533 - DNA, 21 bp, Artificial Sequence
  Description of Artificial Sequence: Synthetic oligonucleotide
gacagcctcacggacctggtg

SEQ ID NO: 534 - DNA, 21 bp, Artificial Sequence
  Description of Artificial Sequence: Synthetic oligonucleotide
ttgttcagttccaacactcgg

SEQ ID NO: 535 - DNA, 21 bp, Artificial Sequence
  Description of Artificial Sequence: Synthetic oligonucleotide
gctgtatcctcggactcctgc

SEQ ID NO: 536 - DNA, 21 bp, Artificial Sequence
  Description of Artificial Sequence: Synthetic oligonucleotide
cagacgctggtgcaagttctt

SEQ ID NO: 537 - DNA, 21 bp, Artificial Sequence
  Description of Artificial Sequence: Synthetic oligonucleotide
ttctctggccgctgcccttcc

SEQ ID NO: 538 - DNA, 21 bp, Artificial Sequence
  Description of Artificial Sequence: Synthetic oligonucleotide
atgtagttggcattgatgtag

SEQ ID NO: 539 - DNA, 21 bp, Artificial Sequence
  Description of Artificial Sequence: Synthetic oligonucleotide
cgtccagaaccagctgctagg

SEQ ID NO: 540 - DNA, 21 bp, Artificial Sequence
  Description of Artificial Sequence: Synthetic oligonucleotide
tccacctctcgggtggtcatg

SEQ ID NO: 541 - DNA, 21 bp, Artificial Sequence
  Description of Artificial Sequence: Synthetic oligonucleotide
ctccacctctcgggtggtcat

SEQ ID NO: 542 - DNA, 21 bp, Artificial Sequence
  Description of Artificial Sequence: Synthetic oligonucleotide
ccagaacaaatgcgtcccata

SEQ ID NO: 543 - DNA, 21 bp, Artificial Sequence
  Description of Artificial Sequence: Synthetic oligonucleotide
cagaacaaatgcgtcccatac

SEQ ID NO: 544 - DNA, 21 bp, Artificial Sequence
  Description of Artificial Sequence: Synthetic oligonucleotide
tattcggttgtgtcatgctcc

SEQ ID NO: 545 - DNA, 21 bp, Artificial Sequence
  Description of Artificial Sequence: Synthetic oligonucleotide
cctgccgctggttgatctggt

SEQ ID NO: 546 - DNA, 21 bp, Artificial Sequence
  Description of Artificial Sequence: Synthetic oligonucleotide
cccagcgccggcatcggccgc

SEQ ID NO: 547 - DNA, 21 bp, Artificial Sequence
  Description of Artificial Sequence: Synthetic oligonucleotide
gtggagatgttctccatgagc

SEQ ID NO: 548 - DNA, 21 bp, Artificial Sequence
  Description of Artificial Sequence: Synthetic oligonucleotide
tactgcgcctccgtctgcacc

SEQ ID NO: 549 - DNA, 21 bp, Artificial Sequence
  Description of Artificial Sequence: Synthetic oligonucleotide
ttcttagtggtttcaatgaac

SEQ ID NO: 550 - DNA, 21 bp, Artificial Sequence
  Description of Artificial Sequence: Synthetic oligonucleotide
gcatgggcattcttcatggct

SEQ ID NO: 551 - DNA, 21 bp, Artificial Sequence
  Description of Artificial Sequence: Synthetic oligonucleotide
gacgaggtgcgggaggccttg

SEQ ID NO: 552 - DNA, 21 bp, Artificial Sequence
  Description of Artificial Sequence: Synthetic oligonucleotide
gagtctagtgcagggaccgtg

SEQ ID NO: 553 - DNA, 21 bp, Artificial Sequence
  Description of Artificial Sequence: Synthetic oligonucleotide
tgtctgcagccgggtgcaggg

SEQ ID NO: 554 - DNA, 21 bp, Artificial Sequence
  Description of Artificial Sequence: Synthetic oligonucleotide
tcctccctcttgttcttagtg

SEQ ID NO: 555 - DNA, 21 bp, Artificial Sequence
  Description of Artificial Sequence: Synthetic oligonucleotide
ctcctccctcttgttcttagt

SEQ ID NO: 556 - DNA, 21 bp, Artificial Sequence
  Description of Artificial Sequence: Synthetic oligonucleotide
ttcactttctcctccctcttg

SEQ ID NO: 557 - DNA, 21 bp, Artificial Sequence
  Description of Artificial Sequence: Synthetic oligonucleotide
cctgaggcgaggggagcctgc

SEQ ID NO: 558 - DNA, 21 bp, Artificial Sequence
  Description of Artificial Sequence: Synthetic oligonucleotide
gtcctcttccctaggaatgat

SEQ ID NO: 559 - DNA, 21 bp, Artificial Sequence
  Description of Artificial Sequence: Synthetic oligonucleotide
tattgtgcctgtcatcattcc

SEQ ID NO: 560 - DNA, 21 bp, Artificial Sequence
  Description of Artificial Sequence: Synthetic oligonucleotide
tctgcagaaatgttccccgtt

SEQ ID NO: 561 - DNA, 21 bp, Artificial Sequence
  Description of Artificial Sequence: Synthetic oligonucleotide
ctctgcagaaatgttccccgt

SEQ ID NO: 562 - DNA, 21 bp, Artificial Sequence
  Description of Artificial Sequence: Synthetic oligonucleotide
tgccgtggtggaggagaggtg

SEQ ID NO: 563 - DNA, 21 bp, Artificial Sequence
  Description of Artificial Sequence: Synthetic oligonucleotide
tggccatttgtaatgctgact

SEQ ID NO: 564 - DNA, 21 bp, Artificial Sequence
  Description of Artificial Sequence: Synthetic oligonucleotide
aaggatggggagatgtgccac

SEQ ID NO: 565 - DNA, 21 bp, Artificial Sequence
  Description of Artificial Sequence: Synthetic oligonucleotide
aaggatcgagtggccccaggt

SEQ ID NO: 566 - DNA, 21 bp, Artificial Sequence
  Description of Artificial Sequence: Synthetic oligonucleotide
tgcatctatcacacctaccct

SEQ ID NO: 567 - DNA, 21 bp, Artificial Sequence
  Description of Artificial Sequence: Synthetic oligonucleotide
taggacctccaggaagattct

SEQ ID NO: 568 - DNA, 21 bp, Artificial Sequence
  Description of Artificial Sequence: Synthetic oligonucleotide
ctaggacctccaggaagattc

SEQ ID NO: 569 - DNA, 21 bp, Artificial Sequence
  Description of Artificial Sequence: Synthetic oligonucleotide
ctccagcaggaatacctgagc

SEQ ID NO: 570 - DNA, 21 bp, Artificial Sequence
  Description of Artificial Sequence: Synthetic oligonucleotide
gagccatggccgcgacgctgg

SEQ ID NO: 571 - DNA, 21 bp, Artificial Sequence
  Description of Artificial Sequence: Synthetic oligonucleotide
tagtcaacgcgaccaccacga

SEQ ID NO: 572 - DNA, 21 bp, Artificial Sequence
  Description of Artificial Sequence: Synthetic oligonucleotide
ctagtcaacgcgaccaccacg

SEQ ID NO: 573 - DNA, 21 bp, Artificial Sequence
  Description of Artificial Sequence: Synthetic oligonucleotide
tagtttgtttgtttttagaag

SEQ ID NO: 574 - DNA, 21 bp, Artificial Sequence
  Description of Artificial Sequence: Synthetic oligonucleotide
tttgtttttagaagaaagccc

SEQ ID NO: 575 - DNA, 21 bp, Artificial Sequence
  Description of Artificial Sequence: Synthetic oligonucleotide
tttttagaagaaagccctcag

SEQ ID NO: 576 - DNA, 21 bp, Artificial Sequence
  Description of Artificial Sequence: Synthetic oligonucleotide
tagaagaaagccctcagaatc

SEQ ID NO: 577 - DNA, 21 bp, Artificial Sequence
  Description of Artificial Sequence: Synthetic oligonucleotide
cacagaatggattctgagggc

SEQ ID NO: 578 - DNA, 21 bp, Artificial Sequence
  Description of Artificial Sequence: Synthetic oligonucleotide
ctgggggtgagggagcactgg

SEQ ID NO: 579 - DNA, 21 bp, Artificial Sequence
  Description of Artificial Sequence: Synthetic oligonucleotide
tgcctggacacagcttcctgg

SEQ ID NO: 580 - DNA, 21 bp, Artificial Sequence
  Description of Artificial Sequence: Synthetic oligonucleotide
tgtaactcaggacattgaagt

SEQ ID NO: 581 - DNA, 21 bp, Artificial Sequence
  Description of Artificial Sequence: Synthetic oligonucleotide
aatgtcctgagttacagaagc

SEQ ID NO: 582 - DNA, 21 bp, Artificial Sequence
  Description of Artificial Sequence: Synthetic oligonucleotide
gtgtttttaatgtgactctca

SEQ ID NO: 583 - DNA, 21 bp, Artificial Sequence
  Description of Artificial Sequence: Synthetic oligonucleotide
cgtaggattttgtgtttttaa

SEQ ID NO: 584 - DNA, 21 bp, Artificial Sequence
  Description of Artificial Sequence: Synthetic oligonucleotide
cttagtgctgagactcattct

SEQ ID NO: 585 - DNA, 21 bp, Artificial Sequence
  Description of Artificial Sequence: Synthetic oligonucleotide
ccttagtgctgagactcattc

SEQ ID NO: 586 - DNA, 21 bp, Artificial Sequence
  Description of Artificial Sequence: Synthetic oligonucleotide
tttggtactttacagtttatt

SEQ ID NO: 587 - DNA, 21 bp, Artificial Sequence
  Description of Artificial Sequence: Synthetic oligonucleotide
cagtttattaaatagatgttt

SEQ ID NO: 588 - DNA, 21 bp, Artificial Sequence
  Description of Artificial Sequence: Synthetic oligonucleotide
ttaaatagatgtttatatgga

SEQ ID NO: 589 - DNA, 21 bp, Artificial Sequence
  Description of Artificial Sequence: Synthetic oligonucleotide
acctgcaaaatgaatatggtg

SEQ ID NO: 590 - DNA, 21 bp, Artificial Sequence
  Description of Artificial Sequence: Synthetic oligonucleotide
gcaggtgaaattcctgagatg

SEQ ID NO: 591 - DNA, 21 bp, Artificial Sequence
  Description of Artificial Sequence: Synthetic oligonucleotide
tggtatgttggcattaagttg

SEQ ID NO: 592 - DNA, 21 bp, Artificial Sequence
  Description of Artificial Sequence: Synthetic oligonucleotide
ccaacttaatgccaacatacc

SEQ ID NO: 593 - DNA, 21 bp, Artificial Sequence
  Description of Artificial Sequence: Synthetic oligonucleotide
ctttgctgggcctttttccca

SEQ ID NO: 594 - DNA, 21 bp, Artificial Sequence
  Description of Artificial Sequence: Synthetic oligonucleotide
tcccatgcctgcctttactct

SEQ ID NO: 595 - DNA, 21 bp, Artificial Sequence
  Description of Artificial Sequence: Synthetic oligonucleotide
cccatgcctgcctttactctg

SEQ ID NO: 596 - DNA, 21 bp, Artificial Sequence
  Description of Artificial Sequence: Synthetic oligonucleotide
tttaataggatcttcttcaaa

SEQ ID NO: 597 - DNA, 21 bp, Artificial Sequence
  Description of Artificial Sequence: Synthetic oligonucleotide
atttaataggatcttcttcaa

SEQ ID NO: 598 - DNA, 21 bp, Artificial Sequence
  Description of Artificial Sequence: Synthetic oligonucleotide
aagaagatcctattaaataaa

SEQ ID NO: 599 - DNA, 21 bp, Artificial Sequence
  Description of Artificial Sequence: Synthetic oligonucleotide
agtgaacgttcacggccaggc

SEQ ID NO: 600 - DNA, 21 bp, Artificial Sequence
  Description of Artificial Sequence: Synthetic oligonucleotide
tttgagaatcaaaatcggtga

SEQ ID NO: 601 - DNA, 21 bp, Artificial Sequence
  Description of Artificial Sequence: Synthetic oligonucleotide
tgacacatttgtttgagaatc

SEQ ID NO: 602 - DNA, 21 bp, Artificial Sequence
  Description of Artificial Sequence: Synthetic oligonucleotide
gattctcaaacaaatgtgtca

SEQ ID NO: 603 - DNA, 21 bp, Artificial Sequence
  Description of Artificial Sequence: Synthetic oligonucleotide
cacatgcaaagtcagatttgt

SEQ ID NO: 604 - DNA, 21 bp, Artificial Sequence
  Description of Artificial Sequence: Synthetic oligonucleotide
gtgccttcgcaggctgtttcc

SEQ ID NO: 605 - DNA, 21 bp, Artificial Sequence
  Description of Artificial Sequence: Synthetic oligonucleotide
agacatcattgaccagagctc

SEQ ID NO: 606 - DNA, 21 bp, Artificial Sequence
  Description of Artificial Sequence: Synthetic oligonucleotide
ttagtaaaaagagggttttgg

SEQ ID NO: 607 - DNA, 21 bp, Artificial Sequence
  Description of Artificial Sequence: Synthetic oligonucleotide
actaagaaacagtgagccttg

SEQ ID NO: 608 - DNA, 21 bp, Artificial Sequence
  Description of Artificial Sequence: Synthetic oligonucleotide
tcccgtgtcattctctggact

SEQ ID NO: 609 - DNA, 21 bp, Artificial Sequence
  Description of Artificial Sequence: Synthetic oligonucleotide
ttcccgtgtcattctctggac

SEQ ID NO: 610 - DNA, 21 bp, Artificial Sequence
  Description of Artificial Sequence: Synthetic oligonucleotide
tccctgtctgccaaaaaatct

SEQ ID NO: 611 - DNA, 21 bp, Artificial Sequence
  Description of Artificial Sequence: Synthetic oligonucleotide
aattcctccacttcaacacct

SEQ ID NO: 612 - DNA, 21 bp, Artificial Sequence
  Description of Artificial Sequence: Synthetic oligonucleotide
tcaaccctgagttaaaacaca

SEQ ID NO: 613 - DNA, 21 bp, Artificial Sequence
  Description of Artificial Sequence: Synthetic oligonucleotide
ctcaaccctgagttaaaacac

SEQ ID NO: 614 - DNA, 21 bp, Artificial Sequence
  Description of Artificial Sequence: Synthetic oligonucleotide
tcctgaaggtagctgttttct

SEQ ID NO: 615 - DNA, 21 bp, Artificial Sequence
  Description of Artificial Sequence: Synthetic oligonucleotide
gtcctgaaggtagctgttttc

SEQ ID NO: 616 - DNA, 21 bp, Artificial Sequence
  Description of Artificial Sequence: Synthetic oligonucleotide
aactcagggttgagaaaacag

SEQ ID NO: 617 - DNA, 21 bp, Artificial Sequence
  Description of Artificial Sequence: Synthetic oligonucleotide
actcagggttgagaaaacagc

SEQ ID NO: 618 - DNA, 21 bp, Artificial Sequence
  Description of Artificial Sequence: Synthetic oligonucleotide
ctggcctggaggctatccagc

SEQ ID NO: 619 - DNA, 21 bp, Artificial Sequence
  Description of Artificial Sequence: Synthetic oligonucleotide
tcccgatattcctcaggtact

SEQ ID NO: 620 - DNA, 21 bp, Artificial Sequence
  Description of Artificial Sequence: Synthetic oligonucleotide
ccgatattcctcaggtactcc

SEQ ID NO: 621 - DNA, 21 bp, Artificial Sequence
  Description of Artificial Sequence: Synthetic oligonucleotide
agtaagtcaacttcaatgtcg

SEQ ID NO: 622 - DNA, 21 bp, Artificial Sequence
  Description of Artificial Sequence: Synthetic oligonucleotide
aattctctctccattcttcag

SEQ ID NO: 623 - DNA, 21 bp, Artificial Sequence
  Description of Artificial Sequence: Synthetic oligonucleotide
caattctctctccattcttca

SEQ ID NO: 624 - DNA, 21 bp, Artificial Sequence
  Description of Artificial Sequence: Synthetic oligonucleotide
cagcaaggactggtctttcta

SEQ ID NO: 625 - DNA, 21 bp, Artificial Sequence
  Description of Artificial Sequence: Synthetic oligonucleotide
agtgggggtgaattcagtgta

SEQ ID NO: 626 - DNA, 21 bp, Artificial Sequence
  Description of Artificial Sequence: Synthetic oligonucleotide
cagtgggggtgaattcagtgt

SEQ ID NO: 627 - DNA, 21 bp, Artificial Sequence
  Description of Artificial Sequence: Synthetic oligonucleotide
ctatctcttgtactacactga

SEQ ID NO: 628 - DNA, 21 bp, Artificial Sequence
  Description of Artificial Sequence: Synthetic oligonucleotide
tactacactgaattcaccccc

SEQ ID NO: 629 - DNA, 21 bp, Artificial Sequence
  Description of Artificial Sequence: Synthetic oligonucleotide
ggctgtgacaaagtcacatgg

SEQ ID NO: 630 - DNA, 21 bp, Artificial Sequence
  Description of Artificial Sequence: Synthetic oligonucleotide
caaaagaatgtaagacttacc

SEQ ID NO: 631 - DNA, 21 bp, Artificial Sequence
  Description of Artificial Sequence: Synthetic oligonucleotide
cctccatgatgctgcttacat

SEQ ID NO: 632 - DNA, 21 bp, Artificial Sequence
  Description of Artificial Sequence: Synthetic oligonucleotide
ttcatagatcgagacatgtaa

SEQ ID NO: 633 - DNA, 21 bp, Artificial Sequence
  Description of Artificial Sequence: Synthetic oligonucleotide
tcatagatcgagacatgtaag

SEQ ID NO: 634 - DNA, 21 bp, Artificial Sequence
  Description of Artificial Sequence: Synthetic oligonucleotide
catagatcgagacatgtaagc

SEQ ID NO: 635 - DNA, 21 bp, Artificial Sequence
  Description of Artificial Sequence: Synthetic oligonucleotide
atagatcgagacatgtaagca

SEQ ID NO: 636 - DNA, 21 bp, Artificial Sequence
  Description of Artificial Sequence: Synthetic oligonucleotide
ccttggggctctgacaggtag

SEQ ID NO: 637 - DNA, 21 bp, Artificial Sequence
  Description of Artificial Sequence: Synthetic oligonucleotide
accttggggctctgacaggta

SEQ ID NO: 638 - DNA, 21 bp, Artificial Sequence
  Description of Artificial Sequence: Synthetic oligonucleotide
ccggcctttttaccttggggc

SEQ ID NO: 639 - DNA, 21 bp, Artificial Sequence
  Description of Artificial Sequence: Synthetic oligonucleotide
ctcccagaacccgacacagac

SEQ ID NO: 640 - DNA, 21 bp, Artificial Sequence
  Description of Artificial Sequence: Synthetic oligonucleotide
tgggctcaggtgcttcctcac

SEQ ID NO: 641 - DNA, 21 bp, Artificial Sequence
  Description of Artificial Sequence: Synthetic oligonucleotide
ctgggctcaggtgcttcctca

SEQ ID NO: 642 - DNA, 21 bp, Artificial Sequence
  Description of Artificial Sequence: Synthetic oligonucleotide
cttgtctgggcagcggaactg

SEQ ID NO: 643 - DNA, 21 bp, Artificial Sequence
  Description of Artificial Sequence: Synthetic oligonucleotide
ctcaaagtagagcacatagga

SEQ ID NO: 644 - DNA, 21 bp, Artificial Sequence
  Description of Artificial Sequence: Synthetic oligonucleotide
tcaaagtagagcacataggac

SEQ ID NO: 645 - DNA, 21 bp, Artificial Sequence
  Description of Artificial Sequence: Synthetic oligonucleotide
tgcccaacttctgctggcatc

SEQ ID NO: 646 - DNA, 21 bp, Artificial Sequence
  Description of Artificial Sequence: Synthetic oligonucleotide
tgacttttctgcccaacttct

SEQ ID NO: 647 - DNA, 21 bp, Artificial Sequence
  Description of Artificial Sequence: Synthetic oligonucleotide
tctgcagccttcccagaggag

SEQ ID NO: 648 - DNA, 21 bp, Artificial Sequence
  Description of Artificial Sequence: Synthetic oligonucleotide
tccaggcgcatctggccggag

SEQ ID NO: 649 - DNA, 21 bp, Artificial Sequence
  Description of Artificial Sequence: Synthetic oligonucleotide
tccagttcctcgttgagctgc

SEQ ID NO: 650 - DNA, 21 bp, Artificial Sequence
  Description of Artificial Sequence: Synthetic oligonucleotide
ccagagcccatggggcagagt

SEQ ID NO: 651 - DNA, 21 bp, Artificial Sequence
  Description of Artificial Sequence: Synthetic oligonucleotide
tccccaccatctccactctgc

SEQ ID NO: 652 - DNA, 21 bp, Artificial Sequence
  Description of Artificial Sequence: Synthetic oligonucleotide
ctcgggaggtcagggcaggtt

SEQ ID NO: 653 - DNA, 21 bp, Artificial Sequence
  Description of Artificial Sequence: Synthetic oligonucleotide
gaagcttgttggagacctctc

SEQ ID NO: 654 - DNA, 21 bp, Artificial Sequence
  Description of Artificial Sequence: Synthetic oligonucleotide
ggaagcttgttggagacctct

SEQ ID NO: 655 - DNA, 21 bp, Artificial Sequence
  Description of Artificial Sequence: Synthetic oligonucleotide
cagagccggtggagcagttct

SEQ ID NO: 656 - DNA, 21 bp, Artificial Sequence
  Description of Artificial Sequence: Synthetic oligonucleotide
cccagcacagcaatcactcgt

SEQ ID NO: 657 - DNA, 21 bp, Artificial Sequence
  Description of Artificial Sequence: Synthetic oligonucleotide
tcttctctgtcccctgccatt

SEQ ID NO: 658 - DNA, 21 bp, Artificial Sequence
  Description of Artificial Sequence: Synthetic oligonucleotide
agccacatcttgaagagacct

SEQ ID NO: 659 - DNA, 21 bp, Artificial Sequence
  Description of Artificial Sequence: Synthetic oligonucleotide
ccagaagaagctgctccgagg

SEQ ID NO: 660 - DNA, 21 bp, Artificial Sequence
  Description of Artificial Sequence: Synthetic oligonucleotide
cagaagaagctgctccgaggt

SEQ ID NO: 661 - DNA, 21 bp, Artificial Sequence
  Description of Artificial Sequence: Synthetic oligonucleotide
agctgtccggcttctccatgg

SEQ ID NO: 662 - DNA, 21 bp, Artificial Sequence
  Description of Artificial Sequence: Synthetic oligonucleotide
agagctcagggatgacagagc

SEQ ID NO: 663 - DNA, 21 bp, Artificial Sequence
  Description of Artificial Sequence: Synthetic oligonucleotide
tgccgggcagtgtgccagctc

SEQ ID NO: 664 - DNA, 21 bp, Artificial Sequence
  Description of Artificial Sequence: Synthetic oligonucleotide
atgtctgcggcccagctccca

SEQ ID NO: 665 - DNA, 21 bp, Artificial Sequence
  Description of Artificial Sequence: Synthetic oligonucleotide
gccatcgcccaggtcctcacg

SEQ ID NO: 666 - DNA, 21 bp, Artificial Sequence
  Description of Artificial Sequence: Synthetic oligonucleotide
gccactcagagccagccacag

SEQ ID NO: 667 - DNA, 21 bp, Artificial Sequence
  Description of Artificial Sequence: Synthetic oligonucleotide
tggctgggctgatcttccagc

SEQ ID NO: 668 - DNA, 21 bp, Artificial Sequence
  Description of Artificial Sequence: Synthetic oligonucleotide
gcagcacgtggtacaggagct

SEQ ID NO: 669 - DNA, 21 bp, Artificial Sequence
  Description of Artificial Sequence: Synthetic oligonucleotide
ctgggcacccgcctcacgcct

SEQ ID NO: 670 - DNA, 21 bp, Artificial Sequence
  Description of Artificial Sequence: Synthetic oligonucleotide
tgggcacccgcctcacgcctc

SEQ ID NO: 671 - DNA, 21 bp, Artificial Sequence
  Description of Artificial Sequence: Synthetic oligonucleotide
cccctctggattggggagcct

SEQ ID NO: 672 - DNA, 21 bp, Artificial Sequence
  Description of Artificial Sequence: Synthetic oligonucleotide
aaaggctcgatggtgaacttc

SEQ ID NO: 673 - DNA, 21 bp, Artificial Sequence
  Description of Artificial Sequence: Synthetic oligonucleotide
ccaggtcttccacatccttca

SEQ ID NO: 674 - DNA, 21 bp, Artificial Sequence
  Description of Artificial Sequence: Synthetic oligonucleotide
aaagccaagtccctgaaggat

SEQ ID NO: 675 - DNA, 21 bp, Artificial Sequence
  Description of Artificial Sequence: Synthetic oligonucleotide
ggtcccgaacagcagggagct

SEQ ID NO: 676 - DNA, 21 bp, Artificial Sequence
  Description of Artificial Sequence: Synthetic oligonucleotide
tttaggtcccgaacagcaggg

SEQ ID NO: 677 - DNA, 21 bp, Artificial Sequence
  Description of Artificial Sequence: Synthetic oligonucleotide
cttacgcaaactccagtttct

SEQ ID NO: 678 - DNA, 21 bp, Artificial Sequence
  Description of Artificial Sequence: Synthetic oligonucleotide
cctcctaggctgggccctgtc

SEQ ID NO: 679 - DNA, 21 bp, Artificial Sequence
  Description of Artificial Sequence: Synthetic oligonucleotide
gggaaagcctgggggcctgag

SEQ ID NO: 680 - DNA, 21 bp, Artificial Sequence
  Description of Artificial Sequence: Synthetic oligonucleotide
cccaaactggtgcggatcctc

SEQ ID NO: 681 - DNA, 21 bp, Artificial Sequence
  Description of Artificial Sequence: Synthetic oligonucleotide
ctccctgcagcatctggagtg

SEQ ID NO: 682 - DNA, 21 bp, Artificial Sequence
  Description of Artificial Sequence: Synthetic oligonucleotide
caaggacttcagctgggggaa

SEQ ID NO: 683 - DNA, 21 bp, Artificial Sequence
  Description of Artificial Sequence: Synthetic oligonucleotide
taggcacccaggtcagtgatg

SEQ ID NO: 684 - DNA, 21 bp, Artificial Sequence
  Description of Artificial Sequence: Synthetic oligonucleotide
cgacagcttgtacaataactg

SEQ ID NO: 685 - DNA, 21 bp, Artificial Sequence
  Description of Artificial Sequence: Synthetic oligonucleotide
tcttgccagcgtccagtacaa

SEQ ID NO: 686 - DNA, 21 bp, Artificial Sequence
  Description of Artificial Sequence: Synthetic oligonucleotide
cccggcctttttaccttgggg

SEQ ID NO: 687 - DNA, 21 bp, Artificial Sequence
  Description of Artificial Sequence: Synthetic oligonucleotide
cctcccaggcagctcacagtg

SEQ ID NO: 688 - DNA, 21 bp, Artificial Sequence
  Description of Artificial Sequence: Synthetic oligonucleotide
tccagccaggtccatctggtc

SEQ ID NO: 689 - DNA, 21 bp, Artificial Sequence
  Description of Artificial Sequence: Synthetic oligonucleotide
ttctccagccaggtccatctg

SEQ ID NO: 690 - DNA, 21 bp, Artificial Sequence
  Description of Artificial Sequence: Synthetic oligonucleotide
atcaccttccatgtcacacaa

SEQ ID NO: 691 - DNA, 21 bp, Artificial Sequence
  Description of Artificial Sequence: Synthetic oligonucleotide
tctgggctcaggtgcttcctc

SEQ ID NO: 692 - DNA, 21 bp, Artificial Sequence
  Description of Artificial Sequence: Synthetic oligonucleotide
tgccaatatcggtgaggaagc

SEQ ID NO: 693 - DNA, 21 bp, Artificial Sequence
  Description of Artificial Sequence: Synthetic oligonucleotide
caggactcccagctggagggc

SEQ ID NO: 694 - DNA, 21 bp, Artificial Sequence
  Description of Artificial Sequence: Synthetic oligonucleotide
tctgacttttctgcccaactt

SEQ ID NO: 695 - DNA, 21 bp, Artificial Sequence
  Description of Artificial Sequence: Synthetic oligonucleotide
cagtgcctttctccagttcct

SEQ ID NO: 696 - DNA, 21 bp, Artificial Sequence
  Description of Artificial Sequence: Synthetic oligonucleotide
gctggcctggggcacctcacc

SEQ ID NO: 697 - DNA, 21 bp, Artificial Sequence
  Description of Artificial Sequence: Synthetic oligonucleotide
gctccatcagccactgacctg

SEQ ID NO: 698 - DNA, 21 bp, Artificial Sequence
  Description of Artificial Sequence: Synthetic oligonucleotide
cctgtcatgtttgctcgggag

SEQ ID NO: 699 - DNA, 21 bp, Artificial Sequence
  Description of Artificial Sequence: Synthetic oligonucleotide
tccatctccagagcacaagac

SEQ ID NO: 700 - DNA, 21 bp, Artificial Sequence
  Description of Artificial Sequence: Synthetic oligonucleotide
ttggagacctctccagctgcc

SEQ ID NO: 701 - DNA, 21 bp, Artificial Sequence
  Description of Artificial Sequence: Synthetic oligonucleotide
gcagagccggtggagcagttc

SEQ ID NO: 702 - DNA, 21 bp, Artificial Sequence
  Description of Artificial Sequence: Synthetic oligonucleotide
ctgctgctcctctccagcctg

SEQ ID NO: 703 - DNA, 21 bp, Artificial Sequence
  Description of Artificial Sequence: Synthetic oligonucleotide
gcagccaacagcacctcagcc

SEQ ID NO: 704 - DNA, 21 bp, Artificial Sequence
  Description of Artificial Sequence: Synthetic oligonucleotide
gcccagcacagcaatcactcg

SEQ ID NO: 705 - DNA, 21 bp, Artificial Sequence
  Description of Artificial Sequence: Synthetic oligonucleotide
agccatcagaagcagagatct

SEQ ID NO: 706 - DNA, 21 bp, Artificial Sequence
  Description of Artificial Sequence: Synthetic oligonucleotide
cgctgtcaagtccagttctac

SEQ ID NO: 707 - DNA, 21 bp, Artificial Sequence
  Description of Artificial Sequence: Synthetic oligonucleotide
ccctgttttctttcagactgt

SEQ ID NO: 708 - DNA, 21 bp, Artificial Sequence
  Description of Artificial Sequence: Synthetic oligonucleotide
ctttcagactgtggcttcacc

SEQ ID NO: 709 - DNA, 21 bp, Artificial Sequence
  Description of Artificial Sequence: Synthetic oligonucleotide
tttcagactgtggcttcacct

SEQ ID NO: 710 - DNA, 21 bp, Artificial Sequence
  Description of Artificial Sequence: Synthetic oligonucleotide
cagactgtggcttcacctccg

SEQ ID NO: 711 - DNA, 21 bp, Artificial Sequence
  Description of Artificial Sequence: Synthetic oligonucleotide
agactgtggcttcacctccgg

SEQ ID NO: 712 - DNA, 21 bp, Artificial Sequence
  Description of Artificial Sequence: Synthetic oligonucleotide
ccggaggtgaagccacagtct

SEQ ID NO: 713 - DNA, 21 bp, Artificial Sequence
  Description of Artificial Sequence: Synthetic oligonucleotide
tcaacagagtcttaccagcaa

SEQ ID NO: 714 - DNA, 21 bp, Artificial Sequence
  Description of Artificial Sequence: Synthetic oligonucleotide
ccagcaaggggtcctgtctgc

SEQ ID NO: 715 - DNA, 21 bp, Artificial Sequence
  Description of Artificial Sequence: Synthetic oligonucleotide
ctagggaaggccaccttgtat

SEQ ID NO: 716 - DNA, 21 bp, Artificial Sequence
  Description of Artificial Sequence: Synthetic oligonucleotide
tatgccgtgctggtcagtgcc

SEQ ID NO: 717 - DNA, 21 bp, Artificial Sequence
  Description of Artificial Sequence: Synthetic oligonucleotide
ccatggccatcagcacgaggg

SEQ ID NO: 718 - DNA, 21 bp, Artificial Sequence
  Description of Artificial Sequence: Synthetic oligonucleotide
cctagcaagatctcatagagg

SEQ ID NO: 719 - DNA, 21 bp, Artificial Sequence
  Description of Artificial Sequence: Synthetic oligonucleotide
cacaggtcaagagaaaggatt

SEQ ID NO: 720 - DNA, 21 bp, Artificial Sequence
  Description of Artificial Sequence: Synthetic oligonucleotide
gagctagcctctggaatcctt

SEQ ID NO: 721 - DNA, 21 bp, Artificial Sequence
  Description of Artificial Sequence: Synthetic oligonucleotide
tagtaccgctcctggaaggtt

SEQ ID NO: 722 - DNA, 21 bp, Artificial Sequence
  Description of Artificial Sequence: Synthetic oligonucleotide
atcttgtagtaccgctcctgg

SEQ ID NO: 723 - DNA, 21 bp, Artificial Sequence
  Description of Artificial Sequence: Synthetic oligonucleotide
cttcatcttgtagtaccgctc

SEQ ID NO: 724 - DNA, 21 bp, Artificial Sequence
  Description of Artificial Sequence: Synthetic oligonucleotide
tgtgttgcagttcagcaggag

SEQ ID NO: 725 - DNA, 21 bp, Artificial Sequence
  Description of Artificial Sequence: Synthetic oligonucleotide
cgttatagagctggttctggc

SEQ ID NO: 726 - DNA, 21 bp, Artificial Sequence
  Description of Artificial Sequence: Synthetic oligonucleotide
cggagggtctacggcgaggct

SEQ ID NO: 727 - DNA, 21 bp, Artificial Sequence
  Description of Artificial Sequence: Synthetic oligonucleotide
ttatctgttataggagctcaa

SEQ ID NO: 728 - DNA, 21 bp, Artificial Sequence
  Description of Artificial Sequence: Synthetic oligonucleotide
tctgttataggagctcaatct

SEQ ID NO: 729 - DNA, 21 bp, Artificial Sequence
  Description of Artificial Sequence: Synthetic oligonucleotide
tccaaaacatcgtactcctct

SEQ ID NO: 730 - DNA, 21 bp, Artificial Sequence
  Description of Artificial Sequence: Synthetic oligonucleotide
cccccatctcagggtcccggc

SEQ ID NO: 731 - DNA, 21 bp, Artificial Sequence
  Description of Artificial Sequence: Synthetic oligonucleotide
gacaagagacgtggccgggac

SEQ ID NO: 732 - DNA, 21 bp, Artificial Sequence
  Description of Artificial Sequence: Synthetic oligonucleotide
tctccctctaacgtcttcccg

SEQ ID NO: 733 - DNA, 21 bp, Artificial Sequence
  Description of Artificial Sequence: Synthetic oligonucleotide
ctgagggttcttccttctctg

SEQ ID NO: 734 - DNA, 21 bp, Artificial Sequence
  Description of Artificial Sequence: Synthetic oligonucleotide
ccgttgtctttcctagcagag

SEQ ID NO: 735 - DNA, 21 bp, Artificial Sequence
  Description of Artificial Sequence: Synthetic oligonucleotide
ctagcagagaaggaagaaccc

SEQ ID NO: 736 - DNA, 21 bp, Artificial Sequence
  Description of Artificial Sequence: Synthetic oligonucleotide
tgcagttcctgcagaagaggg

SEQ ID NO: 737 - DNA, 21 bp, Artificial Sequence
  Description of Artificial Sequence: Synthetic oligonucleotide
tgcaggaactgcagaaagata

SEQ ID NO: 738 - DNA, 21 bp, Artificial Sequence
  Description of Artificial Sequence: Synthetic oligonucleotide
atcccaatctcactgtaggcc

SEQ ID NO: 739 - DNA, 21 bp, Artificial Sequence
  Description of Artificial Sequence: Synthetic oligonucleotide
catcccaatctcactgtaggc

SEQ ID NO: 740 - DNA, 21 bp, Artificial Sequence
  Description of Artificial Sequence: Synthetic oligonucleotide
ctcatttcactcccaaacaac

SEQ ID NO: 741 - DNA, 21 bp, Artificial Sequence
  Description of Artificial Sequence: Synthetic oligonucleotide
tcatttcactcccaaacaacc

SEQ ID NO: 742 - DNA, 21 bp, Artificial Sequence
  Description of Artificial Sequence: Synthetic oligonucleotide
actcccaaacaaccagcgccg

SEQ ID NO: 743 - DNA, 21 bp, Artificial Sequence
  Description of Artificial Sequence: Synthetic oligonucleotide
ttttctgatttgctttcacgc

SEQ ID NO: 744 - DNA, 21 bp, Artificial Sequence
  Description of Artificial Sequence: Synthetic oligonucleotide
tgatttgctttcacgccaggg

SEQ ID NO: 745 - DNA, 21 bp, Artificial Sequence
  Description of Artificial Sequence: Synthetic oligonucleotide
ctttcacgccagggtctcagt

SEQ ID NO: 746 - DNA, 21 bp, Artificial Sequence
  Description of Artificial Sequence: Synthetic oligonucleotide
acgccagggtctcagtacagc

SEQ ID NO: 747 - DNA, 21 bp, Artificial Sequence
  Description of Artificial Sequence: Synthetic oligonucleotide
ctttccaggggagatggatcc

SEQ ID NO: 748 - DNA, 21 bp, Artificial Sequence
  Description of Artificial Sequence: Synthetic oligonucleotide
ccaggggagatggatcctatc

SEQ ID NO: 749 - DNA, 21 bp, Artificial Sequence
  Description of Artificial Sequence: Synthetic oligonucleotide
caggggagatggatcctatct

SEQ ID NO: 750 - DNA, 21 bp, Artificial Sequence
  Description of Artificial Sequence: Synthetic oligonucleotide
ctaaccatcagcattttgagt

SEQ ID NO: 751 - DNA, 21 bp, Artificial Sequence
  Description of Artificial Sequence: Synthetic oligonucleotide
gagttttttctctgtcgctct

SEQ ID NO: 752 - DNA, 21 bp, Artificial Sequence
  Description of Artificial Sequence: Synthetic oligonucleotide
agttttttctctgtcgctctg

SEQ ID NO: 753 - DNA, 21 bp, Artificial Sequence
  Description of Artificial Sequence: Synthetic oligonucleotide
tctgtcgctctgttggtcatc

SEQ ID NO: 754 - DNA, 21 bp, Artificial Sequence
  Description of Artificial Sequence: Synthetic oligonucleotide
cataacacacaggccaagatg

SEQ ID NO: 755 - DNA, 21 bp, Artificial Sequence
  Description of Artificial Sequence: Synthetic oligonucleotide
atgtcctttcacccatcaacc

SEQ ID NO: 756 - DNA, 21 bp, Artificial Sequence
  Description of Artificial Sequence: Synthetic oligonucleotide
cacccatcaacccgtagggat

SEQ ID NO: 757 - DNA, 21 bp, Artificial Sequence
  Description of Artificial Sequence: Synthetic oligonucleotide
acccatcaacccgtagggatg

SEQ ID NO: 758 - DNA, 21 bp, Artificial Sequence
  Description of Artificial Sequence: Synthetic oligonucleotide
ctcatacaccacgaagcgcag

SEQ ID NO: 759 - DNA, 21 bp, Artificial Sequence
  Description of Artificial Sequence: Synthetic oligonucleotide
cctttctgcgcttcgtggtgt

SEQ ID NO: 760 - DNA, 21 bp, Artificial Sequence
  Description of Artificial Sequence: Synthetic oligonucleotide
ctgcgcttcgtggtgtatgag

SEQ ID NO: 761 - DNA, 21 bp, Artificial Sequence
  Description of Artificial Sequence: Synthetic oligonucleotide
tgcgcttcgtggtgtatgagg

SEQ ID NO: 762 - DNA, 21 bp, Artificial Sequence
  Description of Artificial Sequence: Synthetic oligonucleotide
gtggtgtatgaggaagacatg

SEQ ID NO: 763 - DNA, 21 bp, Artificial Sequence
  Description of Artificial Sequence: Synthetic oligonucleotide
gtcctttccacaagatatata

SEQ ID NO: 764 - DNA, 21 bp, Artificial Sequence
  Description of Artificial Sequence: Synthetic oligonucleotide
gatttcttggctttatatatc

SEQ ID NO: 765 - DNA, 21 bp, Artificial Sequence
  Description of Artificial Sequence: Synthetic oligonucleotide
ttggctttatatatcttgtgg

SEQ ID NO: 766 - DNA, 21 bp, Artificial Sequence
  Description of Artificial Sequence: Synthetic oligonucleotide
gctttatatatcttgtggaaa

SEQ ID NO: 767 - DNA, 21 bp, Artificial Sequence
  Description of Artificial Sequence: Synthetic oligonucleotide
atatatcttgtggaaaggacg

SEQ ID NO: 768 - DNA, 21 bp, Artificial Sequence
  Description of Artificial Sequence: Synthetic oligonucleotide
tatatcttgtggaaaggacga

SEQ ID NO: 769 - DNA, 21 bp, Artificial Sequence
  Description of Artificial Sequence: Synthetic oligonucleotide
tggaaaggacgaaacaccgtg

SEQ ID NO: 770 - DNA, 21 bp, Artificial Sequence
  Description of Artificial Sequence: Synthetic oligonucleotide
ttgcagaagcgggcccaggat

SEQ ID NO: 771 - DNA, 21 bp, Artificial Sequence
  Description of Artificial Sequence: Synthetic oligonucleotide
ttgcagaagcgggcccaggat

SEQ ID NO: 772 - DNA, 21 bp, Artificial Sequence
  Description of Artificial Sequence: Synthetic oligonucleotide
tatgctgaacacctgatgccg

SEQ ID NO: 773 - DNA, 21 bp, Artificial Sequence
  Description of Artificial Sequence: Synthetic oligonucleotide
ccagggtcggccggtgcccgt

SEQ ID NO: 774 - DNA, 21 bp, Artificial Sequence
  Description of Artificial Sequence: Synthetic oligonucleotide
gccggtggagccgccgccgga

SEQ ID NO: 775 - DNA, 21 bp, Artificial Sequence
  Description of Artificial Sequence: Synthetic oligonucleotide
tgggcaagggcggctttgcca

SEQ ID NO: 776 - DNA, 21 bp, Artificial Sequence
  Description of Artificial Sequence: Synthetic oligonucleotide
gggcaagggcggctttgccaa

SEQ ID NO: 777 - DNA, 21 bp, Artificial Sequence
  Description of Artificial Sequence: Synthetic oligonucleotide
ggcaagggcggctttgccaag

SEQ ID NO: 778 - DNA, 21 bp, Artificial Sequence
  Description of Artificial Sequence: Synthetic oligonucleotide
ccaagtgcttcgagatctcgg

SEQ ID NO: 779 - DNA, 21 bp, Artificial Sequence
  Description of Artificial Sequence: Synthetic oligonucleotide
catggacatcttctccctctg

SEQ ID NO: 780 - DNA, 21 bp, Artificial Sequence
  Description of Artificial Sequence: Synthetic oligonucleotide
tcgaggacaacgacttcgtgt

SEQ ID NO: 781 - DNA, 21 bp, Artificial Sequence
  Description of Artificial Sequence: Synthetic oligonucleotide
cgaggacaacgacttcgtgtt

SEQ ID NO: 782 - DNA, 21 bp, Artificial Sequence
  Description of Artificial Sequence: Synthetic oligonucleotide
gaggacaacgacttcgtgttc

SEQ ID NO: 783 - DNA, 21 bp, Artificial Sequence
  Description of Artificial Sequence: Synthetic oligonucleotide
cagttaaaaaccaccacaaca

SEQ ID NO: 784 - DNA, 21 bp, Artificial Sequence
  Description of Artificial Sequence: Synthetic oligonucleotide
gctgaatggccgggaggagga

SEQ ID NO: 785 - DNA, 21 bp, Artificial Sequence
  Description of Artificial Sequence: Synthetic oligonucleotide
tggagtggcagatgggagagc

SEQ ID NO: 786 - DNA, 21 bp, Artificial Sequence
  Description of Artificial Sequence: Synthetic oligonucleotide
aaccatgagggaaatcgtgca

SEQ ID NO: 787 - DNA, 21 bp, Artificial Sequence
  Description of Artificial Sequence: Synthetic oligonucleotide
accatgagggaaatcgtgcac

SEQ ID NO: 788 - DNA, 21 bp, Artificial Sequence
  Description of Artificial Sequence: Synthetic oligonucleotide
ttctctgtaggtggcaaatat

SEQ ID NO: 789 - protein, 6 aa, Artificial Sequence
  Description of Artificial Sequence: Synthetic 6xHis tag
HHHHHH

(19) United States
(12) Patent Application Publication
(10) Pub. No.: US 2023/0083383 A1
(43) Pub. Date: Mar. 16, 2023

(54) COMPOSITIONS AND METHODS FOR TARGETING, EDITING OR MODIFYING HUMAN GENES

(71) Applicant: DANMARKS TEKNISKE UNIVERSITET, KGS Lyngby (DK)

(72) Inventors: Ryan T. GILL, Denver, CO (US); Tanya WARNECKE, Boulder, CO (US); Roland Franz BAUMGARTNER, Angern an der March (AT)

(73) Assignee: DANMARKS TEKNISKE UNIVERSITET, KGS Lyngby (DK)

(21) Appl. No.: 17/797,986

(22) PCT Filed: Feb. 5, 2021
(86) PCT No.: PCT/US2021/016823

§ 371 Date: Aug. 5, 2022

Related U.S. Application Data
(60) Provisional application No. 62/970,455, filed on Feb. 5, 2020.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80; C12N 2310/531

(57) ABSTRACT

The present invention relates to engineered Clustered Regularly Interspaced Short Palindromic Repeals (CRISPR) systems and corresponding guide RNAs that target specific nucleotide sequences at certain gene loci in the human genome. Also provided are methods of targeting, editing, and/or modifying of the human genes using the engineered CRISPR systems, and compositions and cells comprising the engineered CRISPR systems.

### CROSS-REFERENCE TO RELATED APPLICATIONS

This application claims the benefit of and priority to U.S. Provisional Patent Application No. 62/970,455, filed Feb. 5, 2020, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jan. 28, 2021, is named ATS-002WO_SL.txt and is 333,008 bytes in size.

### FIELD OF THE INVENTION

The present invention relates to engineered Clustered Regularly Interspaced Short Palindromic Repeats (CRISPR) systems and corresponding guide RNAs that target specific nucleotide sequences at certain gene loci in the human genome, methods of targeting, editing, and/or modifying human genes using the engineered CRISPR systems, and compositions and cells comprising the engineered CRISPR systems.

### BACKGROUND OF THE INVENTION

Recent advances have been made in precise genome targeting technologies. For example, specific loci in genomic DNA can be targeted, edited, or otherwise modified by designer meganucleases, zinc finger nucleases, or transcription activator-like effectors (TALEs). Furthermore, the CRISPR-Cas systems of bacterial and archaeal adaptive immunity have been adapted for precise targeting of genomic DNA in eukaryotic cells. Compared to the earlier generations of genome editing tools, the CRISPR-Cas systems are easy to set up, scalable, and amenable to targeting multiple positions within the eukaryotic genome, thereby providing a major resource for new applications in genome engineering.

Two distinct classes of CRISPR-Cas systems have been identified. Class 1 CRISPR-Cas systems utilize multi-protein effector complexes, whereas class 2 CRISPR-Cas systems utilize single-protein effectors (see, Makarova et al. (2017) CELL, 168: 328). Among the three types of class 2 CRISPR-Cas systems, type II and type V systems typically target DNA and type VI systems typically target RNA (id.). Naturally occurring type II effector complexes consist of Cas9, CRISPR RNA (crRNA), and trans-activating CRISPR RNA (tracrRNA), but the crRNA and tracrRNA can be fused as a single guide RNA in an engineered system for simplicity (see, Wang et al. (2016) ANNU. REV. BIOCHEM., 85: 227). Certain naturally occurring type V systems, such as type V-A, type V-C, and type V-D systems, do not require tracrRNA and use crRNA alone as the guide for cleavage of target DNA (see, Zetsche et al. (2015) CELL, 163: 759; Makarova et al. (2017) CELL, 168: 328).

The CRISPR-Cas systems have been engineered for various purposes, such as genomic DNA cleavage, base editing, epigenome editing, and genomic imaging (see, e.g., Wang et al. (2016) ANNU. REV. BIOCHEM., 85: 227 and Rees et al. (2018) NAT. REV. GENET., 19: 770). Although significant developments have been made, there remains a need for new and useful CRISPR-Cas systems as powerful genome targeting tools.

### SUMMARY OF THE INVENTION

The present invention is based, in part, upon the development of engineered CRISPR-Cas systems (e.g., type V-A CRISPR-Cas systems) that can be used to target, edit, or otherwise modify specific target nucleotide sequences in human ADORA2A, B2M, CD52, CIITA, CTLA4, DCK, FAS, HAVCR2 (also called TIM3), LAG3, PDCD1 (also called PD-1), PTPN6, TIGIT, TRAC, TRBC1, TRBC2, CARD11, CD247, IL7R, LCK, or PLCG1 gene. In particular, guide nucleic acids, such as single guide nucleic acids and dual guide nucleic acids, can be designed to hybridize with the selected target nucleotide sequence and activate a Cas nuclease to edit the human genes. CRISPR-Cas systems comprising such guide nucleic acids are also useful for targeting or modifying the human genes.

A CRISPR-Cas system generally comprises a Cas protein and one or more guide nucleic acids (e.g., RNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence in the target strand of the DNA. Both PAM recognition and target nucleotide sequence hybridization are required for stable binding of a CRISPR-Cas complex to the DNA target and, if the Cas protein has an effector function (e.g., nuclease activity), activation of the effector function. As a result, when creating a CRISPR-Cas system, a guide nucleic acid can be designed to comprise a nucleotide sequence called spacer sequence that hybridizes with a target nucleotide sequence, where target nucleotide sequence is located adjacent to a PAM in an orientation operable with the Cas protein. It has been observed that not all CRISPR-Cas systems designed by these criteria are equally effective. The present invention identifies target nucleotide sequences in particular human genes that can be efficiently edited, and provides CRISPR-Cas systems directed to these target nucleotide sequences.

Accordingly, in one aspect, the present invention provides a guide nucleic acid comprising a targeter stem sequence and a spacer sequence, wherein the spacer sequence comprises a nucleotide sequence listed in Table 1, 2, or 3.

In certain embodiments, the targeter stem sequence comprises a nucleotide sequence of GUAGA. In certain embodiments, the targeter stem sequence is 5′ to the spacer sequence, optionally wherein the targeter stem sequence is linked to the spacer sequence by a linker consisting of 1, 2, 3, 4, or 5 nucleotides.

In certain embodiments, the guide nucleic acid is capable of activating a CRISPR Associated (Cas) nuclease in the absence of a tracrRNA (e.g., the guide nucleic acid being a single guide nucleic acid). In certain embodiments, the guide nucleic acid comprises from 5′ to 3′ a modulator stem sequence, a loop sequence, a targeter stem sequence, and the spacer sequence.

In certain embodiments, the guide nucleic acid is a targeter nucleic acid that, in combination with a modulator nucleic acid, is capable of activating a Cas nuclease. In certain embodiments, the guide nucleic acid comprises from 5′ to 3′ a targeter stem sequence and the spacer sequence.

In certain embodiments, the Cas nuclease is a type V Cas nuclease. In certain embodiments, the Cas nuclease is a type V-A Cas nuclease. In certain embodiments, the Cas nuclease comprises an amino acid sequence at least 80% identical to SEQ ID NO: 1. In certain embodiments, the Cas nuclease is Cpf1. In certain embodiments, the Cas nuclease recognizes a protospacer adjacent motif(PAM) consisting of the nucleotide sequence of TITN or CTTN.

In certain embodiments, the guide nucleic acid comprises a ribonucleic acid (RNA). In certain embodiments, the guide nucleic acid comprises a modified RNA. In certain embodiments, the guide nucleic acid comprises a combination of RNA and DNA. In certain embodiments, the guide nucleic acid comprises a chemical modification. In certain embodiments, the chemical modification is present in one or more nucleotides at the 5′ end of the guide nucleic acid. In certain embodiments, the chemical modification is present in one or more nucleotides at the 3′ end of the guide nucleic acid. In certain embodiments, the chemical modification is selected from the group consisting of 2′-O-methyl, 2′-fluoro, 2′-O-methoxyethyl, phosphorothioate, phosphorodithioate, pseudouridine, and any combinations thereof.

The present invention also provides an engineered, non-naturally occurring system comprising a guide nucleic acid (e.g., a single guide nucleic acid) disclosed herein. In certain embodiments, the engineered, non-naturally occurring system further comprising the Cas nuclease. In certain embodiments, the guide nucleic acid and the Cas nuclease are present in a ribonucleoprotein (RNP) complex.

The present invention also provides an engineered, non-naturally occurring system comprising the guide nucleic acid (e.g., targeter nucleic acid) disclosed herein, wherein the engineered, non-naturally occurring system further comprises the modulator nucleic acid. In certain embodiments, the engineered, non-naturally occurring system, further comprises the Cas nuclease. In certain embodiments, the guide nucleic acid, the modulator nucleic acid, and the Cas nuclease are present in an RNP complex.

In certain embodiments of the engineered, non-naturally occurring system, the spacer sequence comprises a nucleotide sequence selected from the group consisting of SEQ ID NOs: 51 and 131-137, wherein the spacer sequence is capable of hybridizing with the human ADORA2A gene. In certain embodiments, when the system is delivered into a population of human cells ex vivo, the genomic sequence at the ADORA2A gene locus is edited in at least 1.5% of the cells.

In certain embodiments of the engineered, non-naturally occurring system, the spacer sequence comprises a nucleotide sequence selected from the group consisting of SEQ ID NOs: 52, 64-66, 138-145, 622, 625-626, and 634-635, wherein the spacer sequence is capable of hybridizing with the human B2M gene. In certain embodiments, when the system is delivered into a population of human cells ex vivo, the genomic sequence at the B2M gene locus is edited in at least 1.5% of the cells.

In certain embodiments of the engineered, non-naturally occurring system, the spacer sequence comprises a nucleotide sequence selected from the group consisting of SEQ ID NOs: 724, 726-727, 730-732, 735-738, 741-742, and 744-745, wherein the spacer sequence is capable of hybridizing with the human CD247 gene. In certain embodiments, when the system is delivered into a population of human cells ex vivo, the genomic sequence at the CD247 gene locus is edited in at least 1.5% of the cells.

In certain embodiments of the engineered, non-naturally occurring system, the spacer sequence comprises a nucleotide sequence selected from the group consisting of SEQ ID NOs: 53 and 146, wherein the spacer sequence is capable of hybridizing with the human CD52 gene. In certain embodiments, when the system is delivered into a population of human cells ex vivo, the genomic sequence at the CD52 gene locus is edited in at least 1.5% of the cells.

In certain embodiments of the engineered, non-naturally occurring system, the spacer sequence comprises a nucleotide sequence selected from the group consisting of SEQ ID NOs: 54, 147-148, 636-640, 642, 644-648, 650-652, 655-656, 660-663, 666, 668, 670-671, 673-676, 678-679, and 682-685, wherein the spacer sequence is capable of hybridizing with the human CIITA gene. In certain embodiments, when the system is delivered into a population of human cells ex vivo, the genomic sequence at the CIITA gene locus is edited in at least 1.5% of the cells.

In certain embodiments of the engineered, non-naturally occurring system, the spacer sequence comprises a nucleotide sequence selected from the group consisting of SEQ ID NOs: 55, 67-70, and 149-155, wherein the spacer sequence is capable of hybridizing with the human CTLA4 gene. In certain embodiments, when the system is delivered into a population of human cells ex vivo, the genomic sequence at the CTLA4 gene locus is edited in at least 1.5% of the cells.

In certain embodiments of the engineered, non-naturally occurring system, the spacer sequence comprises a nucleotide sequence selected from the group consisting of SEQ ID NOs: 56, 71-74, and 156-159, wherein the spacer sequence is capable of hybridizing with the human DCK gene. In certain embodiments, when the system is delivered into a population of human cells ex vivo, the genomic sequence at the DCK gene locus is edited in at least 1.5% of the cells.

In certain embodiments of the engineered, non-naturally occurring system, the spacer sequence comprises a nucleotide sequence selected from the group consisting of SEQ ID NOs: 57, 75-79, and 160-173, wherein the spacer sequence is capable of hybridizing with the human FAS gene. In certain embodiments, when the system is delivered into a population of human cells ex vivo, the genomic sequence at the FAS gene locus is edited in at least 1.5% of the cells.

In certain embodiments of the engineered, non-naturally occurring system, the spacer sequence comprises a nucleotide sequence selected from the group consisting of SEQ ID NOs: 58, 80-86, and 174-187, wherein the spacer sequence is capable of hybridizing with the human HAVCR2 gene. In certain embodiments, when the system is delivered into a population of human cells ex vivo, the genomic sequence at the HAVCR2 gene locus is edited in at least 1.5% of the cells.

In certain embodiments of the engineered, non-naturally occurring system, the spacer sequence comprises a nucleotide sequence selected from the group consisting of SEQ ID NOs: 748-749 and 753-754, wherein the spacer sequence is capable of hybridizing with the human IL7R gene. In certain embodiments, when the system is delivered into a population of human cells ex vivo, the genomic sequence at the IL7R gene locus is edited in at least 1.5% of the cells.

In certain embodiments of the engineered, non-naturally occurring system, the spacer sequence comprises a nucleotide sequence selected from the group consisting of SEQ ID NOs: 59, 87, 88, and 188-198, wherein the spacer sequence is capable of hybridizing with the human LAG3 gene. In certain embodiments, when the system is delivered into a population of human cells ex vivo, the genomic sequence at the LAG3 gene locus is edited in at least 1.5% of the cells.

In certain embodiments of the engineered, non-naturally occurring system, the spacer sequence comprises the nucleotide sequence of SEQ ID NO: 757, wherein the spacer sequence is capable of hybridizing with the human LCK gene. In certain embodiments, when the system is delivered into a population of human cells ex vivo, the genomic sequence at the LCK gene locus is edited in at least 1.5% of the cells.

In certain embodiments of the engineered, non-naturally occurring system, the spacer sequence comprises a nucleotide sequence selected from the group consisting of SEQ ID NOs: 60, 89-92, and 199-201, wherein the spacer sequence is capable of hybridizing with the human PDCD1 gene. In certain embodiments, when the system is delivered into a population of human cells ex vivo, the genomic sequence at the PDCD1 gene locus is edited in at least 1.5% of the cells.

In certain embodiments of the engineered, non-naturally occurring system, the spacer sequence comprises a nucleotide sequence selected from the group consisting of of SEQ ID NOs: 759 and 761-762, wherein the spacer sequence is capable of hybridizing with the human PLCG1 gene. In certain embodiments, when the system is delivered into a population of human cells ex vivo, the genomic sequence at the PLCG1 gene locus is edited in at least 1.5% of the cells.

In certain embodiments of the engineered, non-naturally occurring system, the spacer sequence comprises a nucleotide sequence selected from the group consisting of SEQ ID NOs: 61, 93-104, and 202-213, wherein the spacer sequence is capable of hybridizing with the human PTPN6 gene. In certain embodiments, when the system is delivered into a population of human cells ex vivo, the genomic sequence at the PTPN6 gene locus is edited in at least 1.5% of the cells.

In certain embodiments of the engineered, non-naturally occurring system, the spacer sequence comprises a nucleotide sequence selected from the group consisting of SEQ ID NOs: 62, 105, and 214-217, wherein the spacer sequence is capable of hybridizing with the human TIGIT gene. In certain embodiments, when the system is delivered into a population of human cells ex vivo, the genomic sequence at the TIGIT gene locus is edited in at least 1.5% of the cells.

In certain embodiments of the engineered, non-naturally occurring system, the spacer sequence comprises a nucleotide sequence selected from the group consisting of SEQ ID NOs: 63, 106-130, and 218-241, wherein the spacer sequence is capable of hybridizing with the human TRAC gene. In certain embodiments, when the system is delivered into a population of human cells ex vivo, the genomic sequence at the TRAC gene locus is edited in at least 1.5% of the cells.

In certain embodiments of the engineered, non-naturally occurring system, the spacer sequence comprises a nucleotide sequence selected from the group consisting of SEQ ID NOs: 705-706, 711-712, 714-715, 717, and 719-720, wherein the spacer sequence is capable of hybridizing with the human TRBC2 gene. In certain embodiments, when the system is delivered into a population of human cells ex vivo, the genomic sequence at the TRBC2 gene locus is edited in at least 1.5% of the cells. In certain embodiments, the spacer sequence comprises a nucleotide sequence selected from the group consisting of SEQ ID NOs: 705-706, wherein the spacer sequence is capable of hybridizing with both the human TRBC1 gene and the human TRBC2 gene. In certain embodiments, when the system is delivered into a population of human cells ex vivo, the genomic sequence at the TRBC1 gene locus is edited in at least 1.5% of the cells.

In certain embodiments of the engineered, non-naturally occurring system, genomic mutations are detected in no more than 2% of the cells at any off-target loci by CIRCLE-Seq. In certain embodiments, genomic mutations are detected in no more than 1% of the cells at any off-target loci by CIRCLE-Seq.

In another aspect, the present invention provides a human cell comprising an engineered, non-naturally occurring system disclosed herein.

In another aspect, the present invention provides a composition comprising a guide nucleic acid, engineered, non-naturally occurring system, or human cell disclosed herein.

In another aspect, the present invention provides a method of cleaving a target DNA comprising the sequence of a preselected target gene or a portion thereof, the method comprising contacting the target DNA with an engineered, non-naturally occurring system disclosed herein, thereby resulting in cleavage of the target DNA. In certain embodiments, the contacting occurs in vitro. In certain embodiments, the contacting occurs in a cell ex vivo. In certain embodiments, the target DNA is genomic DNA of the cell.

In another aspect, the present invention provides a method of editing human genomic sequence at a preselected target gene locus, the method comprising delivering an engineered, non-naturally occurring system disclosed herein into a human cell, thereby resulting in editing of the genomic sequence at the target gene locus in the human cell. In certain embodiments, the cell is an immune cell. In certain embodiments, the immune cell is a T lymphocyte.

In certain embodiments, the method of editing human genomic sequence at a preselected target gene locus comprises delivering an engineered, non-naturally occurring system disclosed herein into a population of human cells, thereby resulting in editing of the genomic sequence at the target gene locus in at least a portion of the human cells. In certain embodiments, the population of human cells comprises human immune cells. In certain embodiments, the population of human cells is an isolated population of human immune cells. In certain embodiments, the immune cells are T lymphocytes.

In certain embodiments of the method of editing human genomic sequence at a preselected target gene locus, the engineered, non-naturally occurring system is delivered into the cell(s) as a pre-formed RNP complex. In certain embodiments, the pre-formed RNP complex is delivered into the cell(s) by electroporation.

In certain embodiments, the target gene is human ADORA2A gene, wherein the spacer sequence comprises a nucleotide sequence selected from the group consisting of SEQ ID NOs: 51 and 131-137. In certain embodiments, the genomic sequence at the ADORA2A gene locus is edited in at least 1.5% of the human cells.

In certain embodiments, the target gene is human B2M gene, wherein the spacer sequence comprises a nucleotide sequence selected from the group consisting of SEQ ID NOs: 52, 64-66, 138-145, 622, 625-626, and 634-635. In certain embodiments, the genomic sequence at the B2M gene locus is edited in at least 1.5% of the human cells.

In certain embodiments, the target gene is human CD52 gene, wherein the spacer sequence comprises a nucleotide sequence selected from the group consisting of SEQ ID NOs: 53 and 146. In certain embodiments, the genomic sequence at the CD52 gene locus is edited in at least 1.5% of the human cells.

In certain embodiments, the target gene is human CD247 gene, wherein the spacer sequence comprises a nucleotide sequence selected from the group consisting of SEQ ID NOs: 724, 726-727, 730-732, 735-738, 741-742, and 744-745. In certain embodiments, the genomic sequence at the CD247 gene locus is edited in at least 1.5% of the human cells.

In certain embodiments, the target gene is human CIITA gene, wherein the spacer sequence comprises a nucleotide sequence selected from the group consisting of SEQ ID NOs: 54, 147-148, 636-640, 642, 644-648, 650-652, 655-656, 660-663, 666, 668, 670-671, 673-676, 678-679, and 682-685. In certain embodiments, the genomic sequence at the CIITA gene locus is edited in at least 1.5% of the human cells.

In certain embodiments, the target gene is human CTLA4 gene, wherein the spacer sequence comprises a nucleotide sequence selected from the group consisting of SEQ ID NOs: 55, 67-70, and 149-155. In certain embodiments, the genomic sequence at the CTLA4 gene locus is edited in at least 1.5% of the human cells.

In certain embodiments, the target gene is human DCK gene, wherein the spacer sequence comprises a nucleotide sequence selected from the group consisting of SEQ ID NOs: 56, 71-74, and 156-159. In certain embodiments, the genomic sequence at the DCK gene locus is edited in at least 1.5% of the human cells.

In certain embodiments, the target gene is human FAS gene, wherein the spacer sequence comprises a nucleotide sequence selected from the group consisting of SEQ ID NOs: 57, 75-79, and 160-173. In certain embodiments, the genomic sequence at the FAS gene locus is edited in at least 1.5% of the human cells.

In certain embodiments, the target gene is human HAVCR2 gene, wherein the spacer sequence comprises a nucleotide sequence selected from the group consisting of SEQ ID NOs: 58, 80-86, and 174-187. In certain embodiments, the genomic sequence at the HAVCR2 gene locus is edited in at least 1.5% of the human cells.

In certain embodiments, the target gene is human IL7R gene, wherein the spacer sequence comprises a nucleotide sequence selected from the group consisting of SEQ ID NOs: 748-749 and 753-754. In certain embodiments, the genomic sequence at the IL7R gene locus is edited in at least 1.5% of the human cells.

In certain embodiments, the target gene is human LAG3 gene, wherein the spacer sequence comprises a nucleotide sequence selected from the group consisting of SEQ ID NOs: 59, 87, 88, and 188-198. In certain embodiments, the genomic sequence at the LAG3 gene locus is edited in at least 1.5% of the human cells.

In certain embodiments, the target gene is human LCK gene, wherein the spacer sequence comprises the nucleotide sequence of SEQ ID NO: 757. In certain embodiments, the genomic sequence at the LCK gene locus is edited in at least 1.5% of the human cells.

In certain embodiments, the target gene is human PDCD1 gene, wherein the spacer sequence comprises a nucleotide sequence selected from the group consisting of SEQ ID NOs: 60, 89-92, and 199-201. In certain embodiments, the genomic sequence at the PDCD1 gene locus is edited in at least 1.5% of the human cells.

In certain embodiments, the target gene is human PLCG1 gene, wherein the spacer sequence comprises a sequence of SEQ ID NO: 759 and 761-762. In certain embodiments, the genomic sequence at the PLCG1 gene locus is edited in at least 1.5% of the human cells.

In certain embodiments, the target gene is human PTPN6 gene, wherein the spacer sequence comprises a nucleotide sequence selected from the group consisting of SEQ ID NOs: 61, 93-104, and 202-213. In certain embodiments, the genomic sequence at the PTPN6 gene locus is edited in at least 1.5% of the human cells.

In certain embodiments, the target gene is human TIGIT gene, wherein the spacer sequence comprises a nucleotide sequence selected from the group consisting of SEQ ID NOs: 62, 105, and 214-217. In certain embodiments, the genomic sequence at the TIGIT gene locus is edited in at least 1.5% of the human cells.

In certain embodiments, the target gene is human TRAC gene, wherein the spacer sequence comprises a nucleotide sequence selected from the group consisting of SEQ ID NOs: 63, 106-130, and 218-241. In certain embodiments, the genomic sequence at the TRAC gene locus is edited in at least 1.5% of the human cells.

In certain embodiments, the target gene is human TRBC2 gene, wherein the spacer sequence comprises a nucleotide sequence selected from the group consisting of SEQ ID NOs: 705-706, 711-712, 714-715, 717, and 719-720. In certain embodiments, the genomic sequence at the TRBC2 gene locus is edited in at least 1.5% of the human cells. In certain embodiments, the method further results in editing of the genomic sequence at human TRBC1 gene locus in the human cell, wherein the spacer sequence comprises a nucleotide sequence selected from the group consisting of SEQ ID NOs: 705-706. In certain embodiments, the genomic sequence at the TRBC1 gene locus is edited in at least 1.5% of the human cells.

In certain embodiments, genomic mutations are detected in no more than 2% of the cells at any off-target loci by CIRCLE-Seq. In certain embodiments, genomic mutations are detected in no more than 1% of the cells at any off-target loci by CIRCLE-Seq.

### BRIEF DESCRIPTION OF THE DRAWINGS

FIG. 1A is a schematic representation showing the structure of an exemplary single guide type V-A CRISPR system. FIG. 1B is a schematic representation showing the structure of an exemplary dual guide type V-A CRISPR system.

FIGS. 2A-2C are a series of schematic representation showing incorporation of a protecting group (e.g., a protective nucleotide sequence or a chemical modification) (FIG. 2A), a donor template-recruiting sequence (FIG. 2B), and an editing enhancer (FIG. 2C) into a type V-A CRISPR-Cas system. These additional elements are shown in the context of a dual guide type V-A CRISPR system, but it is understood that they can also be present other CRISPR systems, including a single guide type V-A CRISPR system, a single guide type II CRISPR system, or a dual guide type II CRISPR system.

### DETAILED DESCRIPTION OF THE INVENTION

The present invention is based, in part, upon the development of engineered CRISPR-Cas systems (e.g., type V-A CRISPR-Cas systems) that can be used to target, edit, or otherwise modify specific target nucleotide sequences in human ADORA2A, B2M, CD52, CIITA, CTLA4, DCK, FAS, HAVCR2 (also called TIM3), LAG3, PDCD1 (also called PD-1), PTPN6, TIGIT, TRAC, TRBC1, TRBC2, CARD11, CD247, IL7R, LCK, or PLCG1 gene. In particular, guide nucleic acids, such as single guide nucleic acids and dual guide nucleic acids, can be designed to hybridize with the selected target nucleotide sequence and activate a Cas nuclease to edit the human genes. CRISPR-Cas systems comprising such guide nucleic acids are also useful for targeting or modifying the human genes.

A CRISPR-Cas system generally comprises a Cas protein and one or more guide nucleic acids (e.g., RNAs). The Cas protein can be directed to a specific location in a double-stranded DNA target by recognizing a protospacer adjacent motif (PAM) in the non-target strand of the DNA, and the one or more guide nucleic acids can be directed to a specific location by hybridizing with a target nucleotide sequence in the target strand of the DNA. Both PAM recognition and target nucleotide sequence hybridization are required for stable binding of a CRISPR-Cas complex to the DNA target and, if the Cas protein has an effector function (e. g. nuclease activity), activation of the effector function. As a result, when creating a CRISPR-Cas system, a guide nucleic acid can be designed to comprise a nucleotide sequence called spacer sequence that hybridizes with a target nucleotide sequence, where target nucleotide sequence is located adjacent to a PAM in an orientation operable with the Cas protein. It has been observed that not all CRISPR-Cas systems designed by these criteria are equally effective. The present invention identifies target nucleotide sequences in particular human genes that can be efficiently edited, and provides CRISPR-Cas systems directed to these target nucleotide sequences.

Naturally occurring Type V-A, type V-C, and type V-D CRISPR-Cas systems lack a tracrRNA and rely on a single crRNA to guide the CRISPR-Cas complex to the target DNA. Dual guide nucleic acids capable of activating type V-A, type V-C, or type V-D Cas nucleases have been developed, for example, by splitting the single crRNA into a targeter nucleic acid and a modulator nucleic acid (see, U.S. Provisional Patent Application No. 62/910,055). Naturally occurring type V-A Cas proteins comprise a RuvC-like nuclease domain but lack an HNH endonuclease domain, and recognize a 5′ T-rich PAM located immediately upstream from the target nucleotide sequence, the orientation determined using the non-target strand (i.e., the strand not hybridized with the spacer sequence) as the coordinate. The CRISPR-Cas systems cleave a double-stranded DNA to generate a staggered double-stranded break rather than a blunt end. The cleavage site is distant from the PAM site (e.g., separated by at least 10, 11, 12, 13, 14, or 15 nucleotides downstream from the PAM on the non-target strand and/or separated by at least 15, 16, 17, 18, or 19 nucleotides upstream from the sequence complementary to PAM on the target strand).

Naturally occurring type II CRISPR-Cas systems (e.g., CRISPR-Cas9 systems) generally comprise two guide nucleic acids, called crRNA and tracrRNA, which form a complex by nucleotide hybridization. Single guide nucleic acids capable of activating type 11 Cas nucleases have been developed, for example, by linking the crRNA and the tracrRNA (see, e.g., U.S. Patent Application Publication Nos. 2014/0242664 and 2014/0068797). Naturally occurring type II Cas proteins comprise a RuvC-like nuclease domain and an HNH endonuclease domain, and recognize a 3′ G-rich PAM located immediately downstream from the target nucleotide sequence, the orientation determined using the non-target strand (i.e., the strand not hybridized with the spacer sequence) as the coordinate. The CRISPR-Cas systems cleave a double-stranded DNA to generate a blunt end. The cleavage site is generally 3-4 nucleotides upstream from the PAM on the non-target strand.

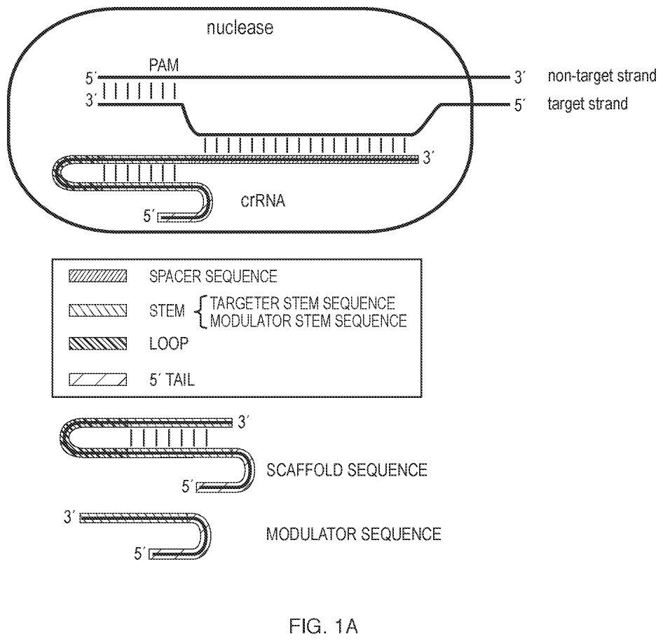

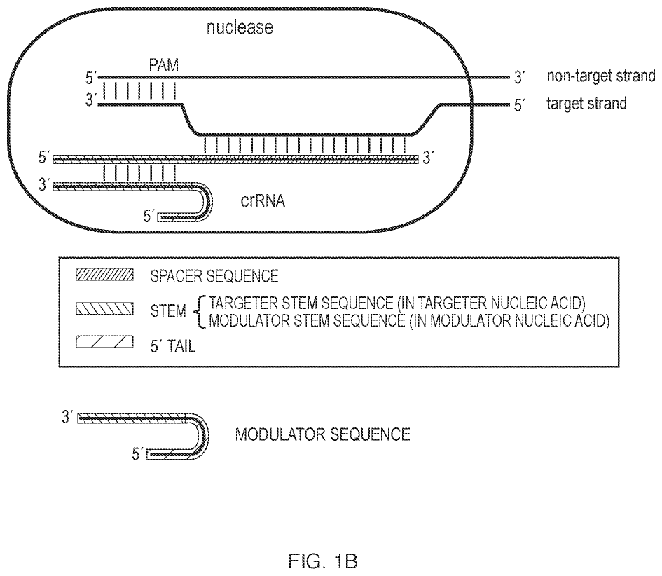

Elements in an exemplary single guide type V-A CRISPR-Cas system are shown in FIG. 1A. The single guide nucleic acid is also called a “crRNA” where it is present in the form of an RNA. It comprises, from 5′ to 3′, an optional 5′ tail, a modulator stem sequence, a loop, a targeter stem sequence complementary to the modulator stem sequence, and a spacer sequence that hybridizes with the target strand of the target DNA. Where a 5′ tail is present, the sequence including the 5′ tail and the modulator stem sequence is also called a “modulator sequence” herein. A fragment of the single guide nucleic acid from the optional 5′ tail to the targeter stem sequence, also called a “scaffold sequence” herein, bind the Cas protein. In addition, the PAM in the non-target strand of the target DNA binds the Cas protein.

Elements in an exemplary dual guide type V-A CRISPR-Cas system are shown in FIG. 1B. The first guide nucleic acid, called “modulator nucleic acid” herein, comprises, from 5′ to 3′, an optional 5′ tail and a modulator stem sequence. Where a 5′ tail is present, the sequence including the 5′ tail and the modulator stem sequence is also called a “modulator sequence” herein. The second guide nucleic acid, called “targeter nucleic acid” herein, comprises, from 5′ to 3′, a targeter stem sequence complementary to the modulator stem sequence and a spacer sequence that hybridizes with the target strand of the target DNA. The duplex between the modulator stem sequence and the targeter stem sequence, plus the optional 5′ tail, constitute a structure that binds the Cas protein. In addition, the PAM in the non-target strand of the target DNA binds the Cas protein.

The terms “targeter stem sequence” and “modulator stem sequence,” as used herein, refer to a pair of nucleotide sequences in one or more guide nucleic acids that hybridize with each other. When a targeter stem sequence and a modulator stem sequence are contained in a single guide nucleic acid, the targeter stem sequence is proximal to a spacer sequence designed to hybridize with a target nucleotide sequence, and the modulator stem sequence is proximal to the targeter stem sequence. When a targeter stem sequence and a modulator stem sequence are in separate nucleic acids, the targeter stem sequence is in the same nucleic acid as a spacer sequence designed to hybridize with a target nucleotide sequence. In a CRISPR-Cas system that naturally includes separate crRNA and tracrRNA (e.g., a type II system), the duplex formed between the targeter stem sequence and the modulator stem sequence corresponds to the duplex formed between the crRNA and the tracrRNA. In a CRISPR-Cas system that naturally includes a single crRNA but no tracrRNA (e.g., a type V-A system), the duplex formed between the targeter stem sequence and the modulator stem sequence corresponds to the stem portion of a stem-loop structure in the scaffold sequence (also called direct repeat sequence) of the crRNA. It is understood that 100% complementarity is not required between the targeter stem sequence and the modulator stem sequence. In a type V-A CRISPR-Cas system, however, the targeter stem sequence is typically 100% complementary to the modulator stem sequence.

The term “targeter nucleic acid,” as used herein in the context of a dual guide CRISPR-Cas system, refers to a nucleic acid comprising (i) a spacer sequence designed to hybridize with a target nucleotide sequence; and (ii) a targeter stem sequence capable of hybridizing with an additional nucleic acid to form a complex, wherein the complex is capable of activating a Cas nuclease (e.g., a type II or type V-A Cas nuclease) under suitable conditions, and wherein the targeter nucleic acid alone, in the absence of the additional nucleic acid, is not capable of activating the Cas nuclease under the same conditions.

The term “modulator nucleic acid,” as used herein in connection with a given targeter nucleic acid and its corresponding Cas nuclease, refers to a nucleic acid capable of hybridizing with the targeter nucleic acid to form a complex, wherein the complex, but not the modulator nucleic acid alone, is capable of activating the type Cas nuclease under suitable conditions.

The term “suitable conditions,” as used in connection with the definitions of “targeter nucleic acid” and “modulator nucleic acid,” refers to the conditions under which a naturally occurring CRISPR-Cas system is operative, such as in a prokaryotic cell, in a eukaryotic (e.g., mammalian or human) cell, or in an in vitro assay.

The features and uses of the guide nucleic acids and CRISPR-Cas systems are discussed in the following sections.

### I. Guide Nucleic Acids and Engineered, Non-Naturally Occurring CRISPR-Cas Systems

The present invention provides a guide nucleic acid comprising a targeter stem sequence and a spacer sequence, wherein the spacer sequence comprises a nucleotide sequence listed Table 1, 2, or 3, or a portion thereof sufficient to hybridize with the corresponding target gene listed in the table. In particular, Table 1 lists the guide nucleic acid that showed the best editing efficiency for each target gene using the method described in Example 1. Table 2 lists the guide nucleic acids that showed at least 10% editing efficiency using the method described in Example 1. Table 3 lists the guide nucleic acids that showed at least 1.5% and lower than 10% editing efficiency using the method described in Example 1.

In certain embodiments, a guide nucleic acid of the present invention is capable of binding the genomic locus of the corresponding target gene in the human genome. In certain embodiments, a guide nucleic acid of the present invention, alone or in combination with a modulator nucleic acid, is capable of directing a Cas protein to the genomic locus of the corresponding target gene in the human genome. In certain embodiments, a guide nucleic acid of the present invention, alone or in combination with a modulator nucleic acid, is capable of directing a Cas nuclease to the genomic locus of the corresponding target gene in the human genome, thereby resulting in cleavage of the genomic DNA at the genomic locus.

TABLE 1
Selected Spacer Sequences
Targeting Human Genes

| Target | | | SEQ ID |
|---|---|---|---|
| Gene | crRNA | Spacer Sequence | NO |
| TRAC | gTRAC006 | TGAGGGTGAAGGATAGACGCT | 63 |
| ADORA2A | gADORA2A_12 | AGGATGTGGTCCCCATGAACT | 51 |
| B2M | gB2M41 | ATAGATCGAGACATGTAAGCA | 635 |
| CARD11 | gCARD11_1 | TAGTACCGCTCCTGGAAGGTT | 721 |
| CD247 | gCD247_12 | CTAGCAGAGAAGGAAGAACCC | 735 |
| CD52 | gCD52_1 | CTCTTCCTCCTACTCACCATC | 53 |
| CIITA | gCIITA_32 | CCTTGGGGCTCTGACAGGTAG | 636 |
| CTLA4 | gCTLA4_4 | AGCGGCACAAGGCTCAGCTGA | 55 |
| DCK | gDCK_6 | CGGAGGCTCCTTACCGATGTT | 56 |
| FAS | gFAS_36 | GTGTTGCTGGTGAGTGTGCAT | 57 |
| HAVCR2 | gTIM3_6 | CTTGTAAGTAGTAGCAGCAGC | 58 |
| IL7R | gIL7R_3 | CAGGGGAGATGGATCCTATCT | 749 |
| LAGS | gLAG3_6 | GGGTGCATACCTGTCTGOCTG | 59 |
| LCK | gLCK1_3 | ACCCATCAACCCGTAGGGATG | 757 |
| PDCD1 | gPD_23 | TCTGCAGGGACAATAGGAGCC | 60 |
| PLCG1 | gPLCG1_2 | CCTTTCTGCGCTTCGTGGTGT | 759 |
| PTPN6 | gPTPN6_6 | TATGACCTGTATGGAGGGGAG | 61 |
| TIGIT | gTIGIT_2 | AGGCCTTACCTGAGGCGAGGG | 62 |
| TRBC1+2 | gTRBC1+2_3 | CGCTGTCAAGTCCAGTTCTAC | 706 |
| TRBC2 | gTRBC2_12 | CCGGAGGTGAAGCCACAGTCT | 712 |

TABLE 2
Selected Spacer Sequences
Targeting Human Genes

| Target | | | SEQ ID |
|---|---|---|---|
| Gene | crRNA | Spacer sequence | NO |
| ADORA2A | gADORA2A_12 | AGGATGTGGTCCCCATGAACT | 51 |
| B2M | gB2M_4 | CTCACGTCATCCAGCAGAGAA | 52 |
| B2M | giGM_7 | ACTTTCCATTCTCTGCTGGAT | 64 |
| B2M | gB2M_2 | TGGCCTOGAGGCTATCCAGCG | 65 |
| B2M | gB2M_17 | TATCTCTTGTACTACACTGAA | 66 |
| B2M | gB2M_30 | AGTGGGGGTGAATTCAGTGTA | 625 |
| B2M | gB2M_41 | ATAGATCGAGACATGTAAGCA | 635 |
| CIITA | gCIITA_32 | CCTTGGGGCTCTGACAGGTAG | 636 |
| CIITA | gCIITA_33 | ACCTTGGGGCTCTGACAGGTA | 637 |
| CIITA | gCIITA_35 | CTCCCAGAACCCGACACAGAC | 639 |
| CIITA | gCIITA_36 | TGGGCTCAGGTGCTTCCTCAC | 640 |
| CIITA | gCIITA_38 | CTTGTCTOGGCAGCGGAACTG | 642 |
| CIITA | gCIITA_40 | TCAAAGTAGAGCACATAGGAC | 644 |
| CIITA | gCIITA_41 | TGCCCAACTTCTGCTGGCATC | 645 |
| CIITA | gCIITA_43 | TCTGCAGCCTTCCCAGAGGAG | 647 |
| CIITA | gCIITA_44 | TCCAGGCGCATCTGGCCGGAG | 648 |
| CIITA | gCIITA_48 | CTCGGGAGGTCAGGGCAGGTT | 652 |
| CIITA | gCIITA_57 | CAGAAGAAGCTGCTCCGAGGT | 660 |
| CIITA | gCIITA_59 | AGAGCTCAGGGATGACAGAGC | 662 |
| CIITA | gCIITA_60 | TGCCGGGCAGTGTGCCAGCTC | 663 |
| CIITA | gCIITA_63 | GCCACTCAGAGCCAGCCACAG | 666 |
| CIITA | gCIITA_65 | GCAGCACGTGGTACAGGAGCT | 668 |
| CIITA | gCIITA_67 | TGGGCACCCGCCTCACOCCTC | 670 |
| CIITA | gCIITA_70 | CCAGGTCTTCCACATCCTTCA | 673 |
| CIITA | gCIITA_71 | AAAGCCAAGTCCCTGAAGGAT | 674 |
| CIITA | gCIITA_72 | GGTCCCGAACAGCAGGGAGCT | 675 |
| CIITA | gCIITA_73 | TTTAGGTCCCGAACAGCAGGG | 676 |
| CIITA | gCIITA_76 | GGGAAAGCCTGGGGGCCTGAG | 679 |
| CIITA | gCIITA_80 | CAAGGACTTCAGCTGGGGGAA | 682 |
| CIITA | gCIITA_81 | TAGGCACCCAGGTCAGTGATG | 683 |
| CIITA | gCIITA_82 | CGACAGCTTGTACAATAACTG | 684 |
| CD247 | gCD247_1 | TGTGTTGCAGTTCAGCAGGAG | 724 |
| CD247 | gCD247_3 | CGGAGGGTCTACGGCGAGGCT | 726 |
| CD247 | gCD247_4 | TTATCTGTTATAGGAGCTCAA | 727 |
| CD247 | gCD247_8 | GACAAGAGACGTGGCCGGGAC | 731 |
| CD247 | gCD247_12 | CTAGCAGAGAAGGAAGAACCC | 735 |
| CD247 | gCD247_15 | ATCCCAATCTCACTGTAGGCC | 738 |
| CD247 | gCD247_18 | TCATTTCACTCCCAAACAACC | 741 |
| CD247 | gCD247_19 | ACTCCCAAACAACCAGCGCCG | 742 |
| CD52 | gCD52_1 | CTCTTCCTCCTACTCACCATC | 53 |
| CIITA | gCIITA_4 | TAGGGGCCCCAACTCCATGGT | 54 |
| CTLA4 | gCTLA4_4 | AGCGGCACAAGGCTCAGCTGA | 55 |
| CTLA4 | gCTLA4_14 | CCTGGAGATGCATACTCACAC | 67 |
| CTLA4 | gCTLA4_6 | CAGAAGACAGGGATGAAGAGA | 68 |
| (TLA4 | gCTLA4_19 | CACTGGAGGTGCCCGTGCAGA | 69 |
| CTLA4 | gCTLA4_13 | TGTGTGAGTATGCATCTCCAG | 70 |
| DCK | gDCK_6 | CGGAGGCTCCTTACCGATGTT | 56 |
| DCK | gDCK_2 | TCAGCCAGCTCTGAGGGGACC | 71 |
| DCK | gDCK_8 | CTCACAACAGCTGCAGGGAAG | 72 |
| DCK | gDCK_26 | AGCTTGCCATTCAGAGAGGCA | 73 |
| DCK | gDCK_30 | TACATACCTGTCACTATACAC | 74 |
| FAS | gFAS_36 | GTGTTOCTGGTGAGTGTGCAT | 57 |
| FAS | gFAS_34 | TTTTTCTAGATGTGAACATGG | 75 |
| FAS | gFAS_35 | ATGATTCCATGTTCACATCTA | 76 |
| FAS | gFAS_12 | GTGTAACATACCTGGAGGACA | 77 |
| FAS | gFAS_1 | GGAGGATTGCTCAACAACCAT | 78 |
| FAS | gFAS_59 | TAGGAAACAGTGGCAATAAAT | 79 |
| HAVCR2 | gTIM3_6 | CTTGTAAGTAGTAGCAGCAGC | 58 |
| HAVCR2 | gTIM3_29 | CAAGGATGCTTACCACCAGGG | 80 |
| HAVCR2 | gTIM3_6 | TAAGTAGTAGCAGCAGCAGCA | 81 |
| HAVCR2 | gTIM3_32 | TATCAGGGAGGCTCCCCAGTG | 82 |
| HAVCR2 | gTIM3_30 | CCACCAGGGGACATGGCCCAG | 83 |
| HAVCR2 | gTIM3_12 | AATGTGGCAACGTGGTGCTCA | 84 |
| HAVCR2 | gTIM3_25 | TGACATTAGCCAAGGTCACCC | 85 |
| HAVCR2 | gGM3_18 | CGCAAAGGAGATGTGTCCCTG | 86 |
| IL7R | gIL7R+3 | CAGGGGAGATGGATCCTATCT | 749 |
| IL7R | gIL7R_8 | CATAACACACAGGCCAAGATG | 754 |
| LAG3 | gLAG3_6 | GGGTGCATACCTGTCTGGCTG | 59 |
| LAG3 | gLAG3_38 | TCAGGACCTTGGCTGGAGGCA | 87 |
| LAG3 | gLAG3_33 | GGTCACCTGGATCCCTGGGGA | 88 |
| LCK | gLCK1_3 | ACCCATCAACCCGTAGGGATG | 757 |
| PDCD1 | gPD_23 | TCTOCAGGGACAATAGGAGCC | 60 |
| PDCD1 | gPD_2 | CCTTCCGCTCACCTCCGCCTG | 89 |
| PDCD1 | gPD_8 | GCACGAAGCTCTCCGATGTGT | 90 |
| PDCD1 | gPD_29 | CTAGCGGAATGGGCACCTCAT | 91 |
| PDCD1 | gPD_27 | CAGTGGCGAGAGAAGACCCCG | 92 |
| PTPN6 | gPTPN6_6 | TATGACCTGTATGGAGGGGAG | 61 |
| PTPN6 | gPTPN6_46 | ACTGCCCCCCACCCAGGCCTG | 93 |
| PTPN6 | gPTPN6_7 | CGACTCTGACAGAGCTGGTGG | 94 |
| PTPN6 | gPTPN6_26 | CAGAAGCAGGAGGTGAAGAAC | 95 |
| PTPN6 | gPTPN6_1 | ACCGAGACCTCAGTGGGCTGG | 96 |
| PTPN6 | gPTPN6_37 | TGGGCCCTACTCTGTGACCAA | 97 |
| PTPN6 | gPTPN6_16 | TGTGCTCAGTGACCAGCCCAA | 98 |
| PTPN6 | gPTPN6_25 | CCCACCCACATCTCAGAGTTT | 99 |
| PTPN6 | gPTPN6_12 | TTGTGCGTGAGAGCCTCAGCC | 100 |
| PTPN6 | gPTPN6_22 | AAGAAGACGGGGATTGAGGAG | 101 |
| PTPN6 | gPTPN6_5 | TCCCCTCCATACAGGTCATAG | 102 |
| PTPN6 | gPTPN6_19 | GCTCCCCCCAGGGTGGACGCT | 103 |
| PTPN6 | gPTPXG 14 | GGCTGGTCACTGAGCACAGAA | 104 |
| TIGIT | gTIGIT_2 | AGGCCTTACCTOAGGCGAGGG | 62 |
| TIGIT | gTIGIT_18 | GTCCTCCCTCTAGTOGCTGAG | 105 |
| TRAC | gTRAC006 | TGAGGGTGAAGGATAGACGCT | 63 |
| TRAC | gTRAC073 | GCAGACAGGGAGAAATAAGGA | 106 |
| TRAC | gTRAC017 | CAGGTGAAATTCCTGAGATGT | 107 |
| TRAC | gTRAC059 | GACATCATTGACCAGAGCTCT | 108 |
| TRAC | gTRAC078 | CCAGCTCACTAAGTCAGTCTC | 109 |
| TRAC | gTRAC012 | TATGGAGAAGCTCTCATTTCT | 110 |
| TRAC | gTRAC039 | TAAGATGCTATTTCCCGTATA | 111 |
| TRAC | gTRAC067 | CCGTGTCATTCTCTGGACTGC | 112 |
| TRAC | gTRAC079 | ATTCCTCCACTTCAACACCTG | 113 |
| TRAC | gTRAC038 | TACGGGAAATAGCATCTTAGA | 114 |
| TRAC | gTRAC061 | GTGGCAATGGATAAGGCCGAG | 115 |
| TRAC | gTRAC058 | CTTGCTTCAGGAATGGCCAGG | 116 |
| TRAC | gTRAC021 | TAGTTCAAAACCTCTATCAAT | 117 |
| TRAC | gTRAC049 | TCTGTGATATACACATCAGAA | 118 |
| TRAC | gTRAC074 | GGCAGACAGGGAGAAATAAGG | 119 |
| TRAC | gTRAC018 | CTCGATATAAGGCCTTGAGCA | 120 |
| TRAC | gTRAC043 | GAGTCTCTCAGCTGGTACACG | 121 |
| TRAC | gTRAC075 | TGGCAGACAGGGAGAAATAAG | 122 |
| TRAC | gTRAC082 | CCAGCTGACAGATGGGCTCCC | 123 |
| TRAC | gTRAC040 | CCGTATAAAGCATGAGACCGT | 124 |
| TRAC | gTRAC041 | CCCCAACCCAGGCTGGAGTCC | 125 |
| TRAC | gTRAC076 | TTGGCAGACAGGGAGAAATAA | 126 |
| TRAC | gTRAC014 | TCAGAAGAGCCTGGCTAGGAA | 127 |
| TRAC | gTRAC029 | CTCTGCCAGAGTTATATTGCT | 128 |
| TRAC | gTRAC028 | CCATGCCTGCCTTTACTCTGC | 129 |
| TRAC | gTRAC050 | GTCTGTGATATACACATCAGA | 130 |
| TRBCl+2 | gTRBC1+2_1 | AGCCATCAGAAGCAGAGATCT | 705 |
| TRBCl+2 | gTRBC1+2_3 | CGCTGTCAAGTCCAGTTCTAC | 706 |
| TRBC2 | gTRBC2_11 | AGACFGTGGCTTCACCTCCGG | 711 |
| TRBC2 | gTRBC2_12 | CCGGAGGTGAAGCCACAGTCT | 712 |
| TRBC2 | gTRBC2_15 | CTAGGGAAGGCCACCTTGTAT | 715 |
| TRBC2 | gTRBC2_21 | GAGCTAGCCTCTGGAATCCTT | 720 |

TABLE 3
Selected Spacer Sequences
Targeting Human Genes

| Target | | | SEQ ID |
|---|---|---|---|
| Gene | crRNA | Spacer sequence | NO |
| ADORA2A | gADORA2A_16 | CGGATCTTCCTGGCGGCGCGA | 131 |
| ADORA2A | gADORA2A_28 | AAGGCAGCTGGCACCAGTGCC | 132 |
| ADORA2A | gADORA2A_2 | TGGTGTCACTGGCGGCGGCCG | 133 |
| ADORA2A | gADORA2A_23 | TTCTGCCCCGACTGCAGCCAC | 134 |
| ADORA2A | gADORA2A_7 | GTGACCGGCACGAGGGCTAAG | 135 |
| ADORA2A | gADORA2A_8 | CCATCGGCCTGACTCCCATGC | 136 |
| ADORA2A | gADORA2A_4 | CCATCACCATCAGCACCGGGT | 137 |
| B2M | gB2M_21 | TCACAGCCCAAGATAGTTAAG | 138 |
| B2M | gB2M_8 | CTGAATTGCTATGTGTCTGGG | 139 |
| B2M | gB2M_11 | CTGAAGAATGGAGAGAGAATT | 140 |
| B2M | gB2M_18 | TCAGTGGGGGTGAATTCAGTG | 141 |
| B2M | gB2M_5 | CATTCTCTGCTGGATGACGTG | 142 |
| B2M | gB2M_10 | ATCCATCCGACATTGAAGTTG | 143 |
| B2M | gB2M_22 | CCCCACTTAACTATCTTGGGC | 144 |
| B2M | gB2M_1 | GCTGTGCTCGCGCTACTCTCT | 145 |
| B2M | gB2M_27 | AATTCTCTCTCCATTCTTCAG | 622 |
| B2M | gB2M_31 | CAGTGGGGGTGAATTCAGTGT | 626 |
| B2M | gB2M_40 | CATAGATCGAGACATGTAAGC | 634 |
| CD247 | gCD247_7 | CCCCCATCTCAGGGTCCCGGC | 730 |
| CD247 | gCD247_9 | TCTCCCTCTAACGTCTTCCCG | 732 |
| CD247 | gCD247_13 | TGCAGTTCCTGCAGAAGAGGG | 736 |
| CD247 | gCD247_14 | TGCAGGAACTGCAGAAAGATA | 737 |
| CD247 | gCD247_21 | TGATTTGCTTTCACGCCAGGG | 744 |
| CD247 | gCD247_22 | CTTTCACGCCAGGGTCTCAGT | 745 |
| CD52 | gCD52_4 | GCTGGTGTCGTTTTGTCCTGA | 146 |
| CIITA | gCIITA_18 | TGCTOGCATCTCCATACTCTC | 147 |
| CIITA | gCIITA_29 | GTCTCTTGCAGTGCCTTTCTC | 148 |
| CIITA | gCIITA_34 | CCGGCCTTTTTACCTTGGGGC | 638 |
| CIITA | gCIITA_42 | TGACTTTTCTGCCCAACTTCT | 646 |
| CIITA | gCIITA_46 | CCAGAGCCCATGGGGCAGAGT | 650 |
| CIITA | gCIITA_47 | TCCCCACCATCTCCACTCTGC | 651 |
| CIITA | gCIITA_51 | CAGAGCCGGTGGAGCAGTTCT | 655 |
| CIITA | gCIITA_52 | CCCAGCACAGCAATCACTCGT | 656 |
| CIITA | gCIITA_55 | AGCCACATCTTGAAGAGACCT | 658 |
| CIITA | gCIITA_58 | AGCTGTCCGGCTTCTCCATGG | 661 |
| CIITA | gCIITA_68 | CCCCTCTGGATTGGGGAGCCT | 671 |
| CIITA | gCIITA_75 | CCTCCTAGGCTGGGCCCTGTC | 678 |
| CIITA | gCIITA_83 | TCTTGCCAGCGTCCAGTACAA | 685 |
| CTLA4 | gCTLA4_27 | CTGTTGCAGATCCAGAACCGT | 149 |
| CTLA4 | gCTLA4_36 | ACAGCTAAAGAAAAGAAGCCC | 150 |
| CTLA4 | gCTLA4_41 | TCAATTGATGGGAATAAAATA | 151 |
| CTLA4 | gCTLA4_28 | CTCCTCTGGATCCTTGCAGCA | 152 |
| CTLA4 | gCTLA4_37 | CACATAGACCCCTGTTGTAAG | 153 |
| CTLA4 | gCTLA4_18 | CTAGATGATTCCATCTGCACG | 154 |
| CTLA4 | gCTLA4_5 | TTCTTCTCTTCATCCCTGTCT | 155 |
| DCK | gDCK_9 | AGGATATTCACAAATGTTGAC | 156 |
| DCK | gDCK_22 | GAAGGTAAAAGACCATCGTTC | 157 |
| DCK | gDCK_21 | TCATACATCATCTGAAGAACA | 158 |
| DCK | gDCK_7 | ATCTTTCCTCACAACAGCTGC | 159 |
| FAS | gFAS_47 | AGTGAAGAGAAAGGAAGTACA | 160 |
| FAS | gFAS_45 | TTTGTTCTTTCAGTGAAGAGA | 161 |
| FAS | gFAS_25 | CTAGGCTTAGAAGTGGAAATA | 162 |
| FAS | gFAS_10 | GAAGGCCTGCATCATGATGGC | 163 |
| FAS | gFAS_32 | GTGCAAGGGTCACAGTGTTCA | 164 |
| FAS | gFAS_5 | GGACGATAATCTAGCAACAGA | 165 |
| FAS | gFAS_14 | TTCCTTGGGCAGGTGAAAGGA | 166 |
| FAS | gFAS_29 | GTTTACATCTGCACTTGGTAT | 167 |
| FAS | gFAS_33 | CTTGGTGCAAGGGTCACAGTG | 168 |
| FAS | gFAS_71 | CTGTTCTGCTGTGTCTTGGAC | 169 |
| FAS | gFAS_38 | CTCTTTGCACTTGGTGTTGCT | 170 |
| FAS | gFAS_70 | TGTTCTGCTGTGTCTTGGACA | 171 |
| FAS | gFAS_4 | ACAGGTTCTTACGTCTGTTGC | 172 |
| FAS | gFAS_15 | GGCAGGTGAAAGGAAAGCTAG | 173 |
| HAVCR2 | gTIM3_42 | CTAGGGTATTCTCATAGCAAA | 174 |
| HAVCR2 | gTIM3_10 | CCCCAGCAGACGGGCACGAGG | 175 |
| HAVCR2 | gTIM3_47 | GCCAACCTCCCTCCCTCAGGA | 176 |
| HAVCR2 | gTIM3 34 | TGTTTCCATAGCAAATATCCA | 177 |
| HAVCR2 | gTIM3_19 | GATCCGGCAGCAGTAGATCCC | 178 |
| HAVCR2 | gTIM3_48 | CCAATCCTGAGGGAGGGAGGT | 179 |
| HAVCR2 | gTIM3_36 | CGGGACTCTGGAGCAACCATC | 180 |
| HAVCR2 | gTIM3_15 | GCCAGTATCTGGATGTCCAAT | 181 |
| HAVCR2 | gTIM3_27 | ACTGCAGCCTTTCCAAGGATG | 182 |
| HAVCR2 | gTIM3_41 | CCCCTTACTAGGGTATTCTCA | 183 |
| HAVCR2 | gTIM3_23 | ACCTGAAGTTGGTCATCAAAC | 184 |
| HAVCR2 | gTIM3_28 | CCAAGGATGCTTACCACCAGG | 185 |
| HAVCR2 | gTIM3_40 | GTTTCCCCCTTACTAGGGTAT | 186 |
| HAVCR2 | gTIM3_13 | ATCAGTCCTGAGCACCACGTT | 187 |
| IL7R | gIL7R_2 | CCAGGGGAGATGGATCCTATC | 748 |
| IL7R | gIL7R_7 | TCTGTCGCTCTOTTGGTCATC | 753 |
| LAG3 | gLAG3_35 | TGAGGTGACTCCAGTATCTGG | 188 |
| LAG3 | gLAG3_41 | CCAGCCTTGGCAATGCCAGCT | 189 |
| LAG3 | gLAG3_37 | TGTGGAGCTCTCTGGACACCC | 190 |
| LAG3 | gLAG3_16 | GGGCAGGAAGAGGAAGCITTC | 191 |
| LAG3 | gLAG3_46 | TCCATAGGTGCCCAACGCTCT | 192 |
| LAG3 | gLAG3_27 | CCACCTGAGGCTGACCTGTGA | 193 |
| LAG3 | gLAG3_31 | CCCAGGGATCCAGGTGACCCA | 194 |
| LAG3 | gLAG3_3 | ACCTGGAGCCACCCAAAGCGG | 195 |
| LAGS | gLAG3_25 | CCCTTCGACTAGAGGATGTGA | 196 |
| LAG3 | gLAG3_13 | CGCTAAGTGGTGATGGGGGGA | 197 |
| LAG3 | gLAG3_22 | GCAGTGAGGAAAGACCGGGTC | 198 |
| PDCD1 | gPD_20 | CAGAGAGAAGGGCAGAAGTGC | 199 |
| PDCD1 | gPD_22 | GAACTGGCCGGCTGGCCTGGG | 200 |
| PDCD1 | gPD_18 | GTGCCCTTCCAGAGAGAAGGG | 201 |
| PLCG1 | gPLCG1_2 | CCTTTCTGCGCTTCGTGGTGT | 759 |
| PLCG1 | gPLCG1_4 | TGCGCTTCGTGGTGTATGAGG | 761 |
| PLCG1 | gPLCG1_5 | GTGGTGTATGAGGAAGACATG | 762 |
| PTPN6 | gPTPN6_20 | GAGACCTTCGACAGCCTCACG | 202 |
| PTPN6 | gPTPN6_41 | CTGGACCAGATCAACCAGCGG | 203 |
| PTPN6 | gPTPN6_53 | CCCCCCTGCACCCGGCTGCAG | 204 |
| PTPN6 | gPTPN6_28 | CACCAGCGTCTGGAAGGGCAG | 205 |
| PTPN6 | gPTPN6_42 | CTGCCGCTOGTTGATCTGGTC | 206 |
| PTPN6 | gPTPN6_32 | TGGCAGATGGCGTGGCAGGAG | 207 |
| PTPN6 | gPTPN6_4 | CTGGCTCGGCCCAGTCGCAAG | 208 |
| PTPN6 | gPTPN6_8 | AGGTGGATGATGGTGCCGTCG | 209 |
| PTPN6 | gPTPN6_40 | GGGAGACCTGATTCGGGAGAT | 210 |
| PTPN6 | gPTPN6_48 | AATGAACTGGGCGATGGCCAC | 211 |
| PTPN6 | gPTPN6_10 | TCTAGGTGGTACCATGGCCAC | 212 |
| PTPN6 | gPTPN6_39 | CAGGTCTCCCCGCTGGACAAT | 213 |
| TIGIT | gTIGIT_1 | GGGTGGCACATCTCCCCATCC | 214 |
| TIGIT | gTIGIT_7 | TGCAGAGAAAGGTGGCTCTAT | 215 |
| TIGIT | gTIGIT_10 | TAATGCTGACTTGGGGTGGCA | 216 |
| TIGIT | gTIGIT_27 | CTCCTGAGGTCACCTTCCACA | 217 |
| TRAC | gTRAC066 | CTAAGAAACAGIGAGCCTTGT | 218 |
| TRAC | gTRAC042 | CCTCTTTGCCCCAACCCAGGC | 219 |
| TRAC | gTRAC035 | AGGTTTCCTTGAGTGGCAGGC | 220 |
| TRAC | gTRAC044 | AGAATCAAAATCGGTGAATAG | 221 |
| TRAC | gTRACO72 | CCCCTTACTGCTCTTCTAGGC | 222 |
| TRAC | gTRAC062 | GGTGGCAATGGATAAGGCCGA | 223 |
| TRAC | gTRAC020 | GAACTATAAATCAGAACACCT | 224 |
| TRAC | gTRAC013 | TTTCTCAGAAGAGCCTOGCTA | 225 |
| TRAC | gTRAC068 | CCCGTGTCATTCTCTGGACTG | 226 |
| TRAC | gTRAC025 | CTGGGCCTTTTTCCCATGCCT | 227 |
| TRAC | gTRAC019 | AACTATAAATCAGAACACCTG | 228 |
| TRAC | gTRAC048 | ATTCTCAAACAAATGTGTCAC | 229 |
| TRAC | gTRAC036 | CTTGAGTGGCAGGCCAGGCCT | 230 |
| TRAC | gTRAC056 | CATGTGCAAACGCCTTCAACA | 231 |
| TRAC | gTRAC064 | TACTAAGAAACAGTGAGCCTT | 232 |
| TRAC | gTRAC071 | CTCAGACTGTTTGCCCCTTAC | 233 |
| TRAC | gTRAC081 | TAATTCCTCCACTTCAACACC | 234 |
| TRAC | gTRAC030 | ATAGGATCTTCTTCAAAACCC | 235 |
| TRAC | gTRAC033 | GAAGAAGATCCTATTAAATAA | 236 |
| TRAC | gTRAC001 | TGTTTTTAATGTGACTCTCAT | 237 |
| TRAC | gTRAC009 | GTACTTTACAGTTTATTAAAT | 238 |
| TRAC | gTRAC007 | ATAAACTGTAAAGTACCAAAC | 239 |
| TRAC | gTRAC084 | GACTTTTCCCAGCTGACAGAT | 240 |
| TRAC | gTRAC083 | CCCAGCTGACAGATGGGCTCC | 241 |
| TRBC2 | gTRBC2_14 | CCAGCAAGGGGTCCTGTCTOC | 714 |
| TRBC2 | gTRBC2 17 | CCATGGCCATCAGCACGAGGG | 717 |
| TRBC2 | gTRBC2_19 | CACAGGICAAGAGAAAGGATT | 719 |

The spacer sequences provided in Tables 1-3 are designed based upon identification of target nucleotide sequences associated with a PAM in a given target gene locus, and are selected based upon the editing efficiency detected in human cells.

To provide sufficient targeting to the target nucleotide sequence, the spacer sequence is generally 16 or more nucleotides in length. In certain embodiments, the spacer sequence is at least 16, 17, 18, 19, 20, 21, 22, 23, 24, 25, 26, 27, 28, 29, 30, 35, 40, 45, 50, or 75 nucleotides in length. In certain embodiments, the spacer sequence is shorter than or equal to 75, 50, 45, 40, 35, 30, 25, or 20 nucleotides in length. Shorter spacer sequence may be desirable for reducing off-target events. Accordingly, in certain embodiments, the spacer sequence is shorter than or equal to 21, 20, 19, 18, or 17 nucleotides. In certain embodiments, the spacer sequence is 17-30 nucleotides in length, e.g., 17-21, 17-22, 17-23, 17-24, 17-25, 17-30, 20-21, 20-22, 20-23, 20-24, 20-25, or 20-30 nucleotides in length. In certain embodiments, the spacer sequence is about 20 nucleotides in length. In certain embodiments, the spacer sequence is about 21 nucleotides in length. In certain embodiments, the spacer sequence is 20 nucleotides in length.

In certain embodiments, the spacer sequence comprises a portion of a spacer sequence listed in Table 1, 2, or 3, wherein the portion is 16, 17, 18, 19, or 20 nucleotides in length. In certain embodiments, the spacer sequence comprises nucleotides 1-16, 1-17, 1-18, 1-19, or 1-20 of a spacer sequence listed in Table 1, 2, or 3. In specific embodiments, the spacer sequence consists of nucleotides 1-16, 1-17, 1-18, 1-19, or 1-20 of a spacer sequence listed in Table 1, 2, or 3.

In certain embodiments, the spacer sequence is 21 nucleotides in length. In certain embodiments, the spacer sequence consists of a spacer sequence shown in Table 1, 2, or 3.

In certain embodiments, the spacer sequence, where it is longer than 21 nucleotides in length, comprises a spacer sequence shown in Table 1, 2, or 3 and one or more nucleotides. In certain embodiments, the one or more nucleotides are 3′ to the spacer sequence shown in Table 1, 2, or 3.

In certain embodiments, the spacer sequence is at least 70%, at least 75%, at least 80%, at least 85%, at least 90%, at least 93%, at least 94%, at least 95%, at least 96%, at least 97%, at least 98%, or at least 99% complementary to the target nucleotide sequence. In certain embodiments, the spacer sequence is 100% complementary to the target nucleotide sequence in the seed region (about 5 base pairs proximal to the PAM). In certain embodiments, the spacer sequence is 100% complementary to the target nucleotide sequence. The spacer sequences listed in Tables 1-3 are designed to be 100% complementary to the wild-type sequence of the corresponding target gene. Accordingly, it is contemplated that a spacer sequence useful for targeting a gene listed in Table 1, 2, or 3 can be at least 70%, at least 75%, at least 80%, at least 85%, at least 90%, at least 93%, at least 94%, at least 95%, at least 96%, at least 97%, at least 98%, or at least 99% identical to a corresponding spacer sequence listed in Table 1, 2, or 3, or a portion thereof disclosed herein. In certain embodiments, the spacer sequence is 1, 2, 3, 4, 5, 6, 7, 8, 9, or 10 nucleotides different from a sequence listed in Table 1, 2, or 3. In certain embodiments, the spacer sequence is 100% identical to a sequence listed in Table 1, 2, or 3 in the seed region (about 5 base pairs proximal to the PAM). It has been reported that compared to DNA binding, DNA cleavage is less tolerant to mismatches between the spacer sequence and the target nucleotide sequence (see, Klein et at (2018) CELL REPORTS, 22: 1413). Accordingly, in certain embodiments, a guide nucleic acid to be used with a Cas nuclease comprises a spacer sequence 100% complementary to the target nucleotide sequence. In certain embodiments, a guide nucleic acid to be used with a Cas nuclease comprises a spacer sequence listed in Table 1, 2, or 3, or a portion thereof disclosed herein.

The present invention also provides guide nucleic acids targeting human DHODH, PLK1, MVD, TUBB, or U6 gene comprising the spacer sequences provided below in Table 25. DHODH, PLK1, MVD, and TUBB are known to be essential genes. It is contemplated that the guide nucleic acids targeting these genes, particularly the ones that edit the respective genomic locus at height efficiency (e.g., at least 50%, at least 60%, at least 70%, at least 80%, or at least 90%), can be used as positive controls for assessing transfection efficiency and other experimental processes. The spacer sequences targeting U6 in Table 25 are designed to hybridize with the promoter region of human U6 gene and can be used to assess expression of an inserted gene from the endogenous U6 promoter.

### Cas Proteins

The guide nucleic acid of the present invention, either as a single guide nucleic acid alone or as a targeter nucleic acid used in combination with a cognate modulator nucleic acid, is capable of binding a CRISPR Associated (Cas) protein. In certain embodiments, the guide nucleic acid, either as a single guide nucleic acid alone or as a targeter nucleic acid used in combination with a cognate modulator nucleic acid, is capable of activating a Cas nuclease.

The terms “CRISPR-Associated protein,” “Cas protein,” and “Cas,” as used interchangeably herein, refer to a naturally occurring Cas protein or an engineered Cas protein. Non-limiting examples of Cas protein engineering includes but are not limited to mutations and modifications of the Cas protein that alter the activity of the Cas, alter the PAM specificity, broaden the range of recognized PAMs, and/or reduce the ability to modify one or more off-target loci as compared to a corresponding unmodified Cas. In certain embodiments, the altered activity of the engineered Cas comprises altered ability (e.g., specificity or kinetics) to bind the naturally occurring crRNA or engineered dual guide nucleic acids, altered ability (e.g., specificity or kinetics) to bind the target nucleotide sequence, altered processivity of nucleic acid scanning, and/or altered effector (e.g., nuclease) activity. A Cas protein having the nuclease activity is referred to as a “CRISPR-Associated nuclease” or “Cas nuclease,” as used interchangeably herein.

In certain embodiments, the Cas protein is a type V-A, type V-C, or type V-D Cas protein. In certain embodiments, the Cas protein is a type V-A Cas protein. In other embodiments, the Cas protein is a type II Cas protein, e.g., a Cas9 protein.

In certain embodiments, the Cas nuclease is a type V-A, type V-C, or type V-D Cas nuclease. In certain embodiments, the Cas nuclease is a type V-A Cas nuclease. In other embodiments, the Cas protein is a type II Cas nuclease, e.g., a Cas9 nuclease.

In certain embodiments, the type V-A Cas protein comprises Cpf1. Cpf1 proteins are known in the art and are described in U.S. Pat. Nos. 9,790,490 and 10,113,179. Cpf1 orthologs can be found in various bacterial and archaeal genomes. For example, in certain embodiments, the Cpf1 protein is derived from Francisella novicida U112 (Fn), Acidaminococcus sp. BV3L6 (As), Lachnospiraceae bacterium ND2006 (Lb), Lachnospiraceae bacterium MA2020 (Lb2). Candidatus Methanoplasma termitum (CMt), Moraxella bovoculi 237 (Mb), Porphyromonas crevioricanis (Pc), Prevotella disiens (Pd), Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Eubacterium eligens, Leptospira inadai, Porphyromonas macacae. Prevotella bryantii (Pb), Proteocatella sphenisci (Ps), Anaerovibrio sp. RM50 (As2), Moraxella caprae (Mc), Lachnospiraceae bacterium COE1 (Lb3), or Eubacterium coprostanoligenes (Ec).

In certain embodiments, the type V-A Cas protein comprises AsCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 3. In certain embodiments, the type V-A Cas protein comprises the amino acid sequence set forth in SEQ ID NO: 3.

AsCpf1
(SEQ ID NO: 3)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRDAEIYKGLFKAELFNGKVLKQLGTVT
TTEHENALLRSFDKFTTYFSGEYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNILSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELLYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSIGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In certain embodiments, the type V-A Cas protein comprises LbCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 4. In certain embodiments, the type V-A Cas protein comprises the amino acid sequence set forth in SEQ ID NO: 4.

LbCpf1
(SEQ ID NO: 4)
MSKLEKFTNCYSLSKTLRFKAIPVGKTQENIDNKRLLVEDEKRAEDYKGV
KKLLDRYYLSFINDVLHSIKLKNLNNYISLFRKKTRTEKENKELENLEIN
LRKEIAKAFKGNEGYKSLFKKDIIETILPEFLDDKDEIALVNSFNGFTTA
FTGFFDNRENMFSEEAKSTSIAFRCINENLTRYISNMDIFEKVDAIFDKH
EVQEIKEKILNSDYDVEDFFEGEFFNFVLTQEGIDVYNAIIGGFVTESGE
KIKGLNEYINLYNQKTKQKLPKFKPLYKQVLSDRESLSFYGEGYTSDEEV
LEVFRNTLNKNSEIFSSIKKLEKLFKNFDEYSSAGIFVKNGPAISTISKD
IFGEWNVIRDKWNAEYDDIHLKKKAVVTEKYEDDRRKSFKKIGSFSLEQL
QEYADADLSVVEKLKEIIIQKVDEIYKVYGSSEKLFDADFVLEKSLKKND
AVVAIMKDLLDSVKSFENYIKAFFGEGKETNRDESFYGDFVLAYDILLKV
DHIYDAIRNYVTQKPYSKDKFKLYFQNPQFMGGWDKDKETDYRATILRYG
SKYYLAIMDKKYAKCLQKIDKDDVNGNYEKINYKLLPGPNKMLPKVFFSK
KWMAYYNPSEDIQKIYKNGTFKKGDMFNLNDCHKLIDFFKDSISRYPKWS
NAYDFNFSETEKYKDIAGFYREVEEQGYKVSFESASKKEVDKLVEEGKLY
MFQIYNKDFSDKSHGTPNLHTMYFKLLFDENNHGQIRLSGGAELFMRRAS
LKKEELVVHPANSPIANKNPDNPKKTTTLSYDVYKDKRFSEDQYELHIPI
AINKCPKNIFKINTEVRVLLKHDDNPYVIGIDRGERNLLYIVVVDGKGNI
VEQYSLNEIINNFNGIRIKTDYHSLLDKKEKERFEARQNWTSIENIKELK
AGYISQVVHKICELVEKYDAVIALEDLNSGFKNSRVKVEKQVYQKFEKML
IDKLNYMVDKKSNPCATGGALKGYQITNKFESFKSMSTQNGFIFYIPAWL
TSKIDPSTGFVNLLKTKYTSIADSKKFISSFDRIMYVPEEDLFEFALDYK
NFSRTDADYIKKWKLYSYGNRIRIRNPKKNNVIDWEEVCLTSAYKELFNK
YGINYQQGDIRALLCEQSDKAFYSSFMALMSLMLQMRNSITGRTDVDFLI
SPVKNSDGIFYDSRNYEAQENAILPKNADANGAYNIARKVLWAIGQFKKA
EDEKLDKVKIAISNKEWLEYAQTSVKH

In certain embodiments, the type V-A Cas protein comprises FnCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 5. In certain embodiments, the type V-A Cas protein comprises the amino acid sequence set forth in SEQ ID NO: 5.

FnCpf1
(SEQ ID NO: 5)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTTKKQISEYIKDSEKFKNLFNQNLIDAKKGQESDLILWLKQSKDNGI
ELFKANSDITDIDEALEIIKSFKGWTIYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDWTT
MQSFYEQIAAFKTVEEKSIKETLSLLFDDLKAQKLDLSKIYFKNDKSLTD
LSQQVFDDYSVIGTAVLEYITQQIAPKNLDNPSKKEQELIAKKTEKAKYL
SLETIKLALEEFNKHRDIDKQCRFEEILANFAAIPMIFDEIAQNKDNLAQ
ISIKYQNQGKKDLLQASAEDDVKAIKDLLDQTNNLLHKLKIFHISQSEDK
ANILDKDEHFYLVFEECYFELANIVPLYNKIRNYITQKPYSDEKFKLNFE
NSTLANGWDKNKEPDNTAILFIKDDKYYLGVMNKKNNKIFDDKAIKENKG
EGYKKIVYKLLPGANKMLPKVFFSAKSIKFYNPSEDILRIRNHSTHTKNG
SPQKGYEKFEFNIEDCRKFIDFYKQSISKHPEWKDFGFRFSDTQRYNSID
EFYREVENQGYKLTFENISESYIDSVVNQGKLYLFQIYNKDFSAYSKGRP
NLHTLYWKALFDERNLQDVVYKLNGEAELFYRKQSIPKKITHPAKEAIAN
KNKDNPKKESVFEYDLIKDKRFTEDKFFFHCPITINFKSSGANKFNDEIN
LLLKEKANDVHILSIDRGERHLAYYTLVDGKGNIIKQDTFNIIGNDRMKT
NYHDKLAAIEKDRDSARKDWKKINNIKEMKEGYLSQVVHEIAKLVIEYNA
IVVFEDLNFGFKRGRFKVEKQVYQKLEKMLIEKLNYLVFKDNEFDKTGGV
LRAYQLTAPFETFKKMGKQTGIIYYVPAGFTSKICPVTGFVNQLYPKYES
VSKSQEFFSKFDKICYNLDKGYFEFSFDYKNFGDKAAKGKWTIASFGSRL
INFRNSDKNHNWDTREVYPTKELEKLLKDYSIEYGHGECIKAAICGESDK
KFFAKLTSVLNTILQMRNSKTGTELDYLISPVADVNGNFFDSRQAPKNMP
QDADANGAYHIGLKGLMLLGRIKNNQEGKKLNLVIKNEEYFEFVQNRNN

In certain embodiments, the type V-A Cas protein comprises PbCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 6. In certain embodiments, the type V-A Cas protein comprises the amino acid sequence set forth in SEQ ID NO: 6.

PbCpf1
(SEQ ID NO: 6)
MQINNLKIIYMKFTDFTGLYSLSKTLRFELKPIGKTLENIKKAGLLEQDQ
HRADSYKKVKKIIDEYHKAFIEKSLSNFELKYQSEDKLDSLEEYLMYYSM
KRIEKTEKDKEAKIQDNLRKQIADHLKGDESYKTIFSKDLIRKNLPDFVK
SDEERTLIKEFKDFTTYFKGFYENRENMYSAEDKSTAISHRIIHENLPKF
VDNINAFSKIILIPELREKLNQIYQDFEEYLNVESIDEIFHLDYFSMVMT
QKQIEVYNAIIGGKSTNDKKIQGLNEYINLYNQKHKDCKLPKLKLLFKQI
LSDRIAISWLPDNFKDDQEALDSIDTCYKNLLNDGNVLGEGNLKLLLENI
DTYNLKGIFIRNDLQLTDISQKMYASWNVIQDAVILDLKKQVSRKKKESA
EDYNDRLKKLYTSQESFSIQYLNDCLRAYGKTENIQDYFAKLGAVNNEHE
QTINLFAQVRNAYTSVQAILTTPYPENANLAQDKETVALIKNLLDSLKRL
QRFIKPLLGKGDESDKDERFYGDFTPLWETLNQITPLYNMVRNYMTRKPY
SQEKIKLNFENSTLLGGWDLNKEHDNTAIILRKNGLYYLAIMKKSANKIF
DKDKLDNSGDCYEKMVYKLLPGANKMLPKVFFSKSRIDEFKPSENIIENY
KKGTHKKGANFNLADCHNLIDFFKSSISKHEDWSKFNFHFSDTSSYEDLS
DFYREVEQQGYSISFCDVSVEYINKMVEKGDLYLFQIYNKDFSEFSKGTP
NMHTLYWNSLFSKENLNNIIYKLNGQAEIFFRKKSLNYKRPTHPAHQAIK
NKNKCNEKKESIFDYDLVKDKRYTVDKFQFHVPITMNFKSTGNTNINQQV
IDYLRTEDDTHIIGIDRGERHLLYLVVIDSHGKIVEQFTLNEIVNEYGGN
IYRTNYHDLLDTREQNREKARESWQTIENIKELKEGYISQVIHKITDLMQ
KYHAVVVLEDLNMGFMRGRQKVEKQVYQKFEEMLINKLNYLVNKKADQNS
AGGLLHAYQLTSKFESFQKLGKQSGFLFYIPAWNTSKIDPVTGFVNLFDT
RYESIDKAKAFFGKFDSIRYNADKDWFEFAFDYNNFTTKAEGTRTNWTIC
TYGSRIRTFRNQAKNSQWDNEEIDLTKAYKAFFAKHGINIYDNIKEAIAM
ETEKSFFEDLLHLLKLTLQMRNSITGTTTDYLISPVHDSKGNFYDSRICD
NSLPANADANGAYNIARKGLMLIQQIKDSTSSNRFKFSPITNKDWLIFAQ
EKPYLND

In certain embodiments, the type V-A Cas protein comprises PsCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 7. In certain embodiments, the type V-A Cas protein comprises the amino acid sequence set forth in SEQ ID NO: 7.

PsCpf1
(SEQ ID NO: 7)
MENFKNLYPINKTLRFELRPYGKTLENFKKSGLLEKDAFKANSRRSMQAI
IDEKFKETIEERLKYTEFSECDLGNMTSKDKKITDKAATNLKKQVILSFD
DEIFNNYLKPDKNIDALFKNDPSNPVISTFKGFTTYFVNFFEIRKHIFKG
ESSGSMAYRIIDENLTTYLNNIEKIKKLPEELKSQLEGIDQIDKLNNYNE
FITQSGITHYNEIIGGISKSENVKIQGINEGINLYCQKNKVKLPRLTPLY
KMILSDRVSNSFVLDTIENDTELIEMISDLINKTEISQDVIMSDIQNIFI
KYKQLGNLPGISYSSIVNAICSDYDNNFGDGKRKKSYENDRKKHLETNVY
SINYISELLTDTDVSSNIKMRYKELEQNYQVCKENFNATNWMNIKNIKQS
EKTNLIKDLLDILKSIQRFYDLFDIVDEDKNPSAEFYTWLSKNAEKLDFE
FNSVYNKSRNYLTRKQYSDKKIKLNFDSPTLAKGWDANKEIDNSTIIMRK
FNNDRGDYDYFLGIWNKSTPANEKIIPLEDNGLFEKMQYKLYPDPSKMLP
KQFLSKIWKAKHPLTPEFDKKYKEGRHKKGPDFEKEFLHELIDCFKHGLV
NHDEKYQDVFGFNLRNTEDYNSYTEFLEDVERCNYNLSFNKIADTSNLIN
DGKLYVFQIWSKDFSIDSKGTKNLNTIYFESLFSEENMIEKMFKLSGEAE
IFYRPASLNYCEDIIKKGHHHAELKDKFDYPIIKDKRYSQDKFFFHVPMV
INYKSEKLNSKSLNNRTNENLGQFTHIIGIDRGERHLIYLTVVDVSTGEI
VEQKHLDEIINTDTKGVEHKTHYLNKLEEKSKTRDNERKSWEAIETIKEL
KEGYISHVINEIQKLQEKYNALIVMENLNYGFKNSRIKVEKQVYQKFETA
LIKKFNYIIDKKDPETYIHGYQLTNPITTLDKIGNQSGIVLYIPAWNTSK
IDPVTGFVNLLYADDLKYKNQEQAKSFIQKIDNIYFENGEFKFDIDFSKW
NNRYSISKTKWTLTSYGTRIQTFRNPQKNNKWDSAEYDLTEEFKLILNID
GTLKSQDVETYKKFMSLFKLMLQLRNSVTGTDIDYMISPVTDKTGTHFDS
RENIKNLPADADANGAYNIARKGIMAIENIMNGISDPLKISNEDYLKYIQ
NQQE

In certain embodiments, the type V-A Cas protein comprises As2Cpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 8. In certain embodiments, the type V-A Cas protein comprises the amino acid sequence set forth in SEQ ID NO: 8.

As2Cpf1
(SEQ ID NO: 8)
MVAFIDEFVGQYPVSKTLRFEARPVPETKKWLESDQCSVLFNDQKRNEYY
GVLKELLDDYYRAYIEDALTSFTLDKALLENAYDLYCNRDTNAFSSCCEK
LRKDLVKAFGNLKDYLLGSDQLKDLVKLKAKVDAPAGKGKKKIEVDSRLI
NWLNNNAKYSAEDREKYIKAIESFEGFVTYLTNYKQARENMFSSEDKSTA
IAFRVIDQNMVTYFGNIRIYEKIKAKYPELYSALKGFEKFFSPTAYSEIL
SQSKIDEYNYQCIGRPIDDADFKGVNSLINEYRQKNGIKARELPVMSMLY
KQILSDRDNSFMSEVINRNEEAIECAKNGYKVSYALFNELLQLYKKIFTE
DNYGNIYVKTQPLTELSQALFGDWSILRNALDNGKYDKDIINLAELEKYF
SEYCKVLDADDAAKIQDKFNLKDYFIQKNALDATLPDLDKITQYKPHLDA
MLQAIRKYKLFSMYNGRKKMDVPENGIDFSNEFNAIYDKLSEFSILYDRI
RNFATKKPYSDEKMKLSFNMPTMLAGWDYNNETANGCFLFIKDGKYFLGV
ADSKSKNIFDFKKNPHLLDKYSSKDIYYKVKYKQVSGSAKMLPKVVFAGS
NEKIFGHLISKRILEIREKKLYTAAAGDRKAVAEWIDFMKSAIAIHPEWN
EYFKFKFKNTAEYDNANKFYEDIDKQTYSLEKVEIPTEYIDEMVSQHKLY
LFQLYTKDFSDKKKKKGTDNLHTMYWHGVFSDENLKAVTEGTQPIIKLNG
EAEMFMRNPSIEFQVTHEHNKPIANKNPLNTKKESVFNYDLIKDKRYTER
KFYFHCPITLNFRADKPIKYNEKINRFVENNPDVCIIGIDRGERHLLYYT
VINQTGDILEQGSLNKISGSYTNDKGEKVNKETDYHDLLDRKEKGKHVAQ
QAWETIENIKELKAGYLSQVVYKLTQLMLQYNAVIVLENLNVGFKRGRTK
VEKQVYQKFEKAMIDKLNYLVFKDRGYEMNGSYAKGLQLTDKFESFDKIG
KQTGCIYYVIPSYTSHIDPKTGFVNLLNAKLRYENITKAQDTIRKFDSIS
YNAKADYFEFAFDYRSFGVDMARNEWVVCTCGDLRWEYSAKTRETKAYSV
TDRLKELFKAHGIDYVGGENLVSHITEVADKHFLSTLLFYLRLVLKMRYT
VSGTENENDFILSPVEYAPGKFFDSREATSTEPMNADANGAYHIALKGLM
TIRGIEDGKLHNYGKGGENAAWFKFMQNQEYKNNG

In certain embodiments, the type V-A Cas protein comprises McCpf1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 9. In certain embodiments, the type V-A Cas protein comprises the amino acid sequence set forth in SEQ ID NO: 9.

McCpf1
(SEQ ID NO: 9)
MLFQDFTHLYPLSKTMRFELKPIGKTLEHIHAKNFLSQDETMADMYQKVK
AILDDYHRDFIADMMGEVKLTKLAEFYDVYLKFRKNPKDDGLQKQLKDLQ
AVLRKEIVKPIGNGGKYKAGYDRLFGAKLFKDGKELGDLAKFVIAQEGES
SPKLAHLAHFEKFSTYFTGFHDNRKNMYSDEDKHTAITYRLIHENLPRFI
DNLQILATIKQKHSALYDQIINELTASGLDVSLASHLDGYHKLITQEGIT
AYNTLLGGISGEAGSRKIQGINEIINSHHNQHCHKSERIAKLRPLHKQIL
SDGMGVSFLPSKFADDSEMCQAVNEFYRHYADVFAKVQSLFDGFDDHQKD
GIYVEHKNLNELSKQAFGDFALLGRVLDGYYVDVVNPEFNERFAKAKTDN
AKAKLTKEKDKFIKGVHSLASLEQATEHYTARHDDESVQAGKLGQYFKHG
LAGVDNPIQKIHNNHSTIKGFLERERPAGERALPKIKSGKNPEMTQLRQL
KELLDNALNVAHFAKLLTTKTTLDNQDGNFYGEFGALYDELAKIPTLYNK
VRDYLSQKPFSTEKYKLNFGNPTLLNGWDLNKEKDNFGIILQKDGCYYLA
LLDKAHKKVFDNAPNTGKNVYQKMIYKLLPGPNKMLPKVFFAKSNLDYYN
PSAELLDKYAQGTHKKGNNFNLKDCHALLDFFKAGINKHPEWQHFGFKFS
PTSSYQDLSDFYREVEPQGYQVKFVDINADYINELVEQGQLYLFQIYNKD
FSPKAHGKPNLHTLYFKALFSKDNLANPIYKLNGEAQIFYRKASLDMNET
TIHRAGEVLENKNPDNPKKRQFVYDIIKDKRYTQDKFMLHVPITMNFGVQ
GMTIKEFNKKVNQSIQQYDEVNVIGIDRGERHLLYLTVINSKGEILEQRS
LNDITTASANGTQMTTPYHKILDKREIERLNARVGWGEIETIKELKSGYL
SHVVHQISQLMLKYNAIVVLEDLNFGFKRGRFKVEKQIYQNEENALIKKL
NHLVLKDEADDEIGSYKNALQLTNNFTDLKSIGKQTGFLFYVPAWNTSKI
DPETGFVDLLKPRYENIAQSQAFFGKFDKICYNADKDYFEFHIDYAKFTD
KAKNSRQIWKICSHGDKRYVYDKTANQNKGATKGINVNDELKSLFAREIF
IINDKQPNLVMDICQNNDKEFHKSLIYLLKTLLALRYSNASSDEDFILSP
VANDEGMFFNSALADDTQPQNADANGAYHIALKGLWVLEQIKNSDDLNKV
KLAIDNQTWINFAQNR

In certain embodiments, the type V-A Cas protein comprises Lb3Cpf1 or a variant thereof. In certain embodiments, the t % p V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least W4%, at least 95%, at least 96%, at least 97%, at least 98%, or at least 99% identical to the amino acid sequence set forth in SEQ ID NO: 10. In certain embodiments, the type V-A Cas protein comprises the amino acid sequence set forth in SEQ ID NO: 10.

LbCpf1
(SEO ID NO: 10)
MHENNGKIADNFIGIYPVSKTLRFELKPVGKTQEYIEKHGILDEDLKRAG
DYKSVKKIIDAYHKYFIDEALNGIQLDGLKNYYELYEKKRDNNEEKEFQK
IQMSLRKQIVKRFSEHPQYKYLFKKELIKNVLPEFTKDNAEEQTLVKSFQ
EFTTYFEGFHQNRKNMYSDEEKSTAIAYRVVHQNLPKYIDNMRIFSMILN
TDIRSDLTELFNNLKTKMDITIVEEYFAIDGFNKVVNQKGIDVYNTILGA
FSTDDNTKIKGLNEYINLYNQKNKAKLPKLKPLFKQILSDRDKISFIPEQ
FDSDTEVLEAVDMFYNRLLQFVIENEGQITISKLLTNFSAYDLNKIYVKN
DTTISAISNDLFDDWSYISKAVRENYDSENVDKNKRAAAYEEKKEKALSK
IKMYSIEELNFFVKKYSCNECHIEGYFERRILEILDKMRYAYESCKILHD
KGLINNISLCQDRQAISELKDFLDSIKEVQWLLKPLMIGQEQADKEEAFY
TELLRIWEELEPITLLYNKVRNYVTKKPYTLEKVKLNFYKSTLLDGWDKN
KEKDNLGIILLKDGQYYLGIMNRRNNKIADDAPLAKTDNVYRKMEYKLLT
KVSANLPRIFLKDKYNPSEEMLEKYEKGTHLKGENFCIDDCRELIDFFKK
GIKQYEDWGQFDFKFSDTESYDDISAFYKEVEHQGYKITFRDIDETYIDS
LVNEGKLYLFQIYNKDFSPYSKGTKNLHTLYWEMLFSQQNLQNIVYKLNG
NAEIFYRKASINQKDVVVHKADLPIKNKDPQNSKKESMFDYDIIKDKRFT
CDKYQFHVPITMNFKALGENHFNRKVNRLIHDAENMHIIGIDRGERNLIY
LCMIDMKGNIVKQISLNEIISYDKNKLEHKRNYHQLLKTREDENKSARQS
WQTIHTIKELKEGYLSQVIHVITDLMVEYNAIVVLEDLNFGFKQGRQKFE
RQWQKFEKMLIDKLMYLVDKSKGMDEDGGLLHAYQLTDEFKSFKQLGKQS
GFLYYIPAWNTSKLDPTTGFVNLFYTKYESVEKSKEFINNFTSILYNQER
EYFEFLFDYSAFTSKAEGSRLKWTVCSKGERVETYRNPKKNNEWDTQKID
LTFELKKLFNDYSISLLDGDLREQMGKIDKADFYKKFMKLFALIVQMRNS
DEREDKLISPVLNKYGAFFETGKNERMPLDADANGAYNIARKGLWIIEKI
KNIDVEQLDKVKLTISNKEWLQYAQEHIL

In certain embodiments, the type V-A Cas protein comprises EcCpf1 or a variant thereof. In certain embodiments, the type V-A Cas protein comprises an amino acid sequence at least 301%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 11. In certain embodiments, the type V-A Cas protein comprises the amino acid sequence set forth in SEQ ID NO: 11.

EcCpf1
(SEO ID NO: 11)
MDFFKNDMYFLCINGIIVISKLFAYLFLMYKRGVVMIKDNFVNVYSLSKT
IRMALIPWGKTEDNFYKKFLLEEDEERAKNYIKVKGYMDEYHKNFIESAL
NSVVLNGVDEYCELYFKQNKSDSEVKKIESLEASMRKQISKAMKEYTVDG
VKIYPLLSKKEFIRELLPEFLTQDEEIETLEQFNDFSTYFQGFWENRKNI
YTDEEKSTGVPYRCINDNLPKFLDNVKSFEKVILALPQKAVDELNANFNG
VYNVDVQDVFSVDYFNFVLSQSGIEKYNNIIGGYSNSDASKVQGLNEKIN
LYNQQIAKSDKSKKLPLLKPLYKQILSDRSSLSFIPEKFKDDNEVLNSIN
VLYDNIAESLEKANDLMSDIANYNTDNIFISSGVAVTDISKKVFGDWSLI
RNNWNDEYESTHKKGKNEEKFYEKEDKEFKKIKSFSVSELQRLANSDLSI
VDYLVDESASLYADIKTAYNNAKDLLSNEYSHSKRLSKNDDAIELIKSFL
DSIKNYEAFLKPLCGTGKEESKDNAFYGAFLECFEEIRQVDAVYNKVRNH
ITQKPYSNDKIKLNFQNPQFLAGWDKNKERAYRSVLLRNGEKYYLAIMEK
GKSKLFEDFPEDESSPFEKIDYKLLPEPSKMIPKVFFATSNKDLFNPSDE
ILNIRATGSFKKGDSFNLDDCHKFIDFYKASIENHPDWSKFDFDFSETND
YEDISKFFKEVSDQGYSIGYRKISESYLEEMVDNGSLYMFQLYNKDFSEN
RKSKGTPNLHTLYFKMLFDERNLEDVVYKLSGGAEMFYRKPSIDKNEMIV
HPKNQPIDNKNPNNVKKTSTFEYDIVKDMRYTKPQFQLHLPIVLNFKANS
KGYINDDVRNVLKNSEDTYVIGIDRGERNLVYACVVDGNGKLVEQVPLNV
IEADNGYKTDYHKLLNDREEKRNEARKSWKTIGNIKELKEGYISQVVHKI
CQLVVKYDAVIAMEDLNSGFVNSRKKVEKQVYQKFERMLTQKLNYLVDKK
LDPNEMGGLLNAYQLTNEATKVRNGRQDGIIFYIPAWLTSKIDPTTGFVN
LLKPKYNSVSASKEFFSKFDEIRYNEKENYFEFSFNYDNFPKCNADFKRE
WTVCTYGDRIRTFRDPENNNKFNSEVVVLNDEFKNLFVEFDIDYTDNLKE
QILAMDEKSFYKKLMGLLSLTLQMRNSISKNVDVDYLISPVKNSNGEFYD
SRNYDITSSLPCDADSNGAYNIARKGLWAINQIKQADDETKANISIKNSE
WLQYAQNCDEV

In certain embodiments, the type V-A Cas protein is not Cpf1. In certain embodiments, the type V-A Cas nuclease is not AsCpf1.

In certain embodiments, the type V-A Cas protein comprises MAD1, MAD2, MAD3, MAD4, MAD5, MAD6, MAD7, MAD8, MAD9, MAD10, MAD11, MAD12, MAD73, MAD14, MAD15, MAD16, MAD17, MAD18, MAD19 or MAD20, or variants thereof. MAD1-MAD20 are known in the art and are described in U.S. Pat. No. 9,982,279.

In certain embodiments, the type V-A Cas protein comprises MAD7 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 1. In certain embodiments, the type V-A Cas protein comprises the amino acid sequence set forth in SEQ ID NO: 1.

MAD7
(SEO ID NO: 1)
MNNGTNNFQNFGISSLQKTLKNALIPTETTQQHVKNGIIKEDELRGENRQ
ILKDIMDDYYRGFISETLSSIDDIDWTSLFEKMEIQLKNGDNKDTLIKEQ
TEYRKAIHKKFANDDRFKNMFSAKLISDILPEFVIHNNNYSASEKEEKTQ
VIKLFSRFATSFKDYFKNRANCFSADDISSSSCHRIVNDNAEIFFSNALV
YRRIVKSLSNDDINKISGDMKDSLKEMSLEEIYSYEKYGEFITQEGISFY
NDICGKVNSFMNLYCQKNKENKNLYKLQKLHKQILCIADTSYEVPYKFES
DEEVYQSVNGFLDNISSKHIVERLRKIGDNYNGYNLDKIYIVSKFYESVS
QKTYRDWETINTALEIHYNNILPGNGKSKADKVKKAVKNDLQKSITEINE
LVSNYKLCSDDNIKAETYIHEISHILNNFEAQELKYNPEIHLVESELKAS
ELKNVLDVIMNAFHWCSVFMTEELVDKDNNFYAELEEIYDEIYPVISIYN
LVRNYVTQKPYSTKKIKLNFGIPTLADGWSKSKEYSNNAIILMRDNLYYL
GIFNAKNKPDKKIIEGNTSENKGDYKKMIYNLLPGPNKMIPKVFLSSKTG
VETYKPSAYILEGYKQNKHIKSSKDFDITFCHDLIDYFKNCIAIHPEWKN
FGFDFSDTSTYEDISGFYREVELQGYKIDWTYISEKDIDLLQEKGQLYLF
QIYNKDFSKKSTGNDNLHTMYLKNLFSEENLKDIVLKLNGEAEIFFRKSS
IKNPIIHKKGSILVNRTYEAEEKDQFGNIQIVRKNIPENIYQELYKYFND
KSDKELSDEAAKLKNVVGHHEAATNIVKDYRYTYDKYFLHMPITINFKAN
KTGFINDRILQYIAKEKDLHVIGIDRGERNLIYVSVIDTCGNIVEQKSFN
IVNGYDYQIKLKQQEGARQIARKEWKEIGKIKEIKEGYLSLVIHEISKMV
IKYNAIIAMEDLSYGFKKGRFKVERQVYQKFETMIINKLNYLVFKDISIT
ENGGLLKGYQLTYIPDKLKNVGHQCGCIFYVPAAYTSKIDPTTGFVNIFK
FKDLTVDAKREFIKKFDSIRYDSEKNLFCFTFDYNNFITQNTVMSKSSWS
VYTYGVRIKRRFVNGRFSNESDTIDITKDMEKTLEMTDINWRDGHDLRQD
IIDYEIVQHIFEIFRLTVQMRNSLSELEDRDYDRLISPVLNENNIFYDSA
KAGDALPKDADANGAYCIALKGLYEIKQITENWKEDGKFSRDKLKISNKD
WFDFIQNKRYL

In certain embodiments, the type V-A Cas protein comprises MAD2 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 2. In certain embodiments, the type V-A Cas protein comprises the amino acid sequence set forth in SEQ ID NO: 2.

MAD2
(SEO ID NO: 2)
MSSLTKFTNKYSKQLTIKNELIPVGKTLENIKENGLIDGDEQLNENYQKA
KIIVDDFLRDFINKALNNTQIGNWRELADALNKEDEDNIEKLQDKIRGII
VSKFETFDLFSSYSIKKDEKIIDDDNDVEEEELDLGKKTSSFKYIFKKNL
FKLVLPSYLKTTNQDKLKIISSFDNFSTYFRGFFENRKNIFTKKPISTSI
AYRIVHDNFPKFLDNIRCFNVWQTECPQLIVKADNYLKSKNVIAKDKSLA
NYFTVGAYDYFLSQNGIDFYNNIIGGLPAFAGHEKIQGLNEFINQECQKD
SELKSKLKNRHAFKMAVLFKQILSDREKSFVIDEFESDAQVIDAVKNFYA
EQCKDNNVIFNLLNLIKNIAFLSDDELDGIFIEGKYLSSVSQKLYSDWSK
LRNDIEDSANSKQGNKELAKKIKTNKGDVEKAISKYEFSLSELNSIVHDN
TKFSDLLSCTLHKVASEKLVKVNEGDWPKHIKNNEEKQKIKEPIDALLEI
YNTLLIINCKSFNKNGNFYVDYDRCINELSSVVYLYNKTRNYCTKKPYNT
DKFKLNFNSPQLGEGFSKSKENDCLTLLFKKDDNYYVGIIRKGAKINFDD
TQAIADNTDNCIFKMNYFLLKDAKKFIPKCSIQLKEVKAHFKKSEDDYIL
SDKEKFASPLVIKKSTFLLATAHVKGKKGNIKKFQKEYSKENPTEYRNSL
NEWIAFCKEFLKTYKAATIFDITTLKKAEEYADIVEFYKDVDNLCYKLEF
CPIKTSFIENLIDNGDLYLFRINNKDFSSKSTGTKNLHTLYLQAIFDERN
LNNPTIMLNGGAELFYRKESIEQKNRITHKAGSILVNKVCKDGTSLDDKI
RNEIYQYENKFIDTLSDEAKKVLPNVIKKEATHDITKDKRFTSDKFFFHC
PLTINYKEGDTKQFNNEVLSFLRGNPDINIIGIDRGERNLIYVTVINQKG
EILDSVSFNTVTNKSSKIEQTVDYEEKLAVREKERIEAKRSWDSISKIAT
LKEGYLSAIVHEICLLMIKHNAIVVLENLNAGFKRIRGGLSEKSVYQKFE
KMLINKLNYFVSKKESDWNKPSGLLNGLQLSDQFESFEKLGIQSGFIFYV
PAAYTSKIDPTTGFANVLNLSKVRNVDAIKSFFSNFNEISYSKKEALFKF
SFDLDSLSKKGFSSFVLTSANLKDTFWKELFFIFKTTLQLRNSVTNGKED
VLISPVKNAKGEFFVSGTHNKTLPQDCDANGAYHIALKGLMILERNNLVR
EEKDTKKIMAISNVDWFEYVQKRRGVL

In certain embodiments, the type V-A Cas protein comprises Csm1. Csm1 proteins are known in the art and are described in U.S. Pat. No. 9,896,696. Csm1 orthologs can be found in various bacterial and archaeal genomes. For example, in certain embodiments, the Csm1 protein is derived from Smithella sp. SCADC (Sm), Sulfuricurvum sp. (Ss), or Microgenomates (Roizmanbacteria) bacterium (Mb).

In certain embodiments, the type V-A Cas protein comprises SmCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 12. In certain embodiments, the type V-A Cas protein comprises the amino acid sequence set forth in SEQ ID NO: 12.

SmCsm1
(SEO ID NO: 12)
MEKYKITKTIRFKLLPDKIQDISRQVAVLQNSTNAEKKNNLLRLVQRGQE
LPKLLNEYIRYSDNHKLKSNVTVHFRWLRLFTKDLFYNWKKDNTEKKIKI
SDVVYLSHVFEAFLKEWESTIERVNADCNKPEESKTRDAEIALSIRKLGI
KHQLPFIKGFVDNSNDKNSEDTKSKLTALLSEFEAVLKICEQNYLPSQSS
GIAIAKASFNYYTINKKQKDFEAEIVALKKQLHARYGNKKYDQLLRELNL
IPLKELPLKELPLIEFYSEIKKRKSTKKSEFLEAVSNGLVFDDLKSKFPL
FQTESNKYDEYLKLSNKITQKSTAKSLLSKDSPEAQKLQTEITKLKKNRG
EYFKKAFGKYVQLCELYKEIAGKRGKLKGQIKGIENERIDSQRLQYWALV
LEDNLKHSLILIPKEKTNELYRKVWGAKDDGASSSSSSTLYYFESMTYRA
LRKLCFGINGNTFLPEIQKELPQYNQKEFGEFCFHKSNDDKEIDEPKLIS
FYQSVLKTDFVKNTLALPQSVFNEVAIQSFETRQDFQIALEKCCYAKKQI
ISESLKKEILENYNTQIFKITSLDLQRSEQKNLKGHTRIWNRFWTKQNEE
INYNLRLNPEIAIVWRKAKKTRIEKYGERSVLYEPEKRNRYLHEQYTLCT
TVTDNALNNEITFAFEDTKKKGTEIVKYNEKINQTLKKEFNKNQLWFYGI
DAGEIELATLALMNKDKEPQLFTVYELKKLDFFKHGYIYNKERELVIREK
PYKAIQNLSYFLNEELYEKTFRDGKFNETYNELFKEKHVSAIDLTTAKVI
NGKIILNGDMITFLNLRILHAQRKIYEELIENPHAELKEKDYKLYFEIEG
KDKDIYISRLDFEYIKPYQEISNYLFAYFASQQINEAREEEQINQTKRAL
AGNMIGVIYYLYQKYRGIISIEDLKQTKVESDRNKFEGNIERPLEWALYR
KFQQEGYVPPISELIKLRELEKFPLKDVKQPKYENIQQFGIIKFVSPEET
STTCPKCLRRFKDYDKNKQEGFCKCQCGFDTRNDLKGFEGLNDPDKVAAF
NIAKRGFEDLQKYK

In certain embodiments, the type V-A Cas protein comprises SsCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 13. In certain embodiments, the type V-A Cas protein comprises the amino acid sequence set forth in SEQ ID NO: 13.

SsCsm1
(SEQ ID NO: 13)
MLHAFTNQYQLSKTLRFGATLKEDEKKCKSHEELKGFVDISYENMKSSAT
IAESLNENELVKKCERCYSEIVKFHNAWEKIYYRTDQIAVYKDFYRQLSR
KARFDAGKQNSQLITLASLCGMYQGAKLSRYITNYWKDNITRQKSFLKDF
SQQLHQYTRALEKSDKAHTKPNLINFNKTFMVLANLVNEIVIPLSNGAIS
FPNISKLEDGEESHLIEFALNDYSQLSELIGELKDAIATNGGYTPPAKVT
INHYTAEQKPHVIKNDIDAKIRELKLIGIVETLKGKSSEQIEEYFSNLDK
FSTYNDRNQSVIVRTQCFKYKPIPFLVKHQLAKYISEPNGWDEDAVAKVL
DAVGAIRSPAHDYANNQEGFDLNHYPIKVAFDYAWEQLANSLYTTVTFPQ
EMCEKYLNSIYGCEVSKEPVFKFYADLLYIRKNLAVLEHKNNLPSNQEEF
ICKINNTFENIVLPYKISQFETYKKDILAWINDGHDHKKYTDAKQQLGFI
RGGLKGRIKAEEVSQKDKYGKIKSYYENPYTKLTNEFKQISSTYGKTFAE
LRDKFKEKNEITKITHFGIIEDKNRDRYLIASELKHEQINHVSTILNKLD
KSSEIITYQVKSITSKTLIKLIKNHTTKKGAISPYADFHTSKTGFNKNEI
EKNWDNYKREQVLVEYVKDCLTDSTMAKNQNWAEFGWNFEKCNSYEDIEH
EIDQKSYLLQSDTISKQSIASLVEGGCLLLPIINQDITSKERKDKNQFSK
DWNHIFEGSKEFRLHPEFAVSYRTPIEGYPVQKRYGRLQFVCAFNAHIVP
QNGEFINLKKQIENFNDEDVQKRNVTEFNKKVNHALSDKEYVVIGIDRGL
KQLATLCVLDKRGKILGDFEIYKKEFVRAEKRSESHWEHTQAETRHILDL
SNLRVETTTEGKKVLVDQSLTLVKKNRDTPDEEATEENKQKIKLKQLSYI
RKLQHKMQTNEQDVLDLINNEPSDEEFKKRIEGLISSFGEGQKYADLPIN
TMREMISDLQGVIARGNNQTEKNKIIELDAADNLKQGIVANMIGIVNYIF
AKYSYKAYISLEDLSRAYGGAKSGYDGRYLPSTSQDEDVDFKEQQNQMLA
GLGTYQFFEMQLLKKLQKIQSDNTVLRFVPAFRSADNYRNILRLEETKYK
SKPFGVVHFIDPKFTSKKCPVCSKTNVYRDKDDILVCKECGFRSDSQLKE
RENNIHYIHNGDDNGAYHIALKSVENLIQMK

In certain embodiments, the type V-A Cas protein comprises MbCsm1 or a variant thereof. In certain embodiments, the type V-A Cas protein comprises an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 989%, or at least 99% identical to the amino acid sequence set forth in SEQ ID NO: 14. In certain embodiments, the type V-A Cas protein comprises the amino acid sequence set forth in SEQ ID NO: 14.

MbCsm1
(SEO ID NO: 14)
MEIQELKNLYEVKKTVRFELKPSKKKIFEGGDVIKLQKDFEKVQKFFLDI
FVYKNEHTKLEFKKKREIKYTWLRTNTKNEFYNWRGKSDTGKNYALNKIG
FLAEEILRWLNEWQELTKSLKDLTQREEHKQERKSDIAFVLRNFLKRQNL
PFIKDFFNAVIDIQGKQGKESDDKIRKFREEIKEIEKNLNACSREYLPTQ
SNGVLLYKASFSYYTLNKTPKEYEDLKKEKESELSSVLLKEIYRRKRFNR
TTNQKDTLFECTSDWLVKIKLGKDIYEWTLDEAYQKMKIWKANQKSNFIE
AVAGDKLTHQNFRKQFPLFDASDEDFETFYRLTKALDKNPENAKKIAQKR
GKFFNAPNETVQTKNYHELCELYKRIAVKRGKIIAEIKGIENEEVQSQLL
THWAVIAEERDKKFIVLIPRKNGGKLENHKNAHAFLQEKDRKEPNDIKVY
HFKSLTLRSLEKLCFKEAKNTFAMEIKKETNPKIWPIYKQEWNSTPERLI
KEYKQVLQSNYAQIYLDLVDFGNLNTFLETHFTTLEEFESDLEKTCYTKV
PVYFAKKELETFADEFEAEVFEITTRSISTESKRKENAHAEIWRDFWSRE
NEEENHITRLNPEVSVLYRDEIKEKSNTSRKNRKSNANNRFSDPRFTLAT
TITLNADKKKSNLAFKTVEDINIHIDNFNKKFSKNFSGEWVYGIDRGLKE
LATLNVVKFSDVKNVFGVSQPKEFAKIPIYKLRDEKAILKDENGLSLKNA
KGEARKVIDNISDVLEEGKEPDSTLFEKREVSSIDLTRAKLIKGHIISNG
DQKTYLKLKETSAKRRIFELFSTAKIDKSSQFHVRKTIELSGTKIYWLCE
WQRQDSWRTEKVSLRNTLKGYLQNLDLKNRFENIETIEKINHLRDAITAN
MVGILSHLQNKLEMQGVIALENLDTVREQSNKKMIDEHFEQSNEHVSRRL
EWALYCKFANTGEVPPQIKESIFLRDEFKVCQIGILNFIDVKGTSSNCPN
CDQESRKTGSHFICNFQNNCIFSSKENRNLLEQNLHNSDDVAAFNIAKRG
LEIVKV

More type V-A Cas proteins and their corresponding naturally occurring CRISPR-Cas systems can be identified by computational and experimental methods known in the art, e.g., as described in U.S. Pat. No. 9,790,490 and Shmakov et al. (2015) MOL. CELL, 60: 385. Exemplary computational methods include analysis of putative Cas proteins by homology modeling, structural BLAST, PSI-BLAST, or HHPred, and analysis of putative CRISPR loci by identification of CRISPR arrays. Exemplary experimental methods include in vitro cleavage assays and in-cell nuclease assays (e.g., the Surveyor assay) as described in Zetsche et al. (2015) CELL, 163: 759.

In certain embodiments, the Cas protein is a Cas nuclease that directs cleavage of one or both strands at the target locus, such as the target strand (i.e., the strand having the target nucleotide sequence that hybridizes with a single guide nucleic acid or dual guide nucleic acids) and/or the non-target strand. In certain embodiments, the Cas nuclease directs cleavage of one or both strands within about 1, 2, 3, 4, 5, 6, 7, 8, 9, 10, 15, 20, 25, 50, 100, 200, 500, or more nucleotides from the first or last nucleotide of the target nucleotide sequence or its complementary sequence. In certain embodiments, the cleavage is staggered, i.e. generating sticky ends. In certain embodiments, the cleavage generates a staggered cut with a 5′ overhang. In certain embodiments, the cleavage generates a staggered cut with a 5′ overhang of 1 to 5 nucleotides, e.g., of 4 or 5 nucleotides. In certain embodiments, the cleavage site is distant from the PAM, e.g., the cleavage occurs after the 18th nucleotide on the non-target strand and after the 23rd nucleotide on the target strand.

In certain embodiments, the Cas protein lacks substantially all DNA cleavage activity. Such a Cas protein can be generated by introducing one or more mutations to an active Cas nuclease (e.g., a naturally occurring Cas nuclease). A mutated Cas protein is considered to substantially lack all DNA cleavage activity when the DNA cleavage activity of the protein has no more than about 25%, 10%, 5%, 1%, 0.1%, 0.01%, or less of the DNA cleavage activity of the corresponding non-mutated form, for example, nil or negligible as compared with the non-mutated form. Thus, the Cas protein may comprise one or more mutations (e.g., a mutation in the RuvC domain of a type V-A Cas protein) and be used as a generic DNA binding protein with or without fusion to an effector domain. Exemplary mutations include D908A. E993A, and D1263A with reference to the amino acid positions in AsCpf1; D832A, E925A, and D1180A with reference to the amino acid positions in LbCpf1; and D917A, E1006A, and D1255A with reference to the amino acid position numbering of the FnCpf1. More mutations can be designed and generated according to the crystal structure described in Yamano er al. (2016) CELL, 165: 949.

It is understood that the Cas protein, rather than losing nuclease activity to cleave all DNA, may lose the ability to cleave only the target strand or only the non-target strand of a double-stranded DNA, thereby being functional as a nickase (see, Gao et al. (2016) CELL RES., 26: 901). Accordingly, in certain embodiments, the Cas nuclease is a Cas nickase. In certain embodiments, the Cas nuclease has the activity to cleave the non-target strand but substantially lacks the activity to cleave the target strand, e.g., by a mutation in the Nuc domain. In certain embodiments, the Cas nuclease has the cleavage activity to cleave the target strand but substantially lacks the activity to cleave the non-target strand.

In other embodiments, the Cas nuclease has the activity to cleave a double-stranded DNA and result in a double-strand break.

Cas proteins that lack substantially all DNA cleavage activity or have the ability to cleave only one strand may also be identified from naturally occurring systems. For example, certain naturally occurring CRISPR-Cas systems may retain the ability to bind the target nucleotide sequence but lose entire or partial DNA cleavage activity in eukaryotic (e.g., mammalian or human) cells. Such type V-A proteins are disclosed, for example, in Kim et al. (2017) ACS SYNTH. BIOL. 6(7): 1273-82 and Zhang et al. (2017) CELL DISCOV. 3:17018.

The activity of the Cas protein (e.g., Cas nuclease) can be altered, thereby creating an engineered Cas protein. In certain embodiments, the altered activity of the engineered Cas protein comprises increased targeting efficiency and/or decreased off-target binding. While not wishing to be bound by theory, it is hypothesized that off-target binding can be recognized by the Cas protein, for example, by the presence of one or more mismatches between the spacer sequence and the target nucleotide sequence, which may affect the stability and/or conformation of the CRISPR-Cas complex. In certain embodiments, the altered activity comprises modified binding, e.g., increased binding to the target locus (e.g., the target strand or the non-target strand) and/or decreased binding to off-target loci. In certain embodiments, the altered activity comprises altered charge in a region of the protein that associates with a single guide nucleic acid or dual guide nucleic acids. In certain embodiments, the altered activity of the engineered Cas protein comprises altered charge in a region of the protein that associates with the target strand and/or the non-target strand. In certain embodiments, the altered activity of the engineered Cas protein comprises altered charge in a region of the protein that associates with an off-target locus. The altered charge can include decreased positive charge, decreased negative charge, increased positive charge, and increased negative charge. For example, decreased negative charge and increased positive charge may generally strengthen the binding to the nucleic acid(s) whereas decreased positive charge and increased negative charge may weaken the binding to the nucleic acid(s). In certain embodiments, the altered activity comprises increased or decreased steric hindrance between the protein and a single guide nucleic acid or dual guide nucleic acids. In certain embodiments, the altered activity comprises increased or decreased steric hindrance between the protein and the target strand and/or the non-target strand. In certain embodiments, the altered activity comprises increased or decreased steric hindrance between the protein and an off-target locus. In certain embodiments, the modification or mutation comprises a substitution of Lys, His, Arg, Glu, Asp, Ser, Gly, or Thr. In certain embodiments, the modification or mutation comprises a substitution with Gly, Ala, Ile, Glu, or Asp. In certain embodiments, the modification or mutation comprises an amino acid substitution in the groove between the WED and RuvC domain of the Cas protein (e.g., a type V-A Cas protein).

In certain embodiments, the altered activity of the engineered Cas protein comprises increased nuclease activity to cleave the target locus. In certain embodiments, the altered activity of the engineered Cas protein comprises decreased nuclease activity to cleave an off-target locus. In certain embodiments, the altered activity of the engineered Cas protein comprises altered helicase kinetics. In certain embodiments, the engineered Cas protein comprises a modification that alters formation of the CRISPR complex.

In certain embodiments, a protospacer adjacent motif (PAM) or PAM-like motif directs binding of the Cas protein complex to the target locus. Many Cas proteins have PAM specificity. The precise sequence and length requirements for the PAM differ depending on the Cas protein used. PAM sequences are typically 2-5 base pairs in length and are adjacent to (but located on a different strand of target DNA from) the target nucleotide sequence. PAM sequences can be identified using a method known in the art, such as testing cleavage, targeting, or modification of oligonucleotides having the target nucleotide sequence and different PAM sequences.

Exemplary PAM sequences are provided in Tables 4 and 5. In one embodiment, the Cas protein is MAD7 and the PAM is TITN, wherein N is A, C. G. or T. In another embodiment, the Cas protein is MAD7 and the PAM is CTTN, wherein N is A, C, G, or T. In another embodiment, the Cas protein is AsCpf1 and the PAM is TITN, wherein N is A, C, G, or T. In another embodiment, the Cas protein is FnCpf1 and the PAM is 5′ TTN, wherein N is A, C, G, or T. PAM sequences for certain other type V-A Cas proteins are disclosed in Zetsche et al. (2015) CELL, 163: 759 and U.S. Pat. No. 9,982,279. Further, engineering of the PAM Interacting (PI) domain of a Cas protein may allow programing of PAM specificity, improve target site recognition fidelity, and increase the versatility of the engineered, non-naturally occurring system. Exemplary approaches to alter the PAM specificity of Cpf1 is described in Gao et al. (2017) NAT. BIOTECHNOL., 35: 789.

In certain embodiments, the engineered Cas protein comprises a modification that alters the Cas protein specificity in concert with modification to targeting range. Cas mutants can be designed to have increased target specificity as well as accommodating modifications in PAM recognition, for example by choosing mutations that alter PAM specificity (e.g., in the Pi domain) and combining those mutations with groove mutations that increase (or if desired, decrease) specificity for the on-target locus versus off-target loci. The Cas modifications described herein can be used to counter loss of specificity resulting from alteration of PAM recognition, enhance gain of specificity resulting from alteration of PAM recognition, counter gain of specificity resulting from alteration of PAM recognition, or enhance loss of specificity resulting from alteration of PAM recognition.

In certain embodiments, the engineered Cas protein comprises one or more nuclear localization signal (NLS) motifs. In certain embodiments, the engineered Cas protein comprises at least 2 (e.g., at least 3, at least 4, at least 5, at least 6, at least 7, at least 8, at least 9, or at least 10) NLS motifs. Non-limiting examples of NLS motifs include: the NLS of SV40 large T-antigen, having the amino acid sequence of PKKKRKV (SEQ ID NO: 35); the NLS from nucleoplasmin, e.g., the nucleoplasmin bipartite NLS having the amino acid sequence of KRPAATKKAGQAKKKK (SEQ ID NO: 36); the c-myc NLS, having the amino acid sequence of PAAKRVKLD (SEQ ID NO: 37) or RQRRNELKRSP (SEQ ID NO: 38); the hRNPA1 M9 NLS, having the amino acid sequence of NQSSNFGPMKGGNFGGRSSGPYGGGGQYFAKPRNQGGY (SEQ ID NO: 39); the importin-α IBB domain NLS, having the amino acid sequence of RMRIZFKNKGKDTAELRRRRVEVSVELRKAKKDEQILKRRNV (SEQ ID NO: 40); the myoma T protein NLS, having the amino acid sequence of VSRKRPRP (SEQ ID NO: 41) or PPKKARED (SEQ ID NO: 42); the human p53 NLS, having the amino acid sequence of PQPKKKPL (SEQ ID NO: 43); the mouse c-abl IV NLS, having the amino acid sequence of SALIKKKKKMAP (SEQ ID NO: 44); the influenza virus NS1 NLS, having the amino acid sequence of DRLRR (SEQ ID NO: 45) or PKQKKRK (SEQ ID NO: 46); the hepatitis virus δ antigen NLS, having the amino acid sequence of RKLKKKIKKL (SEQ ID NO: 47); the mouse M×1 protein NLS, having the amino acid sequence of REKKKFLKRR (SEQ ID NO: 48): the human poly(ADP-ribose) polymerase NLS, having the amino acid sequence of KRKGDEVDGVDEVAKKKSKK (SEQ ID NO: 49); the human glucocorticoid receptor NLS, having the amino acid sequence of RKCLQAGMNLEARKTKK (SEQ ID NO: 33), and synthetic NLS motifs such as PAAKKKKLD (SEQ ID NO: 34).

In general, the one or more NLS motifs are of sufficient strength to drive accumulation of the Cas protein in a detectable amount in the nucleus of a cukaryotic cell. The strength of nuclear localization activity may derive from the number of NLS motifs) in the Cas protein, the particular NLS motifs) used, the position(s) of the NLS motifs), or a combination of these factors. In certain embodiments, the engineered Cas protein comprises at least 1 (e.g., at least 2, at least 3, at least 4, at least 5, at least 6, at least 7, at least 8, at least 9, or at least 10) NLS motifs) at or near the N-terminus (e.g., within about 1, 2, 3, 4, 5, 10, 15, 20, 25, 30, 40, 50, or more amino acids along the polypeptide chain from the N-terminus). In certain embodiments, the engineered Cas protein comprises at least 1 (e.g., at least 2, at least 3, at least 4, at least 5, at least 6, at least 7, at least 8, at least 9, or at least 10) NLS motif(s) at or near the C-terminus (e.g., within about 1, 2, 3, 4, 5, 10, 15, 20, 25, 30, 40, 50, or more amino acids along the polypeptide chain from the C-terminus). In certain embodiments, the engineered Cas protein comprises at least 1 (e.g., at least 2, at least 3, at least 4, at least 5, at least 6, at least 7, at least 8, at least 9, or at least 10) NLS motifs) at or near the C-terminus and at least 1 (e.g., at least 2, at least 3, at least 4, at least 5, at least 6, at least 7, at least 8, at least 9, or at least 10) NLS motif(s) at or near the N-terminus. In certain embodiments, the engineered Cas protein comprises one, two, or three NLS motifs at or near the C-terminus. In certain embodiments, the engineered Cas protein comprises one NLS motif at or near the N-terminus and one, two, or three NLS motifs at or near the C-terminus. In certain embodiments, the engineered Cas protein comprises a nucleoplasmin NLS at or near the C-terminus.

Detection of accumulation in the nucleus may be performed by any suitable technique. For example, a detectable marker may be fused to the nucleic acid-targeting protein, such that location within a cell may be visualized. Cell nuclei may also be isolated from cells, the contents of which may then be analyzed by any suitable process for detecting the protein, such as immunohistochemistry, Western blot, or enzyme activity assay. Accumulation in the nucleus may also be determined indirectly, such as by an assay that detects the effect of the nuclear import of a Cas protein complex (e.g., assay for DNA cleavage or mutation at the target locus, or assay for altered gene expression activity) as compared to a control not exposed to the Cas protein or exposed to a Cas protein lacking one or more of the NLS motifs.

In certain embodiments, the Cas protein is a chimeric Cas protein, e.g., a Cas protein having enhanced function by being a chimera. Chimeric Cas proteins may be new Cas proteins containing fragments from more than one naturally occurring Cas proteins or variants thereof. For example, fragments of multiple type V-A Cas homologs (e.g., orthologs) may be fused to form a chimeric Cas protein. In certain embodiments, the chimeric Cas protein comprises fragments of Cpf1 orthologs from multiple species and/or strains.

In certain embodiments, the Cas protein comprises one or more effector domains. The one or more effector domains may be located at or near the N-terminus of the Cas protein and/or at or near the C-terminus of the Cas protein. In certain embodiments, an effector domain comprised in the Cas protein is a transcriptional activation domain (e.g., VP64), a transcriptional repression domain (e.g., a KRAB domain or an SID domain), an exogenous nuclease domain (e.g., FokI), a deaminase domain (e.g., cytidine deaminase or adenine deaminase), or a reverse transcriptase domain (e.g., a high fidelity reverse transcriptase domain). Other activities of effector domains include but are not limited to methylase activity, demethylase activity, transcription release factor activity, translational initiation activity, translational activation activity, translational repression activity, histone modification (e.g., acetylation or demethylation) activity, single-stranded RNA cleavage activity, double-strand RNA cleavage activity, single-strand DNA cleavage activity, double-strand DNA cleavage activity, and nucleic acid binding activity.

In certain embodiments, the Cas protein comprises one or more protein domains that enhance homology-directed repair (HDR) and/or inhibit non-homologous end joining (NHEJ). Exemplary protein domains having such functions are described in Jayavaradhan et al. (2019) NAT. COMMUN. 10(1): 2866 and Janssen et al. (2019) MOL. THER. NUCLEIC ACIDS 16: 141-54. In certain embodiments, the Cas protein comprises a dominant negative version of p53-binding protein 1 (53BP1), for example, a fragment of 53BP1 comprising a minimum focus forming region (e.g., amino acids 1231-1644 of human 53BP1). In certain embodiments, the Cas protein comprises a motif that is targeted by APC-Cdh1, such as amino acids 1-110 of human Geminin, thereby resulting in degradation of the fusion protein during the HDR non-permissive G1 phase of the cell cycle.

In certain embodiments, the Cas protein comprises an inducible or controllable domain. Non-limiting examples of inducers or controllers include light, hormones, and small molecule drugs. In certain embodiments, the Cas protein comprises a light inducible or controllable domain. In certain embodiments, the Cas protein comprises a chemically inducible or controllable domain.

In certain embodiments, the Cas protein comprises a tag protein or peptide for ease of tracking or purification. Non-limiting examples of tag proteins and peptides include fluorescent proteins (e.g., green fluorescent protein (GFP), YFP, RFP, CFP, mCherry, tdTomato), HIS tags (e.g., 6×His tag, (SEQ ID NO: 789)), hemagglutinin (HA) tag, FLAG tag, and Myc tag.

In certain embodiments, the Cas protein is conjugated to a non-protein moiety, such as a fluorophore useful for genomic imaging. In certain embodiments, the Cas protein is covalently conjugated to the non-protein moiety. The terms “CRISPR-Associated protein,” “Cas protein,” “Cas,” “CRISPR-Associated nuclease.” and “Cas nuclease” are used herein to include such conjugates despite the presence of one or more non-protein moieties.

### Guide Nucleic Acids

In certain embodiments, the guide nucleic acid of the present invention is a guide nucleic acid that is capable of binding a Cas protein alone (e.g., in the absence of a tracrRNA). Such guide nucleic acid is also called a single guide nucleic acid. In certain embodiments, the single guide nucleic acid is capable of activating a Cas nuclease alone (e.g., in the absence of a tracrRNA). The present invention also provides an engineered, non-naturally occurring system comprising the single guide nucleic acid. In certain embodiments, the system further comprises the Cas protein that the single guide nucleic acid is capable of binding or the Cas nuclease that the single guide nucleic acid is capable of activating.

In other embodiments, the guide nucleic acid of the present invention is a targeter nucleic acid that, in combination with a modulator nucleic acid, is capable of binding a Cas protein. In certain embodiments, the guide nucleic acid is a targeter nucleic acid that, in combination with a modulator nucleic acid, is capable of activating a Cas nuclease. The present invention also provides an engineered, non-naturally occurring system comprising the targeter nucleic acid and the cognate modulator nucleic acid. In certain embodiments, the system further comprises the Cas protein that the targeter nucleic acid and the modulator nucleic acid are capable of binding or the Cas nuclease that the targeter nucleic acid and the modulator nucleic acid are capable of activating.

It is contemplated that the single or dual guide nucleic acids need to be the compatible with a Cas protein (e.g., Cas nuclease) to provide an operative CRISPR system. For example, the targeter stem sequence and the modulator stem sequence can be derived from a naturally occurring crRNA capable of activating a Cas nuclease in the absence of a tracrRNA. Alternatively, the targeter stem sequence and the modulator stem sequence can be derived from a naturally occurring set of crRNA and tracrRNA, respectively, that are capable of activating a Cas nuclease. In certain embodiments, the nucleotide sequences of the targeter stem sequence and the modulator stem sequence are identical to the corresponding stem sequences of a stem-loop structure in such naturally occurring crRNA.

Guide nucleic acid sequences that are operative with a type 11 or type V Cas protein are known in the art and are disclosed, for example, in U.S. Pat. Nos. 9,790,490, 9,896,696, and 10,113,179, and U.S. Patent Application Publication Nos. 2014/0242664 and 2014/0068797. Exemplary single guide and dual guide sequences that are operative with certain type V-A Cas proteins are provided in Tables 4 and 5, respectively. It is understood that these sequences are merely illustrative, and other guide nucleic acid sequences may also be used with these Cas proteins.

TABLE 4
Type V-A Cas Protein and Corresponding Single Guide Nucleic Acid
Sequences

| Cas Protein | Scaffold Sequence¹ | PAM² |
|---|---|---|
| MAD7 (SEQ ID | UAAUUUCUACUCUUGUAGA (SEQ ID NO: 15), | 5′ ths |
| NO: 1) | AUCUACAACAGUAGA (SEO ID NO: 16), | or 5′ |
| | AUCUACAAAAGUAGA (SEQ ID NO: 17), | CTTN |
| | GGAAUUUCUACUCUUGUAGA (SEQ ID NO: 18), | |
| | UAAUUCCCACUCUUGUGGG (SEQ ID NO: 19) | |
| MAD2 (SEQ ID | AUCUACAAGAGUAGA (SEQ ID NO: 20), | 5′ TTTN |
| NO: 2) | AUCUACAACAGUAGA (SEO ID NO: 16), | |
| | AUCUACAAAAGUAGA (SEQ ID NO: 17), | |
| | AUCUACACUAGUAGA (SEQ ID NO: 21) | |
| AsCpf1 (SEQ ID | UAAUUUCUACUCUUGUAGA (SEQ ID NO: 15) | 5′ TTTN |
| NO: 3) | | |
| LbCpf1 (SEQ ID | UAAUUUCUACUAAGUGUAGA (SEQ ID NO: 22) | 5′ TTTN |
| NO: 4) | | |
| FnCpf1 (SEQ ID | UAAUUUUCUACUUGUUGUAGA (SEQ ID NO: | 5′ TTN |
| NO: 5) | 23) | |
| PbCpf1 (SEQ ID | AAUUUCUACUGUUGUAGA (SEQ ID NO: 24) | 5′ TTTC |
| NO: 6) | | |
| PsCpf1 (SEQ ID | AAUUUCUACUGUUGUAGA (SEQ ID NO: 24) | 5′ TTTC |
| NO: 7) | | |
| As2Cpf1 (SEQ ID | AAUUUCUACUGUUGUAGA (SEQ ID NO: 24) | 5′ TTTC |
| NO: 8) | | |
| McCpf 1 (SEQ ID | GAAUUUCUACUGUUGUAGA (SEQ ID NO: 25) | 5′ TTTC |
| NO: 9) | | |
| 1b3Cpf1 (SEQ ID | GAAUUUCUACUGUUGUAGA (SEQ ID NO: 25) | 5′ TTTC |
| NO: 10) | | |
| EcCpf1 (SEQ ID | GAAUUUCUACUGUUGUAGA (SEQ ID NO: 25) | 5′ TTTC |
| NO: 11) | | |
| SmCsm1 (SEQ ID | GAAUUUCUACUGUUGUAGA (SEQ ID NO: 25) | 5′ TTTC |
| NO: 12) | | |
| SsCsm1 (SEQ ID | GAAUUUCUACUGUUGUAGA (SEQ ID NO: 25) | 5′ TTTC |
| NO: 13) | | |
| MbCsm1 (SEQ ID | GAAUUUCUACUGUUGUAGA (SEQ ID NO: 25) | 5′ TTTC |
| NO: 14) | | |

¹The modulator sequence in the scaffold sequence is underlined; the targeter stem sequence in the scaffold sequence is bold-underlined. It is understood that a “scaffold sequence” listed herein constitutes a portion of a single guide nucleic acid. Additional nucleotide sequences, oilier than the spacer sequence, can be comprised in the single guide nucleic acid.
²In the consensus PAM sequences, N represents A, C, G, or T. Where the PAM sequence is preceded by “5′,” it means that the PAM is located immediately upstream of the target nucleotide sequence when using the non-target strand (i.e., the strand not hybridized with the spacer sequence) as the coordinate.

TABLE 5
Type V-A Cas Protein and Corresponding Dual Guide Nucleic Acid
Sequences

| | | Targeter | |
|---|---|---|---|
| | | Stem | |
| Cas Protein | Modulator Sequence¹ | Sequence | PAM² |
| MAD7 (SEQ ID NO: 1) | UAAUUUCUAC (SEQ ID NO: 26) | GUAGA | 5′ TTTN |
| | AUCUAC (SEQ ID NO: 27) | GUAGA | or 5′ |
| | GGAAUUUCUAC (SEQ ID NO: | GUAGA | CTTN |
| | 28) | | |
| | UAAUUCCCAC (SEQ ID NO: 29) | GUGGG | |
| MAD2 (SEQ ID NO: 2) | AUCUAC (SEQ ID NO: 27) | GUAGA | 5′ TTTN |
| AsCpf1 (SEQ ID NO: 3) | UAAUUUCUAC (SEQ ID NO: 26) | GUAGA | 5′ TTTN |
| LbCpf1 (SEQ ID NO: 4) | UAAUUUCUAC (SEQ ID NO: 26) | GUAGA | 5′ TTTN |
| FnCpf1 (SEQ ID NO: 5) | UAAUUUUCUACU (SEQ ID NO: | GUAGA | 5′ TTN |
| | 30) | | |
| PbCpf1 (SEQ ID NO: 6) | AAUUUCUAC (SEQ ID NO: 31) | GUAGA | 5′ TTTC |
| PsCpf1 (SEQ ID NO: 7) | AAUUUCUAC (SEQ ID NO: 31) | GUAGA | 5′ TTTC |
| As2Cpf1 (SEQ ID NO: 8) | AAUUUCUAC (SEQ ID NO: 31) | GUAGA | 5′ TTTC |
| McCpf1 (SEQ ID NO: 9) | GAAUUUCUAC (SEQ ID NO: 32) | GUAGA | 5′ TTTC |
| Lb3Cpf1 (SEQ ID NO: 10) | GAAUUUCUAC (SEQ ID NO: 32) | GUAGA | 5′ TTTC |
| EcCpf1 (SEQ ID NO: 11) | GAAUUUCUAC (SEQ ID NO: 32) | GUAGA | 51 TTTC |
| SmCsm1 (SEQ ID NO: 12) | GAAUUUCUAC (SEQ ID NO: 32) | GUAGA | 5′ TTTC |
| SsCsm1 (SEQ ID NO: 13) | GAAUUUCUAC (SEQ ID NO: 32) | GUAGA | 5′ TTTC |
| MbCsm1 (SEQ ID NO: 14) | GAAUUUCUAC (SEQ ID NO: 32) | GUAGA | 5′ TTTC |

¹It is understood that a “modulator sequence” listed herein may constitute the nucleotide sequence of a modulator nucleic acid. Alternati vely, additional nucleotide sequences can be comprised in the modulator nucleic acid 5′ and/or 3′ to a “modulator sequence” listed herein.
²In the consensus PAM sequences, N represents A, C, G, or T. Where the PAM sequence is preceded by “5′,” it means that the PAM is located immediately upstream of the target nucleotide sequence when using the non-target strand (z.e., the strand not hybridized with the
spacer sequence) as the coordinate.

In certain embodiments, the guide nucleic acid of the present invention, in the context of a type V-A CRISPR-Cas system, comprises a targeter stem sequence listed in Table 5. The same targeter stem sequences, as a portion of scaffold sequences, are bold-underlined in Table 4.

In certain embodiments, the guide nucleic acid is a single guide nucleic acid that comprises, from 5′ to 3′, a modulator stem sequence, a loop sequence, a targeter stem sequence, and a spacer sequence disclosed herein. In certain embodiments, the targeter stem sequence in the single guide nucleic acid is listed in Table 4 as a bold-underlined portion of scaffold sequence, and the modulator stem sequence is complementary (e.g., 100% complementary) to the targeter stem sequence. In certain embodiments, the single guide nucleic acid comprises, from 5′ to 3′, a modulator sequence listed in Table 4 as an underlined portion of a scaffold sequence, a loop sequence, a targeter stem sequence a bold-underlined portion of the same scaffold sequence, and a spacer sequence disclosed herein. In certain embodiments, an engineered, non-naturally occurring system of the present invention comprises the single guide nucleic acid comprising a scaffold sequence listed in Table 4. In certain embodiments, the system further comprises a Cas protein (e.g., Cas nuclease) comprising an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in the SEQ ID NO listed in the same line of Table 4. In certain embodiments, the system further comprises a Cas protein (e.g., Cas nuclease) comprising the amino acid sequence set forth in the SEQ ID NO listed in the same line of Table 4. In certain embodiments, the system is useful for targeting, editing, or modifying a nucleic acid comprising a target nucleotide sequence close or adjacent to (e. g., immediately downstream of) a PAM listed in the same line of Table 4 when using the non-target strand (i.e., the strand not hybridized with the spacer sequence) as the coordinate.

In certain embodiments, the guide nucleic acid is a targeter guide nucleic acid that comprises, from 5′ to 3′, a targeter stem sequence and a spacer sequence disclosed herein. In certain embodiments, the targeter stem sequence in the targeter nucleic acid is listed in Table 5. In certain embodiments, an engineered, non-naturally occurring system of the present invention comprises the targeter nucleic acid and a modulator stem sequence complementary (e.g., 100% complementary) to the targeter stem sequence. In certain embodiments, the modulator nucleic acid comprises a modulator sequence listed in the same line of Table 5. In certain embodiments, the system further comprises a Cas protein (e.g., Cas nuclease) comprising an amino acid sequence at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in the SEQ ID NO listed in the same line of Table 5. In certain embodiments, the system further comprises a Cas protein (e.g., Cas nuclease) comprising the amino acid sequence set forth in the SEQ ID NO listed in the same line of Table 5. In certain embodiments, the system is useful for targeting, editing, or modifying a nucleic acid comprising a target nucleotide sequence close or adjacent to (e.g., immediately downstream of) a PAM listed in the same line of Table 5 when using the non-target strand (i.e., the strand not hybridized with the spacer sequence) as the coordinate.

The single guide nucleic acid, the targeter nucleic acid, and/or the modulator nucleic acid can be synthesized chemically or produced in a biological process (e.g., catalyzed by an RNA polymerase in an in vitro reaction). Such reaction or process may limit the lengths of the single guide nucleic acid, targeter nucleic acid, and modulator nucleic acid. In certain embodiments, the single guide nucleic acid is no more than 100, 90, 80, 70, 60, 50, 40, 30, or 25 nucleotides in length. In certain embodiments, the single guide nucleic acid is at least 20, 25, 30, 40, 50, 60, 70, 80, or 90 nucleotides in length. In certain embodiments, the single guide nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, the targeter nucleic acid is no more than 100, 90, 80, 70, 60, 50, 40, 30, or 25 nucleotides in length. In certain embodiments, the targeter nucleic acid is at least 20, 25, 30, 40, 50, 60, 70, 80, or 90 nucleotides in length. In certain embodiments, the targeter nucleic acid is 20-100, 20-90, 20-80, 20-70, 20-60, 20-50, 20-40, 20-30, 20-25, 25-100, 25-90, 25-80, 25-70, 25-60, 25-50, 25-40, 25-30, 30-100, 30-90, 30-80, 30-70, 30-60, 30-50, 3040, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length. In certain embodiments, the modulator nucleic acid is no more than 100, 90, 80, 70, 60, 50, 40, 30, or 20 nucleotides in length. In certain embodiments, the modulator nucleic acid is at least 10, 15, 20, 25, 30, 40, 50, 60, 70, 80, or 90 nucleotides in length. In certain embodiments, the modulator nucleic acid is 10-100, 10-90, 10-80, 10-70, 10-60, 10-50, 10-40, 10-30, 10-20, 15-100, 15-90, 15-80, 15-70, 15-60, 15-50, 15-40, 15-30, 15-20, 20-100, 20-90, 20-80, 20-70, 20-60, 20-50, 2040, 20-30, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleotides in length.

It is contemplated that the length of the duplex formed within the single guide nuclei acid or formed between the targeter nucleic acid and the modulator nucleic acid may be a factor in providing an operative CRISPR system. In certain embodiments, the targeter stem sequence and the modulator stem sequence each consist of 4-10 nucleotides that base pair with each other. In certain embodiments, the targeter stem sequence and the modulator stem sequence each consist of 4-9, 4-8, 4-7, 4-6, 4-5, 5-10, 5-9, 5-8, 5-7, or 5-6 nucleotides that base pair with each other. In certain embodiments, the targeter stem sequence and the modulator stem sequence each consist of 4, 5, 6, 7, 8, 9, or 10 nucleotides. It is understood that the composition of the nucleotides in each sequence affects the stability of the duplex, and a C-G base pair confers greater stability than an A-U base pair. In certain embodiments, 20%-80%, 20%-70%, 20%-60%, 20%-50%, 20%-40%, 20%-30%, 30%-80%, 30%-70%, 30%-60%, 30%-50%, 30%-40%, 40%-80%, 40%-70%, 40%-60%, 40%-50%, 50%-80%, 50%-70%, 50%-60%, 60%-80%, 60%-70%, or 70%-80% of the base pairs are C-G base pairs.

In certain embodiments, the targeter stem sequence and the modulator stem sequence each consist of 5 nucleotides. As such, the targeter stem sequence and the modulator stem sequence form a duplex of 5 base pairs. In certain embodiments, 0-4, 0-3, 0-2, 0-1, 1-5, 1-4, 1-3, 1-2, 2-5, 2-4, 2-3, 3-5, 3-4, or 4-5 out of the 5 base pairs are C-G base pairs. In certain embodiments, 0, 1, 2, 3, 4, or 5 out of the 5 base pairs are C-G base pairs. In certain embodiments, the targeter stem sequence consists of 5′-GUAGA-3′ and the modulator stem sequence consists of 5′-UCUAC-3′. In certain embodiments, the targeter stem sequence consists of 5′-GUGGG-3′ and the modulator stem sequence consists of 5′-CCCAC-3′.

In certain embodiments, in a type V-A system, the 3′ end of the targeter stem sequence is linked by no more than 1, 2, 3, 4, 5, 6, 7, 8, 9, or 10 nucleotides to the 5′ end of the spacer sequence. In certain embodiments, the targeter stem sequence and the spacer sequence are adjacent to each other, directly linked by an internucleotide bond. In certain embodiments, the targeter stem sequence and the spacer sequence are linked by one nucleotide, e.g., a uridine. In certain embodiments, the targeter stem sequence and the spacer sequence are linked by two or more nucleotides. In certain embodiments, the targeter stem sequence and the spacer sequence are linked by 1, 2, 3, 4, 5, 6, 7, 8, 9, or 10 nucleotides.

In certain embodiments, the targeter nucleic acid further comprises an additional nucleotide sequence 5′ to the targeter stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 2 nucleotides. In certain embodiments, the additional nucleotide sequence is reminiscent to the loop or a fragment thereof (e.g., one, two, three, or four nucleotides at the 3′ end of the loop) in a crRNA of a corresponding single guide CRISPR-Cas system. It is understood that an additional nucleotide sequence 5′ to the targeter stem sequence is dispensable. Accordingly, in certain embodiments, the targeter nucleic acid does not comprise any additional nucleotide 5′ to the targeter stem sequence.

In certain embodiments, the targeter nucleic acid or the single guide nucleic acid further comprises an additional nucleotide sequence containing one or more nucleotides at the 3′ end that does not hybridize with the target nucleotide sequence. The additional nucleotide sequence may protect the targeter nucleic acid from degradation by 3′-5′ exonuclease. In certain embodiments, the additional nucleotide sequence is no more than 100 nucleotides in length. In certain embodiments, the additional nucleotide sequence is no more than 90, 80, 70, 60, 50, 40, 30, 20, or 10 nucleotides in length. In certain embodiments, the additional nucleotide sequence is at least 1, 2, 3, 4, 5, 6, 7, 8, 9, 10, 15, 20, 25, 30, 35, 40, 45, or 50 nucleotides in length. In certain embodiments, the additional nucleotide sequence is 5-100, 5-50, 5-40, 5-30, 5-25, 5-20, 5-15, 5-10, 10-100, 10-50, 10-40, 10-30, 10-25, 10-20, 10-15, 15-100, 15-50, 15-40, 15-30, 15-25, 15-20, 20-100, 20-50, 20-40, 20-30, 20-25, 25-100, 25-50, 25-40, 25-30, 30-100, 30-50, 30-40, 40-100, 40-50, or 50-100 nucleotides in length.

In certain embodiments, the additional nucleotide sequence forms a hairpin with the spacer sequence. Such secondary structure may increase the specificity of guide nucleic acid or the engineered, non-naturally occurring system (see. Kocak et al. (2019) NAT. BIOTECH. 37: 657-66). In certain embodiments, the free energy change during the hairpin formation is greater than or equal to −20 kcal/mol, −15 kcal/mol, −14 kcal/mol, −13 kcal/mol, −12 kcal/mol, −11 kcal/mol, or −10 kcal/mol. In certain embodiments, the free energy change during the hairpin formation is greater than or equal to −5 kcal/mol, −6 kcal/mol, −7 kcal/mol, −8 kcal/mol, −9 kcal/mol, −10 kcal/mol, −11 kcal/mol, −12 kcal/mol, −13 kcal/mol, −14 kcal/mol, or −15 kcal/mol. In certain embodiments, the free energy change during the hairpin formation is in the range of −20 to −10 kcal/mol, −20 to −11 kcal/mol, −20 to −12 kcal/mol, −20 to −13 kcal/mol, −20 to −14 kcal/mol, −20 to −15 kcal/mol, −15 to −10 kcal/mol, −15 to −11 kcal/mol, −15 to −12 kcal/mol, −15 to −13 kcal/mol, −15 to −14 kcal/mol, −14 to −10 kcal/mol, −14 to −11 kcal/mol, −14 to −12 kcal/mol, −14 to −13 kcal/mol, −13 to −10 kcal/mol, −13 to −11 kcal/mol, −13 to −12 kcal/mol, −12 to −10 kcal/mol, −12 to −11 kcal/mol, or −11 to −10 kcal/mol. In other embodiments, the targeter nucleic acid or the single guide nucleic acid does not comprise any nucleotide 3′ to the spacer sequence.

In certain embodiments, the modulator nucleic acid further comprises an additional nucleotide sequence 3′ to the modulator stem sequence. In certain embodiments, the additional nucleotide sequence comprises at least 1 (e.g., at least 2, at least 3, at least 4, at least 5, at least 6, at least 7, at least 8, at least 9, at least 10, at least 15, at least 20, at least 25, at least 30, at least 35, at least 40, at least 45, or at least 50) nucleotides. In certain embodiments, the additional nucleotide sequence consists of 1, 2, 3, 4, 5, 6, 7, 8, 9, 10, 15, 20, 25, 30, 35, 40, 45, or 50 nucleotides. In certain embodiments, the additional nucleotide sequence consists of 1 nucleotide (e.g., uridine). In certain embodiments, the additional nucleotide sequence consists of 2 nucleotides. In certain embodiments, the additional nucleotide sequence is reminiscent to the loop or a fragment thereof (e.g., one, two, three, or four nucleotides at the 5′ end of the loop) in a crRNA of a corresponding single guide CRISPR-Cas system. It is understood that an additional nucleotide sequence 3′ to the modulator stem sequence is dispensable. Accordingly, in certain embodiments, the modulator nucleic acid does not comprise any additional nucleotide 3′ to the modulator stem sequence.

It is understood that the additional nucleotide sequence 5′ to the targeter stem sequence and the additional nucleotide sequence 3′ to the modulator stem sequence, if present, may interact with each other. For example, although the nucleotide immediately 5′ to the targeter stem sequence and the nucleotide immediately 3′ to the modulator stem sequence do not form a Watson-Crick base pair (otherwise they would constitute part of the targeter stem sequence and part of the modulator stem sequence, respectively), other nucleotides in the additional nucleotide sequence 5′ to the targeter stem sequence and the additional nucleotide sequence 3′ to the modulator stem sequence may form one, two, three, or more base pairs (e.g., Watson-Crick base pairs). Such interaction may affect the stability of the complex comprising the targeter nucleic acid and the modulator nucleic acid.

The stability of a complex comprising a targeter nucleic acid and a modulator nucleic acid can be assessed by the Gibbs free energy change (ΔG) during the formation of the complex, either calculated or actually measured. Where all the predicted base pairing in the complex occurs between a base in the targeter nucleic acid and a base in the modulator nucleic acid, i.e., there is no intra-strand secondary structure, the ΔG during the formation of the complex correlates generally with the ΔG during the formation of a secondary structure within the corresponding single guide nucleic acid. Methods of calculating or measuring the ΔG are known in the art. An exemplary method is RNAfold (ma.tbi.univie.ac.at/cgi-bin/RNAWebSuite/RNAfold.cgi) as disclosed in Gruber et al. (2008) NUCLEIC ACIDS RES., 36(Web Server issue): W70-W74. Unless indicated otherwise, the ΔG values in the present disclosure are calculated by RNAfold for the formation of a secondary structure within a corresponding single guide nucleic acid. In certain embodiments, the ΔG is lower than or equal to −1 kcal/mol, e.g., lower than or equal to −2 kcal/mol, lower than or equal to −3 kcal/mol, lower than or equal to −4 kcal/mol, lower than or equal to −5 kcal/mol, lower than or equal to −6 kcal/mol, lower than or equal to −7 kcal/mol, lower than or equal to −7.5 kcal/mol, or lower than or equal to −8 kcal/mol. In certain embodiments, the ΔG is greater than or equal to −10 kcal/mol, e.g., greater than or equal to −9 kcal/mol, greater than or equal to −8.5 kcal/mol, or greater than or equal to −8 kcal/mol. In certain embodiments, the ΔG is in the range of −10 to −4 kcal/mol. In certain embodiments, the ΔG is in the range of −8 to −4 kcal/mol, −7 to −4 kcal/mol, −6 to −4 kcal/mol, −5 to −4 kcal/mol, −8 to −4.5 kcal/mol, −7 to −4.5 kcal/mol, −6 to −4.5 kcal/mol, or −5 to −4.5 kcal/mol. In certain embodiments, the ΔG is about −8 kcal/mol, −7 kcal/mol, −6 kcal/mol, −5 kcal/mol, −4.9 kcal/mol, −4.8 kcal/mol, −4.7 kcal/mol, −4.6 kcal/mol, −4.5 kcal/mol, −4.4 kcal/mol, −4.3 kcal/mol, −4.2 kcal/mol, −4.1 kcal/mol, or −4 kcal/mol.

It is understood that the ΔG may be affected by a sequence in the targeter nucleic acid that is not within the targeter stem sequence, and/or a sequence in the modulator nucleic acid that is not within the modulator stem sequence. For example, one or more base pairs (e.g., Watson-Crick base pair) between an additional sequence 5′ to the targeter stem sequence and an additional sequence 3′ to the modulator stem sequence may reduce the ΔG, i.e., stabilize the nucleic acid complex. In certain embodiments, the nucleotide immediately 5′ to the targeter stem sequence comprises a uracil or is a uridine, and the nucleotide immediately 3′ to the modulator stem sequence comprises a uracil or is a uridine, thereby forming a nonconventional U-U base pair.

In certain embodiments, the modulator nucleic acid or the single guide nucleic acid comprises a nucleotide sequence referred to herein as a “5′ tail” positioned 5′ to the modulator stem sequence. In a naturally occurring type V-A CRISPR-Cas system, the 5′ tail is a nucleotide sequence positioned 5′ to the stem-loop structure of the crRNA. A 5′ tail in an engineered type V-A CRISPR-Cas system, whether single guide or dual guide, can be reminiscent to the 5′ tail in a corresponding naturally occurring type V-A CRISPR-Cas system.

Without being bound by theory, it is contemplated that the 5′ tail may participate in the formation of the CRISPR-Cas complex. For example, in certain embodiments, the 5′ tail forms a pseudoknot structure with the modulator stem sequence, which is recognized by the Cas protein (see, Yamano et al. (2016) CELL, 165: 949). In certain embodiments, the 5′ tail is at least 3 (e.g., at least 4 or at least 5) nucleotides in length. In certain embodiments, the 5′ tail is 3, 4, or 5 nucleotides in length. In certain embodiments, the nucleotide at the 3′ end of the 5′ tail comprises a uracil or is a uridine. In certain embodiments, the second nucleotide in the 5′ tail, the position counted from the 3′ end, comprises a uracil or is a uridine. In certain embodiments, the third nucleotide in the 5′ tail, the position counted from the 3′ end, comprises an adenine or is an adenosine. This third nucleotide may form a base pair (e.g., a Watson-Crick base pair) with a nucleotide 5′ to the modulator stem sequence. Accordingly, in certain embodiments, the modulator nucleic acid comprises a uridine or a uracil-containing nucleotide 5′ to the modulator stem sequence. In certain embodiments, the 5′ tail comprises the nucleotide sequence of 5′-AUU-3′. In certain embodiments, the 5′ tail comprises the nucleotide sequence of 5′-AAUU-3′. In certain embodiments, the 5′ tail comprises the nucleotide sequence of 5′-UAAUU-3′. In certain embodiments, the 5′ tail is positioned immediately 5′ to the modulator stem sequence.

In certain embodiments, the single guide nucleic acid, the targeter nucleic acid, and/or the modulator nucleic acid are designed to reduce the degree of secondary structure other than the hybridization between the targeter stem sequence and the modulator stem sequence. In certain embodiments, no more than about 75%, 50%, 40%, 30%, 25%, 20%, 15%, 10%, 5%, 1%, or fewer of the nucleotides of the single guide nucleic acid other than the targeter stem sequence and the modulator stem sequence participate in self-complementary base pairing when optimally folded. In certain embodiments, no more than about 75%, 50%, 40%, 30%, 25%, 20%, 15%, 10%, 5%, 1%, or fewer of the nucleotides of the targeter nucleic acid and/or the modulator nucleic acid participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24: and PA Carr and GM Church, 2009, Nature Biotechnology 27(12): 1151-62).

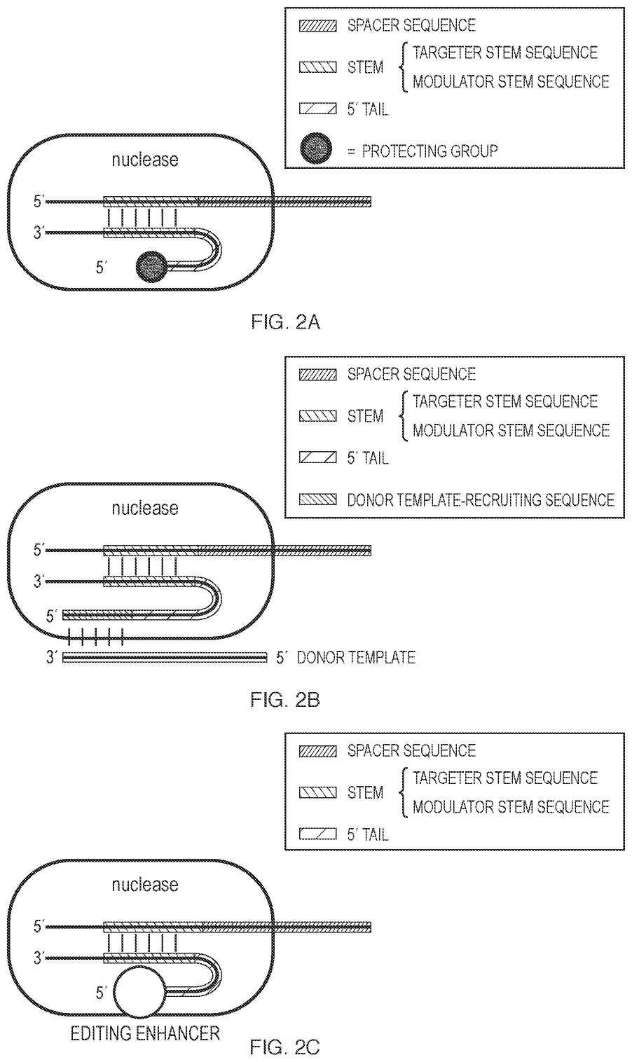

The targeter nucleic acid is directed to a specific target nucleotide sequence, and a donor template can be designed to modify the target nucleotide sequence or a sequence nearby. It is understood, therefore, that association of the single guide nucleic acid, the targeter nucleic acid, or the modulator nucleic acid with a donor template can increase editing efficiency and reduce off-targeting. Accordingly, in certain embodiments, the single guide nucleic acid or the modulator nucleic acid further comprises a donor template-recruiting sequence capable of hybridizing with a donor template (see FIG. 2B). Donor templates are described in the “Donor Templates” subsection of section II infra. The donor template and donor template-recruiting sequence can be designed such that they bear sequence complementarity. In certain embodiments, the donor template-recruiting sequence is at least 90% (e.g., at least 91%, at least 92%, at least 93%, at least 94%, at least 95%, at least 96%, at least 97%, at least 98%, or at least 99%) complementary to at least a portion of the donor template. In certain embodiments, the donor template-recruiting sequence is 100% complementary to at least a portion of the donor template. In certain embodiments, where the donor template comprises an engineered sequence not homologous to the sequence to be repaired, the donor template-recruiting sequence is capable of hybridizing with the engineered sequence in the donor template. In certain embodiments, the donor template-recruiting sequence is at least 20, 25, 30, 35, 40, 45, 50, 55, 60, 65, 70, 75, 80, 85, 90, 95, or 100 nucleotides in length. In certain embodiments, the donor template-recruiting sequence is positioned at or near the 5′ end of the single guide nucleic acid or at or near the 5′ end of the modulator nucleic acid. In certain embodiments, the donor template-recruiting sequence is linked to the 5′ tail, if present, or to the modulator stem sequence, of the single guide nucleic acid or the modulator nucleic acid through an internucleotide bond or a nucleotide linker.

In certain embodiments, the single guide nucleic acid or the modulator nucleic acid further comprises an editing enhancer sequence, which increases the efficiency of gene editing and/or homology-directed repair (HDR) (see FIG. 2C). Exemplary editing enhancer sequences are described in Park et al. (2018) NAT. COMMUN. 9: 3313. In certain embodiments, the editing enhancer sequence is positioned 5′ to the 5′ tail, if present, or 5′ to the single guide nucleic acid or the modulator stem sequence. In certain embodiments, the editing enhancer sequence is 1-50, 4-50, 9-50, 15-50, 25-50, 1-25, 4-25, 9-25, 15-25, 1-15, 4-15, 9-15, 1-9, 4-9, or 1-4 nucleotides in length. In certain embodiments, the editing enhancer sequence is about 1, 2, 3, 4, 5, 6, 7, 8, 9, 10, 15, 20, 25, 30, 35, 40, 45, 50, or 55 nucleotides in length. The editing enhancer sequence is designed to minimize homology to the target nucleotide sequence or any other sequence that the engineered, non-naturally occurring system may be contacted to, e.g., the genome sequence of a cell into which the engineered, non-naturally occurring system is delivered. In certain embodiments, the editing enhancer is designed to minimize the presence of hairpin structure. The editing enhancer can comprise one or more of the chemical modifications disclosed herein.

The single guide nucleic acid, the modulator nucleic acid, and/or the targeter nucleic acid can further comprise a protective nucleotide sequence that prevents or reduces nucleic acid degradation. In certain embodiments, the protective nucleotide sequence is at least 5 (e.g., at least 10, at least 15, at least 20, at least 25, at least 30, at least 35, at least 40, at least 45, or at least 50) nucleotides in length. The length of the protective nucleotide sequence increases the time for an exonuclease to reach the 5′ tail, modulator stem sequence, targeter stem sequence, and/or spacer sequence, thereby protecting these portions of the single guide nucleic acid, the modulator nucleic acid, and/or the targeter nucleic acid from degradation by an exonuclease. In certain embodiments, the protective nucleotide sequence forms a secondary structure, such as a hairpin or a tRNA structure, to reduce the speed of degradation by an exonuclease (see, for example, Wu et al. (2018) CELL. MOL. LIFE SCI., 75(19): 3593-3607). Secondary structures can be predicted by methods known in the art, such as the online webserver RNAfold developed at University of Vienna using the centroid structure prediction algorithm (see. Gruber et al. (2008) NUCLEIC ACIDS RES., 36: W70). Certain chemical modifications, which may be present in the protective nucleotide sequence, can also prevent or reduce nucleic acid degradation, as disclosed in the “RNA Modifications” subsection infra.

A protective nucleotide sequence is typically located at the 5′ or 3′ end of the single guide nucleic acid, the modulator nucleic acid, and/or the targeter nucleic acid. In certain embodiments, the single guide nucleic acid comprises a protective nucleotide sequence at the 5′ end, at the 3′ end, or at both ends, optionally through a nucleotide linker. In certain embodiments, the modulator nucleic acid comprises a protective nucleotide sequence at the 5′ end, at the 3′ end, or at both ends, optionally through a nucleotide linker. In particular embodiments, the modulator nucleic acid comprises a protective nucleotide sequence at the 5′ end (see FIG. 2A). In certain embodiments, the targeter nucleic acid comprises a protective nucleotide sequence at the 5′ end, at the 3′ end, or at both ends, optionally through a nucleotide linker.

As described above, various nucleotide sequences can be present in the 5′ portion of a single nucleic acid or a modulator nucleic acid, including but not limited to a donor template-recruiting sequence, an editing enhancer sequence, a protective nucleotide sequence, and a linker connecting such sequence to the 5′ tail, if present, or to the modulator stem sequence. It is understood that the functions of donor template recruitment, editing enhancement, protection against degradation, and linkage are not exclusive to each other, and one nucleotide sequence can have one or more of such functions. For example, in certain embodiments, the single guide nucleic acid or the modulator nucleic acid comprises a nucleotide sequence that is both a donor template-recruiting sequence and an editing enhancer sequence. In certain embodiments, the single guide nucleic acid or the modulator nucleic acid comprises a nucleotide sequence that is both a donor template-recruiting sequence and a protective sequence. In certain embodiments, the single guide nucleic acid or the modulator nucleic acid comprises a nucleotide sequence that is both an editing enhancer sequence and a protective sequence. In certain embodiments, the single guide nucleic acid or the modulator nucleic acid comprises a nucleotide sequence that is a donor template-recruiting sequence, an editing enhancer sequence, and a protective sequence. In certain embodiments, the nucleotide sequence 5′ to the 5′ tail, if present, or 5′ to the modulator stem sequence is 1-90, 1-80, 1-70, 1-60, 1-50, 1-40, 1-30, 1-20, 1-10, 10-90, 10-80, 10-70, 10-60, 10-50, 10-40, 10-30, 10-20, 20-90, 20-80, 20-70, 20-60, 20-50, 20-40, 20-30, 30-90, 30-80, 30-70, 30-60, 30-50, 30-40, 40-90, 40-80, 40-70, 40-60, 40-50, 50-90, 50-80, 50-70, 50-60, 60-90, 60-80, 60-70, 70-90, 70-80, or 80-90 nucleotides in length.

In certain embodiments, the engineered, non-naturally occurring system further comprises one or more compounds (e.g., small molecule compounds) that enhance HDR and/or inhibit NHEJ. Exemplary compounds having such functions are described in Maruyama et al. (2015) NAT BIOTECHNOL. 33(5): 538-42; Chu et al. (2015) NAT BIOTECHNOL. 33(5): 543-48; Yu et al. (2015) CELL STEM CELL 16(2): 142-47; Pinder et al. (2015) NUCLEIC ACIDS RES. 43(19): 9379-92; and Yagiz et al. (2019) COMMUN. BIOL. 2: 198. In certain embodiments, the engineered, non-naturally occurring system further comprises one or more compounds selected from the group consisting of DNA ligase IV antagonists (e.g., SCR7 compound, Ad4 EIB55K protein, and Ad4 E4orf6 protein), RAD51 agonists (e.g., RS-1), DNA-dependent protein kinase (DNA-PK) antagonists (e.g., NU7441 and KU0060648), β3-adrenergic receptor agonists (e.g., L755507), inhibitors of intracellular protein transport from the ER to the Golgi apparatus (e.g., brefeldin A), and any combinations thereof.

In certain embodiments, the engineered, non-naturally occurring system comprising a targeter nucleic acid and a modulator nucleic acid is tunable or inducible. For example, in certain embodiments, the targeter nucleic acid, the modulator nucleic acid, and/or the Cas protein can be introduced to the target nucleotide sequence at different times, the system becoming active only when all components are present. In certain embodiments, the amounts of the targeter nucleic acid, the modulator nucleic acid, and/or the Cas protein can be titrated to achieve desired efficiency and specificity. In certain embodiments, excess amount of a nucleic acid comprising the targeter stem sequence or the modulator stem sequence can be added to the system, thereby dissociating the complex of the targeter nucleic and modulator nucleic acid and turning off the system.

### RNA Modifications

The guide nucleic acids disclosed herein, including a single guide nucleic acid, a targeter nucleic acid, and/or a modulator nucleic acid, may comprise a DNA (e.g., modified DNA), an RNA (e.g., modified RNA), or a combination thereof. In certain embodiments, the single guide nucleic acid comprises a DNA (e.g., modified DNA), an RNA (e.g., modified RNA), or a combination thereof. In certain embodiments, the targeter nucleic acid comprises a DNA (e.g., modified DNA), an RNA (e.g., modified RNA), or a combination thereof. In certain embodiments, the modulator nucleic acid comprises a DNA (e.g., modified DNA), an RNA (e.g., modified RNA), or a combination thereof. The spacer sequences disclosed herein are presented as DNA sequences by including thymidines (T) rather than uridines (U). It is understood that corresponding RNA sequences and DNA/RNA chimeric sequences are also contemplated. For example, where the spacer sequence is an RNA, its sequence can be derived from a DNA sequence disclosed herein by replacing each T with U. As a result, for the purpose of describing a nucleotide sequence, T and U are used interchangeably herein.

In certain embodiments, the single guide nucleic acid is an RNA. A single guide nucleic acid in the form of an RNA is also called a single guide RNA. In certain embodiments, the targeter nucleic acid is an RNA and the modulator nucleic acid is an RNA. A targeter nucleic acid in the form of an RNA is also called targeter RNA, and a modulator nucleic acid in the form of an RNA is also called modulator RNA.

In certain embodiments, the single guide nucleic acid, the targeter nucleic acid, and/or the modulator nucleic acid are RNAs with one or more modifications in a ribose group, one or more modifications in a phosphate group, one or more modifications in a nucleobase, one or more terminal modifications, or a combination thereof. Exemplary modifications are disclosed in U.S. Patent Application Publication Nos. 2016/0289675, 2017/0355985, 2018/0119140. Watts et al. (2008) Drug Discov. Today 13: 842-55, and Hendel et al. (2015) NAT. BIOTECHNOL. 33: 985.

Modifications in a ribose group include but are not limited to modifications at the 2′ position or modifications at the 4′ position. For example, in certain embodiments, the ribose comprises 2′-O-C1-4alkyl, such as 2′-O-methyl (2′-OMe). In certain embodiments, the ribose comprises 2′-O-C1-3alkyl-O-C1-3alkyl, such as 2′-methoxyethoxy (2′-O—CH₂CH₂OCH₃) also known as 2′-O-(2-methoxyethyl) or 2′-MOE. In certain embodiments, the ribose comprises 2′-O-allyl. In certain embodiments, the ribose comprises 2′-O-2,4-Dinitrophenol (DNP). In certain embodiments, the ribose comprises 2′-halo, such as 2′-F, 2′-Br, 2′-Cl, or 2′-I. In certain embodiments, the ribose comprises 2′-NH₂. In certain embodiments, the ribose comprises 2′-H (e.g., a deoxynucleotide). In certain embodiments, the ribose comprises 2′-arabino or 2′-F-arabino. In certain embodiments, the ribose comprises 2′-LNA or 2′-ULNA. In certain embodiments, the ribose comprises a 4′-thioribosyl.

Modifications in a phosphate group include but are not limited to a phosphorothioate internucleotide linkage, a chiral phosphorothioate internucleotide linkage, a phosphorodithioate internucleotide linkage, a boranophosphonate internucleotide linkage, a C₁₋₄alkyl phosphonate internucleotide linkage such as a methylphosphonate internucleotide linkage, a boranophosphonate internucleotide linkage, a phosphonocarboxylate internucleotide linkage such as a phosphonoacetate internucleotide linkage, a phosphonocarboxylate ester internucleotide linkage such as a phosphonoacetate ester internucleotide linkage, an amide linkage, a thiophosphonocarboxylate internucleotide linkage such as a thiophosphonoacetate internucleotide linkage, a thiophosphonocarboxylate ester internucleotide linkage such as a thiophosphonoacetate ester internucleotide linkage, and a 2′,5′-linkage having a phosphodiester linker or any of the linkers above. Various salts, mixed salts and free acid forms are also included.

Modifications in a nucleobase include but are not limited to 2-thiouracil, 2-thiocytosine, 4-thiouracil, 6-thioguanine, 2-aminoadenine, 2-aminopurine, pseudouracil, hypoxanthine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dihydrouracil, 5-propynylcytosine, 5-propynyluracil, 5-ethynylcytosine, 5-ethynyluracil, 5-allyluracil, 5-allylcytosine, 5-aminoallyluracil, 5-aminoallyl-cytosine, 5-bromouracil, 5-iodouracil, diaminopurine, difluorotoluene, dihydrouracil, an abasic nucleotide, Z base, P base, Unstructured Nucleic Acid, isoguanine, isocytosine (see, Piccirilli et al. (1990) NATURE, 343: 33), 5-methyl-2-pyrimidine (see, Rappaport (1993) BIOCHEMNSTRY, 32: 3047), x(A,G,C,T), and y(A,G,C,T).

Terminal modifications include but are not limited to polyethyleneglycol (PEG), hydrocarbon linkers (such as heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers), spermine linkers, dyes such as fluorescent dyes (for example, fluoresceins, rhodamines, cyanines), quenchers (for example, dabcyl, BHQ), and other labels (for example biotin, digoxigenin, acridine, streptavidin, avidin, peptides and/or proteins). In certain embodiments, a terminal modification comprises a conjugation (or ligation) of the RNA to another molecule comprising an oligonucleotide (such as deoxyribonucleotides and/or ribonucleotides), a peptide, a protein, a sugar, an oligosaccharide, a steroid, a lipid, a folic acid, a vitamin and/or other molecule. In certain embodiments, a terminal modification incorporated into the RNA is located internally in the RNA sequence via a linker such as 2-(4-butylamidofluorescein)propane-1,3-diol bis(phosphodiester) linker, which is incorporated as a phosphodiester linkage and can be incorporated anywhere between two nucleotides in the RNA.

The modifications disclosed above can be combined in the single guide RNA, the targeter RNA, and/or the modulator RNA. In certain embodiments, the modification in the RNA is selected from the group consisting of incorporation of 2′-O-methyl-3′phosphorothioate, 2′-O-methyl-3′-phosphonoacetate, 2′-O-methyl-3′-thiophosphonoacetate, 2′-halo-3′-phosphorothioate (e.g., 2′-fluoro-3′-phosphorothioate), 2′-halo-3′-phosphonoacetate (e.g., 2′-fluoro-3′-phosphonoacetate), and 2′-halo-3′-thiophosphonoacetate (e.g., 2′-fluoro-3′-thiophosphonoacetate).

In certain embodiments, the modification alters the stability of the RNA. In certain embodiments, the modification enhances the stability of the RNA, e.g., by increasing nuclease resistance of the RNA relative to a corresponding RNA without the modification. Stability-enhancing modifications include but are not limited to incorporation of 2′-O-methyl, a 2′-O—C₁₋₄alkyl, 2′-halo (e.g., 2′-F, 2′-Br, 2′-Cl, or 2′-I), 2′MOE, a 2′-O—C₁₋₃alkyl-O—C₁₋₃alkyl, 2′-NH₂, 2′-H (or 2′-deoxy), 2′-arabino, 2′-F-arabino, 4′-thioribosyl sugar moiety, 3′-phosphorothioate, 3′-phosphonoacetate, 3′-thiophosphonoacetate, 3′-methylphosphonate, 3′-boranophosphate, 3′-phosphorodithioate, locked nucleic acid (“LNA”) nucleotide which comprises a methylene bridge between the 2′ and 4′ carbons of the ribose ring, and unlocked nucleic acid (“ULNA”) nucleotide. Such modifications are suitable for use as a protecting group to prevent or reduce degradation of the 5′ tail, modulator stem sequence, targeter stem sequence, and/or spacer sequence (see, the “Guide Nucleic Acids” subsection supra).

In certain embodiments, the modification alters the specificity of the engineered, non-naturally occurring system. In certain embodiments, the modification enhances the specification of the engineered, non-naturally occurring system, e.g., by enhancing on-target binding and/or cleavage, or reducing off-target binding and/or cleavage, or a combination thereof. Specificity-enhancing modifications include but are not limited to 2-thiouracil, 2-thiocytosine, 4-thiouracil, 6-thioguanine, 2-aminoadenine, and pseudouracil.

In certain embodiments, the modification alters the immunostimulatory effect of the RNA relative to a corresponding RNA without the modification. For example, in certain embodiments, the modification reduces the ability of the RNA to activate TLR7, TLR8, TLR9, TLR3, RIG-I, and/or MDA5.

In certain embodiments, the single guide nucleic acid, the targeter nucleic acid, and/or the modulator nucleic acid comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides. The modification can be made at one or more positions in the single guide nucleic acid, the targeter nucleic acid, and/or the modulator nucleic acid such that these nucleic acids retain functionality. For example, the modified nucleic acids can still direct the Cas protein to the target nucleotide sequence and allow the Cas protein to exert its effector function. It is understood that the particular modification(s) at a position may be selected based on the functionality of the nucleotide at the position. For example, a specificity-enhancing modification may be suitable for a nucleotide in the spacer sequence, the targeter stem sequence, or the modulator stem sequence. A stability-enhancing modification may be suitable for one or more terminal nucleotides in the single guide nucleic acid, the targeter nucleic acid, and/or the modulator nucleic acid. In certain embodiments, at least 1 (e.g., at least 2, at least 3, at least 4, or at least 5) terminal nucleotides at the 5′ end and/or at least 1 (e.g., at least 2, at least 3, at least 4, or at least 5) terminal nucleotides at the 3′ end of the single guide nucleic acid are modified nucleotides. In certain embodiments, 5 or fewer (e.g., 1 or fewer, 2 or fewer, 3 or fewer, or 4 or fewer) terminal nucleotides at the 5′ end and/or 5 or fewer (e.g., 1 or fewer, 2 or fewer, 3 or fewer, or 4 or fewer) terminal nucleotides at the 3′ end of the single guide nucleic acid are modified nucleotides. In certain embodiments, at least 1 (e.g., at least 2, at least 3, at least 4, or at least 5) terminal nucleotides at the 5′ end and/or at least 1 (e.g., at least 2, at least 3, at least 4, or at least 5) terminal nucleotides at the 3′ end of the targeter nucleic acid are modified nucleotides. In certain embodiments, 5 or fewer (e.g., 1 or fewer, 2 or fewer, 3 or fewer, or 4 or fewer) terminal nucleotides at the 5′ end and/or 5 or fewer (e.g., 1 or fewer, 2 or fewer, 3 or fewer, or 4 or fewer) terminal nucleotides at the 3′ end of the targeter nucleic acid are modified nucleotides. In certain embodiments, at least 1 (e.g., at least 2, at least 3, at least 4, or at least 5) terminal nucleotides at the 5′ end and/or at least 1 (e.g., at least 2, at least 3, at least 4, or at least 5) terminal nucleotides at the 3′ end of the modulator nucleic acid are modified nucleotides. In certain embodiments, 5 or fewer (e.g., 1 or fewer, 2 or fewer, 3 or fewer, or 4 or fewer) terminal nucleotides at the 5′ end and/or 5 or fewer (e.g., 1 or fewer, 2 or fewer, 3 or fewer, or 4 or fewer) terminal nucleotides at the 3′ end of the modulator nucleic acid are modified nucleotides. Selection of positions for modifications is described in U.S. Patent Application Publication Nos. 2016/0289675 and 2017/0355985. As used in this paragraph, where the targeter or modulator nucleic acid is a combination of DNA and RNA, the nucleic acid as a whole is considered as an RNA, and the DNA nucleotide(s) are considered as modification(s) of the RNA, including a 2′-H modification of the ribose and optionally a modification of the nucleobase.

It is understood that the targeter nucleic acid and the modulator nucleic acid, while not in the same nucleic acids, i.e., not linked end-to-end through a traditional internucleotide bond, can be covalently conjugated to each other through one or more chemical modifications introduced into these nucleic acids, thereby increasing the stability of the double-stranded complex and/or improving other characteristics of the system.

II. Methods of Targeting, Editing, and/or Modifying Genomic DNA

The engineered, non-naturally occurring system disclosed herein are useful for targeting, editing, and/or modifying a target nucleic acid, such as a DNA (e.g., genomic DNA) in a cell or organism. For example, in certain embodiments, with respect to a given target gene listed in Table 1, 2, or 3, an engineered, non-naturally occurring system disclosed herein that comprises a guide nucleic acid comprising a corresponding spacer sequence, when delivered into a population of human cells (e.g., Jurkat cells) ex vivo, edits the genomic sequence at the locus of the target gene in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

The present invention provides a method of cleaving a target nucleic acid (e.g., DNA) comprising the sequence of a preselected target gene or a portion thereof, the method comprising contacting the target DNA with an engineered, non-naturally occurring system disclosed herein, thereby resulting in cleavage of the target DNA.

In addition, the present invention provides a method of binding a target nucleic acid (e.g., DNA) comprising the sequence of a preselected target gene or a portion thereof, the method comprising contacting the target DNA with an engineered, non-naturally occurring system disclosed herein, thereby resulting in binding of the system to the target DNA. This method is useful for detecting the presence and/or location of the preselected target gene, for example, if a component of the system (e.g., the Cas protein) comprises a detectable marker.

In addition, the present invention provides a method of modifying a target nucleic acid (e.g., DNA) comprising the sequence of a preselected target gene or a portion thereof, or a structure (e.g., protein) associated with the target DNA (e.g., a histone protein in a chromosome), the method comprising contacting the target DNA with an engineered, non-naturally occurring system disclosed herein, wherein the Cas protein comprises an effector domain or is associated with an effector protein, thereby resulting in modification of the target DNA or the structure associated with the target DNA. The modification corresponds to the function of the effector domain or effector protein. Exemplary functions described in the “Cas Proteins” subsection in Section 1 supra are applicable hereto.

The engineered, non-naturally occurring system can be contacted with the target nucleic acid as a complex. Accordingly, in certain embodiments, the method comprises contacting the target nucleic acid with a CRISPR-Cas complex comprising a targeter nucleic acid, a modulator nucleic acid, and a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

The preselected target genes include human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, and PLCG1 genes. Accordingly, the present invention also provides a method of editing a human genomic sequence at one of these preselected target gene loci, the method comprising delivering the engineered, non-naturally occurring system disclosed herein into a human cell, thereby resulting in editing of the genomic sequence at the target gene locus in the human cell. In addition, the present invention provides a method of detecting a human genomic sequence at one of these preselected target gene loci, the method comprising delivering the engineered, non-naturally occurring system disclosed herein into a human cell, wherein a component of the system (e.g., the Cas protein) comprises a detectable marker, thereby detecting the target gene locus in the human cell. In addition, the present invention provides a method of modifying a human chromosome at one of these preselected target gene loci, the method comprising delivering the engineered, non-naturally occurring system disclosed herein into a human cell, wherein the Cas protein comprises an effector domain or is associated with an effector protein, thereby resulting in modification of the chromosome at the target gene locus in the human cell.

The CRISPR-Cas complex may be delivered to a cell by introducing a pre-formed ribonucleoprotein (RNP) complex into the cell. Alternatively, one or more components of the CRISPR-Cas complex may be expressed in the cell. Exemplary methods of delivery are known in the art and described in, for example, U.S. Pat. Nos. 10,113,167 and 8,697,359 and U.S. Patent Application Publication Nos. 2015/0344912, 2018/0044700, 2018/0003696, 2018/0119140, 2017/0107539, 2018/0282763, and 2018/0363009.

It is understood that contacting a DNA (e.g., genomic DNA) in a cell with a CRISPR-Cas complex does not require delivery of all components of the complex into the cell. For examples, one or more of the components may be pre-existing in the cell. In certain embodiments, the cell (or a parental/ancestral cell thereof) has been engineered to express the Cas protein, and the single guide nucleic acid (or a nucleic acid comprising a regulatory element operably linked to a nucleotide sequence encoding the single guide nucleic acid), the targeter nucleic acid (or a nucleic acid comprising a regulatory element operably linked to a nucleotide sequence encoding the targeter nucleic acid), and/or the modulator nucleic acid (or a nucleic acid comprising a regulatory element operably linked to a nucleotide sequence encoding the modulator nucleic acid) are delivered into the cell. In certain embodiments, the cell (or a parental/ancestral cell thereof) has been engineered to express the modulator nucleic acid, and the Cas protein (or a nucleic acid comprising a regulatory element operably linked to a nucleotide sequence encoding the Cas protein) and the targeter nucleic acid (or a nucleic acid comprising a regulatory element operably linked to a nucleotide sequence encoding the targeter nucleic acid) are delivered into the cell. In certain embodiments, the cell (or a parental/ancestral cell thereof) has been engineered to express the Cas protein and the modulator nucleic acid, and the targeter nucleic acid (or a nucleic acid comprising a regulatory element operably linked to a nucleotide sequence encoding the targeter nucleic acid) is delivered into the cell.

In certain embodiments, the target DNA is in the genome of a target cell. Accordingly, the present invention also provides a cell comprising the non-naturally occurring system or a CRISPR expression system described herein. In addition, the present invention provides a cell whose genome has been modified by the CRISPR-Cas system or complex disclosed herein.

The target cells can be mitotic or post-mitotic cells from any organism, such as a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. agardh, and the like, a fungal cell (e.g., a yeast cell), an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal, a cell from a rodent, or a cell from a human. The types of target cells include but are not limited to a stem cell (e.g., an embryonic stem (ES) cell, an induced pluripotent stem (iPS) cell, a germ cell), a somatic cell (e.g., a fibroblast, a hematopoietic cell, a T lymphocyte (e.g., CD8⁺ T lymphocyte), an NK cell, a neuron, a muscle cell, a bone cell, a hepatocyte, a pancreatic cell), an in vitro or in vivo embryonic cell of an embryo at any stage (e.g., a 1-cell, 2-cell, 4-cell, 8-cell; stage zebrafish embryo). Cells may be from established cell lines or may be primary cells (i.e., cells and cells cultures that have been derived from a subject and allowed to grow in vitro for a limited number of passages of the culture). For example, primary cultures are cultures that may have been passaged within 0 times, 1 time, 2 times, 4 times, 5 times, 10 times, or 15 times, but not enough times to go through the crisis stage. Typically, the primary cell lines of the present invention are maintained for fewer than 10 passages in vitro. If the cells are primary cells, they may be harvest from an individual by any suitable method. For example, leukocytes may be harvested by apheresis, leukocytapheresis, or density gradient separation, while cells from tissues such as skin, muscle, bone marrow, spleen, liver, pancreas, lung, intestine, or stomach can be harvested by biopsy. The harvested cells may be used immediately, or may be stored under frozen conditions with a cryopreservative and thawed at a later time in a manner as commonly known in the art.

### Ribonucleoprotein (RNP) Delivery and “Cas RNA” Delivery

The engineered, non-naturally occurring system disclosed herein can be delivered into a cell by suitable methods known in the art, including but not limited to ribonucleoprotein (RNP) delivery and “Cas RNA” delivery described below.

In certain embodiments, a CRISPR-Cas system including a single guide nucleic acid and a Cas protein, or a CRISPR-Cas system including a targeter nucleic acid, a modulator nucleic acid, and a Cas protein, can be combined into a RNP complex and then delivered into the cell as a pre-formed complex. This method is suitable for active modification of the genetic or epigenetic information in a cell during a limited time period. For example, where the Cas protein has nuclease activity to modify the genomic DNA of the cell, the nuclease activity only needs to be retained for a period of time to allow DNA cleavage, and prolonged nuclease activity may increase off-targeting. Similarly, certain epigenetic modifications can be maintained in a cell once established and can be inherited by daughter cells.

A “ribonucleoprotein” or “RNP,” as used herein, refers to a complex comprising a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions, and the like). In certain embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA.

To ensure efficient loading of the Cas protein, the single guide nucleic acid, or the combination of the targeter nucleic acid and the modulator nucleic acid, can be provided in excess molar amount (e.g., about 2 fold, about 3 fold, about 4 fold, or about 5 fold) relative to the Cas protein. In certain embodiments, the targeter nucleic acid and the modulator nucleic acid are annealed under suitable conditions prior to complexing with the Cas protein. In other embodiments, the targeter nucleic acid, the modulator nucleic acid, and the Cas protein are directly mixed together to form an RNP.

A variety of delivery methods can be used to introduce an RNP disclosed herein into a cell. Exemplary delivery methods or vehicles include but are not limited to microinjection, liposomes (see, e.g., U.S. Patent Publication No. 2017/0107539) such as molecular trojan horses liposomes that delivers molecules across the blood brain barrier (see, Pardridge et al. (2010) COLD SPRING HARB. PROTOC., doi:10.1101/pdb.prot5407), immunoliposomes, virosomes, microvesicles (e.g., exosomes and ARMMs), polycations, lipid:nucleic acid conjugates, electroporation, cell permeable peptides (see, U.S. Patent Publication No. 2018/0363009), nanoparticles, nanowires (see, Shalek et al. (2012) NANO LETTERS, 12: 6498), exosomes, and perturbation of cell membrane (e.g., by passing cells through a constriction in a microfluidic system, see, U.S. Patent Publication No. 2018/0003696). Where the target cell is a proliferating cell, the efficiency of RNP delivery can be enhanced by cell cycle synchronization (see, U.S. Patent Publication No. 2018/0044700).

In other embodiments, the dual guide CRISPR-Cas system is delivered into a cell in a “Cas RNA” approach, i.e., delivering (a) a single guide nucleic acid, or a combination of a targeter nucleic acid and a modulator nucleic acid, and (b) an RNA (e.g., messenger RNA (mRNA)) encoding a Cas protein. The RNA encoding the Cas protein can be translated in the cell and form a complex with the single guide nucleic acid or combination of the targeter nucleic acid and the modulator nucleic acid intracellularly. Similar to the RNP approach, RNAs have limited half-lives in cells, even though stability-increasing modification(s) can be made in one or more of the RNAs. Accordingly, the “Cas RNA” approach is suitable for active modification of the genetic or epigenetic information in a cell during a limited time period, such as DNA cleavage, and has the advantage of reducing off-targeting.

The mRNA can be produced by transcription of a DNA comprising a regulatory element operably linked to a Cas coding sequence. Given that multiple copies of Cas protein can be generated from one mRNA, the targeter nucleic acid and the modulator nucleic acid are generally provided in excess molar amount (e.g., at least 5 fold, at least 10 fold, at least 20 fold, at least 30 fold, at least 50 fold, or at least 100 fold) relative to the mRNA. In certain embodiments, the targeter nucleic acid and the modulator nucleic acid are annealed under suitable conditions prior to delivery into the cells. In other embodiments, the targeter nucleic acid and the modulator nucleic acid are delivered into the cells without annealing in vitro.

A variety of delivery systems can be used to introduce an “Cas RNA” system into a cell. Non-limiting examples of delivery methods or vehicles include microinjection, biolistic particles, liposomes (see, e.g., U.S. Patent Publication No. 2017/0107539) such as molecular trojan horses liposomes that delivers molecules across the blood brain barrier (see, Pardridge et al. (2010) COLD SPRING HARB. PROTC., doi:10.1101/pdb.prot5407), immunoliposomes, virosomes, polycations, lipid:nucleic acid conjugates, electroporation, nanoparticles, nanowires (see, Shalek et al. (2012) NANO LETTERS, 12: 6498), exosomes, and perturbation of cell membrane (e.g., by passing cells through a constriction in a microfluidic system, see, U.S. Patent Publication No. 2018/0003696). Specific examples of the “nucleic acid only” approach by electroporation are described in International (PCT) Publication No. WO2016/164356.

In other embodiments, the CRISPR-Cas system is delivered into a cell in the form of (a) a single guide nucleic acid or a combination of a targeter nucleic acid and a modulator nucleic acid, and (b) a DNA comprising a regulatory element operably linked to a Cas coding sequence. The DNA can be provided in a plasmid, viral vector, or any other form described in the “CRISPR Expression Systems” subsection. Such delivery method may result in constitutive expression of Cas protein in the target cell (e.g., if the DNA is maintained in the cell in an episomal vector or is integrated into the genome), and may increase the risk of off-targeting which is undesirable when the Cas protein has nuclease activity. Notwithstanding, this approach is useful when the Cas protein comprises a non-nuclease effector (e.g., a transcriptional activator or repressor). It is also useful for research purposes and for genome editing of plants.

### CRISPR Expression Systems

The present invention also provides a nucleic acid comprising a regulatory element operably linked to a nucleotide sequence encoding a guide nucleic acid disclosed herein. In certain embodiments, the nucleic acid comprises a regulatory element operably linked to a nucleotide sequence encoding a single guide nucleic acid disclosed herein; this nucleic acid alone can constitute a CRISPR expression system. In certain embodiments, the nucleic acid comprises a regulatory element operably linked to a nucleotide sequence encoding a targeter nucleic acid disclosed herein. In certain embodiments, the nucleic acid further comprises a nucleotide sequence encoding a modulator nucleic acid disclosed herein, wherein the nucleotide sequence encoding the modulator nucleic acid is operably linked to the same regulatory element as the nucleotide sequence encoding the targeter nucleic acid or a different regulatory element; this nucleic acid alone can constitute a CRISPR expression system.

In addition, the present invention provides a CRISPR expression system comprising: (a) a nucleic acid comprising a first regulatory element operably linked to a nucleotide sequence encoding a targeter nucleic acid disclosed herein and (b) a nucleic acid comprising a second regulatory element operably linked to a nucleotide sequence encoding a modulator nucleic acid disclosed herein.

In certain embodiments, the CRISPR expression system disclosed herein further comprises a nucleic acid comprising a third regulatory element operably linked to a nucleotide sequence encoding a Cas protein disclosed herein. In certain embodiments, the Cas protein is a type V-A, type V-C, or type V-D Cas protein (e.g., Cas nuclease). In certain embodiments, the Cas protein is a type V-A Cas protein (e.g., Cas nuclease).

As used in this context, the term “operably linked” is intended to mean that the nucleotide sequence of interest is linked to the regulatory element in a manner that allows for expression of the nucleotide sequence (e.g., in an in vitro transcription/translation system or in a host cell when the vector is introduced into the host cell).

The nucleic acids of the CRISPR expression system described above may be independently selected from various nucleic acids such as DNA (e.g., modified DNA) and RNA (e.g., modified RNA). In certain embodiments, the nucleic acids comprising a regulatory element operably linked to one or more nucleotide sequences encoding the guide nucleic acids are in the form of DNA. In certain embodiments, the nucleic acid comprising a third regulatory element operably linked to a nucleotide sequence encoding the Cas protein is in the form of DNA. The third regulatory element can be a constitutive or inducible promoter that drives the expression of the Cas protein. In other embodiments, the nucleic acid comprising a third regulatory element operably linked to a nucleotide sequence encoding the Cas protein is in the form of RNA (e.g., mRNA).

The nucleic acids of the CRISPR expression system can be provided in one or more vectors. The term “vector,” as used herein, refers to a nucleic acid molecule capable of transporting another nucleic acid to which it has been linked. Conventional viral and non-viral based gene transfer methods can be used to introduce nucleic acids in cells, such as prokaryotic cells, eukaryotic cells, mammalian cells, or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. Gene therapy procedures are known in the art and disclosed in Van Brunt (1988) BIOTECHNOLOGY, 6: 1149; Anderson (1992) SCIENCE, 256: 808; Nabel & Feigner (1993) TIBTECH, 11: 211; Mitani & Caskey (1993) TIBTECH, 11: 162; Dillon (1993) TIBTECH, 11: 167; Miller (1992) NATURE, 357: 455; Vigne, (1995) RESTORATIVE NEUROLOGY AND NEUROSCIENCE, 8: 35; Kremer & Perricaudet (1995) BRITISH MEDICAL BULLETIN, 51: 31; Haddada et al. (1995) CURRENT TOPICS IN MICROBIOLOGY AND IMMUNOLOGY, 199: 297; Yu et al. (1994) GENE THERAPY, 1: 13; and Doerfler and Bohm (Eds.) (2012) The Molecular Repertoire of Adenoviruses II: Molecular Biology of Virus-Cell Interactions. In certain embodiments, at least one of the vectors is a DNA plasmid. In certain embodiments, at least one of the vectors is a viral vector (e.g., retrovirus, adenovirus, or adeno-associated virus).

Certain vectors are capable of autonomous replication in a host cell into which they are introduced (e.g., bacterial vectors having a bacterial origin of replication and episomal mammalian vectors). Other vectors (e.g., non-episomal mammalian vectors and replication defective viral vectors) do not autonomously replicate in the host cell. Certain vectors, however, may be integrated into the genome of the host cell and thereby are replicated along with the host genome. A skilled person in the art will appreciate that different vectors may be suitable for different delivery methods and have different host tropism, and will be able to select one or more vectors suitable for the use.

The term “regulatory element,” as used herein, refers to a transcriptional and/or translational control sequence, such as a promoter, enhancer, transcription termination signal (e.g., polyadenylation signal), internal ribosomal entry sites (IRES), protein degradation signal, and the like, that provide for and/or regulate transcription of a non-coding sequence (e.g., a targeter nucleic acid or a modulator nucleic acid) or a coding sequence (e.g., a Cas protein) and/or regulate translation of an encoded polypeptide. Such regulatory elements are described, for example, in Goeddel, GENE EXPRESSION TECHNOLOGY: METHODS IN ENZYMOLOGY, 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In certain embodiments, a vector comprises one or more pol III promoter (e. g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers: the R-U5′ segment in LTR of HTLV-I (see. Takebe et al. (1988) MOL. CELL. BIOL., 8: 466): SV40 enhancer; and the intron sequence between exons 2 and 3 of rabbit β-globin (see, O'Hare et al. (1981) PROC. NATL. ACAD. SCI. USA., 78: 1527). It will be appreciated by those skilled in the art that the design of the expression vector can depend on factors such as the choice of the host cell to be transformed, the level of expression desired, etc. A vector can be introduced into host cells to produce transcripts, proteins, or peptides, including fusion proteins or peptides, encoded by nucleic acids as described herein (e.g., CRISPR transcripts, proteins, enzymes, mutant forms thereof, or fusion proteins thereof).

In certain embodiments, the nucleotide sequence encoding the Cas protein is codon optimized for expression in a eukaryotic host cell, e.g., a yeast cell, a mammalian cell (e.g., a mouse cell, a rat cell, or a human cell), or a plant cell. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.or.jp/codon/ and these tables can be adapted in a number of ways (see. Nakamura et al. (2000) NUCL. ACIDS RES., 28: 292). Computer algorithms for codon optimizing a particular sequence for expression in a particular host cell, such as Gene Forge (Aptagen; Jacobus, Pa.), are also available. In certain embodiments, the codon optimization facilitates or improves expression of the Cas protein in the host cell.

### Donor Templates

Cleavage of a target nucleotide sequence in the genome of a cell by the CRISPR-Cas system or complex disclosed herein can activate the DNA damage pathways, which may rejoin the cleaved DNA fragments by NHEJ or HDR. HDR requires a repair template, either endogenous or exogenous, to transfer the sequence information from the repair template to the target.

In certain embodiments, the engineered, non-naturally occurring system or CRISPR expression system further comprises a donor template. As used herein, the term “donor template” refers to a nucleic acid designed to serve as a repair template at or near the target nucleotide sequence upon introduction into a cell or organism. In certain embodiments, the donor template is complementary to a polynucleotide comprising the target nucleotide sequence or a portion thereof. When optimally aligned, a donor template may overlap with one or more nucleotides of a target nucleotide sequences (e.g. about or more than about 1, 5, 10, 15, 20, 25, 30, 35, 40, or more nucleotides). The nucleotide sequence of the donor template is typically not identical to the genomic sequence that it replaces. Rather, the donor template may contain one or more substitutions, insertions, deletions, inversions or rearrangements with respect to the genomic sequence, so long as sufficient homology is present to support homology-directed repair. In certain embodiments, the donor template comprises a non-homologous sequence flanked by two regions of homology (i.e., homology arms), such that homology-directed repair between the target DNA region and the two flanking sequences results in insertion of the non-homologous sequence at the target region. In certain embodiments, the donor template comprises a non-homologous sequence 10-100 nucleotides, 50-500 nucleotides, 100-1,000 nucleotides, 200-2,000 nucleotides, or 500-5,000 nucleotides in length positioned between two homology arms.

Generally, the homologous region(s) of a donor template has at least 50% sequence identity to a genomic sequence with which recombination is desired. The homology arms are designed or selected such that they are capable of recombining with the nucleotide sequences flanking the target nucleotide sequence under intracellular conditions. In certain embodiments, where HDR of the non-target strand is desired, the donor template comprises a first homology arm homologous to a sequence 5′ to the target nucleotide sequence and a second homology arm homologous to a sequence 3′ to the target nucleotide sequence. In certain embodiments, the first homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 5′ to the target nucleotide sequence. In certain embodiments, the second homology arm is at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%) identical to a sequence 3′ to the target nucleotide sequence. In certain embodiments, when the donor template sequence and a polynucleotide comprising a target nucleotide sequence are optimally aligned, the nearest nucleotide of the donor template is within about 1, 5, 10, 15, 20, 25, 50, 75, 100, 200, 300, 400, 500, 1000, 2000, 3000, 4000, or more nucleotides from the target nucleotide sequence.

In certain embodiments, the donor template further comprises an engineered sequence not homologous to the sequence to be repaired. Such engineered sequence can harbor a barcode and/or a sequence capable of hybridizing with a donor template-recruiting sequence disclosed herein.

In certain embodiments, the donor template further comprises one or more mutations relative to the genomic sequence, wherein the one or more mutations reduce or prevent cleavage, by the same CRISPR-Cas system, of the donor template or of a modified genomic sequence with at least a portion of the donor template sequence incorporated. In certain embodiments, in the donor template, the PAM adjacent to the target nucleotide sequence and recognized by the Cas nuclease is mutated to a sequence not recognized by the same Cas nuclease. In certain embodiments, in the donor template, the target nucleotide sequence (e.g., the seed region) is mutated. In certain embodiments, the one or more mutations are silent with respect to the reading frame of a protein-coding sequence encompassing the mutated sites.

The donor template can be provided to the cell as single-stranded DNA, single-stranded RNA, double-stranded DNA, or double-stranded RNA. It is understood that the CRISPR-Cas system disclosed herein may possess nuclease activity to cleave the target strand, the non-target strand, or both. When HDR of the target strand is desired, a donor template having a nucleic acid sequence complementary to the target strand is also contemplated.

The donor template can be introduced into a cell in linear or circular form. If introduced in linear form, the ends of the donor template may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends (see, for example. Chang et al. (1987) PROC. NATL. ACAD SCI USA, 84: 4959; Nehls et al. (1996) SCIENCE, 272: 886; see also the chemical modifications for increasing stability and/or specificity of RNA disclosed supra). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor template, additional lengths of sequence may be included outside of the regions of homology that can be degraded without impacting recombination.

A donor template can be a component of a vector as described herein, contained in a separate vector, or provided as a separate polynucleotide, such as an oligonucleotide, linear polynucleotide, or synthetic polynucleotide. In certain embodiments, the donor template is a DNA. In certain embodiments, a donor template is in the same nucleic acid as a sequence encoding the single guide nucleic acid, a sequence encoding the targeter nucleic acid, a sequence encoding the modulator nucleic acid, and/or a sequence encoding the Cas protein, where applicable. In certain embodiments, a donor template is provided in a separate nucleic acid. A donor template polynucleotide may be of any suitable length, such as about or at least about 50, 75, 100, 150, 200, 500, 1000, 2000, 3000, 4000, or more nucleotides in length.

A donor template can be introduced into a cell as an isolated nucleic acid. Alternatively, a donor template can be introduced into a cell as part of a vector (e.g., a plasmid) having additional sequences such as, for example, replication origins, promoters and genes encoding antibiotic resistance, that are not intended for insertion into the DNA region of interest. Alternatively, a donor template can be delivered by viruses (e.g., adenovirus, adeno-associated virus (AAV)). In certain embodiments, the donor template is introduced as an AAV, e.g., a pseudotyped AAV. The capsid proteins of the AAV can be selected by a person skilled in the art based upon the tropism of the AAV and the target cell type. For example, in certain embodiments, the donor template is introduced into a hepatocyte as AAV8 or AAV9. In certain embodiments, the donor template is introduced into a hematopoietic stem cell, a hematopoietic progenitor cell, or a T lymphocyte (e.g., CD8⁺ T lymphocyte) as AAV6 or an AAVHSC (see, U.S. Pat. No. 9,890,396). It is understood that the sequence of a capsid protein (VP1, VP2, or VP3) may be modified from a wild-type AAV capsid protein, for example, having at least 50% (e.g., at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to a wild-type AAV capsid sequence.

The donor template can be delivered to a cell (e.g., a primary cell) by various delivery methods, such as a viral or non-viral method disclosed herein. In certain embodiments, a non-viral donor template is introduced into the target cell as a naked nucleic acid or in complex with a liposome or poloxamer. In certain embodiments, a non-viral donor template is introduced into the target cell by electroporation. In other embodiments, a viral donor template is introduced into the target cell by infection. The engineered, non-naturally occurring system can be delivered before, after, or simultaneously with the donor template (see, International (PCT) Application Publication No. WO2017/053729). A skilled person in the art will be able to choose proper timing based upon the form of delivery (consider, for example, the time needed for transcription and translation of RNA and protein components) and the half-life of the molecule(s) in the cell. In particular embodiments, where the CRISPR-Cas system including the Cas protein is delivered by electroporation (e.g., as an RNP), the donor template (e.g., as an AAV) is introduced into the cell within 4 hours (e.g., within 1, 2, 3, 4, 5, 6, 7, 8, 9, 10, 11, 12, 13, 14, 15, 16, 17, 18, 19, 20, 25, 30, 35, 40, 45, 50, 55, 60, 90, 120, 150, 180, 210, or 240 minutes) after the introduction of the engineered, non-naturally occurring system.

In certain embodiments, the donor template is conjugated covalently to the modulator nucleic acid. Covalent linkages suitable for this conjugation are known in the art and are described, for example, in U.S. Pat. No. 9,982,278 and Savic et al. (2018) ELIFE 7:e33761. In certain embodiments, the donor template is covalently linked to the modulator nucleic acid (e.g., the 5′ end of the modulator nucleic acid) through an internucleotide bond. In certain embodiments, the donor template is covalently linked to the modulator nucleic acid (e.g., the 5′ end of the modulator nucleic acid) through a linker.

### Efficiency and Specificity

The engineered, non-naturally occurring system of the present invention has the advantage of high efficiency and/or high specificity in nucleic acid targeting, cleavage, or modification.

In certain embodiments, the engineered, non-naturally occurring system has high efficiency. For example, in certain embodiments, at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of nucleic acids having the target nucleotide sequence and a cognate PAM, when contacted with the engineered, non-naturally occurring system, is targeted, cleaved, or modified. In certain embodiments, the genomes of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of cells, when the engineered, non-naturally occurring system is delivered into the cells, are targeted, cleaved, or modified.

In certain embodiments, where the engineered, non-naturally occurring system comprises a guide nucleic acid comprising a spacer sequence listed in Table 2 or a portion thereof, the genomes of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are targeted, cleaved, edited, or modified when the engineered, non-naturally occurring system is delivered into the cells. In certain embodiments, where the engineered, non-naturally occurring system comprises a guide nucleic acid comprising a spacer sequence listed in Table 2 or a portion thereof, the genomes of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are edited when the engineered, non-naturally occurring system is delivered into the cells.

In certain embodiments, where the engineered, non-naturally occurring system comprises a guide nucleic acid comprising a spacer sequence listed in Table 3 or a portion thereof, the genomes of at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are targeted, cleaved, edited, or modified when the engineered, non-naturally occurring system is delivered into the cells. In certain embodiments, where the engineered, non-naturally occurring system comprises a guide nucleic acid comprising a spacer sequence listed in Table 3 or a portion thereof, the genomes of at least 1%, at least 1.5%, at least 2%, at least 2.5%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of a population of human cells are edited when the engineered, non-naturally occurring system is delivered into the cells.

In certain embodiments, when an engineered, non-naturally occurring system comprising a guide nucleic acid comprising a spacer sequence set forth in SEQ ID NO: 51 is delivered into a population of human cells ex vivo, the genome sequence at the ADORA2A gene locus is edited in at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 52 is delivered into a population of human cells ex vivo, the genome sequence at the B2M gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 53 is delivered into a population of human cells ex vivo, the genome sequence at the CD52 gene locus is edited in at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 54 is delivered into a population of human cells ex vivo, the genome sequence at the CIITA gene locus is edited in at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 55, 67, 68, or 69 is delivered into a population of human cells ex vivo, the genome sequence at the CTLA4 gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 56, 71, or 72 is delivered into a population of human cells ex vivo, the genome sequence at the DCK gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 57, 75, 76, 77, or 78 is delivered into a population of human cells ex vivo, the genome sequence at the FAS gene locus is edited in at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 58, 80, or 81 is delivered into a population of human cells ex vivo, the genome sequence at the HAVCR2 gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 59 is delivered into a population of human cells ex vivo, the genome sequence at the LAG3 gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 60, 89, 90, 91, or 92 is delivered into a population of human cells ex vivo, the genome sequence at the PDCD1 gene locus is edited in at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 61, 93, 94, 95, 96, 97, 98, or 99 is delivered into a population of human cells ex vivo, the genome sequence at the PTPN6 gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 62 or 105 is delivered into a population of human cells ex vivo, the genome sequence at the TIGIT gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

In certain embodiments, when an engineered, non-naturally occurring system comprising a guide nucleic acid comprising a spacer sequence set forth in SEQ ID NO: 63, 106, 107, 108, 109, 110, 111, 112, 113, 114, or 115 is delivered into a population of human cells ex vivo, the genome sequence at the TRAC gene locus is edited in at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% of the cells.

It has been observed that for a given spacer sequence, the occurrence of on-target events and the occurrence of off-target events are generally correlated. For certain therapeutic purposes, lower on-target efficiency can be tolerated and low off-target frequency is more desirable. For example, when editing or modifying a proliferating cell that will be delivered to a subject and proliferate n vivo, tolerance to off-target events is low. Prior to delivery, it is possible to assess the on-target and off-target events, thereby selecting one or more colonies that have the desired edit or modification and lack any undesired edit or modification. Notwithstanding, the on-target efficiency needs to meet a certain standard to be suitable for therapeutic use. The high editing efficiency observed with the spacer sequences disclosed herein in a standard CRISPR-Cas system allows tuning of the system, for example, by reducing the binding of the guide nucleic acids to the Cas protein, without losing therapeutic applicability.

In certain embodiments, when a population of nucleic acids having the target nucleotide sequence and a cognate PAM is contacted with the engineered, non-naturally occurring system disclosed herein, the frequency of off-target events (e.g., targeting, cleavage, or modification, depending on the function of the CRISPR-Cas system) is reduced. Methods of assessing off-target events were summarized in Lazzarotto er al. (2018) NAT PROTOC. 13(11): 2615-42, and include discovery of in situ Cas off-targets and verification by sequencing (DISCOVER-seq) as disclosed in Wienert et al. (2019) SCIENCE 364(6437): 286-89: genome-wide unbiased identification of double-stranded breaks (DSBs) enabled by sequencing (GUIDE-seq) as disclosed in Kleinstiver et al. (2016) NAT. BIOICH. 34: 869-74: circularization for in vitro reporting of cleavage effects by sequencing (CIRCLE-seq) as described in Kocak et al. (2019) NAT. BIOTECH. 37: 657-66. In certain embodiments, the off-target events include targeting, cleavage, or modification at a given off-target locus (e.g., the locus with the highest occurrence of off-target events detected). In certain embodiments, the off-target events include targeting, cleavage, or modification at all the loci with detectable off-target events, collectively.

In certain embodiments, genomic mutations are detected in no more than 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% of the cells at any off-target loci (in aggregate). In certain embodiments, the ratio of the percentage of cells having an on-target event to the percentage of cells having any off-target event (e.g., the ratio of the percentage of cells having an on-target editing event to the percentage of cells having a mutation at any off-target loci) is at least 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000. It is understood that genetic variation may be present in a population of cells, for example, by spontaneous mutations, and such mutations are not included as off-target events.

### Multiplex Methods

The method of targeting, editing, and/or modifying a genomic DNA disclosed herein can be conducted in multiplicity. For example, a library of targeter nucleic acids can be used to target multiple genomic loci: a library of donor templates can also be used to generate multiple insertions, deletions, and/or substitutions. The multiplex assay can be conducted in a screening method wherein each separate cell culture (e.g., in a well of a 96-well plate or a 384-well plate) is exposed to a different guide nucleic acid having a different targeter stem sequence and/or a different donor template. The multiplex assay can also be conducted in a selection method wherein a cell culture is exposed to a mixed population of different guide nucleic acids and/or donor templates, and the cells with desired characteristics (e.g., functionality) are enriched or selected by advantageous survival or growth, resistance to a certain agent, expression of a detectable protein (e.g., a fluorescent protein that is detectable by flow cytometry), etc.

In certain embodiments, the plurality of guide nucleic acids and/or the plurality of donor templates are designed for saturation editing. For example, in certain embodiments, each nucleotide position in a sequence of interest is systematically modified with each of all four traditional bases, A, T, G and C. In other embodiments, at least one sequence in each gene from a pool of genes of interest is modified, for example, according to a CRISPR design algorithm. In certain embodiments, each sequence from a pool of exogenous elements of interest (e.g., protein coding sequences, non-protein coding genes, regulatory elements) is inserted into one or more given loci of the genome.

It is understood that the multiplex methods suitable for the purpose of carrying out a screening or selection method, which is typically conducted for research purposes, may be different from the methods suitable for therapeutic purposes. For example, constitutive expression of certain elements (e.g., a Cas nuclease and/or a guide nucleic acid) may be undesirable for therapeutic purposes due to the potential of increased off-targeting. Conversely, for research purposes, constitutive expression of a Cas nuclease and/or a guide nucleic acid may be desirable. For example, the constitutive expression provides a large window during which other elements can be introduced. When a stable cell line is established for the constitutive expression, the number of exogenous elements that need to be co-delivered into a single cell is also reduced. Therefore, constitutive expression of certain elements can increase the efficiency and reduce the complexity of a screening or selection process. Inducible expression of certain elements of the system disclosed herein may also be used for research purposes given similar advantages. Expression may be induced by an exogenous agent (e.g., a small molecule) or by an endogenous molecule or complex present in a particular cell type (e.g., at a particular stage of differentiation). Methods known in the art, such as those described in the “CRISPR Expression Systems” subsection supra, can be used for constitutively or inducibly expressing one or more elements.

It is further understood that despite the need to introduce multiple elements—the single guide nucleic acid and the Cas protein; or the targeter nucleic acid, the modulator nucleic acid, and the Cas protein—these elements can be delivered into the cell as a single complex of pre-formed RNP. Therefore, the efficiency of the screening or selection process can also be achieved by pre-assembling a plurality of RNP complexes in a multiplex manner.

In certain embodiments, the method disclosed herein further comprises a step of identifying a guide nucleic acid, a Cas protein, a donor template, or a combination of two or more of these elements from the screening or selection process. A set of barcodes may be used, for example, in the donor template between two homology arms, to facilitate the identification. In specific embodiments, the method further comprises harvesting the population of cells; selectively amplifying a genomic DNA or RNA sample including the target nucleotide sequence(s) and/or the barcodes; and/or sequencing the genomic DNA or RNA sample and/or the barcodes that has been selectively amplified.

In addition, the present invention provides a library comprising a plurality of guide nucleic acids disclosed herein. In another aspect, the present invention provides a library comprising a plurality of nucleic acids each comprising a regulatory element operably linked to a different guide nucleic acid disclosed herein. These libraries can be used in combination with one or more Cas proteins or Cas-coding nucleic acids disclosed herein, and/or one or more donor templates as disclosed herein for a screening or selection method.

### III. Pharmaceutical Compositions

The present invention provides a composition (e.g., pharmaceutical composition) comprising a guide nucleic acid, an engineered, non-naturally occurring system, or a eukaryotic cell disclosed herein. In certain embodiments, the composition comprises an RNP comprising a guide nucleic acid disclosed herein and a Cas protein (e.g., Cas nuclease). In certain embodiments, the composition comprises a complex of a targeter nucleic acid and a modulator nucleic acid disclosed herein. In certain embodiments, the composition comprises an RNP comprising the targeter nucleic acid, the modulator nucleic acid, and a Cas protein (e.g., Cas nuclease).

In addition, the present invention provides a method of producing a composition, the method comprising incubating a single guide nucleic acid disclosed herein with a Cas protein, thereby producing a complex of the single guide nucleic acid and the Cas protein (e.g., an RNP). In certain embodiments, the method further comprises purifying the complex (e.g., the RNP).

In addition, the present invention provides a method of producing a composition, the method comprising incubating a targeter nucleic acid and a modulator nucleic acid disclosed herein under suitable conditions, thereby producing a composition (e.g., pharmaceutical composition) comprising a complex of the targeter nucleic acid and the modulator nucleic acid. In certain embodiments, the method further comprises incubating the targeter nucleic acid and the modulator nucleic acid with a Cas protein (e.g., the Cas nuclease that the targeter nucleic acid and the modulator nucleic acid are capable of activating or a related Cas protein), thereby producing a complex of the targeter nucleic acid, the modulator nucleic acid, and the Cas protein (e.g., an RNP). In certain embodiments, the method further comprises purifying the complex (e.g., the RNP).

For therapeutic use, a guide nucleic acid, an engineered, non-naturally occurring system, a CRISPR expression system, or a cell comprising such system or modified by such system disclosed herein is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit-to-risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition disclosed herein comprises a salt, e.g., NaCl, MgCl₂, KCl, MgSO₄, etc.; a buffering agent, e.g., a Tris buffer. N-(2-Hydroxyethyl)piperazine-N′-(2-ethanesulfonic acid) (HEPES), 2-(N-Morpholino)ethanesulfonic acid (MES), MES sodium salt, 3-(N-Morpholino)propanesulfonic acid (MOPS), N-tris[Hydroxymethyl]methyl-3-aminopropanesulfonic acid (TAPS), etc.: a solubilizing agent; a detergent, e.g., a non-ionic detergent such as Tween-20, etc.: a nuclease inhibitor; and the like. For example, in certain embodiments, a subject composition comprises a subject DNA-targeting RNA and a buffer for stabilizing nucleic acids.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins): coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents: surfactants or wetting agents (such as pluronics, PEG, sorbitan esters, polysorbates such as polysorbate 20, polysorbate, triton, tromethamine, lecithin, cholesterol, tyloxapol); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29). In certain embodiment, the pharmaceutical composition comprises an inorganic nanoparticle. Exemplary inorganic nanoparticles include, e.g., magnetic nanoparticles (e.g., Fe₃MnO₂) or silica. The outer surface of the nanoparticle can be conjugated with a positively charged polymer (e.g., polyethylenimine, polylysine, polyserine) which allows for attachment (e.g., conjugation or entrapment) of payload. In certain embodiment, the pharmaceutical composition comprises an organic nanoparticle (e.g., entrapment of the payload inside the nanoparticle). Exemplary organic nanoparticles include, e.g., SNALP liposomes that contain cationic lipids together with neutral helper lipids which are coated with polyethylene glycol (PEG) and protamine and nucleic acid complex coated with lipid coating. In certain embodiment, the pharmaceutical composition comprises a liposome, for example, a liposome disclosed in International Application Publication No. WO 2015/148863.

In certain embodiments, the pharmaceutical composition comprises a targeting moiety to increase target cell binding or update of nanoparticles and liposomes. Exemplary targeting moieties include cell specific antigens, monoclonal antibodies, single chain antibodies, aptamers, polymers, sugars, and cell penetrating peptides. In certain embodiments, the pharmaceutical composition comprises a fusogenic or endosome-destabilizing peptide or polymer.

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(-)-3-hydroxybutyric acid. Sustained release compositions may also include liposomes that can be prepared by any of several methods known in the art.

A pharmaceutical composition of the invention can be administered by a variety of methods known in the art. The route and/or mode of administration vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound (e.g., the guide nucleic acid, engineered, non-naturally occurring system, or CRISPR expression system of the invention) may be coated in a material to protect the compound from the action of acids and other natural conditions that may inactivate the compound.

Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution. In certain embodiments, the pharmaceutical composition is lyophilized, and then reconstituted in buffered saline, at the time of administration.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker. Inc., New York, 1978. Pharmaceutical compositions are preferably manufactured under GMP conditions. Typically, a therapeutically effective dose or efficacious dose of the guide nucleic acid, engineered, non-naturally occurring system, or CRISPR expression system of the invention is employed in the pharmaceutical compositions of the invention. The multispecific antibodies of the invention are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### IV. Therapeutic Uses

The guide nucleic acids, the engineered, non-naturally occurring systems, and the CRISPR expression systems disclosed herein are useful for targeting, editing, and/or modifying the genomic DNA in a cell or organism. These guide nucleic acids and systems, as well as a cell comprising one of the systems or a cell whose genome has been modified by one of the systems, can be used to treat a disease or disorder in which modification of genetic or epigenetic information is desirable. Accordingly, the present invention provides a method of treating a disease or disorder, the method comprising administering to a subject in need thereof a guide nucleic acid, a non-naturally occurring system, a CRISPR expression system, or a cell disclosed herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treatment”, “treating”, “treat” “treated”, and the like, as used herein, refer to obtaining a desired pharmacologic and/or physiologic effect. The effect may be therapeutic in terms of a partial or complete cure for a disease and/or adverse effect attributable to the disease or delaying the disease progression. “Treatment”, as used herein, covers any treatment of a disease in a mammal, e.g., in a human, and includes: (a) inhibiting the disease, i.e., arresting its development: and (b) relieving the disease, i.e., causing regression of the disease. It is understood that a disease or disorder may be identified by genetic methods and treated prior to manifestation of any medical symptom.

For minimization of toxicity and off-target effect, it is important to control the concentration of the CRISPR-Cas system delivered. Optimal concentrations can be determined by testing different concentrations in a cellular, tissue, or non-human eukaryote animal model and using deep sequencing to analyze the extent of modification at potential off-target genomic loci. The concentration that gives the highest level of on-target modification while minimizing the level of off-target modification should be selected for ex vivo or n vivo delivery.

It is understood that the guide nucleic acid, the engineered, non-naturally occurring system, and the CRISPR expression system disclosed herein can be used to treat any disease or disorder that can be improved by editing or modifying human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, or PLCG1 gene in a cell. In certain embodiments, the guide nucleic acid, the engineered, non-naturally occurring system, and the CRISPR expression system disclosed herein can be used to engineer an immune cell. Immune cells include but are not limited to lymphocytes (e.g., B lymphocytes or B cells, T lymphocytes or T cells, and natural killer cells), myeloid cells (e.g., monocytes, macrophages, eosinophils, mast cells, basophils, and granulocytes), and the stem and progenitor cells that can differentiate into these cell types (e.g., hematopoietic stem cells, hematopoietic progenitor cells, and lymphoid progenitor cells). The cells can include autologous cells derived from a subject to be treated, or alternatively allogenic cells derived from a donor.

In certain embodiments, the immune cell is a T cell, which can be, for example, a cultured T cell, a primary T cell, a T cell from a cultured T cell line (e.g., Jurkat, SupTi), or a T cell obtained from a mammal, for example, from a subject to be treated. If obtained from a mammal, the T cell can be obtained from numerous sources, including but not limited to blood, bone marrow, lymph node, the thymus, or other tissues or fluids. T cells can also be enriched or purified. The T cell can be any type of T cell and can be of any developmental stage, including but not limited to, CD4⁺/CD8⁺ double positive T cells, CD4⁺ helper T cells (e.g., Th1 and Th2 cells), CD8⁺ T cells (e.g., cytotoxic T cells), tumor infiltrating lymphocytes (TILs), memory T cells (e.g., central memory T cells and effector memory T cells), regulatory T cells, naïve T cells, and the like.

In certain embodiments, an immune cell, e.g., a T cell, is engineered to express an exogenous gene. For example, in certain embodiments, the guide nucleic acid, the engineered, non-naturally occurring system, and the CRISPR expression system disclosed herein may be used to engineer an immune cell to express an exogenous gene at the locus of a human ADORA2A, B2M, CD52, CIITA, CTLA4, DCK, FAS, HAVCR2, LAG3, PDCD1, PTPN6, TIGIT, TRAC, TRBC1, TRBC2, CARD11, CD247, IL7R, LCK, or PLCG1 gene. For example, in certain embodiments, an engineered CRISPR system disclosed herein may catalyze DNA cleavage at the gene locus, allowing for site-specific integration of the exogenous gene at the gene locus by HDR.

In certain embodiments, an immune cell, e.g., a T cell, is engineered to express a chimeric antigen receptor (CAR), i.e., the T cell comprises an exogenous nucleotide sequence encoding a CAR. As used herein, the term “chimeric antigen receptor” or “CAR” refers to any artificial receptor including an antigen-specific binding moiety and one or more signaling chains derived from an immune receptor. CARs can comprise a single chain fragment variable (scFv) of an antibody specific for an antigen coupled via hinge and transmembrane regions to cytoplasmic domains of T cell signaling molecules, e.g. a T cell costimulatory domain (e.g., from CD28, CD137, OX40, ICOS, or CD27) in tandem with a T cell triggering domain (e.g. from CD3ζ). A T cell expressing a chimeric antigen receptor is referred to as a CAR T cell. Exemplary CART cells include CD19 targeted CTL019 cells (see, Grupp et al. (2015) BLOOD, 126: 4983), 19-28z cells (see, Park et al. (2015) J. CLN. ONCOL., 33: 7010), and KTE-C19 cells (see, Locke et al. (2015) BLOOD, 126: 3991). Additional exemplary CAR T cells are described in U.S. Pat. Nos. 8,399,645, 8,906,682, 7,446,190, 9,181,527, 9,272,002, and 9,266,960, U.S. Patent Publication Nos. 2016/0362472, 2016/0200824, and 2016/0311917, and International (PCT) Publication Nos. WO2013/142034, WO2015/120180, WO2015/188141, WO2016/120220, and WO2017/040945. Exemplary approaches to express CARs using CRISPR systems are described in Hale et al. (2017) MOL THER METHODS CLIN DEV., 4: 192, MacLeod et al. (2017) MOL THER, 25: 949, and Eyquem et al. (2017) NATURE, 543: 113.

In certain embodiments, an immune cell, e.g., a T cell, binds an antigen, e.g., a cancer antigen, through an endogenous T cell receptor (TCR). In certain embodiments, an immune cell, e.g., a T cell, is engineered to express an exogenous TCR, e.g., an exogenous naturally occurring TCR or an exogenous engineered TCR. T cell receptors comprise two chains referred to as the α- and β-chains, that combine on the surface of a T cell to form a heterodimeric receptor that can recognize MHC-restricted antigens. Each of α- and β-chain comprises a constant region and a variable region. Each variable region of the α- and β-chains defines three loops, referred to as complementary determining regions (CDRs) known as CDR₁, CDR₂, and CDR₃ that confer the T cell receptor with antigen binding activity and binding specificity.

In certain embodiments, a CAR or TCR binds a cancer antigen selected from B-cell maturation antigen (BCMA), mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD70, CD74, CD123, CD133, CD138, epithelial glycoprotein2 (EGP 2), epithelial glycoprotein-40 (EGP-40), epithelial cell adhesion molecule (EpCAM), receptor-type tyrosine-protein kinase (FLT3), folate-binding protein (FBP), fetal acetylcholine receptor (AChR), folate receptor-α and β (FRα and β), Ganglioside G2 (GD2), Ganglioside G3 (GD3), epidermal growth factor receptor 2 (HER-2/ERB2), epidermal growth factor receptor vIII (EGFRvIII), ERB3, ERB4, human telom erase reverse transcriptase (hTERT). Interleukin-13 receptor subunit alpha-2 (IL-13Ra2), K-light chain, kinase insert domain receptor (KDR), Lewis A (CA19.9), Lewis Y (LeY), LI cell adhesion molecule (LICAM), melanoma-associated antigen 1 (melanoma antigen family A1, MAGE-A1), Mucin 16 (MUC-16), Mucin 1 (MUC-1; e.g., a truncated MUC-1), KG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosme-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-IH, HERK-V, IL-1 IRa, Latent Membrane Protein 1 (LMP1), Neural cell-adhesion molecule (N-CAM/CD56), and Trail Receptor (TRAIL-R).

Genetic loci suitable for insertion of a CAR- or exogenous TCR-encoding sequence include but are not limited to TCR subunit loci (e.g., the TCRα constant (TRAC) locus, the TCRβ constant 1 (TRBC1) locus, and the TCRβ constant 2 (TRBC2) locus). It is understood that insertion in the TRAC locus reduces tonic CAR signaling and enhances T cell potency (see, Eyquem et al. (2017) NATURE, 543: 113). Furthermore, inactivation of the endogenous TRAC, TRBC1, or TRBC2 gene may reduce a graft-versus-host disease (GVHD) response, thereby allowing use of allogeneic T cells as starting materials for preparation of CAR-T cells. Accordingly, in certain embodiments, an immune cell, e.g., a T cell, is engineered to have reduced expression of an endogenous TCR or TCR subunit, e.g., TRAC, TRBC1, and/or TRBC2. The cell may be engineered to have partially reduced or no expression of the endogenous TCR or TCR subunit. For example, in certain embodiments, the immune cell, e.g., a T cell, is engineered to have less than 80% (e.g., less than 70%, less than 60%, less than 50%, less than 40%, less than 30%, less than 20%, less than 10%, or less than 5%) of the expression of the endogenous TCR or TCR subunit relative to a corresponding unmodified or parental cell. In certain embodiments, the immune cell, e.g., a T cell, is engineered to have no detectable expression of the endogenous TCR or TCR subunit. Exemplary approaches to reduce expression of TCRs using CRISPR systems are described in U.S. Pat. No. 9,181,527, Liu et al. (2017) CELL RES, 27: 154, Ren et al. (2017) CLIN CANCER RES, 23: 2255, Cooper et al. (2018) LEUKEMIA, 32: 1970, and Ren et al. (2017) ONCOTARGET, 8: 17002.

It is understood that certain immune cells, such as T cells, also express major histocompatibility complex (MHC) or human leukocyte antigen (HLA) genes, and inactivation of these endogenous gene may reduce a GVHD response, thereby allowing use of allogeneic T cells as starting materials for preparation of CAR-T cells. Accordingly, in certain embodiments, an immune cell, e.g., a T-cell, is engineered to have reduced expression of one or more endogenous class I or class II MHCs or HLAs (e.g., beta 2-microglobulin (B2M), class 11 major histocompatibility complex transactivator (CIITA), HLA-E, and/or HLA-G). The cell may be engineered to have partially reduced or no expression of an endogenous MHC or HLA. For example, in certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous MHC (e.g., B2M. CIITA, HLA-E, or HLA-G) relative to a corresponding unmodified or parental cell. In certain embodiments, the immune cell, e.g., a T cell, is engineered to have no detectable expression of an endogenous MHC (e.g., B2M, CIITA, HLA-E, or HLA-G). Exemplary approaches to reduce expression of MHCs using CRISPR systems are described in Liu et al. (2017) CELL RES. 27: 154, Ren et al. (2017) CLIN CANCER RES, 23: 2255, and Ren et al. (2017) ONCOTARGET, 8: 17002.

Other genes that may be inactivated to reduce a GVHD response include but are not limited to CD3, CD52, and deoxycytidine kinase (DCK). For example, inactivation of DCK may render the immune cells (e.g., T cells) resistant to purine nucleotide analogue (PNA) compounds, which are often used to compromise the host immune system in order to reduce a GVHD response during an immune cell therapy. In certain embodiments, the immune cell, e.g., a T-cell, is engineered to have less than less than 80% (e.g., less than 70%, less than 60%, less than 50%, less than 40%, less than 30%, less than 20%, less than 10%, or less than 5%) of the expression of endogenous CD52 or DCK relative to a corresponding unmodified or parental cell.

It is understood that the activity of an immune cell (e.g., T cell) may be enhanced by inactivating or reducing the expression of an immune suppressor such as an immune checkpoint protein. Accordingly, in certain embodiments, an immune cell, e.g., a T cell, is engineered to have reduced expression of an immune checkpoint protein. Exemplary immune checkpoint proteins expressed by wild-type T cells include but are not limited to PDCD1 (PD-1), CTLA4, ADORA2A (A2AR), B7-H3, B7-H4, BTLA, KIR, LAG3, HAVCR2 (TIM3), TIGIT, VISTA, PTPN6 (SHP-1), and FAS. The cell may be modified to have partially reduced or no expression of the immune checkpoint protein. For example, in certain embodiments, the immune cell. e.g., a T cell, is engineered to have less than 80% (e.g., less than 70%, less than 60%, less than 50%, less than 40%, less than 30%, less than 20%, less than 10%, or less than 5%) of the expression of the immune checkpoint protein relative to a corresponding unmodified or parental cell. In certain embodiments, the immune cell, e.g., a T cell, is engineered to have no detectable expression of the immune checkpoint protein. Exemplary approaches to reduce expression of immune checkpoint proteins using CRISPR systems are described in International (PCT) Publication No. WO2017/017184, Cooper et al. (2018) LEUKEMIA, 32: 1970, Su et al. (2016) ONCOINIMUNOLOGY, 6: e1249558, and Zhang et al. (2017) FRONT MED. 11: 554.

The immune cell can be engineered to have reduced expression of an endogenous gene, e.g., an endogenous genes described above, by gene editing or modification. For example, in certain embodiments, an engineered CRISPR system disclosed herein may result in DNA cleavage at a gene locus, thereby inactivating the targeted gene. In other embodiments, an engineered CRISPR system disclosed herein may be fused to an effector domain (e.g., a transcriptional repressor or histone methylase) to reduce the expression of the target gene.

The immune cell can also be engineered to express an exogenous protein (besides an antigen-binding protein described above) at the locus of a human ADORA2A, B2M, CD52, CIITA, CTLA4, DCK, FAS, HAVCR2, LAG3, PDCD1, PTPN6, TIGIT, TRAC. TRBC1, TRBC2, CARD11, CD247, IL7R, LCK, or PLCG1 gene.

In certain embodiments, an immune cell, e.g., a T cell, is modified to express a dominant-negative form of an immune checkpoint protein. In certain embodiments, the dominant-negative form of the checkpoint inhibitor can act as a decoy receptor to bind or otherwise sequester the natural ligand that would otherwise bind and activate the wild-type immune checkpoint protein. Examples of engineered immune cells, for example, T cells containing dominant-negative forms of an immune suppressor are described, for example, in International (PCT) Publication No. WO2017/040945.

In certain embodiments, an immune cell, e.g., a T cell, is modified to express a gene (e.g., a transcription factor, a cytokine, or an enzyme) that regulates the survival, proliferation, activity, or differentiation (e.g., into a memory cell) of the immune cell. In certain embodiments, the immune cell is modified to express TET2, FOXO1, IL-12, IL-15, IL-18, IL-21, IL-7, GLUT1, GLUT3, HK1, HK2, GAPDH, LDHA, PDK1, PKM2, PFKFB3. PGK1, ENO1, GYS1, and/or ALDOA. In certain embodiments, the modification is an insertion of a nucleotide sequence encoding the protein operably linked to a regulatory element. In certain embodiments, the modification is a substitution of a single nucleotide polymorphism (SNP) site in the endogenous gene. In certain embodiments, an immune cell. e.g., a T cell, is modified to express a variant of a gene, for example, a variant that has greater activity than the respective wild-type gene. In certain embodiments, the immune cell is modified to express a variant of CARD11, CD247, IL7R, LCK, or PLCG1. For example, certain gain-of-function variants of IL7R were disclosed in Zenatti et al., (2011) NAT. GENET. 43(10):932-39. The variant can be expressed from the native locus of the respective wild-type gene by delivering an engineered system described herein for targeting the native locus in combination with a donor template that carries the variant or a portion thereof.

In certain embodiments, an immune cell, e.g., a T cell, is modified to express a protein (e.g., a cytokine or an enzyme) that regulates the microenvironment that the immune cell is designed to migrate to (e.g., a tumor microenvironment). In certain embodiments, the immune cell is modified to express CA9, CA12, a V-ATPase subunit, NHE1, and/or MCT-1.

### V. Kits

It is understood that the guide nucleic acid, the engineered, non-naturally occurring system, the CRISPR expression system, and the library disclosed herein can be packaged in a kit suitable for use by a medical provider. Accordingly, in another aspect, the invention provides kits containing any one or more of the elements disclosed in the above systems, libraries, methods, and compositions. In certain embodiments, the kit comprises an engineered, non-naturally occurring system as disclosed herein and instructions for using the kit. The instructions may be specific to the applications and methods described herein. In certain embodiments, one or more of the elements of the system are provided in a solution. In certain embodiments, one or more of the elements of the system are provided in lyophilized form, and the kit further comprises a diluent. Elements may be provided individually or in combinations, and may be provided in any suitable container, such as a vial, a bottle, a tube, or immobilized on the surface of a solid base (e.g., chip or microarray). In certain embodiments, the kit comprises one or more of the nucleic acids and/or proteins described herein. In certain embodiments, the kit provides all elements of the systems of the invention.

In certain embodiments of a kit comprising the engineered, non-naturally occurring dual guide system, the targeter nucleic acid and the modulator nucleic acid are provided in separate containers. In other embodiments, the targeter nucleic acid and the modulator nucleic acid are pre-complexed, and the complex is provided in a single container.

In certain embodiments, the kit comprises a Cas protein or a nucleic acid comprising a regulatory element operably linked to a nucleic acid encoding a Cas protein provided in a separate container. In other embodiments, the kit comprises a Cas protein pre-complexed with the single guide nucleic acid or a combination of the targeter nucleic acid and the modulator nucleic acid, and the complex is provided in a single container.

In certain embodiments, the kit further comprises one or more donor templates provided in one or more separate containers. In certain embodiments, the kit comprises a plurality of donor templates as disclosed herein (e.g., in separate tubes or immobilized on the surface of a solid base such as a chip or a microarray), one or more guide nucleic acids disclosed herein, and optionally a Cas protein or a regulatory element operably linked to a nucleic acid encoding a Cas protein as disclosed herein. Such kits are useful for identifying a donor template that introduces optimal genetic modification in a multiplex assay. The CRISPR expression systems as disclosed herein are also suitable for use in a kit.

In certain embodiments, a kit further comprises one or more reagents and/or buffers for use in a process utilizing one or more of the elements described herein. Reagents may be provided in any suitable container and may be provided in a form that is usable in a particular assay, or in a form that requires addition of one or more other components before use (e.g., in concentrate or lyophilized form). A buffer may be a reaction or storage buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In certain embodiments, the buffer has a pH from about 7 to about 10. In certain embodiments, the kit further comprises a pharmaceutically acceptable carrier. In certain embodiments, the kit further comprises one or more devices or other materials for administration to a subject.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” and “the” and similar references in the context of describing the invention (especially in the context of the following claims) are to be construed to cover both the singular and the plural, unless otherwise indicated herein or clearly contradicted by context. For example, the term “a cell” includes a plurality of cells, including mixtures thereof. Where the plural form is used for compounds, salts, and the like, this is taken to mean also a single compound, salt, or the like.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain.” “contains.” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a 10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1. Cleavage of Genomic DNA by Single Guide MAD7 CRISPR-Cas Systems

MAD7 is a type V-A Cas protein that has endonuclease activity when complexed with a single guide RNA, also known as a crRNA in a type V-A system (see, U.S. Pat. No. 9,982,279). This example describes cleavage of the genomic DNA of Jurkat cells using MAD7 in complex with single guide nucleic acids targeting human ADORA2A, B2M, CARD11, CD247, CD52, CIITA, CTLA4, DCK, DHODH, FAS, HAVCR2, IL7R, LAG3, LCK, MDV, PDCD1, PLCG1, PLK1, PTPN6, TIGIT, TRAC, TRBC1, TRBC2, TUBB, or U6 gene.

Briefly, Jurkat cells were grown in RPMI 1640 medium (Thermo Fisher Scientific, A1049101) supplemented with 10% fetus bovine serum at 37° C. in a 5% CO2 environment, and split every 2-3 days to a density of 100,000 cells/mL. MAD7 protein, which contained a nucleoplasmin NLS at the C-terminus, was expressed in E. coli and purified by fast protein liquid chromatography (FPLC). RNP complexes were prepared by incubating 66 pmol MAD7 protein with 100 pmol chemically synthesized single guide RNA for 10 minutes at room temperature. The RNPs were mixed with 200,000 Jurkat cells in a final volume of 25 μL. Electroporation was carried out on a 4D-Nucleofector (Lonza) using program CL-120. Following electroporation, the cells were cultured for three days.

Genomic DNA of the cells was extracted using the Quick Extract DNA extraction solution 1.0 (Epicentre). The genes were amplified from the genomic DNA samples in a PCR reaction with primers with or without overhang adaptors and processed using the Nestera XT Index Kit v2 Set A (Illumina, FC-131-2001) or the KAPA HyperPlus kit (Roche, cat. no. KK8514), respectively. The final PCR products were analyzed by next-generation sequencing, and the data were analyzed with the AmpliCan package (see, Labun et al. (2019), Accurate analysis of genuine CRISPR editing events with ampliCan. Genome Res., electronically published in advance). Editing efficiency was determined by the number of edited reads relative to the total number of reads obtained under each condition.

The nucleotide sequence of each single guide RNA used in this example consisted of, from 5′ to 3′, UAAUUUCUACUCUUGUAGAU (SEQ ID NO: 50) and a spacer sequence. In SEQ ID NO: 50, the modulator stem sequence (UCUAC) and the targeter stem sequence (GUAGA) are underlined. The editing efficiency of each single guide RNA was measured as the percentage of cells having one or more insertion or deletion at the target site (% indel). The spacer sequences tested for targeting human ADORA2A, B2M CARD11, CD247, CD52, CIITA, CTLA4, DCK, DHODH, FAS, HAVCR2, IL7R, LAG3, LCK, MVD, PDCD1, PLCG1, PLK1, PTPN6, TIGIT, TRAC, TRBC1, TRBC2, TUBB, or U6 gene and the editing efficiency of each single guide RNA are shown in Tables 6-25 and illustrated in FIGS. 3-15, respectively. In Tables 6-25, N.D. means not determined.

TABLE 6
Tested crRNAs Targeting Human ADORA2A Gene

| crRNA | Spacer Sequence | SEQ ID NO | % Indel |
|---|---|---|---|
| gADORA2A_1 | GTGGTGTCACTGGCGGCGGCC | 242 | 0.3 |
| gADORA2A_2 | TGGTGTCACTGGCGGCGGCCG | 133 | 3.9 |
| gADORA2A_3 | GCCATCACCATCAGCACCGGG | 243 | 0.5 |
| gADORA2A 4 | CCATCACCATCAGCACCGGGT | 137 | 2.1 |
| gADORA2A_5 | GTCCTGGTCCTCACGCAGAGC | 244 | 0.1 |
| gADORA2A_6 | GCCCTCGTGCCGGTCACCAAG | 245 | 0.9 |
| gADORA2A 7 | GTGACCGGCACGAGGGCTAAG | 135 | 2.8 |
| gADORA2A_8 | CCATCGGCCTGACTCCCATGC | 136 | 2.2 |
| gADORA2A_9 | GCTGACCGCAGTTGTTCCAAC | 246 | 1.1 |
| gADORA2A_10 | GGCTGACCGCAGTTGTTCCAA | 247 | 0.5 |
| gADORA2A_11 | GCCCTCCCCGCAGCCCTGGGA | 248 | 1.3 |
| gADORA2A_12 | AGGATGTGGTCCCCATGAACT | 51 | 18 2 |
| gADORA2A_13 | AACTTCTTTGCCTGTGTGCTG | 249 | 0.1 |
| gADORA2A_14 | TTTGCCTGTGTGCTGGTGCCC | 250 | 0.2 |
| gADORA2A_15 | CCTGTGTGCTGGTGCCCCTGC | 251 | 1.1 |
| gADORA2A_16 | CGGATCTTCCTGGCGGCGCGA | 131 | 7.8 |
| gADORA2A_17 | AGCTGTCGTCGCGCCGCCAGG | 252 | 0.1 |
| gADORA2A_18 | TGCAGTGTGGACCGTGCCCGC | 253 | 0.2 |
| gADORA2A_19 | GCAGCATGGACCTCCTTCTGC | 254 | 0.4 |
| gADORA2A_20 | CCCTCTGCTGGCTGCCCCTAC | 255 | 0.6 |
| gADORA2A_21 | ACTTTCTTCTGCCCCGACTGC | 256 | 0.6 |
| gADORA2A_22 | CTTCTGCCCCGACTGCAGCCA | 257 | 1.0 |
| gADORA2A_23 | TTCTGCCCCGACTGCAGCCAC | 134 | 2.8 |
| gADORA2A_24 | ATCTACGCCTACCGTATCCGC | 258 | 0.0 |
| gADORA2A_25 | CGCAAGATCATTCGCAGCCAC | 259 | 0.1 |
| gADORA2A_26 | AAAGGTTCTTGCTGCCTCAGG | 260 | 0.1 |
| gADORA2A_27 | CAAGGCAGCTGGCACCAGTGC | 261 | 0.1 |
| gADORA2A_28 | AAGGCAGCTGGCACCAGTGCC | 132 | 5.8 |
| gADORA2A_29 | AGCTCATGGCTAAGGAGCTCC | 262 | 0.2 |
| gADORA2A_30 | GCCATGAGCTCAAGGGAGTGT | 263 | 0.5 |

TABLE 7
Tested crRNAs Targeting Human B2M Gene

| crRNA Name | Spacer Sequence | SEQ ID NO | % Indel |
|---|---|---|---|
| gB2M_1 | GCTGTGCTCGCGCTACTCTCT | 145 | 1.8 |
| gB2M_2 | TGGCCTGGAGGCTATCCAGCG | 65 | 17.4 |
| gB2M_3 | CCCGATATTCCTCAGGTACTC | 264 | 0.1 |
| gB2M_4 | CTCACGTCATCCAGCAGAGAA | 52 | 74.1 |
| gB2M_5 | CATTCTCTGCTGGATGACGTG | 142 | 2.2 |
| gB2M_6 | CCATTCTCTGCTGGATGACGT | 265 | 1.0 |
| gB2M_7 | ACTTTCCATTCTCTGCTGGAT | 64 | 17.9 |
| gB2M_8 | CTGAATTGCTATGTGTCTGGG | 139 | 3.5 |
| gB2M_9 | AATGTCGGATGGATGAAACCC | 266 | 0.5 |
| gB2M_10 | ATCCATCCGACATTGAAGTTG | 143 | 2.0 |
| gB2M 11 | CTGAAGAATGGAGAGAGAATT | 140 | 3.4 |
| gB2M_12 | TCAATTCTCTCTCCATTCTTC | 267 | 0.7 |
| gB2M 13 | TTCAATTCTCTCTCCATTCTT | 268 | 0.7 |
| gB2M_14 | CTGAAAGACAAGTCTGAATGC | 269 | 0.4 |
| gB2M_15 | TCTTTCAGCAAGGACTGGTCT | 270 | 0.9 |
| gB2M_16 | AGCAAGGACTGGTCTTTCTAT | 271 | 0.3 |
| gB2M_17 | TATCTCTTGTACTACACTGAA | 66 | 15.3 |
| gB2M_18 | TCAGTGGGGGTGAATTCAGTG | 141 | 3.0 |
| gB2M_19 | ACTATCTTGGGCTGTGACAAA | 272 | 0.1 |
| gB2M_20 | GTCACAGCCCAAGATAGTTAA | 273 | 0.8 |
| gB2M_21 | TCACAGCCCAAGATAGTTAAG | 138 | 5.3 |
| gB2M_22 | CCCCACTTAACTATCTTGGGC | 144 | 2.0 |
| gB2M_23 | CTGGCCTGGAGGCTATCCAGC | 618 | 0.77 |
| gB2M_24 | TCCCGATATTCCTCAGGTACT | 619 | 0.54 |
| gB2M_25 | CCGATATTCCTCAGGTACTCC | 620 | 0.14 |
| gB2M_26 | AGTAAGTCAACTTCAATGTCG | 621 | 0.11 |
| gB2M_27 | AATTCTCTCTCCATTCTTCAG | 622 | 2.70 |
| gB2M_28 | CAATTCTCTCTCCATTCTTCA | 623 | 0.26 |
| gB2M_29 | CAGCAAGGACTGGTCTTTCTA | 624 | 0.19 |
| gB2M_30 | AGTGGGGGTGAATTCAGTGTA | 625 | 91.96 |
| gB2M_31 | CAGTGGGGGTGAATTCAGTGT | 626 | 8.10 |
| gB2M_33 | CTATCTCTTGTACTACACTGA | 627 | 0.21 |
| gB2M_34 | TACTACACTGAATTCACCCCC | 628 | 0.80 |
| gB2M_35 | GGCTGTGACAAAGTCACATGG | 629 | 0.18 |
| gB2M_36 | CAAAAGAATGTAAGACTTACC | 630 | 0.13 |
| gB2M_37 | CCTCCATGATGCTGCTTACAT | 631 | 0.81 |
| gB2M_38 | TTCATAGATCGAGACATGTAA | 632 | 0.18 |
| gB2M_39 | TCATAGATCGAGACATGTAAG | 633 | 0.20 |
| gB2M_40 | CATAGATCGAGACATGTAAGC | 634 | 4.25 |
| gB2M_41 | ATAGATCGAGACATGTAAGCA | 635 | 93.92 |

TABLE 8
Tested crRNAs Targeting Human CD52 Gene

| crRNA Name | Spacer Sequence | SEQ ID NO | % Indel |
|---|---|---|---|
| gCD52_1 | CTCTTCCTCCTACTCACCATC | 53 | 28.4 |
| gCD52_2 | TCCTCCTACAGATACAAACTG | 274 | N.D. |
| gCD52_3 | GTCCTGAGAGTCCAGTTTGTA | 275 | N.D. |
| gCD52_4 | GCTGGTGTCGTTTTGTCCTGA | 146 | 4.1 |
| gCD52_5 | TGTTGCTGGATGCTOAGGGGC | 276 | 1.1 |
| gCD52_6 | CCTTTTCTTCGTGGCCAATGC | 277 | 0.2 |
| gCD52_7 | TCTTCGTGGCCAATGCCATAA | 278 | 0.2 |
| gCD52_8 | CTTCGTGGCCAATGCCATAAT | 279 | 0.15 |

TABLE 9
Tested crRNAs Targeting Human CHITA Gene

| crRNA | Spacer Sequence | SEQ ID NO | % Indel |
|---|---|---|---|
| gCIITA_1 | GGGCTCTGACAGGTAGGACCC | 280 | 0.5 |
| gCIITA_2 | TACCTTGGGGCTCTGACAGGT | 281 | 0.0 |
| gCIITA_3 | TTACCTTGGGGCTCTGACAGG | 282 | 0.0 |
| gCIITA_4 | TAGGGGCCCCAACTCCATGGT | 54 | 13.5 |
| gCIITA_5 | TTAACAGCGATGCTGACCCCC | 284 | 0.1 |
| gCIITA_6 | TATGACCAGATGGACCTGGCT | 285 | 0.2 |
| gCIITA_7 | TCCTCCCAGAACCCGACACAG | 286 | 0.1 |
| gCIITA_8 | CCTCCCAGAACCCGACACAGA | 287 | 0.1 |
| gCIITA_9 | CATGTCACACAACAGCCTGCT | 288 | 0.1 |
| gCIITA_10 | CTCACCGATATTGGCATAAGC | 289 | 0.1 |
| gCIITA_11 | TCCTTGTCTGGGCAGCGGAAC | 290 | 0.1 |
| gCIITA_12 | CCTTGTCTGGGCAGCGGAACT | 291 | 0.4 |
| gCIITA_13 | TCTGGGCAGCGGAACTGGACC | 292 | 0.1 |
| gCIITA_14 | CTCAGGCCCTCCAGCTGGGAG | 293 | 0.2 |
| gCIITA_15 | CTGAAAATGTCCTTGCTCAGG | 294 | 0.2 |
| gCIITA_16 | TCTCAAAGTAGAGCACATAGG | 295 | 0.1 |
| gCIITA_17 | ATCTOGTCCTATGTGCTCTAC | 296 | 0.2 |
| gCIITA_18 | TGCTGGCATCTCCATACTCTC | 147 | 4.8 |
| gCIITA_19 | CTGCCCAACTTCTGCTGGCAT | 297 | 0.5 |
| gCIITA_20 | TOTGCCCAACTTCTGCTGGCA | 298 | 0.1 |
| gCIITA_21 | CTGACTTTTCTGCCCAACTTC | 299 | 0.1 |
| gCIITA_22 | CTCTGCAGCCTTCCCAGAGGA | 300 | 0.6 |
| gCIITA_23 | CCAGAGGAGCTTCCGGCAGAC | 301 | 0.9 |
| gCIITA_24 | AGGTCTGCCGGAAGCTCCTCT | 302 | 0.1 |
| gCIITA_25 | CAGTGCTTCAGGTCTGCCGGA | 303 | 0.2 |
| gCIITA_26 | CGGCAGACCTGAAGCACTGGA | 304 | 0.3 |
| gCIITA_27 | CTCACAGCTGAGCCCCCCACT | 305 | 0.4 |
| gCIITA_28 | CTCCAGGCGCATCTGGCCGGA | 306 | 0.7 |
| gCIITA_29 | GTCTCTTGCAGTGCCTTTCTC | 148 | 2.4 |
| gCIITA_30 | TCTCTTGCAGTGCCTTTCTCC | 307 | 0.1 |
| gCIITA_31 | CTCCAGTTCCTCGTTGAGCTG | 308 | 0.1 |
| gCIITA_32 | CCTTGGGGCTCTGACAGGTAG | 636 | 93.85 |
| gCIITA_33 | ACCTTGGGGCTCTGACAGGTA | 637 | 11.83 |
| gCIITA_34 | CCGGCCTTTTTACCTTGGGGC | 638 | 2.26 |
| gCIITA_35 | CTCCCAGAACCCGACACAGAC | 639 | 48.70 |
| gCIITA_36 | TGGGCTCAGGTGCTTCCTCAC | 640 | 85.46 |
| gCIITA_37 | CTGGGCTCAGGTGCTTCCTCA | 641 | 0.45 |
| gCIITA_38 | CTTGTCTGGGCAGCGGAACTG | 642 | 38.38 |
| gCIITA_39 | CTCAAAGTAGAGCACATAGGA | 643 | 0.25 |
| gCIITA_40 | TCAAAGTAGAGCACATAGGAC | 644 | 15.68 |
| gCIITA_41 | TGCCCAACTTCTGCTGGCATC | 645 | 46.21 |
| gCIITA_42 | TGACTTTTCTGCCCAACTTCT | 646 | 2.72 |
| gCIITA_43 | TCTGCAGCCTTCCCAGAGGAG | 647 | 55.09 |
| gCIITA_44 | TCCAGGCGCATCTGGCCGGAG | 648 | 39.16 |
| gCIITA 45 | TCCAGTTCCTCGTTGAGCTGC | 649 | 0.22 |
| gCIITA_46 | CCAGAGCCCATGGGGCAGAGT | 650 | 1.51 |
| gCIITA_47 | TCCCCACCATCTCCACTCTGC | 651 | 2.05 |
| gCIITA_48 | CTCGGGAGGTCAGGGCAGGTT | 652 | 61.63 |
| gCIITA_49 | GAAGCTTGTTGGAGACCTCTC | 653 | 0.67 |
| gCIITA_50 | GGAAGCTTGTTGGAGACCTCT | 654 | 0.57 |
| gCIITA_51 | CAGAGCCGGTGGAGCAGTTCT | 655 | 8.94 |
| gCIITA_52 | CCCAGCACAGCAATCACTCGT | 656 | 2.63 |
| gCIITA_53 | TCTTCTCTGTCCCCTGCCATT | 657 | 0.28 |
| gCIITA_55 | AGCCACATCTTGAAGAGACCT | 658 | 5.71. |
| gCIITA_56 | CCAGAAGAAGCTGCTCCGAGG | 659 | 0.52 |
| gCIITA_57 | CAGAAGAAGCTGCTCCGAGGT | 660 | 12.02 |
| gCIITA_58 | AGCTGTCCGGCTTCTCCATGG | 661 | 3.25 |
| gCIITA_59 | AGAGCTCAGGGATGACAGAGC | 662 | 16.35 |
| gCIITA_60 | TGCCGGGCAGTGTGCCAGCTC | 663 | 11.98 |
| gCIITA_61 | ATGTCTGCGGCCCAGCTCCCA | 664 | 1.25 |
| gCIITA_62 | GCCATCGCCCAGGTCCTCACG | 665 | 1.29 |
| gCIITA_63 | GCCACTCAGAGCCAGCCACAG | 666 | 35.47 |
| gCIITA_64 | TGGCTGGGCTGATCTTCCAGC | 667 | 0.50 |
| gCIITA_65 | GCAGCACGTGGTACAGGAGCT | 668 | 70.73 |
| gCIITA_66 | CTGGGCACCCGCCTCACGCCT | 669 | 0.31 |
| gCIITA_67 | TGGGCACCCGCCTCACGCCTC | 670 | 12.57 |
| gCIITA_68 | CCCCTCTGGATTGGGGAGCCT | 671 | 4.61 |
| gCIITA_69 | AAAGGCTCGATGGTGAACTTC | 672 | 1.17 |
| gCIITA_70 | CCAGGTCTTCCACATCCTTCA | 673 | 38.98 |
| gCIITA_71 | AAAGCCAAGTCCCTGAAGGAT | 674 | 39.50 |
| gCIITA_72 | GGTCCCGAACAGCAGGGAGCT | 675 | 89.25 |
| gCIITA_73 | TTTAGGTCCCGAACAGCAGGG | 676 | 10.88 |
| gCIITA_74 | CTTACGCAAACTCCAGTTTCT | 677 | 0.79 |
| gCIITA_75 | CCTCCTAGGCTGGGCCCTGTC | 678 | 2.78 |
| gCIITA_76 | GGGAAAGCCTGGGGGCCTGAG | 679 | 68.93 |
| gCIITA_77 | CCCAAACTGGTGCOGATCCTC | 680 | 0.57 |
| gCIITA_79 | CTCCCTGCAGCATCTGGAGTG | 681 | 1.12 |
| gCIITA_80 | CAAGGACTTCAGCTGGGGGAA | 682 | 87.87 |
| gCIITA_81 | TAGGCACCCAGGTCAGTGATG | 683 | 44.56 |
| gCIITA_82 | CGACAGCTTGTACAATAACTG | 684 | 34.37 |
| gCIITA_83 | TCTTGCCAGCGTCCAGTACAA | 685 | 5.62 |
| gCIITA_84 | CCCGGCCTTTTTACCTTGGGG | 686 | 0.38 |
| gCIITA_85 | CCTCCCAGGCAGCTCACAGTG | 687 | 0.74 |
| gCIITA_87 | TCCAGCCAGGTCCATCTGGTC | 688 | 0.15 |
| gCIITA_88 | TTCTCCAGCCAGGTCCATCTG | 689 | 0.21 |
| gCIITA_89 | ATCACCTTCCATGTCACACAA | 690 | 0.31 |
| gCIITA_90 | TCTGGGCTCAGGTGCTTCCTC | 691 | 0.25 |
| gCIITA_91 | TGCCAATATCGGTGAGGAAGC | 692 | 0.17 |
| gCIITA_92 | CAGGACTCCCAGCTGGAGGGC | 693 | 0.61 |
| gCIITA_93 | TCTGACTTTTCTGCCCAACTT | 694 | 0.21 |
| gCIITA_94 | CAGTGCCTTTCTCCAGTTCCT | 695 | 0.25 |
| gCIITA_95 | GCTGGCCTGGGGCACCTCACC | 696 | 0.59 |
| gCIITA_96 | GCTCCATCAGCCACTGACCTG | 697 | 0.29 |
| gCIITA_97 | CCTGTCATGTTTGCTCGGGAG | 698 | 0.27 |
| gCIITA_98 | TCCATCTCCAGAGCACAAGAC | 699 | 0.23 |
| gCIITA_99 | TTGGAGACCTCTCCAGCTGCC | 700 | 0.99 |
| gCIITA_100 | GCAGAGCCGGTGGAGCAGTTC | 701 | 0.46 |
| gCIITA_101 | CTGCTGCTCCTCTCCAGCCTG | 702 | 0.23 |
| gCIITA_103 | GCAGCCAACAGCACCTCAGCC | 703 | 0.22 |
| gCIITA_104 | GCCCAGCACAGCAATCACTCG | 704 | 0.07 |

TABLE 10
Tested crRNAs Targeting Human CTLA4 Gene

| crRNA | Spacer Sequence | SEQ ID NO | % Indel |
|---|---|---|---|
| gCTLA4_1 | TGCCGCTGAAATCCAAGGCAA | 309 | 1.3 |
| gCTLA4_2 | CCTTGGATTTCAGCGGCACAA | 310 | 0.8 |
| gCTLA4_3 | GATTTCAGCGGCACAAGGCTC | 311 | 0.6 |
| gCTLA4_4 | AGCGGCACAAGGCTCAGCTGA | 55 | 58.4 |
| gCTLA4_5 | TTCTTCTCTTCATCCCTGTCT | 155 | 1.7 |
| gCTLA4_6 | CAGAAGACAGGGATGAAGAGA | 68 | 44.6 |
| gCTLA4_7 | GCAGAAGACAGGGATGAAGAG | 312 | 0.2 |
| gCTLA4_8 | GGCTTTTCCATOCTAGCAATG | 313 | 0.1 |
| gCTLA4_9 | GCTTTTCCATGCTAGCAATGC | 314 | 0.2 |
| gCTLA4_10 | TCCATGCTAGCAATGCACGTG | 315 | 0.1 |
| gCTLA4_11 | CCATGCTAGCAATGCACGTGG | 316 | 0.1 |
| gCTLA4_12 | GTGTGTGAGTATGCATCTCCA | 317 | 0.8 |
| gCTLA4_13 | TGTGTGAGTATGCATCTCCAG | 70 | 12.6 |
| gCTLA4_14 | CCTGGAGATGCATACTCACAC | 67 | 47.4 |
| gCTLA4_15 | GCCTGGAGATGCATACTCACA | 318 | 0.2 |
| gCTLA4_16 | GGCAGGCTGACAGCCAGGTGA | 319 | 1.2 |
| gCTLA4_17 | AGTCACCTGGCTGTCAGCCTG | 320 | 0.4 |
| gCTLA4_18 | CTAGATGATTCCATCTGCACG | 154 | 2.0 |
| gCTLA4_19 | CACTGGAGGTGCCCGTGCAGA | 69 | 42.5 |
| gCTLA4_20 | ATTTCCACTGGAGGTGCCCGT | 321 | 0.1 |
| gCTLA4_21 | GATAGTGAGGTTCACTTGATT | 322 | 0.6 |
| gCTLA4_22 | CAGATGTAGAGTCCCGTGTCC | 323 | 0.6 |
| gCTLA4_23 | CTCACCAATTACATAAATCTG | 324 | 0.8 |
| gCTLA4_24 | GCTCACCAATTACATAAATCT | 325 | 1.0 |
| gCTLA4_25 | GTTTTCTGTTGCAGATCCAGA | 326 | 0.1 |
| gCTLA4_26 | TTTTCTGTTGCAGATCCAGAA | 327 | 0.1 |
| gCTLA4_27 | CTGTTOCAGATCCAGAACCGT | 149 | 5.0 |
| gCTLA4_28 | CTCCTCTGGATCCTTGCAGCA | 152 | 3.0 |
| gCTLA4_29 | CAGCAGTTAGTTCGGGGTTGT | 328 | 0.7 |
| gCTLA4_30 | TTTATAGCTTTCTCCTCACAG | 329 | 0.6 |
| gCTLA4_31 | CTCCTCACAGCTGTTTCTTTG | 330 | 1.0 |
| gCTLA4_32 | TCCTCACAGCTGTTTCTTTGA | 331 | 0.7 |
| gCTLA4_33 | GCTCAAAGAAACAGCTGTGAG | 332 | 0.8 |
| gCTLA4_34 | TTTTTGTGTTTGACAGCTAAA | 333 | 0.5 |
| gCTLA4_35 | TGTGTTTGACAGCTAAAGAAA | 334 | 0.1 |
| gCTLA4_36 | ACAGCTAAAGAAAAGAAGCCC | 150 | 3.9 |
| gCTLA4_37 | CACATAGACCCCTGTTGTAAG | 153 | 2.9 |
| gCTLA4_38 | CACATTCTGGCTCTGTTGGGG | 335 | 0.2 |
| gCTLA4_39 | TCACATTCTGGCTCTGTTGGG | 336 | 0.3 |
| gCTLA4_40 | AGCCTTATTTTATTCCCATCA | 337 | 0.3 |
| gCTLA4_41 | TCAATTGATGGGAATAAAATA | 151 | 3.0 |

TABLE IL
Tested crRNAs Targeting Human DCK Gene

| crRNA | Spacer Sequence | SEQ ID NO | % Indel |
|---|---|---|---|
| gDCK_1 | TCTTGGGCGGGGTGGCCATTC | 338 | 0.1 |
| gDCK 2 | TCAGCCAGCTCTGAGGGGACC | 71 | 50.4 |
| gDCK_3 | CTTGATGCGGGTCCCCTCAGA | 339 | 0.3 |
| gDCK_4 | GATGGAGATTTTCTTGATGCG | 340 | 0.3 |
| gDCK_5 | CCGATGTTCCCTTCGATGGAG | 341 | 0.5 |
| gDCK_6 | CGGAGGCTCCTTACCGATGTT | 56 | 85.1 |
| gDCK_7 | ATCTTTCCTCACAACAGCTGC | 159 | 1.5 |
| gDCK_8 | CTCACAACAGCTGCAGGGAAG | 72 | 31.7 |
| gDCK_9 | AGGATATTCACAAATGTTGAC | 156 | 8.1 |
| gDCK_10 | TGAATATCCTTAAACAATTGT | 342 | 1.0 |
| gDCK_11 | CCAATCTTCACACAATTGTTT | 343 | 0.1 |
| gDCK_12 | AACAATTGTGTGAAGATTGGG | 344 | 0.8 |
| gDCK_13 | AACATTGCACCATCTGGCAAC | 345 | 1.2 |
| gDCK_14 | GAACATTGCACCATCTGGCAA | 346 | 0.6 |
| gDCK_15 | CATACCTCAAATTCATCTTGA | 347 | 0.3 |
| gDCK_16 | ATTTTCATACCTCAAATTCAT | 348 | 0.1 |
| gDCK_17 | AATTTTATTTTCATACCTCAA | 349 | 0.0 |
| gDCK_18 | TGCACATTCAAAATAGGAACT | 350 | 0.4 |
| gDCK_19 | TCTGAGACATTGTAAGTTCCT | 351 | 0.7 |
| gDCK_20 | CAATGTCTCAGAAAAATGGTG | 352 | 0.6 |
| gDCK_21 | TCATACATCATCTGAAGAACA | 158 | 3.6 |
| gDCK_22 | GAAGGTAAAAGACCATCGTTC | 157 | 5.6 |
| gDCK_23 | ACCTTCCAAACATATGCCTGT | 353 | 1.2 |
| gDCK_24 | CAAACATATGCCTGTCTCAGT | 354 | 1.1 |
| gDCK_25 | CCATTCAGAGAGGCAAGCTGA | 355 | 0.9 |
| gDCK_26 | AGCTTGCCATTCAGAGAGGCA | 73 | 13.3 |
| gDCK_27 | CCTCTCTGAATOGCAAGCTCA | 356 | 1.1 |
| gDCK_28 | TCTGCATCTTTGAGCTTGCCA | 357 | 0.1 |
| gDCK_29 | TTGAACGATCTGTGTATAGTG | 358 | 0.2 |
| gDCK_30 | TACATACCTGTCACTATACAC | 74 | 12.8 |
| gDCK_31 | AGGTATATTTTTGCATCTAAT | 359 | 0.05 |

TABLE 12
Tested crRNAs Targeting
Human FAS Gene

| | | SEQ ID | % |
|---|---|---|---|
| crRNA | Spacer Sequence | NO | Indel |
| gFAS_1 | GGAGGATTGCTCAACAACCAT | 78 | 22.6 |
| gFAS_2 | TATTTTACAGGTTCTTACGTC | 360 | 0.1 |
| gFAS_3 | ATTTTACAGGTTCTTACGTCT | 361 | 0.7 |
| gFAS_4 | ACAGGTTCTTACGTCTGTTGC | 172 | 1.5 |
| gFAS_5 | GGACGATAATCTAGCAACAGA | 165 | 1.9 |
| gFAS_6 | TGGACGATAATCTAGCAACAG | 362 | 0.0 |
| gFAS_7 | GGCATTAACACTTTTGGACGA | 363 | 0.1 |
| gFAS_8 | GAGTTGATGTCAGTCACTTGG | 364 | 0.1 |
| gFAS_9 | CAAGTTCTGAGTCTCAACTGT | 365 | 0.1 |
| gFAS_10 | GAAGGCCTGCATCATGATGGC | 163 | 2.4 |
| gFAS_11 | TGGCAGAATTGGCCATCATGA | 366 | 0.8 |
| gFAS_12 | GTGTAACATACCTGGAGGACA | 77 | 29.9 |
| gFAS_13 | TTTCCTTGGGCAGGTGAAAGG | 367 | 1.1 |
| gFAS_14 | TTCCTTGGGCAGGTGAAAGGA | 166 | 1.7 |
| gFAS_15 | GGCAGGTGAAAGGAAAGCTAG | 173 | 1.5 |
| gFAS_16 | TTGGCAGGGCACGCAGTCTGG | 368 | 0.7 |
| gFAS_17 | CCTTCTTGGCAGGGCACGCAG | 369 | 0.8 |
| gFAS_18 | TCTGTGTACTCCTTCCCTTCT | 370 | 1.0 |
| gFAS_19 | GTCTGTGTACTCCTTCCCTTC | 371 | 0.6 |
| gFAS_20 | GAAGAAAAATGGGCTTTGTCT | 372 | 0.7 |
| gFAS_21 | TCTTCCAAATGCAGAAGATGT | 373 | 0.7 |
| gFAS_22 | ATCACACAATCTACATCTTCT | 374 | 0.5 |
| gFAS_23 | AAGACTCTTACCATGTCCTTC | 375 | 0.6 |
| gFAS_24 | CAAACTGATTTTCTAGGCTTA | 376 | 0.1 |
| gFAS_25 | CTAGGCTTAGAAGTGGAAATA | 162 | 3.5 |
| gFAS_26 | GAAGTGGAAATAAACTGCACC | 377 | 0.3 |
| gFAS_27 | GTATTCTGGGTCCGGGTGCAG | 378 | 1.3 |
| gFAS_28 | CATCTGCACTTGGTATTCTGG | 379 | 1.2 |
| gFAS_29 | GTTTACATCTGCACTTGGTAT | 167 | 1.6 |
| gFAS_30 | TTTTGTAACTCTACTGTATGT | 380 | 0.8 |
| gFAS_31 | TTTGTAACTCTACTGTATGTG | 381 | 1.4 |
| gFAS_32 | GTGCAAGGGTCACAGTGTTCA | 164 | 2.4 |
| gFAS_33 | CTTGGTGCAAGGGTCACAGTG | 168 | 1.6 |
| gFAS_34 | TTTTTCTAGATGTGAACATGG | 75 | 59.1 |
| gFAS_35 | ATGATTCCATGTTCACATCTA | 76 | 58.5 |
| gFAS_36 | GTGTTGCTGGTGAGTGTGCAT | 57 | 61.9 |
| gFAS_37 | CACTTGGTGTTGCTGGTGAGT | 382 | 1.3 |
| gFAS_38 | CTCTTTGCACTTGGTGTTGCT | 170 | 1.5 |
| gFAS_39 | GGGTGGCTTTGTCTTCTTCTT | 383 | 0.1 |
| gFAS_40 | GTCTTCTTCTTTTGCCAATTC | 384 | 0.6 |
| gFAS_41 | TCTTCTTCTTTTGCCAATTCC | 385 | 0.1 |
| gFAS_42 | GCCAATTCCACTAATTGTTTG | 386 | 0.4 |
| gFAS_43 | CCCCAAACAATTAGTGGAATT | 387 | 0.4 |
| gFAS_44 | AACAAAGCAAGAACTTACCCC | 388 | 0.3 |
| gFAS_45 | TTTGTTCTTTCAGTGAAGAGA | 161 | 6.0 |
| gFAS_46 | TTCTTTCAGTGAAGAGAAAGG | 389 | 0.9 |
| gFAS_47 | AGTGAAGAGAAAGGAAGTACA | 160 | 9.8 |
| gFAS_48 | CTGTACTTCCTTTCTCTTCAC | 390 | 0.8 |
| gFAS_49 | TGCATGTTTTCTGTACTTCCT | 391 | 0.6 |
| gFAS_50 | CTGCATGTTTTCTGTACTTCC | 392 | 0.4 |
| gFAS_51 | TGTGCTTTCTGCATGTTTTCT | 393 | 0.3 |
| gFAS_52 | CTGTGCTTTCTGCATGTTTTC | 394 | 0.3 |
| gFAS_53 | CCTTTCTGTGCTTTCTGCATG | 395 | 0.3 |
| gFAS_54 | GTTTTCCTTTCTGTGCTTTCT | 396 | 0.4 |
| gFAS_55 | AAGTTGGAGATTCATGAGAAC | 397 | 0.4 |
| gFAS_56 | AATACCTACAGGATTTAAAGT | 398 | 0.3 |
| gFAS_57 | TTOCTTTCTAGGAAACAGTGG | 399 | 1.1 |
| gFAS_58 | CTAGGAAACAGTGGCAATAAA | 400 | 1.3 |
| gFAS_59 | TAGGAAACAGTGGCAATAAAT | 79 | 11.0 |
| gFAS_60 | CCAGATAAATTTATTGCCACT | 401 | 0.7 |
| gFAS_61 | CTATTTTTCAGATGTTGACTT | 402 | 0.1 |
| gFAS_62 | TCAGATGTTGACTTGAGTAAA | 403 | 0.6 |
| gFAS_63 | AGTAAATATATCACCACTATT | 404 | 0.8 |
| gFAS_64 | AACTTGACTTAGTGTCATGAC | 405 | 0.4 |
| gFAS_65 | GAACAAAGCCTTTAACTTGAC | 406 | 0.5 |
| gFAS_66 | GTTCGAAAGAATGGTGTCAAT | 407 | 0.9 |
| gFAS_67 | ATTGACACCATTCTTTCGAAC | 408 | 0.5 |
| gFAS_68 | TTCGAAAGAATGGTGTCAATG | 409 | 0.7 |
| gFAS_64 | GGCTTCATTGACACCATTCTT | 410 | 0.4 |
| gFAS_70 | TGTTCTGCTGTGTCTTGGACA | 171 | 1.5 |
| gFAS_71 | CTGTTCTGCTGTGTCTTGGAC | 169 | 1.5 |
| gFAS_72 | GTAATTGGCATCAACTTCATG | 411 | 0.3 |
| gFAS_73 | CATOAAGTTGATGCCAATTAC | 412 | 0.8 |
| gFAS_74 | TTTCCATGAAGTTGATGCCAA | 413 | 0.4 |
| gFAS_75 | TTTCTTTCCATGAAGTTGATG | 414 | 0.5 |
| gFAS_76 | ATGGAAAGAAAGAAGCGTATG | 415 | 1.3 |
| gFAS_77 | ATCAATGTGTCATACGCTTCT | 416 | 0.8 |
| gFAS_78 | TTGAGATCTTTAATCAATGTG | 417 | 1.0 |
| gFAS_79 | TTTGAGATCTTTAATOAATGT | 418 | 0.9 |
| gFAS_80 | CTCTGCAAGAGTACAAAGATT | 419 | 0.2 |
| gFAS_81 | TACTCTTGCAGAGAAAATTCA | 420 | 0.2 |
| gFAS_82 | AGGATGATAGTCTGAATTTTC | 421 | 0.4 |
| gFAS_83 | CTGAGTCACTAGTAATGTCCT | 422 | 0.7 |
| gFAS_84 | AATTTTCTGAGTCACTAGTAA | 423 | 0.6 |
| gFAS_85 | TGAAGTTTGAATTTTCTGAGT | 424 | 0.4 |
| gFAS_86 | ATTTCTGAAGTTTGAATTTTC | 425 | 0.3 |
| gFAS_87 | GATTTCATTTCTGAAGTTTGA | 426 | 0.5 |
| gFAS_88 | GGATTTCATTTCTGAAGTTTG | 427 | 0.5 |
| gFAS_89 | AGAAATGAAATCCAAAGCTTG | 428 | 0.5 |
| gFAS_90 | TCACTCTAGACCAAGCTTTGG | 429 | 0.5 |
| gFAS_91 | TTGTTTTTCACTCTAGACCAA | 430 | 0.7 |
| gFAS_92 | GTCTAGAGTGAAAAACAACAA | 431 | 0.5 |

TABLE 13
Tested crRNAs Targeting
Human HAVCR2 Gene

| | | SEQ ID | % |
|---|---|---|---|
| crRNA | Spacer Sequence | NO | Indel |
| gTIM3_1 | TCTTCTGCAAGCTCCATGTTT | 432 | 0.1 |
| gTIM3_2 | TCTTCTGCAAGCTCCATGTTT | 433 | 0.07 |
| gTIM3_3 | CTTCTGCAAGCTCCATGTTTT | 434 | 0.1 |
| gTIM3_4 | CACATCTTCCCTTTGACTGTG | 435 | 0.8 |
| gTIM3_5 | GACTGTGTCCTGCTGCTGCTG | 436 | 0.8 |
| gTIM3_6 | TAAGTAGTAGCAGCAGCAGCA | 81 | 53.7 |
| gTIM3_7 | CTTGTAAGTAGTAGCAGCAGC | 58 | 64.4 |
| gTIM3_8 | TCTCTCTATGCAGGGTCCTCA | 437 | 0.1 |
| gTIM3_9 | TACACCCCAGCCGCCCCAGGG | 438 | 1.0 |
| gTIM3_10 | CCCCAGCAGACGGGCACGAGG | 175 | 7.3 |
| gTIM3_11 | GCCCCAGCAGACGGGCACGAG | 439 | 0.6 |
| gTIM3_12 | AATGTGGCAACGTGGTGCTCA | 84 | 21.9 |
| gTIM3_13 | ATCAGTCCTGAGCACCACGTT | 187 | 1.5 |
| gTIM3_14 | CATCAGTCCTGAGCACCACGT | 440 | 0.1 |
| gTIM3_15 | GCCAGTATCTGGATGTCCAAT | 181 | 2.9 |
| gTIM3_16 | CGGAAATCCCCATTTAGCCAG | 441 | 0.4 |
| gTIM3_17 | GCGGAAATCCCCATTTAGCCA | 442 | 0.1 |
| gTIM3_18 | CGCAAAGGAGATGTGTCCCTG | 86 | 14.4 |
| gTIM3_19 | GATCCGGCAGCAGTAGATCCC | 178 | 5.1 |
| gTIM3_20 | TCATCATTCATTATGCCTGGG | 443 | 0.1 |
| gTIM3_21 | AGGTTAAATTTTTCATCATTC | 444 | 0.1 |
| gTIM3_22 | ATGACCAACTTCAGGTTAAAT | 445 | 0.1 |
| gTIM3_23 | ACCTGAAGTTGGTCATCAAAC | 184 | 2.2 |
| gTIM3_24 | TGTTGTTTCTGACATTAGCCA | 446 | 0.7 |
| gTIM3_25 | TGACATTAGCCAAGGTCACCC | 85 | 15.7 |
| gTIM3_26 | GAAAGGCTGCAGTGAAGTCTC | 447 | 0.1 |
| gTIM3_27 | ACTGCAGCCTTTCCAAGGATG | 182 | 2.6 |
| gTIM3_28 | CCAAGGATGCTTACCACCAGG | 185 | 1.9 |
| gTIM3_29 | CAAGGATGCTTACCACCAGGG | 80 | 59.8 |
| gTIM3_30 | CCACCAGGGGACATGGCCCAG | 83 | 22 1 |
| gTIM3_31 | TATAGCAGAGACACAGACACT | 448 | 0.3 |
| gTIM3_32 | TATCAGGGAGGCTCCCCAGTG | 82 | 22.4 |
| gTIM3_33 | CTGTTAGATTTATATCAGGGA | 449 | 1.4 |
| gTIM3_34 | TGTTTCCATAGCAAATATCCA | 177 | 5.6 |
| gTIM3_35 | CATAGCAAATATCCACATTGG | 450 | 1.0 |
| gTIM3_36 | CGGGACTCTGGAGCAACCATC | 180 | 3.3 |
| gTIM3_37 | AAAATTAAAGCGCCGAAGATA | 451 | 0.2 |
| gTIM3_38 | CATTTGAAAATTAAAGCGCCG | 452 | 0.1 |
| gTIM3_39 | TGTTTCCCCCTTACTAGGGTA | 453 | 0.7 |
| gTIM3_40 | GTTTCCCCCTTACTAGGGTAT | 186 | 1.7 |
| gTIM3_41 | CCCCTTACTAGGGTATTCTCA | 183 | 2.2 |
| gTIM3_42 | CTAGGGTATTCTCATAGCAAA | 174 | 8.5 |
| gTIM3_43 | AATTCTGTATCTTCTCTTTGC | 454 | 0.7 |
| gTIM3_44 | ATTTCCACAGCCTCATCTCTT | 455 | 0.4 |
| gTIM3_45 | TTTCCACAGCCTCATCTCTTT | 456 | 1.0 |
| gTIM3_46 | CACAGCCTCATCTCTTTGGCC | 457 | 0.5 |
| gTIM3_47 | GCCAACCTCCCTCCCTCAGGA | 176 | 6.0 |
| gTIM3_48 | CCAATCCTGAGGGAGGGAGGT | 179 | 4.5 |
| gTIM3_49 | CTTCTGAGCGAATTCCCTCTG | 458 | 0.7 |
| gTIM3_50 | ATATACGTTCTCTTCAATGGT | 459 | 0.5 |
| gTIM3_51 | GGGTTGTCGCTTTGCAATGCC | 460 | 0.5 |

TABLE 14
Tested crRNAs Targeting
Human LAG3 Gene

| | | SEQ ID | % |
|---|---|---|---|
| crRNA | Spacer Sequence | NO | Indel |
| gLAG3_1 | CTGTTTCTGCAGCCGCTTTGG | 461 | 0.2 |
| gLAG3_2 | TGCAGCCGCTTTGGGTGGCTC | 462 | 0.2 |
| gLAG3_3 | ACCTGGAGCCACCCAAAGCGG | 195 | 3.1 |
| gLAG3_4 | GCTCACCTAGTGAAGCCTCTC | 463 | 1.3 |
| gLAG3_5 | TGCGAAGAGCAGGGGTCACTT | 464 | 0.8 |
| gLAG3_6 | GGGTGCATACCTGTCTGGCTG | 59 | 52.4 |
| gLAG3_7 | CCGCCCAGTGGCCCGCCCGCT | 465 | N.D. |
| gLAG3_8 | TCGCTATGGCTGCGCCCAGCC | 466 | 0.1 |
| gLAG3_9 | TCCTTGCACAGTGACTGCCAG | 467 | N.D. |
| gLAG3_10 | CACAGTGACTGCCAGCCCCCC | 468 | N.D. |
| gLAG3_11 | GAACTGCTCCTTCAGCCGCCC | 469 | 0.1 |
| gLAG3_12 | AGCCGCCCTGACCGCCCAGCC | 470 | 0.1 |
| gLAG3_13 | CGCTAAGTGGTGATGGGGGGA | 197 | 2.3 |
| gLAG3_14 | CCGCTAAGTGGTGATGGGGGG | 471 | 0.3 |
| gLAG3_15 | GCGGAAAGCTTCCTCTTCCTG | 472 | 1.0 |
| gLAG3_16 | GGGCAGGAAGAGGAAGCTTTC | 191 | 6.4 |
| gLAG3_17 | CTCTTCCTGCCCCAAGTCAGC | 473 | 1.3 |
| gLAG3_18 | AACGTCTCCATCATGTATAAC | 474 | 1.1 |
| gLAG3_19 | CTTTTCTCTTCAGGTCTGGAG | 475 | 0.2 |
| gLAG3_20 | CTCTTCAGGTCTGGAGCCCCC | 476 | 0.2 |
| gLAG3_21 | ACAGTGTACGCTGGAGCAGGT | 477 | 0.1 |
| gLAG3_22 | GCAGTGAGGAAAGACCGGGTC | 198 | 2.1 |
| gLAG3_23 | CTCACTGCCAAGTGGACTCCT | 478 | 0.4 |
| gLAG3_24 | ACCCTTCGACTAGAGGATGTG | 479 | 0.8 |
| gLAG3_25 | CCCTTCGACTAGAGGATGTGA | 196 | 2.7 |
| gLAG3_26 | GACTAGAGGATGTGAGCCAGG | 480 | 1.0 |
| gLAG3_27 | CCACCTGAGGCTGACCTGTGA | 193 | 3.4 |
| gLAG3_28 | CCCACCTGAGGCTGACCTGTG | 481 | 0.8 |
| gLAG3_29 | TACTCTTTTCAGTGACTCCCA | 482 | 0.3 |
| gLAG3_30 | CAGTGACTCCCAAATCCTTTG | 483 | 0.1 |
| gLAG3_31 | CCCAGGGATCCAGGTGACCCA | 194 | 3.1 |
| gLAG3_32 | GGGTCACCTGGATCCCTGGGG | 484 | 0.2 |
| gLAG3_33 | GGTCACCTGGATCCCTGGGGA | 88 | 17.1 |
| gLAG3_34 | GTGAGGTGACTCCAGTATCTG | 485 | 0.7 |
| gLAG3_35 | TGAGGTGACTCCAGTATCTGG | 188 | 9.3 |
| gLAG3_36 | GTGTGGAGCTCTCTGGACACC | 486 | 0.9 |
| gLAG3_37 | TGTGGAGCTCTCTGGACACCC | 190 | 6.9 |
| gLAG3_38 | TCAGGACCTTGGCTGGAGGCA | 87 | 17.7 |
| gLAG3_39 | GCTGGAGGCACAGGAGGCCCA | 487 | 0.3 |
| gLAG3_40 | CCCAGCCTTGGCAATGCCAGC | 488 | 0.8 |
| gLAG3_41 | CCAGCCTTOGCAATOCCAGCT | 189 | 8.3 |
| gLAG3_42 | GCAATGCCAGCTGTACCAGGG | 489 | 0.6 |
| gLAG3_43 | TTGGAGCAGCAGTGTACTTCA | 490 | 0.8 |
| gLAG3_44 | ACAGAGCTGTCTAGCCCAGGT | 491 | 0.4 |
| gLAG3_45 | CTCCATAGGTGCCCAACGCTC | 492 | 1.3 |
| gLAG3_46 | TCCATAGGTGCCCAACGCTCT | 192 | 4.0 |
| gLAG3_47 | TCATCCTTGGTGTCCTTTCTC | 493 | 0.4 |
| gLAG3_48 | GTGTCCTTTCTCTGCTCCTTT | 494 | 0.1 |
| gLAG3_49 | CTCTGCTCCTTTTGGTGACTG | 495 | 0.2 |
| gLAG3_50 | TCTGCTCCTTTTGGTGACTGG | 496 | 0.1 |
| gLAG3_51 | TGGTGACTOOAGCCTTTGGCT | 497 | 0.6 |
| gLAG3_52 | GGTOACTGGAGCCTTTGGCTT | 498 | 0.2 |
| gLAG3_53 | GGCTTTCACCTTTGGAGAAGA | 499 | 0.1 |
| gLAG3_54 | GCTTTCACCTTTGGAGAAGAC | 500 | 0.2 |
| gLAG3_55 | CTCTAAGGCAGAAAATCGTCT | 501 | 0.1 |
| gLAG3_56 | CTGCCTTAGAGCAAGGGATTC | 502 | 0.1 |
| gLAG3_57 | GAGCAAGGGATTCACCCTCCG | 503 | 0.2 |

TABLE 15
Tested crRNAs Targeting Human
PDCD1 Gene

| | | SEQ ID | % |
|---|---|---|---|
| crRNA | Spacer Sequence | NO | Indel |
| gPD_1 | AACCTGACCTGGGACAGTTTC | 504 | 0.2 |
| gPD_2 | CCTTCCGCTCACCTCCGCCTG | 89 | 46.9 |
| gPD_3 | CGCTCACCTCCGCCTGAGCAG | 505 | 1.0 |
| gPD_4 | TCCACTGCTCAGGCGGAGGTG | 506 | 0.6 |
| gPD_5 | TCCCCAGCCCTGCTCGTGGTG | 507 | 1.2 |
| gPD_6 | GGTCACCACGAGCAGGGCTGG | 508 | 0.7 |
| gPD_7 | ACCTGCAGCTTCTCCAACACA | 509 | 0.2 |
| gPD_8 | GCACGAAGCTCTCCGATGTGT | 90 | 41.7 |
| gPD_9 | TCCAACACATCGGAGAGCTTC | 510 | 0.2 |
| gPD_10 | GTGCTAAACTGGTACCGCATG | 511 | 0.2 |
| gPD_11 | TCCGTCTGGTTGCTGGGGCTC | 512 | 0.1 |
| gPD_12 | CCCGAGGACCGCAGCCAGCCC | 513 | 0.4 |
| gPD_13 | CGTGTCACACAACTGCCCAAC | 514 | 0.5 |
| gPD_14 | CACATGAGCGTGGTCAGGGCC | 515 | 0.1 |
| gPD_15 | GATCTGCGCCTTGGGGGCCAG | 516 | 0.1 |
| gPD_16 | ATCTGCGCCTTGGGGGCCAGG | 517 | 1.2 |
| gPD_17 | GGGGCCAGGGAGATGGCCCCA | 518 | 0.6 |
| gPD_18 | GTGCCCTTCCAGAGAGAAGGG | 201 | 1.7 |
| gPD_19 | TGCCCTTCCAGAGAGAAGGGC | 519 | 0.9 |
| gPD_20 | CAGAGAGAAGGGCAGAAGTGC | 199 | 2.5 |
| gPD_21 | TGCCCTTCTCTCTGGAAGGGC | 520 | 1.4 |
| gPD_22 | GAACTGGCCGGCTGGCCTGGG | 200 | 1.7 |
| gPD_23 | TCTGCAGGGACAATAGGAGCC | 60 | 57.6 |
| gPD_24 | CTCCTCAAAGAAGGAGGACCC | 521 | 0.1 |
| gPD_25 | TCCTCAAAGAAGGAGGACCCC | 522 | 0.5 |
| gPD_26 | TCTCGCCACTGGAAATCCAGC | 523 | 0.2 |
| gPD_27 | CAGTGGCGAGAGAAGACCCCG | 92 | 23.7 |
| gPD_28 | CCTAGCGGAATGGGCACCTCA | 524 | 0.1 |
| gPD_29 | CTAGCGGAATGGGCACCTCAT | 91 | 30.3 |
| gPD_30 | GCCCCTCTGACCGGCTTCCTT | 525 | 0.3 |

TABLE 16
Tested crRNAs Targeting
Human PTPN6 Gene

| | | SEQ ID | % |
|---|---|---|---|
| crRNA | Spacer Sequence | NO | Indel |
| gPTPN6_1 | ACCGAGACCTCAGTGGGCTGG | 96 | 58.2 |
| gPTPN6_2 | AGCAGGGTCTCTGCATCCAGC | 526 | 0.3 |
| gPTPN6_4 | CTGGCTCGGCCCAGTCGCAAG | 208 | 4.3 |
| gPTPN6_5 | TCCCCTCCATACAGGTCATAG | 102 | 14.8 |
| gPTPN6_6 | TATGACCTGTATGGAGGGGAG | 61 | 83.4 |
| gPTPN6_7 | CGACTCTGACAGAGCTGGTGG | 94 | 78.1 |
| gPTPN6_8 | AGGTGGATGATGGTGCCGTCG | 209 | 3.5 |
| gPTPN6_9 | CCTGACGCTGCCTTCTCTAGG | 527 | 0.8 |
| gPTPN6_10 | TCTAGGTGGTACCATGGCCAC | 212 | 2.4 |
| gPTPN6_11 | GCCTGCAGCAGCGTCTCTGCC | 528 | 0.2 |
| gPTPN6_12 | TTGTGCGTGAGAGCCTCAGCC | 100 | 29.4 |
| gPTPN6_13 | GTGCTTTCTGTGCTCAGTGAC | 529 | 0.8 |
| gPTPN6_14 | GGCTGGTCACTGAGCACAGAA | 104 | 10.4 |
| gP1PN6_15 | CTGTGCTCAGTGACCAGCCCA | 530 | 0.5 |
| gPTPN6_16 | TGTGCTCAGTGACCAGCCCAA | 98 | 37.5 |
| gPTPN6_17 | ATGTGGGTGACCCTGAGCGGG | 531 | 0.9 |
| gPTPN6_18 | CCTCGCACATGACCTTGATGT | 532 | 1.4 |
| gPTPN6_19 | GCTCCCCCCAGGGTGGACGCT | 103 | 13.5 |
| gPTPN6_20 | GAGACCTTCGACAGCCTCACG | 202 | 9.7 |
| gPTPN6_21 | GACAGCCTCACGGACCTGGTG | 533 | 0.5 |
| gPTPN6_22 | AAGAAGACGGGGATTGAGGAG | 101 | 22.3 |
| gPPPN6_23 | TTGTTCAGTTCCAACACTCGG | 534 | 0.1 |
| gPTPN6_24 | GCTGTATCCTCGGACTCCTGC | 535 | 0.4 |
| gPTPN6_25 | CCCACCCACATCTCAGAGTTT | 99 | 34.8 |
| gPTPN6_26 | CAGAAGCAGGAGGTGAAGAAC | 95 | 77.5 |
| gPTPN6_27 | CAGACGCTGGTGCAAGTTCTT | 536 | 0.3 |
| gPTPN6_28 | CACCAGCGTCTGGAAGGGCAG | 205 | 5.4 |
| gPTPN6_29 | TTCTCTGGCCGCTGCCCTTCC | 537 | 0.1 |
| gPTPN6_30 | ATGTAGTTGGCATTGATGTAG | 538 | 0.2 |
| gPTPN6_31 | CGTCCAGAACCAGCTGCTAGG | 539 | 0.3 |
| gPTPN6_32 | TGGCAGATGGCGTGGCAGGAG | 207 | 4.4 |
| gPTPN6_33 | TCCACCTCTCGGGTGGTCATG | 540 | 0.7 |
| gPTPN6_34 | CTCCACCTCTCGGGTGGTCAT | 541 | 1.2 |
| gPTPN6_35 | CCAGAACAAATGCGTCCCATA | 542 | 0.2 |
| gPTPN6_36 | CAGAACAAATGCGTCCCATAC | 543 | 0.5 |
| gPTPN6_37 | TGGGCCCTACTCTGTGACCAA | 97 | 51.3 |
| gPTPN6_38 | TATTCGGTTGTGTCATGCTCC | 544 | 0.1 |
| gPTPN6_39 | CAGGTCTCCCCGCTGGACAAT | 213 | 1.6 |
| gPTPN6_40 | GGGAGACCTGATTCGGGAGAT | 210 | 3.4 |
| gPTPN6_41 | CTGGACCAGATCAACCAGCGG | 203 | 8.4 |
| gPTPN6_42 | CTGCCGCTGGTTGATCTGGTC | 206 | 5.3 |
| gPTPN6_43 | CCTGCCGCTGGTTGATCTGGT | 545 | 0.3 |
| gPTPN6_44 | CCCAGCGCCGGCATCGGCCGC | 546 | N.D. |
| gPTPN6_45 | GTGGAGATGTTCTCCATGAGC | 547 | N.D. |
| gPTPN6_46 | ACTGCCCCCCACCCAGGCCTG | 93 | 80.3 |
| gPTPN6_47 | TACTGCGCCTCCGTCTGCACC | 548 | 0.1 |
| gPTPN6_48 | AATGAACTGGGCGATGGCCAC | 211 | 3.3 |
| gPTPN6_49 | TTCTTAGTGGTTTCAATGAAC | 549 | 0.1 |
| gPTPN6_50 | GCATGGGCATTCTTCATGGCT | 550 | N.D. |
| gPTPN6_51 | GACGAGGTGCGGGAGGCCTTG | 551 | N.D. |
| gPTPN6_52 | GAGTCTAGTGCAGGGACCGTG | 552 | 0.1 |
| gPTPN6_53 | CCCCCCTGCACCCGGCTGCAG | 204 | 7.0 |
| gPTPN6_54 | TGTCTGCAGCCGGGTGCAGGG | 553 | 0.9 |
| gPTPN6_55 | TCCTCCCTCTTGTTCTTAGTG | 554 | 0.0 |
| gPTPN6_56 | CTCCTCCCTCTTGTTCTTAGT | 555 | 0.1 |
| gPTPN6_57 | TTCACTTTCTCCTCCCTCTTG | 556 | 0.2 |

TABLE 17
Tested crRNAs Targeting
Human TIGIT Gene

| | | SEQ ID | % |
|---|---|---|---|
| crRNA | Spacer Sequence | NO | Indel |
| gTIGIT_1 | CCTGAGGCGAGGGGAGCCTGC | 557 | 0.2 |
| gTIGIT_2 | AGGCCTTACCTGAGGCGAGGG | 62 | 81.7 |
| gTIGIT_3 | GTCCTCTTCCCTAGGAATGAT | 558 | 1.3 |
| gTIGIT_4 | TATTGTGCCTGTCATCATTCC | 559 | 1.0 |
| gTIGIT_5 | TCTGCAGAAATGTTCCCCGTT | 560 | 1.1 |
| gTIGIT_6 | CTCTGCAGAAATGTTCCCCGT | 561 | 0.1 |
| gTIGIT_7 | TGCAGAGAAAGGTGGCTCTAT | 215 | 6.0 |
| gTIGIT_8 | TGCCGTGGTGGAGGAGAGGTG | 562 | 0.3 |
| gTIGIT_9 | TGGCCATTTGTAATGCTGACT | 563 | 0.8 |
| gTIGIT_10 | TAATGCTGACTTGGGGTGGCA | 216 | 1.6 |
| gTIGIT_11 | GGGTGGCACATCTCCCCATCC | 214 | 9.7 |
| gTIGIT_12 | AAGGATGGGGAGATGTGCCAC | 564 | 0.4 |
| gTIGIT_13 | AAGGATCGAGTGGCCCCAGGT | 565 | 0.2 |
| gTIGIT_14 | TGCATCTATCACACCTACCCT | 566 | 1.4 |
| gTIGIT_15 | TAGGACCTCCAGGAAGATTCT | 567 | 0.4 |
| gTIGIT_16 | CTAGGACCTCCAGGAAGATTC | 568 | 0.5 |
| gTIGIT_17 | CTCCAGCAGGAATACCTGAGC | 569 | 0.8 |
| gTIGIT_18 | GTCCTCCCTCTAGTGGCTGAG | 105 | 72.4 |
| gTIGIT_19 | GAGCCATGGCCGCGACGCTGG | 570 | 0.9 |
| gTIGIT_20 | TAGTCAACGCGACCACCACGA | 571 | 0.1 |
| gTIGIT_21 | CTAGTCAACGCGACCACCACG | 572 | 0.1 |
| gTIGIT_22 | TAGTTTGTTTGTTTTTAGAAG | 573 | 0.6 |
| gTIGIT_23 | TTTGTTTTTAGAAGAAAGCCC | 574 | 1.0 |
| gTIGIT_24 | TTTTTAGAAGAAAGCCCTCAG | 575 | 0.4 |
| gTIGIT_25 | TAGAAGAAAGCCCTCAGAATC | 576 | 1.2 |
| gTIGIT_26 | CACAGAATGGATTCTGAGGGC | 577 | 0.3 |
| gTIGIT_27 | CTCCTGAGGTCACCTTCCACA | 217 | 1.6 |
| gTIGIT_28 | CTGGGGGTGAGGGAGCACTGG | 578 | 0.5 |
| gTIGIT_29 | TGCCTGGACACAGCTTCCTGG | 579 | 0.3 |
| gTIGIT_30 | TGTAACTCAGGACATTGAAGT | 580 | 0.5 |
| gTIGIT_31 | AATGTCCTGAGTTACAGAAGC | 581 | 0.5 |

TABLE 18
Tested crRNAs Targeting
Human TRAC Gene

| | | SEQ ID | % |
|---|---|---|---|
| crRNA | Spacer Sequence | NO | Indel |
| gTRAC00l | TGTTTTTAATGTGACTCTCAT | 237 | 1.8 |
| gTRAC002 | GTGTTTTTAATGTGACTCTCA | 582 | 0.4 |
| gTRAC003 | CGTAGGATTTTGTGTTTTTAA | 583 | 0.1 |
| gTRAC004 | CTTAGTGCTGAGACTCATTCT | 584 | 0.7 |
| gTRAC005 | CCTTAGTGCTGAGACTCATTC | 585 | 0.6 |
| gTRAC006 | TGAGGGTGAAGGATAGACGCT | 63 | 81.8 |
| gTRAC007 | ATAAACTGTAAAGTACCAAAC | 239 | 1.7 |
| gTRAC008 | TTTGGTACTTTACAGTTTATT | 586 | 0.2 |
| gTRAC009 | GTACTTTACAGTTTATTAAAT | 238 | 1.7 |
| gTRAC010 | CAGTTTATTAAATAGATGTTT | 587 | 0.5 |
| gTRAC011 | TTAAATAGATGTTTATATGGA | 588 | 0.0 |
| gTRAC012 | TATGGAGAAGCTCTCATTTCT | 110 | 46.7 |
| gTRAC013 | TTTCTCAGAAGAGCCTGGCTA | 225 | 5.8 |
| gTRAC014 | TCAGAAGAGCCTOGCTAGGAA | 127 | 16.6 |
| gTRAC015 | ACCTGCAAAATGAATATGGTG | 589 | 0.0 |
| gTRAC016 | GCAGGTGAAATTCCTGAGATG | 590 | 0.2 |
| gTRAC017 | CAGGTOAAATTCCTGAGATGT | 107 | 63.6 |
| gTRAC018 | CTCGATATAAGGCCTTGAGCA | 120 | 26.0 |
| gTRAC019 | AACTATAAATCAGAACACCTG | 228 | 4.5 |
| gTRAC020 | GAACTATAAATCAGAACACCT | 224 | 6.4 |
| gTRAC021 | TAGTTCAAAACCTCTATCAAT | 117 | 27.7 |
| gTRAC022 | TGGTATGTTGGCATTAAGTTG | 591 | 1.0 |
| gTRAC023 | CCAACTTAATGCCAACATACC | 592 | 1.4 |
| gTRAC024 | CTTTGCTGGGCCTTTTTCCCA | 593 | 1.0 |
| gTRAC025 | CTGGGCCTTTTTCCCATGCCT | 227 | 4.6 |
| gTRAC026 | TCCCATGCCTGCCTTTACTCT | 594 | 0.6 |
| gTRAC027 | CCCATGCCTGCCTTTACTCTG | 595 | 0.7 |
| gTRAC028 | CCATGCCTGCCTTTACTCTGC | 129 | 15.3 |
| gTRAC029 | CTCTGCCAGAGTTATATTGCT | 128 | 15.8 |
| gTRAC030 | ATAGGATCTTCTTCAAAACCC | 235 | 2.2 |
| gTRAC031 | TTTAATAGGATCTTCTTCAAA | 596 | 0.3 |
| gTRAC032 | ATTTAATAGGATCTTCTTCAA | 597 | 0.1 |
| gTRAC033 | GAAGAAGATCCTATTAAATAA | 236 | 2.0 |
| gTRAC034 | AAGAAGATCCTATTAAATAAA | 598 | 0.1 |
| gTRAC035 | AGGTTTCCTTGAGTGGCAGGC | 220 | 75 |
| gTRAC036 | CTTGAGTGGCAGGCCAGGCCT | 230 | 4.4 |
| gTRAC037 | AGTGAACGTTCACGGCCAGGC | 599 | 0.7 |
| gTRAC038 | TACOGGAAATAGCATCTTAGA | 114 | 40.7 |
| gTRAC039 | TAAGATGCTATTTCCCGTATA | 111 | 45.8 |
| gTRAC040 | CCGTATAAAGCATGAGACCGT | 124 | 21.5 |
| gTRAC041 | CCCCAACCCAGGCTGGAGTCC | 125 | 18.7 |
| gTRAC042 | CCTCTTTGCCCCAACCCAGGC | 219 | 7.6 |
| gTRAC043 | GAGTCTCTCAGCTGGTACACG | 121 | 25.9 |
| gTRAC044 | AGAATCAAAATCGGTGAATAG | 221 | 7.4 |
| gTRAC045 | TTTGAGAATCAAAATCGGTGA | 600 | 1.3 |
| gTRAC046 | TGACACATTTGTTTGAGAATC | 601 | 0.2 |
| gTRAC047 | GATTCTCAAACAAATGTGTCA | 602 | 0.1 |
| gTRAC048 | ATTCTCAAACAAATGTGTCAC | 229 | 4.5 |
| gTRAC049 | TCTGTGATATACACATCAGAA | 118 | 27.6 |
| gTRAC050 | GTCTGTGATATACACATCAGA | 130 | 11.4 |
| gTRAC055 | CACATGCAAAGTCAGATTTGT | 603 | 1.0 |
| gTRAC056 | CATGTGCAAACGCCTTCAACA | 231 | 3.9 |
| gTRAC057 | GTGCCTTCGCAGGCTGTTTCC | 604 | 0.9 |
| gTRAC058 | CTTGCTTCAGGAATGGCCAGG | 116 | 27.8 |
| gTRAC059 | GACATCATTGACCAGAGCTCT | 108 | 50.1 |
| gTRAC060 | AGACATCATTGACCAGAGCTC | 605 | 1.3 |
| gTRAC061 | GTGGCAATGGATAAGGCCGAG | 115 | 38.8 |
| gTRAC062 | GGTGGCAATGGATAAGGCCGA | 223 | 6.5 |
| gTRAC063 | TTAGTAAAAAGAGGGTTTTGG | 606 | 1.4 |
| gTRAC064 | TACTAAGAAACAGTGAGCCTT | 232 | 5 3 |
| gTRAC065 | ACTAAGAAACAGTGAGCCTTG | 607 | 0.2 |
| gTRAC066 | CTAAGAAACAGTGAGCCTTGT | 218 | 9.5 |
| gTRAC067 | CCGTGTCATTCTCTGGACTGC | 112 | 45.4 |
| gTRAC068 | CCCGTGTCATTCTCTGGACTG | 226 | 5.3 |
| gTRAC069 | TCCCGTGTCATTCTCTGGACT | 608 | 1.0 |
| gTRAC070 | TTCCCGTGTCATTCTCTGGAC | 609 | 0.3 |
| gTRAC071 | CTCAGACTGTTTGCCCCTTAC | 233 | 3.4 |
| gTRAC072 | CCCCTTACTGCTCTTCTAGGC | 222 | 6.9 |
| gTRAC073 | GCAGACAGGGAGAAATAAGGA | 106 | 66.9 |
| gTRAC074 | GGCAGACAGGGAGAAATAAGG | 119 | 27.1 |
| gTRAC075 | TGGCAGACAGGGAGAAATAAG | 122 | 25.2 |
| gTRAC076 | TTGGCAGACAGGGAGAAATAA | 126 | 16.7 |
| gTRAC077 | TCCCTGTCTGCCAAAAAATCT | 610 | 1.1 |
| gTRAC078 | CCAGCTCACTAAGTCAGTCTC | 109 | 47.4 |
| gTRAC079 | ATTCCTCCACTTCAACACCTG | 113 | 45.4 |
| gTRAC080 | AATTCCTCCACTTCAACACCT | 611 | 0.5 |
| gTRAC081 | TAATTCCTCCACTTCAACACC | 234 | 2.3 |
| gTRAC082 | CCAGCTGACAGATGGGCTCCC | 123 | 21.5 |
| gTRAC083 | CCCAGCTGACAGATGGGCTCC | 241 | 1.6 |
| gTRAC084 | GACTTTTCCCAGCTGACAGAT | 240 | 1.6 |
| gTRAC085 | TCAACCCTGAGTTAAAACACA | 612 | 0.5 |
| gTRAC086 | CTCAACCCTGAGTTAAAACAC | 613 | 0.2 |
| gTRAC087 | TCCTGAAGGTAGCTGTTTTCT | 614 | 0.2 |
| gTRAC088 | GTCCTGAAGGTAGCTGTTTTC | 615 | 0.1 |
| gTRAC089 | AACTCAGGGTTGAGAAAACAG | 616 | 0.7 |
| gTRAC090 | ACTCAGGGTTGAGAAAACAGC | 617 | 0.1 |

TABLE 19
Tested crRNAs Targeting Human
TRBC1/TRBC2 Genes

| | | SEQ ID | % |
|---|---|---|---|
| crRNA | Spacer Sequence | NO | Indel |
| gTRBC1+2_1 | AGCCATCAGAAGCAGAGATCT | 705 | 66.40 |
| | | | (TRBC1); |
| | | | 74.7 |
| | | | (TRBC2) |
| gTRBC1+2_3 | COCTGTCAAGTCCAGTTCTAC | 706 | 71.28 |
| | | | (TRBC1) |
| gTRBC2_7 | CCCTGTTTTCTTTCAGACTGT | 707 | 0.09 |
| gTRBC2_8 | CTTTCAGACTGTGGCTTCACC | 708 | 0.24 |
| gTRBC2_9 | TTTCAGACTGTGGCTTCACCT | 709 | 0.24 |
| gTRBC2_10 | CAGACTGTGGCTTCACCTCCG | 710 | 0.16 |
| gTRBC2_11 | AGACTGTGGCTTCACCTCCGG | 711 | 19.97 |
| gTRBC2_12 | CCGGAGGTGAAGCCACAGTCT | 712 | 33.14 |
| gTRBC2_13 | TCAACAGAGTCTTACCAGCAA | 713 | 1.20 |
| gTRBC2_14 | CCAGCAAGGGGTCCTGTCTGC | 714 | 6.69 |
| gTRBC2_15 | CTAGGGAAGGCCACCTTGTAT | 715 | 21.74 |
| gTRBC2_16 | TATGCCGTGCTGGTCAGTOCC | 716 | 0.20 |
| gTRBC2_17 | CCATGGCCATCAGCACGAGGG | 717 | 1.75 |
| gTRBC2_18 | CCTAGCAAGATCTCATAGAGG | 718 | 0.37 |
| gTRBC2_19 | CACAGGTCAAGAGAAAGGATT | 719 | 1.58 |
| gTRBC2_21 | GAGCTAGCCTCTGGAATCCTT | 720 | 11.89 |

TABLE 20
Tested crRNAs Targeting
Human CARD11 Gene

| | | SEQ ID | % |
|---|---|---|---|
| crRNA | Spacer Sequence | NO | Indel |
| gCARD11_1 | TAGTACCGCTCCTGGAAGGTT | 721 | 1.37 |
| gCARD11_2 | ATCTTGTAGTACCGCTCCTGG | 722 | 0.07 |
| gCARD11_3 | CTTCATCTTGTAGTACCGCTC | 723 | 0.08 |

TABLE 21
Tested crRNAs Targeting
Human CD247 gene

| | | SEQ ID | % |
|---|---|---|---|
| crRNA | Spacer Sequence | NO | Indel |
| gCD247_1 | TGTGTTOCAGTTCAGCAGGAG | 724 | 55.77 |
| gCD247_2 | CGTTATAGAGCTOGTTCTGGC | 725 | 0.20 |
| gCD247_3 | CGGAGGGTCTACGGCGAGGCT | 726 | 20.79 |
| gCD247_4 | TTATCTGTTATAGGAGCTCAA | 727 | 12.31 |
| gCD247_5 | TCTGTTATAGGAGCTCAATCT | 728 | 0.24 |
| gCD247 6 | TCCAAAACATCGTACTCCTCT | 729 | 0.34 |
| gCD247_7 | CCCCCATCTCAGGGTCCCGGC | 730 | 6.43 |
| gCD247_8 | GACAAGAGACGTGGCCGGGAC | 731 | 40.95 |
| gCD247_9 | TCTCCCTCTAACGTCTTCCCG | 732 | 4.13 |
| gCD247_10 | CTGAGGGTTCTTCCTTCTCTG | 733 | 0.05 |
| gCD247_11 | CCGTTGTCTTTCCTAGCAGAG | 734 | 1.18 |
| gCD247_12 | CTAGCAGAGAAGGAAGAACCC | 735 | 70.64 |
| gCD247_13 | TGCAGTTCCTGCAGAAGAGGG | 736 | 4.93 |
| gCD247_14 | TGCAGGAACTGCAGAAAGATA | 737 | 2.91 |
| gCD247_15 | ATCCCAATCTCACTGTAGGCC | 738 | 31.12 |
| gCD247_16 | CATCCCAATCTCACTGTAGGC | 739 | 0.10 |
| gCD247_17 | CTCATTTCACTCCCAAACAAC | 740 | 0.30 |
| gCD247_18 | TCATTTCACTCCCAAACAACC | 741 | 44.34 |
| gCD247_19 | ACTCCCAAACAACCAGCGCCG | 742 | 43.17 |
| gCD247_20 | TTTTCTGATTTGCTTTCACGC | 743 | 0.10 |
| gCD247_21 | TGATTTGCTTTCACGCCAGGG | 744 | 5.23 |
| gCD247_22 | CTTTCACGCCAGGGTCTCAGT | 745 | 8.24 |
| gCD247_23 | ACGCCAGGGTCTCAGTACAGC | 746 | 0.30 |

TABLE 22
Tested crRNAs Targeting
Human IL7R Gene

| | | SEQ ID | % |
|---|---|---|---|
| crRNA | Spacer Sequence | NO | Indel |
| gIL7R_1 | CTTTCCAGGGGAGATGGATCC | 747 | 0.25 |
| gIL7R_2 | CCAGGGGAGATGGATCCTATC | 748 | 8.35 |
| gll7R_3 | CAGGGGAGATGGATCCTATCT | 749 | 87.87 |
| gIL7R_4 | CTAACCATCAGCATTTTGAGT | 750 | 0.11 |
| gIL7R_5 | GAGTTTTTTCTCTGTCGCTCT | 751 | 0.07 |
| gIL7R_6 | AGTTTTTTCTCTGTCGCTCTG | 752 | 0.06 |
| gIL7R_7 | TCTGTCGCTCTGTTGGTCATC | 753 | 2.61 |
| gIL7R_8 | CATAACACACAGGCCAAGATG | 754 | 25.83 |

TABLE 23
Tested crRNAs Targeting
Human LCK Gene

| | | SEQ ID | % |
|---|---|---|---|
| crRNA | Spacer Sequence | NO | Indel |
| gLCK1_1 | ATGTCCTTTCACCCATCAACC | 755 | 0.06 |
| gLCK1_2 | CACCCATCAACCCGTAGGGAT | 756 | 0.17 |
| gLCK1_3 | ACCCATCAACCCGTAGGGATG | 757 | 16.21 |

TABLE 24
Tested crRNAs Targeting
Human PLCG1 Gene

| | | SEQ ID | % |
|---|---|---|---|
| crRNA | Spacer Sequence | NO | Indel |
| gPLCG1_1 | CTCATACACCACGAAGCGCAG | 758 | 0.09 |
| gPLCG1_2 | CCTTTCTGCGCTTCGTGGTGT | 759 | 5.14 |
| gPLCG1_3 | CTGCGCTTCGTGGTGTATGAG | 760 | 0.05 |
| gPLCG1_4 | TGCGCTTCGTGGTGTATGAGG | 761 | 1.91 |
| gPLCG1_5 | GTGGTGTATGAGGAAGACATG | 762 | 3.53 |

TABLE 25
Tested crRNAs Targeting
Certain Other Human Genes

| | | SEQ ID | % |
|---|---|---|---|
| crRNA | Spacer Sequence | NO | Indel |
| gDHODH_1 | TTGCAGAAGCGGGCCCAGGAT | 770 | 0.60 |
| gDHODH_2 | TTGCAGAAGCGGGCCCAGGAT | 771 | 0.59 |
| gDHODH_3 | TATGCTGAACACCTGATGCCG | 772 | 74.94 |
| gPLK1_1 | CCAGGGTCGGCCGGTGCCCGT | 773 | 29.06 |
| gPLK1_2 | GCCGGTGGAGCCGCCGCCGGA | 774 | 2.01 |
| gPLK1_3 | TGGGCAAGGGCGGCTTTGCCA | 775 | 2.26 |
| gPLK1_4 | GGGCAAGGGCGGCTTTGCCAA | 776 | 28.24 |
| gPIK1_5 | GGCAAGGGCGGCTTTGCCAAG | 777 | 28.41 |
| gPLK1_6 | CCAAGTGCTTCGAGATCTCGG | 778 | 2.07 |
| gPLK1_7 | CATGGACATCTTCTCCCTCTG | 779 | 90.07 |
| gPLK1_8 | TCGAGGACAACGACTTCGTGT | 780 | 0.16 |
| gPLK1_9 | CGAGGACAACGACTTCGTGTT | 781 | 6.84 |
| gPLK1_10 | GAGGACAACGACTTCGTGTTC | 782 | 8.52 |
| gMVD_1 | CAGTTAAAAACCACCACAACA | 783 | 1.42 |
| gMVD_2 | GCPGAATGGCCGGGAGGAGGA | 784 | 14.06 |
| gMVD_3 | TGGAGTGGCAGATGGGAGAGC | 785 | 63.22 |
| gTUBB_1 | AACCATGAGGGAAATCGTGCA | 786 | 2.61 |
| gTUBB_2 | ACCATGAGGGAAATCGTGCAC | 787 | 68.40 |
| gTUBB_3 | TTCTCTGTAGGTGGCAAATAT | 788 | 18.67 |
| gU6_1 | GTCCTTTCCACAAGATATATA | 763 | 68.1 |
| gU6_2 | GATTTCTTGGCTTTATATATC | 764 | 0.71 |
| gU6_3 | TTGGCTTTATATATCTTGTGG | 765 | 2.83 |
| gU6_4 | GCTTTATATATCTTGTGGAAA | 766 | 0.37 |
| gU6_5 | ATATATCTTGTGGAAAGGACG | 767 | 0.39 |
| gU6_6 | TATATCTTGTGGAAAGGACGA | 768 | 0.39 |
| gU6_7 | TGGAAAGGACGAAACACCGTG | 769 | 0.24 |

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A guide nucleic acid comprising a targeter stem sequence and a spacer sequence, wherein the spacer sequence comprises a nucleotide sequence listed in Table 1, 2, or 3.

2. The guide nucleic acid of claim 1, wherein the targeter stem sequence comprises a nucleotide sequence of GUAGA.

3. The guide nucleic acid of claim 1 or 2, wherein the targeter stem sequence is 5′ to the spacer sequence, optionally wherein the targeter stem sequence is linked to the spacer sequence by a linker consisting of 1, 2, 3, 4, or 5 nucleotides.

4. The guide nucleic acid of any one of claims 1-3, wherein the guide nucleic acid is capable of activating a CRISPR Associated (Cas) nuclease in the absence of a tracrRNA.

5. The guide nucleic acid of claim 4, wherein the guide nucleic acid comprises from 5′ to 3′ a modulator stem sequence, a loop sequence, a targeter stem sequence, and the spacer sequence.

6. The guide nucleic acid of any one of claims 1-3, wherein the guide nucleic acid is a targeter nucleic acid that, in combination with a modulator nucleic acid, is capable of activating a Cas nuclease.

7. The guide nucleic acid of claim 6, wherein the guide nucleic acid comprises from 5′ to 3′ a targeter stem sequence and the spacer sequence.

8. The guide nucleic acid of any one of claims 4-7, wherein the Cas nuclease is a type V Cas nuclease.

9. The guide nucleic acid of claim 8, wherein the Cas nuclease is a type V-A Cas nuclease.

10. The guide nucleic acid of claim 9, wherein the Cas nuclease comprises an amino acid sequence at least 80% identical to SEQ ID NO: 1.

11. The guide nucleic acid of claim 9, wherein the Cas nuclease is Cpf1.

12. The guide nucleic acid of any one of claims 4-11, wherein the Cas nuclease recognizes a protospacer adjacent motif (PAM) consisting of the nucleotide sequence of TITN or CTIN.

13. The guide nucleic acid of any one of the proceeding claims, wherein the guide nucleic acid comprises a ribonucleic acid (RNA).

14. The guide nucleic acid of claim 13, wherein the guide nucleic acid comprises a modified RNA.

15. The guide nucleic acid of claim 13 or 14, wherein the guide nucleic acid comprises a combination of RNA and DNA.

16. The guide nucleic acid of any one of claims 13-15, wherein the guide nucleic acid comprises a chemical modification.

17. The guide nucleic acid of claim 16, wherein the chemical modification is present in one or more nucleotides at the 5′ end of the guide nucleic acid.

18. The guide nucleic acid of claim 16 or 17, wherein the chemical modification is present in one or more nucleotides at the 3′ end of the guide nucleic acid.

19. The guide nucleic acid of any one of claims 16-18, wherein the chemical modification is selected from the group consisting of 2′-O-methyl, 2′-fluoro, 2′-O-methoxyethyl, phosphorothioate, phosphorodithioate, pseudouridine, and any combinations thereof.

20. An engineered, non-naturally occurring system comprising the guide nucleic acid of any one of claims 4-5 and 8-19.

21. The engineered, non-naturally occurring system of claim 20, further comprising the Cas nuclease.

22. The engineered, non-naturally occurring system of claim 21, wherein the guide nucleic acid and the Cas nuclease are present in a ribonucleoprotein (RNP) complex.

23. An engineered, non-naturally occurring system comprising the guide nucleic acid of any one of claims 6-19, further comprising the modulator nucleic acid.

24. The engineered, non-naturally occurring system of claim 23, further comprising the Cas nuclease.

25. The engineered, non-naturally occurring system of claim 24, wherein the guide nucleic acid, the modulator nucleic acid, and the Cas nuclease are present in an RNP complex.

26. The engineered, non-naturally occurring system of any one of claims 1-25, wherein the spacer sequence comprises a nucleotide sequence selected from the group consisting of SEQ ID NOs: 63, 106-130, and 218-241, and wherein the spacer sequence is capable of hybridizing with the human TRAC gene.

27. The engineered, non-naturally occurring system of claim 26, wherein, when the system is delivered into a population of human cells ex vivo, the genomic sequence at the TRAC gene locus is edited in at least 1.5% of the cells.

28. The engineered, non-naturally occurring system of any one of claims 1-25, wherein the spacer sequence comprises a nucleotide sequence selected from the group consisting of SEQ ID NOs: 51 and 131-137, and wherein the spacer sequence is capable of hybridizing with the human ADORA2A gene.

29. The engineered, non-naturally occurring system of claim 28, wherein, when the system is delivered into a population of human cells ex vivo, the genomic sequence at the ADORA2A gene locus is edited in at least 1.5% of the cells.

30. The engineered, non-naturally occurring system of any one of claims 1-25, wherein the spacer sequence comprises a nucleotide sequence selected from the group consisting of SEQ ID NOs: 52, 64-66, 138-145, 622, 625-626, and 634-635, and wherein the spacer sequence is capable of hybridizing with the human B2M gene.

31. The engineered, non-naturally occurring system of claim 30, wherein, when the system is delivered into a population of human cells ex vivo, the genomic sequence at the B2M gene locus is edited in at least 1.5% of the cells.

32. The engineered, non-naturally occurring system of any one of claims 1-25, wherein the spacer sequence comprises a nucleotide sequence selected from the group consisting of SEQ ID NOs: 724, 726-727, 730-732, 735-738, 741-742, and 744-745, and wherein the spacer sequence is capable of hybridizing with the human CD247 gene.

33. The engineered, non-naturally occurring system of claim 32, wherein, when the system is delivered into a population of human cells ex vivo, the genomic sequence at the CD247 gene locus is edited in at least 1.5% of the cells.

34. The engineered, non-naturally occurring system of any one of claims 1-25, wherein the spacer sequence comprises a nucleotide sequence selected from the group consisting of SEQ ID NOs: 53 and 146, and wherein the spacer sequence is capable of hybridizing with the human CD52 gene.

35. The engineered, non-naturally occurring system of claim 34, wherein, when the system is delivered into a population of human cells ex vivo, the genomic sequence at the CD52 gene locus is edited in at least 1.5% of the cells.

36. The engineered, non-naturally occurring system of any one of claims 1-25, wherein the spacer sequence comprises a nucleotide sequence selected from the group consisting of SEQ ID NOs: 54, 147-148, 636-640, 642, 644-648, 650-652, 655-656, 660-663, 666, 668, 670-671, 673-676, 678-679, and 682-685 and wherein the spacer sequence is capable of hybridizing with the human CIITA gene.

37. The engineered, non-naturally occurring system of claim 36, wherein, when the system is delivered into a population of human cells ex vivo, the genomic sequence at the CIITA gene locus is edited in at least 1.5% of the cells.

38. The engineered, non-naturally occurring system of any one of claims 1-25, wherein the spacer sequence comprises a nucleotide sequence selected from the group consisting of SEQ ID NOs: 55, 67-70, and 149-155, and wherein the spacer sequence is capable of hybridizing with the human CTLA4 gene.

39. The engineered, non-naturally occurring system of claim 38, wherein, when the system is delivered into a population of human cells ex vivo, the genomic sequence at the CTLA4 gene locus is edited in at least 1.5% of the cells.

40. The engineered, non-naturally occurring system of any one of claims 1-25, wherein the spacer sequence comprises a nucleotide sequence selected from the group consisting of SEQ ID NOs: 56, 71-74, and 156-159, and wherein the spacer sequence is capable of hybridizing with the human DCK gene.

41. The engineered, non-naturally occurring system of claim 40, wherein, when the system is delivered into a population of human cells ex vivo, the genomic sequence at the DCK gene locus is edited in at least 1.5% of the cells.

42. The engineered, non-naturally occurring system of any one of claims 1-25, wherein the spacer sequence comprises a nucleotide sequence selected from the group consisting of SEQ ID NOs: 57, 75-79, and 160-173, and wherein the spacer sequence is capable of hybridizing with the human FAS gene.

43. The engineered, non-naturally occurring system of claim 42, wherein, when the system is delivered into a population of human cells ex vivo, the genomic sequence at the FAS gene locus is edited in at least 1.5% of the cells.

44. The engineered, non-naturally occurring system of any one of claims 1-25, wherein the spacer sequence comprises a nucleotide sequence selected from the group consisting of SEQ ID NOs: 58, 80-86, and 174-187, and wherein the spacer sequence is capable of hybridizing with the human HAVCR2 gene.

45. The engineered, non-naturally occurring system of claim 44, wherein, when the system is delivered into a population of human cells ex vivo, the genomic sequence at the HAVCR2 gene locus is edited in at least 1.5% of the cells.

46. The engineered, non-naturally occurring system of any one of claims 1-25, wherein the spacer sequence comprises a nucleotide sequence selected from the group consisting of SEQ ID NOs: 748-749 and 753-754, and wherein the spacer sequence is capable of hybridizing with the human IL7R gene.

47. The engineered, non-naturally occurring system of claim 46, wherein, when the system is delivered into a population of human cells ex vivo, the genomic sequence at the IL7R gene locus is edited in at least 1.5% of the cells.

48. The engineered, non-naturally occurring system of any one of claims 1-25, wherein the spacer sequence comprises a nucleotide sequence selected from the group consisting of SEQ ID NOs: 59, 87, 88, and 188-198, and wherein the spacer sequence is capable of hybridizing with the human LAG3 gene.

49. The engineered, non-naturally occurring system of claim 48, wherein, when the system is delivered into a population of human cells ex vivo, the genomic sequence at the LAG3 gene locus is edited in at least 1.5% of the cells.

50. The engineered, non-naturally occurring system of any one of claims 1-25, wherein the spacer sequence comprises the nucleotide sequence of SEQ ID NO: 757, and wherein the spacer sequence is capable of hybridizing with the human LCK gene.

51. The engineered, non-naturally occurring system of claim 50, wherein, when the system is delivered into a population of human cells ex vivo, the genomic sequence at the LCK gene locus is edited in at least 1.5% of the cells.

52. The engineered, non-naturally occurring system of any one of claims 1-25, wherein the spacer sequence comprises a nucleotide sequence selected from the group consisting of SEQ ID NOs: 60, 89-92, and 199-201, and wherein the spacer sequence is capable of hybridizing with the human PDCD1 gene.

53. The engineered, non-naturally occurring system of claim 52, wherein, when the system is delivered into a population of human cells ex vivo, the genomic sequence at the PDCD1 gene locus is edited in at least 1.5% of the cells.

54. The engineered, non-naturally occurring system of any one of claims 1-25, wherein the spacer sequence comprises a nucleotide sequence selected from the group consisting of of SEQ ID NOs: 759 and 761-762, and wherein the spacer sequence is capable of hybridizing with the human PLCG1 gene.

55. The engineered, non-naturally occurring system of claim 54, wherein, when the system is delivered into a population of human cells ex vivo, the genomic sequence at the PLCG1 gene locus is edited in at least 1.5% of the cells.

56. The engineered, non-naturally occurring system of any one of claims 1-25, wherein the spacer sequence comprises a nucleotide sequence selected from the group consisting of SEQ ID NOs: 61, 93-104, and 202-213, and wherein the spacer sequence is capable of hybridizing with the human PTPN6 gene.

57. The engineered, non-naturally occurring system of claim 56, wherein, when the system is delivered into a population of human cells ex vivo, the genomic sequence at the PTPN6 gene locus is edited in at least 1.5% of the cells.

58. The engineered, non-naturally occurring system of any one of claims 1-25, wherein the spacer sequence comprises a nucleotide sequence selected from the group consisting of SEQ ID NOs: 62, 105, and 214-217, and wherein the spacer sequence is capable of hybridizing with the human TIGIT gene.

59. The engineered, non-naturally occurring system of claim 58, wherein, when the system is delivered into a population of human cells ex vivo, the genomic sequence at the TIGIT gene locus is edited in at least 1.5% of the cells.

60. The engineered, non-naturally occurring system of any one of claims 1-25, wherein the spacer sequence comprises a nucleotide sequence selected from the group consisting of SEQ ID NOs: 705-706, 711-712, 714-715, 717, and 719-720, and wherein the spacer sequence is capable of hybridizing with the human TRBC2 gene.

61. The engineered, non-naturally occurring system of claim 60, wherein, when the system is delivered into a population of human cells ex vivo, the genomic sequence at the TRBC2 gene locus is edited in at least 1.5% of the cells.

62. The engineered, non-naturally occurring system of any one of claims 1-25, wherein the spacer sequence comprises a nucleotide sequence selected from the group consisting of SEQ ID NOs: 705-706, and wherein the spacer sequence is capable of hybridizing with both the human TRBC1 gene and the human TRBC2 gene.

63. The engineered, non-naturally occurring system of claim 62, wherein, when the system is delivered into a population of human cells ex vivo, the genomic sequence at the TRBC1 gene locus is edited in at least 1.5% of the cells.

64. The engineered, non-naturally occurring system of any one of claims 20-63, wherein genomic mutations are detected in no more than 2% of the cells at any off-target loci by CIRCLE-Seq.

65. The engineered, non-naturally occurring system of claim 64, wherein genomic mutations are detected in no more than 1% of the cells at any off-target loci by CIRCLE-Seq.

66. A human cell comprising the engineered, non-naturally occurring system of any one of claims 20-65.

67. A composition comprising the guide nucleic acid of any one of claims 1-19, the engineered, non-naturally occurring system of any one of claims 20-65, or the human cell of claim 66.

68. A method of cleaving a target DNA comprising the sequence of a preselected target gene or a portion thereof, the method comprising contacting the target DNA with the engineered, non-naturally occurring system of any one of claims 20-65, thereby resulting in cleavage of the target DNA.

69. The method of claim 68, wherein the contacting occurs in vitro.

70. The method of claim 68, wherein the contacting occurs in a cell ex vivo.

71. The method of claim 70, wherein the target DNA is genomic DNA of the cell.

72. A method of editing human genomic sequence at a preselected target gene locus, the method comprising delivering the engineered, non-naturally occurring system of any one of claims 20-65 into a human cell, thereby resulting in editing of the genomic sequence at the target gene locus in the human cell.

73. The method of any one of claims 70-72, wherein the cell is an immune cell.

74. The method of claim 73, wherein the immune cell is a T lymphocyte.

75. The method of claim 72, the method comprising delivering the engineered, non-naturally occurring system of any one of claims 20-65 into a population of human cells, thereby resulting in editing of the genomic sequence at the target gene locus in at least a portion of the human cells.

76. The method of claim 75, wherein the population of human cells comprises human immune cells.

77. The method of claim 75 or 76, wherein the population of human cells is an isolated population of human immune cells.

78. The method of claim 76 or 77, wherein the immune cells are T lymphocytes.

79. The method of any one of claims 72-78, wherein the engineered, non-naturally occurring system is delivered into the cell(s) as a pre-formed RNP complex.

80. The method of claim 79, wherein the pre-formed RNP complex is delivered into the cell(s) by electroporation.

81. The method of any one of claims 72-80, wherein the target gene is human TRAC gene, and wherein the spacer sequence comprises a nucleotide sequence selected from the group consisting of SEQ ID NOs: 63, 106-130, and 218-241.

82. The method of any one of claims 75-81, wherein the genomic sequence at the TRAC gene locus is edited in at least 1.5% of the human cells.

83. The method of any one of claims 72-80, wherein the target gene is human ADORA2A gene, and wherein the spacer sequence comprises a nucleotide sequence selected from the group consisting of SEQ ID NOs: 51 and 131-137.

84. The method of any one of claims 75-80 and 83, wherein the genomic sequence at the ADORA2A gene locus is edited in at least 1.5% of the human cells.

85. The method of any one of claims 72-80, wherein the target gene is human B2M gene, and wherein the spacer sequence comprises a nucleotide sequence selected from the group consisting of SEQ ID NOs: 52, 64-66, 138-145, 622, 625-626, and 634-635.

86. The method of any one of claims 75-80 and 85, wherein the genomic sequence at the B2M gene locus is edited in at least 1.5% of the human cells.

87. The method of any one of claims 72-80, wherein the target gene is human CD52 gene, and wherein the spacer sequence comprises a nucleotide sequence selected from the group consisting of SEQ ID NOs: 53 and 146.

88. The method of any one of claims 75-80 and 87, wherein the genomic sequence at the CD52 gene locus is edited in at least 1.5% of the human cells.

89. The method of any one of claims 72-80, wherein the target gene is human CD247 gene, and wherein the spacer sequence comprises a nucleotide sequence selected from the group consisting of SEQ ID NOs: 724, 726-727, 730-732, 735-738, 741-742, and 744-745.

90. The method of any one of claims 75-80 and 89, wherein the genomic sequence at the CD247 gene locus is edited in at least 1.5% of the human cells.

91. The method of any one of claims 72-80, wherein the target gene is human CIITA gene, and wherein the spacer sequence comprises a nucleotide sequence selected from the group consisting of SEQ ID NOs: 54, 147-148, 636-640, 642, 644-648, 650-652, 655-656, 660-663, 666, 668, 670-671, 673-676, 678-679, and 682-685.

92. The method of any one of claims 75-80 and 91, wherein the genomic sequence at the CIITA gene locus is edited in at least 1.5% of the human cells.

93. The method of any one of claims 72-80, wherein the target gene is human CTLA4 gene, and wherein the spacer sequence comprises a nucleotide sequence selected from the group consisting of SEQ ID NOs: 55, 67-70, and 149-155.

94. The method of any one of claims 75-80 and 93, wherein the genomic sequence at the CTLA4 gene locus is edited in at least 1.5% of the human cells.

95. The method of any one of claims 72-80, wherein the target gene is human DCK gene, and wherein the spacer sequence comprises a nucleotide sequence selected from the group consisting of SEQ ID NOs: 56, 71-74, and 156-159.

96. The method of any one of claims 75-80 and 95, wherein the genomic sequence at the DCK gene locus is edited in at least 1.5% of the human cells.

97. The method of any one of claims 72-80, wherein the target gene is human FAS gene, and wherein the spacer sequence comprises a nucleotide sequence selected from the group consisting of SEQ ID NOs: 57, 75-79, and 160-173.

98. The method of any one of claims 75-80 and 97, wherein the genomic sequence at the FAS gene locus is edited in at least 1.5% of the human cells.

99. The method of any one of claims 72-80, wherein the target gene is human HAVCR2 gene, and wherein the spacer sequence comprises a nucleotide sequence selected from the group consisting of SEQ ID NOs: 58, 80-86, and 174-187.

100. The method of any one of claims 75-80 and 99, wherein the genomic sequence at the HAVCR2 gene locus is edited in at least 1.5% of the human cells.

101. The method of any one of claims 72-80, wherein the target gene is human IL7R gene, and wherein the spacer sequence comprises a nucleotide sequence selected from the group consisting of SEQ ID NOs: 748-749 and 753-754.

102. The method of any one of claims 75-80 and 101, wherein the genomic sequence at the IL7R gene locus is edited in at least 1.5% of the human cells.

103. The method of any one of claims 72-80, wherein the target gene is human LAG3 gene, and wherein the spacer sequence comprises a nucleotide sequence selected from the group consisting of SEQ ID NOs: 59, 87, 88, and 188-198.

104. The method of any one of claims 75-80 and 103, wherein the genomic sequence at the LAG3 gene locus is edited in at least 1.5% of the human cells.

105. The method of any one of claims 72-80, wherein the target gene is human LCK gene, and wherein the spacer sequence comprises the nucleotide sequence of SEQ ID NO: 757.

106. The method of any one of claims 75-80 and 105, wherein the genomic sequence at the LCK gene locus is edited in at least 1.5% of the human cells.

107. The method of any one of claims 72-80, wherein the target gene is human PDCD1 gene, and wherein the spacer sequence comprises a nucleotide sequence selected from the group consisting of SEQ ID NOs: 60, 89-92, and 199-201.

108. The method of any one of claims 75-80 and 107, wherein the genomic sequence at the PDCD1 gene locus is edited in at least 1.5% of the human cells.

109. The method of any one of claims 69-77, wherein the target gene is human PLCG1 gene, and wherein the spacer sequence comprises a sequence of SEQ ID NO: 759 and 761-762.

110. The method of any one of claims 75-80 and 109, wherein the genomic sequence at the PLCG1 gene locus is edited in at least 1.5% of the human cells.

111. The method of any one of claims 72-80, wherein the target gene is human PTPN6 gene, and wherein the spacer sequence comprises a nucleotide sequence selected from the group consisting of SEQ ID NOs: 61, 93-104, and 202-213.

112. The method of any one of claims 75-80 and 111, wherein the genomic sequence at the PTPN6 gene locus is edited in at least 1.5% of the human cells.

113. The method of any one of claims 72-80, wherein the target gene is human TIGIT gene, and wherein the spacer sequence comprises a nucleotide sequence selected from the group consisting of SEQ ID NOs: 62, 105, and 214-217.

114. The method of any one of claims 75-80 and 113, wherein the genomic sequence at the TIGIT gene locus is edited in at least 1.5% of the human cells.

115. The method of any one of claims 72-80, wherein the target gene is human TRBC2 gene, and wherein the spacer sequence comprises a nucleotide sequence selected from the group consisting of SEQ ID NOs: 705-706, 711-712, 714-715, 717, and 719-720.

116. The method of any one of claims 75-80 and 115, wherein the genomic sequence at the TRBC2 gene locus is edited in at least 1.5% of the human cells.

117. The method of claim 115 or 116, wherein the method further results in editing of the genomic sequence at human TRBC1 gene locus in the human cell, and wherein the spacer sequence comprises a nucleotide sequence selected from the group consisting of SEQ ID NOs: 705-706.

118. The method of claim 117, wherein the genomic sequence at the TRBC1 gene locus is edited in at least 1.5% of the human cells.

119. The method of any one of claims 75-118, wherein genomic mutations are detected in no more than 2% of the cells at any off-target loci by CIRCLE-Seq.

120. The method of any one of claims 75-119, wherein genomic mutations are detected in no more than 1% of the cells at any off-target loci by CIRCLE-Seq.
